(19) United States
(12) Patent Application Publication
(10) Pub. No.: US 2024/0110246 A1
(43) Pub. Date: Apr. 4, 2024

(54) TARGETING CHROMOSOMAL INSTABILITY AND DOWNSTREAM CYTOSOLIC DNA SIGNALING FOR CANCER TREATMENT

(71) Applicants: Cornell University, Ithaca, NY (US); Memorial Sloan-Kettering Cancer Center, New York, NY (US)

(72) Inventors: Lewis C. Cantley, New York, NY (US); Bryan Ngo, New York, NY (US); Samuel F. Bakhoum, New York, NY (US)

(21) Appl. No.: 18/299,558

(22) Filed: Apr. 12, 2023

Related U.S. Application Data
(63) Continuation of application No. 16/629,512, filed on Jan. 8, 2020, now Pat. No. 11,821,042.
(60) Provisional application No. 62/530,661, filed on Jul. 10, 2017.

(51) Int. Cl. C12Q 1/6886; A61K 31/04; A61K 48/00; C12N 15/113; G01N 33/50

(52) U.S. Cl. CPC C12Q 1/6886; A61K 31/04; A61K 48/0066; C12N 15/113; G01N 33/5011; C12Q 2600/118; C12Q 2600/136; C12Q 2600/158; G01N 33/5005; G01N 2400/00

(57) ABSTRACT

As described herein, chromosomal missegregations, chromosomal micromodel, cytosolic DNA, and combinations thereof are indicative of metastatic cancer. Methods and compositions are described herein that are useful for detection and treatment of patients with chromosomal instabilities such as chromosomal missegregations, chromosomal micromilei, cytosilic DNA, and combinations thereof. For example, some of the methods and compositions include use of kinesin-13 proteins such as Kif2b, MCAK/Kif2 or KIF13A. The methods and compositions can also include is of STING, ENPPI, cGAS, NF-kB transcription factor p52, NF-kB transcription factor ReIB, or any combination thereof. Methods are also described for identifying compounds that are effective for treatment of cancer, including metastic cancer.

### PRIORITY CLAIM TO RELATED APPLICATIONS

This application is a continuation of U.S. application Ser. No. 16/629,512, filed Jan. 8, 2020, which is a U.S. national stage filing under 35 U.S.C. § 371 from International Application No. PCT/US2018/041480, filed on 10 Jul. 2018, and published as WO2019/014246 on 17 Jan. 2019, which claims the benefit of priority to the filing date of U.S. Provisional Application Ser. No. 62/530,661, filed Jul. 10, 2017, the contents of which are specifically incorporated by reference herein in their entirety.

### FEDERAL FUNDING

This invention was made with government support under grant number CA197588 awarded by the National Institutes of Health and grant number W81XWH-16-1-0315 awarded by the ARMY/MRMC. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Dec. 14, 2023, is named “2353710.xml” and is 117,482 bytes in size.

### BACKGROUND

Cancer is an uncontrolled growth of abnormal cells in various parts of the body. Presently cancer may be treated by surgery, radiotherapy, chemotherapy, immunotherapy, etc., with varying degrees of success. However, surgical therapy cannot completely remove extensively metastasized tumor cells. Radiotherapy and chemotherapy do not have sufficient selectivity to kill cancer cells in the presence of rapidly proliferating normal cells. Immunotherapy is largely limited to the use of cytokines or therapeutic cancer vaccines. Cytokines may cause serious toxicity and continuous use of vaccines may lead to immune tolerance.

### SUMMARY

Previously, one of the major concerns regarding cytosolic DNA was that it induces immune responses. However, as described herein, chromosomal instability can generate cytosolic DNA, which increases the incidence and potential for metastasis of cancer cells. As further illustrated herein, chromosomal instabilities such as chromosomal missegregation, and micronuclei can also increase the incidence and potential for metastasis of cancer cells.

Methods compositions described are useful for treatment of patients with increased levels of chromosomal instability, increased levels of cytosolic DNA, chromosomal missegregation, or a combination thereof. The compositions and methods can also reduce and/or inhibit metastasis, cancer drug resistance, or combinations thereof. In some cases, the compositions and methods are useful for modulating kinesin-13 expression, and the compositions and methods can reduce chromosomal instability.

For example, methods and compositions are described herein that can increase the expression and/or activity of kinesin-13 proteins such as Kif2b, MCAK/Kif2c, or KIF13A in cells. In some cases, the methods and compositions can increase the expression and/or activity of ABCC4, ABCG2. The methods can also include inhibiting STING, cGAS, NF-κB transcription factor p52, NF-κB transcription factor Re1B, ENPP1, LTβR, BAFFR, CD40, RANK, FN14, NIK (MAP3K14), MST1, or any combination thereof in a mammalian cell. Such compositions and methods are useful for treating and inhibiting the progression of cancer, including the development and progression of metastatic cancer.

Other methods are described herein that include assays for the design and development of new compounds that are useful for treatment of cancer, including metastatic cancer.

### BRIEF DESCRIPTION OF THE FIGURES

FIG. 1A-1M illustrates that chromosomal aberrations are prevalent in human metastases. FIG. 1A graphically illustrates the Weighted Genomic Instability Index (wGII) of matched primary tumors (P) and brain metastases (M), where n=61 primary tumors-metastasis matched pairs, boxes span the 25^{th}-75^{th} percentiles, bars span 10^{th}-90^{th} percentile, and significance was tested using Wilcoxon matched-pairs signed rank test. RCC, renal cell carcinoma. FIG. 1B-1 graphically illustrates differences in wGII between metastases and matched primary breast tumors. FIG. 1B-2 graphically illustrates differences in wGII between metastases and matched primary lung tumors. FIG. 1B-3 graphically illustrates differences in wGII between metastases and matched renal cell carcinoma primary tumors. FIG. 1B-4 graphically illustrates differences in wGII between metastases and matched primary tumors. FIG. 1C graphically illustrates the number of clones (based on karyotypes) in primary (P) breast tumors (n=637) or metastases (M, n=131) found in the Mitelman Database. FIG. 1D graphically illustrates the Log2 of the number of chromosomes per clone found in primary breast tumors (n=983 clones) or metastases (n=186 clones). FIG. 1E graphically illustrates the number of chromosomal aberrations per clone found in primary breast tumors (n=983 clones) or metastases (n=186 clones). In FIGS. 1C-1E the boxes span the 25^{th}-75^{th} percentiles, bars span 10^{th}-90^{th} percentile, significance tested using two-tailed Mann Whitney test. FIG. 1F shows images of formalin-fixed paraffin-embedded head and neck squamous cell carcinoma cells undergoing anaphase. Arrows point examples of chromosome missegregation, scale bar 5-μm. FIG. 1G graphically illustrates the percentage of anaphase cells exhibiting evidence of chromosome missegregation in tumors from patients with (N+, n=22 patients) or without (N−, n=18 patients) clinically detectable lymph node metastases. Boxes span the 25^{th}-75^{th} percentiles, bars span 10^{th}-90^{th} percentile, significance tested using two-tailed Mann Whitney test.

FIG. 1H graphically illustrates the weighted genomic instability index (wGII) of brain metastases as a function of the wGII of the matched primary tumor. The red line represents linear regression. FIG. 1I graphically illustrates the number of chromosome aberrations per clone as a function of the total number of chromosomes in a given clone in samples derived from primary and metastatic breast cancer and depicted in FIGS. 1D-1E, data points represent average ±SD. FIG. 1J graphically illustrates the percentage of N− or N+ patients as a function of chromosome missegregation frequency (n=20 patients for CIN-low and CIN-high), significance tested using Fisher Exact test. FIG. 1K graphically illustrates cell confluence as a function of time of MDA-MB-231 cells that express various kinesin-13 proteins. The data points represent average ±SD, n=4 experiments. FIG. 1L shows immunoblots of cells expressing various GFP-tagged kinesin-13 proteins stained using anti-GFP antibody, β-actin used as a loading control. FIG. 1M shows cells expressing MCAK and dnMCAK stained for microtubules (DM1A), centrosomes (pericentrin) and DNA (DAPI), scale bar 5-μm.

FIG. 2A-2J illustrate that chromosomal instability (CIN) is a driver of metastasis. FIG. 2A illustrates anaphase cells stained for anti-centromere protein (ACA) and DNA (DAPI), scale bar, 5-μm. FIG. 2B-1 graphically illustrates the percentage of MDA-MB-231 anaphase cells exhibiting evidence of chromosome missegregation in control cells or cells expressing kinesin-13 proteins, bars represent mean±SD, n=150 cells, 3 experiments, significance tested using two-tailed t-test. FIG. 2B-2 graphically illustrates the percentage of anaphase H2030 cells exhibiting evidence of chromosome missegregation in control cells or cells expressing kinesin-13 proteins, bars represent mean±SD, n=150 cells, 3 experiments, significance tested using two-tailed t-test. FIG. 2C graphically illustrates photon flux (p/s) of whole animals imaged 5 weeks after intracardiac injection with MDA-MB-231 cells expressing different kinesin-13 proteins. Significance tested using two-sided Mann Whitney test, n=7-14 mice per group, 4 independent experiments. FIG. 2D illustrates images of photon flux (p/s) of whole animals imaged 5 weeks after intracardiac injection with MDA-MB-231 cells expressing different kinesin-13 proteins. FIG. 2E graphically illustrates the disease-specific survival of mice injected with MDA-MB-231 cells with various levels of chromosomal instability: CIN-high (dnMCAK; left-most graph showing least survival over time), CIN-medium (control, Kif2a, or tubulin; middle graph showing middle levels of survival over time), or CIN-low (MCAK or Kif2b; right-most graph showing most survival over time), n=10 mice for CIN-high, 23 mice for CIN-medium, and 20 mice for CIN-low, pairwise significance tested with log-rank test. FIG. 2F-1 shows representative karyotypes (DAP1607 banding) from parental MDA-MB-231 cell #2 that were allowed to divide for 30 days. FIG. 2F-2 shows representative karyotypes (DAP1607 banding) from parental MDA-MB-231 cell #4 that were allowed to divide for 30 days. FIG. 2G shows representative karyotypes (DAP1607 banding) of a cell derived from a single MCAK expressing cell that was allowed to divide for 30 days. FIG. 2H shows representative karyotypes (DAP1607 banding) of a cell derived from a single Kif2a expressing cell that was allowed to divide for 30 days. FIG. 2I graphically illustrates the number of non-clonal (present in <25% of the cells in a single clone) neochromosomes in CIN-low (MCAK; left bar for each chromosome) or CIN-medium/high (control, Kif2a, dnMCAK; right bar for each chromosome) MDA-MB-231 cells. ‘Mar’ denotes structurally abnormal chromosomes that cannot be unambiguously identified by conventional banding, bars represent mean±SD,n=140 cells from 7 clonal populations, significance tested using two-way ANOVA test. FIG. 2J shows examples of chromosomes taken from 6 distinct cells belonging to the same clonal population—derived from a single Kif2a-expressing cell—showing convergent translocations involving chromosome 22 with other distinct chromosomes.

FIG. 3A-3M illustrates opposing roles for chromosomal instability (CIN) in primary tumors and metastases. FIG. 3A is a schematic illustrating the method of collection for samples shown in FIGS. 3B-3E, where in the original the colors of the cells in the schematic matches the color of the bars in FIGS. 3B-3E. FIG. 3B-1 graphically illustrates the percentage of anaphase cells arising from metastasis-competent patient-derived xenografts (PDX) belonging to the ER breast cancer subtype, to illustrate evidence of chromosome missegregation in first-passage cells derived from primary tumors, and from liver metastases. FIG. 3B-2 graphically illustrates the percentage of anaphase cells arising from metastasis-competent patient-derived xenografts (PDX) belonging to the TNBC breast cancer subtype, to illustrate evidence of chromosome missegregation in first-passage cells derived from primary tumors, and from liver metastases. FIG. 3C graphically illustrates the percentage of anaphase cells arising from CIN-low cells, to illustrate evidence of chromosome missegregation in injected cells, first-passage cells derived from primary tumors, spontaneous metastases arising from primary tumors in the same animal, and metastases obtained from direct intracardiac implantation. FIG. 3D graphically illustrates the percentage of anaphase cells arising from CIN-medium (Kif2a) cells, to illustrate evidence of chromosome missegregation in injected cells, first-passage cells derived from primary tumors, spontaneous metastases arising from primary tumors in the same animal, and metastases obtained from direct intracardiac implantation.

FIG. 3E graphically illustrates the percentage of anaphase cells arising from CIN-high (dnMCAK) cells, to illustrate evidence of chromosome missegregation in injected cells, first-passage cells derived from primary tumors, spontaneous metastases arising from primary tumors in the same animal, and metastases obtained from direct intracardiac implantation. For FIGS. 2B-2E the bars represent mean±SD, n=150 cells, 3 independent experiments, * p<0.05 and denotes samples with higher missegregation rates than the injected lines, #p<0.05 and denotes samples with lower missegregation rates than the injected lines, ** p<0.05 and it denotes significant differences between metastases and matched primary tumors from the same animals, two-tailed t-test. ST met, soft tissue metastasis. FIG. 3F shows a Volcano plot illustrating changes in differentially expressed genes between CIN-low (MCAK and Kif2b) and CIN-medium/high (control, Kif2a, and dnMCAK) MDA-MB-231 cells. Data points in the right upper area (Log2 of greater than 2.6) correspond to genes subsequently used for determining the chromosomal instability (CIN) signature. FIG. 3G is an enrichment plot for TAVAZOIE_METASTASIS gene set. FIG. 3H shows a distant metastasis-free survival (DMFS) plot of patients with high (CIN-High; lower graph line) or low (CIN-Low; upper graph line) expression of the CIN signature genes in a meta-analysis of patients. FIG. 3I shows a distant metastasis-free survival (DMFS) plot of patients with high (CIN-High; lower graph line) or low (CIN-Low; upper graph line) expression of the CIN signature genes in a validation cohort of 171 patients. As noted in Example 1, the CIN signature genes include PELI2, BMP2, SHH, TNS4, RAB3B, ROBO1, ARHGAP28, CHN2, CST1, F13A1, CPVL, SEMA6D, NHSL2, GTF21P7, DPYSL3, PCDH7, KHDRBS3, TRAC, TMEM156, CST4, CD24, FGF5, NTN4. FIGS. 3J-3M illustrate that chromosomal instability promotes formation and maintenance of metastasis. FIG. 3J-1 graphically illustrates a normalized photon flux plot over time of whole animals injected with MDA-MB-231 cells expressing kinesin-13 proteins Bars represent mean±s.e.m. n=7-14 mice per group. FIG. 3J-2 shows images of a mouse injected with MDA-MB-231 cells expressing dnMCAK where disease burden was tracked using bioluminescence. FIG. 3J-3 shows images of a mouse injected with MDA-MB-231 cells expressing Kif2b where disease burden was tracked using BLI. FIG. 3K illustrates photon flux (p/s) of whole animals imaged 5 weeks after intracardiac injection with control or MCAK expressing H2030 cells. Significance tested using two-sided Mann Whitney test, n=10 mice in the MCAK group and 5 mice in the control group. FIG. 3L shows representative BLI images of mice orthotopically transplanted with MDA-MB-231 cells before (Day 33) and after (Day 90) tumor excision. Metastasis can be detected in the mouse transplanted with dnMCAK expressing cells at day 90. FIG. 3M shows a distant metastasis-free survival (DMFS) of mice orthotopically transplanted with MDA-MB-231 cells with various levels of chromosomal instability. As illustrated the animals that received CIN-low cells all survived (top graph line), while most of the animals that received CIN-medium cells survived (middle graph line), but most animals that received CIN-high cells did not survive (bottom graph line), n=5-9 mice group, pairwise significance tested with log-rank test.

FIG. 4A-4H illustrate that chromosomal instability enriches for mesenchymal cell traits. FIG. 4A shows a gene expression heat map of 6,821 cells (columns) and genes involved in epithelial-to-mesenchymal transition (EMT, rows). Black rectangle denotes a gene-cell cluster enriched for mesenchymal traits. FIG. 4B shows a t-stochastic neighbor embedding (tSNE) projection of 6,821 MCAK, Kif2b, and dnMCAK expressing cells with 12 subpopulations identified using unsupervised K-nearest neighbor graph theory. Heatmap shows normalized enrichment score (NES) for gene sets with FDRq <0.05 inferred from gene set enrichment analysis of differentially expressed genes of each subpopulation. FIG. 4C shows representative images of cells expressing MCAK or dnMCAK stained for β-actin, Vimentin, and DNA scale bar 50-μm. FIG. 4D shows representative images of cells which invaded through a collagen membrane within 18 hours of culture. FIG. 4E graphically illustrates the numbers of cells which invaded through a collagen membrane within 18 hours of culture (see FIG. 4D). Bars represent mean±s.e.m., * p<0.05. ** p<0.01, two-sided Mann Whitney test, n=10 high-power fields, 2 independent experiments. FIG. 4F shows a principle component analysis (PCA) plot of MDA-MB-231 cells expressing different kinesin-13 proteins based on bulk RNA expression data. FIG. 4G shows results of a gene set enrichment analysis (GSEA) of HALLMARK gene sets highly enriched in CIN-medium/high (control, Kif2a, and dnMCAK) compared with CIN-low cells (MCAK and Kif2b). FIG. 4H shows a plot of normalized enrichment score versus False Discovery Rate (FDR).

FIG. 5A-5I illustrate cell-intrinsic inflammation from cytosolic DNA in chromosomally unstable cells. FIG. 5A shows a gene-gene correlation heat-map showing expression modules and the HALLMARKS gene sets most significantly correlated with Module 2. NES, normalized enrichment score. FIG. 5B shows a tSNE projection (above) of 6,821 MCAK, Kif2b, and dnMCAK expressing cells labeled either with their kinesin-13 expression status or expression level of key gene signatures. Single-cell correlation plots between key gene signatures are shown below. FIG. 5C-1 shows a representative image of a micronucleus near a primary nucleus in a cell stained with ACA and DAPI, scale bar 5-μm. FIG. 5C-2 graphically illustrates the percentage of micronuclei in MDA-MB-231 cells that express various kinesin-13 proteins. FIG. 5C-3 graphically illustrates the percentage of micronuclei in H2030 cells that express various kinesin-13 proteins. The boxes in FIGS. 5C-2 and 5C-3 span the median and inter-quartile range, bars span the 5^{th}-95^{th} percentile, n=638-1127 cells, 10 high-power fields, 3 independent experiments, significance tested using two-sided Mann Whitney test. FIG. 5D graphically illustrates the percentage of micronuclei in cells derived from primary tumors and metastases previously depicted in FIGS. 3C-3E. Bars represent median and inter-quartile range, n=10 primary tumors and 28 metastases, 500-1500 cells/sample, significance tested using two-sided Mann Whitney test. FIG. 5E graphically illustrates a correlation between the percentage of cells exhibiting evidence of chromosome missegregation and percentage of micronuclei in all injected cell lines as well as cells derived from primary tumors and metastases. FIG. 5F shows MCAK and dnMCAK expressing cells stained for DNA (DAPI), cytosolic double-stranded DNA (using anti-dsDNA antibody), or single-stranded DNA (using anti-ssDNA antibody), scale bar 20-μm. FIG. 5G graphically illustrates normalized cytosolic-to-nuclear DNA ratios in CIN-medium/high and CIN-low MDA-MB-231 and H2030 cells. Bars represent mean±SD, significance tested using two-sided Mann Whitney test. FIG. 5H shows cells stained for DNA (DAPI), cytosolic DNA (dsDNA), or Dnase2 (RFP reporter), scale bar 10-μm, arrows denote Dnase2 expressing cells. FIG. 5I shows cells stained for DNA (DAPI), cytosolic DNA (dsDNA), or mCherry-Lamin B2, scale bar 10-μm, arrows denote mCherry-Lamin B2 expressing cells.

FIG. 6A-6J illustrate metastasis from cellular responses to cytosolic DNA. FIG. 6A shows a cell stained using DAPI (DNA), cytosolic DNA (dsDNA), or anti-cGAS antibody, scale bar 5-μm. FIG. 6B graphically illustrates the percentages of micronuclei with (cGAS+) or without (cGAS−) cGAS localization in cells expressing kinesin-13 proteins (or Lamin B2 and dnMCAK), n=400 cells, 4 experiments, significance tested using two-sided Mann Whitney test. FIG. 6C shows immunoblots of lysates from cells expressing different kinesin-13 proteins or STING shRNA (dnMCAK), β-actin used as a loading control. FIG. 6D illustrates normalized ratios of phosphorylated p100-to-total p100 (above) and p52-to-p100 (below) protein levels from CIN-med/high cells (Control, Kif2a, and dnMCAK), CIN-low cells (Kif2b and MCAK) or STING-depleted dnMCAK expressing cells (STING shRNA). Bars represent mean±s.e.m., * p<0.05, ** p<0.01, two-tailed Mann-Whitney test, n=4 biological replicates. FIG. 6E shows MCAK, dnMCAK expressing cells, and cells expressing control or STING shRNA, stained for ReIB and DNA (DAPI), arrows point to ReIB-positive nuclei, scale bar 20-μm. FIG. 6F graphically illustrates the average z-normalized expression of CIN-responsive noncanonical NF-κB target genes in breast cancer patients with low (<30^{th} percentile) or high (>30^{th} percentile) chromosomal instability gene expression signature, boxes span interquartile range, bars span 10^{th}-90^{th} percentile, significance tested using two-sided Mann Whitney test. FIG. 6G-1 graphically illustrates the photon flux (p/s) of whole animals imaged 5 weeks after intracardiac injection with cells expressing control shRNA or STING shRNA. Significance tested using two-sided Mann Whitney test, n=9 mice in the control group and 16 mice in the STING shRNA group. FIG. 6G-2 shows whole animals imaged 5 weeks after intracardiac injection with cells expressing control shRNA or STING shRNA. FIG. 6H graphically illustrates the number of cells expressing shRNA targeting genes in the DNA sensing or noncanonical NF-κB pathways which invaded through a collagen membrane within 24 hours of culture. Bars represent mean±s.e.m., ** p<0.0001, two-sided Mann Whitney test, n=10 high-power fields, 2 experiments. FIG. 6I-6J illustrate single-cell sequencing and population detection. FIG. 6I illustrates the cellular composition of every subpopulation presented in FIG. 4B. FIG. 6J shows violin plots illustrating expression of key metastasis and invasion genes in a subpopulation of cells enriched for epithelial-to-mesenchymal transition (EMT) and chromosomal instability genes (subpopulation ‘M’) compared with the remaining subpopulations, subpopulations were identified using unsupervised K-nearest neighbor graph theory.

FIG. 7A-7F illustrate that chromosomal instability promotes a viral-like immune response that promotes metastasis yet at the same time recruits a large amount of an immune infiltrate. FIG. 7A shows that chromosomal instability promotes a viral-like immune response that promotes a large amount of an immune infiltrate. FIG. 7B is a schematic diagram illustrating that chromosomal instability (CIN) is linked to metastasis and tumor immune infiltrate through tumor-cell intrinsic inflammatory response to cytosolic DNA. FIG. 7C-1 shows representative phase contrast images of cells in the wound area, 36-hours after wound creation. FIG. 7C-2 graphically illustrates the length-to-width ratio of cells expressing different kinesin-13 proteins. For FIGS. 7C-1 and 7C-2, the bars span the interquartile range, n=100 cells, 2 experiments, ** p<0.0001, Mann Whitney test. FIG. 7D-1 shows representative cells that express MCAK (CIN-low) stained with β-catenin or DNA (DAPI), scale bar 30-μm. FIG. 7D-2 shows representative cells that express dnMCAK (CIN-high) stained with β-catenin or DNA (DAPI), scale bar 30-μm. FIG. 7E-1 shows phase-contrast images of a wound-healing assay of cells expressing kinesin-13 proteins, scale bar 800-μm. FIG. 7E-2 graphically illustrates the wound area (normalized to the 0 h time point) 24h and 45h after wound creation. * p<0.05, two-tailed t-test. FIG. 7F-1 shows images of cells which invaded through a polycarbonate membrane containing 8-μm pores within 18 hours of culture. FIG. 7F-2 graphically illustrates the normalized optical density (O.D.) of cells scraped from the bottom of the membrane, bars represent mean±s.e.m., * p<0.05, two-sided t-test, n=3 experiments.

FIG. 8A-8C illustrate that chromosomal instability generates micronuclei and cytosolic dsDNA. FIG. 8A graphically illustrate the percentage of micronuclei in CIN-low samples depicted in FIG. 3C. FIG. 8B graphically illustrate the percentage of micronuclei in CIN-low samples depicted in FIG. 3D. FIG. 8C graphically illustrate the percentage of micronuclei in CIN-low samples depicted in FIG. 3E. For FIGS. 8A-8C: injected cells, first-passage cells derived from primary tumors, or metastases (some spontaneous metastases arising from primary tumors, some metastases obtained from direct intracardiac implantation).

Bars represent mean±s.e.m., n=10 high-power fields encompassing 500-1500 cells/sample, 3 experiments, * p<0.05 and denotes samples with higher missegregation rates than the injected lines, #p<0.05 and denotes samples with lower missegregation rates than the injected lines, ** p<0.05 and it denotes significant differences between metastases and matched primary tumors from the same animals, two-tailed t-test.

FIG. 9A-9M illustrate the effects of cytosolic DNA sensing pathways on prognosis. FIG. 9A graphically illustrates disease-specific survival of mice injected with dnMCAK expressing cells co-expressing either control shRNA or STING shRNA n=9 mice in the control group and 16 mice in the STING shRNA group, significance tested with log-rank test. As shown, reducing STING expression by expression of STING shRNA increases the survival of dnMCAK expressing cells. FIG. 9B graphically illustrates distant metastasis-free survival (DMFS) over time of breast cancer patients expressing high and lower levels of regulators of noncanonical NF-κB (where NFKB2, ReIB, MAP3K14 positively regulate NF-KB, and TRAF2, TRAF3, BIRC2, BIRC3 negatively regulate NF-KB). As shown, expression of lower levels of such regulators of noncanonical NF-KB improves survival. FIG. 9C graphically illustrates distant metastasis-free survival (DMFS) over time of breast cancer patients expressing high and lower levels of CIN-responsive non-canonical NF-KB targets (where PPARG, DDIT3, NUPR1, RAB3B, IGFBP4, LRRC8C, TCP11L2, MAFK, NRG1, F2R, KRT19, CTGF, ZFC3H1 positively regulate, and MACROD1, GSTA4, SCN9A, BDNF, LACTB negatively regulate CIN-responsive non-canonical NF-κB targets). As shown, down regulation of such CIN-responsive non-canonical NF-κB targets improves survival. FIG. 9D graphically illustrates distant metastasis-free survival (DMFS) over time of breast cancer patients expressing high and lower levels of regulators of canonical NF-κB (NFKB1, ReIA, TRAF1, TRAF4, TRAF5, TRAF6). As shown, increased expression of such regulators of canonical NF-κB improves survival. FIG. 9E graphically illustrates distant metastasis-free survival (DMFS) over time of breast cancer patients expressing high and lower levels of regulators of interferon signaling (IRF1, IRF3, IRF7, TBK1). As shown, increased expression of such regulators of interferon signaling improves survival. FIG. 9F graphically illustrates relapse-free survival (RFS) over time of breast cancer patients expressing high and lower levels of regulators of noncanonical NF-κB. As shown, expression of lower levels of regulators of noncanonical NF-κB improves survival. FIG. 9G graphically illustrates relapse-free survival (RFS) over time of breast cancer patients expressing high and lower levels of CIN-responsive non-canonical NF-κB targets. As shown expression of slightly higher levels of CIN-responsive non-canonical NF-κB targets improves survival somewhat. FIG. 9H graphically illustrates relapse-free survival (RFS) over time of breast cancer patients expressing high and lower levels of regulators of canonical NF-κB. As illustrated, increased expression of regulators of canonical NF-κB improves survival. FIG. 9I graphically illustrates relapse-free survival (RFS) over time of breast cancer patients expressing high and lower levels of regulators of interferon signaling. As illustrated, increased expression of regulators of interferon signaling improves survival. FIG. 9J graphically illustrates progression-free survival (PFS) over time of lung cancer patients expressing high and lower levels of regulators of noncanonical NF-κB. As illustrated, reduced expression of regulators of noncanonical NF-κB improves survival. FIG. 9K graphically illustrates progression-free survival (PFS) over time of lung cancer patients expressing high and lower levels of CIN-responsive non-canonical NF-κB targets. As illustrated, reduced expression of CIN-responsive non-canonical NF-κB targets improves survival. FIG. 9L graphically illustrates progression-free survival (PFS) over time of lung cancer patients expressing high and lower levels of regulators of canonical NF-κB. As illustrated, increased expression of regulators of canonical NF-κB improves survival. FIG. 9M graphically illustrates progression-free survival (PFS) over time of lung cancer patients expressing high and lower levels of regulators of interferon signaling. As illustrated, increased expression of regulators of interferon signaling improves survival.

FIG. 10A-10B illustrate quantification of cGAMP FIG. 10A illustrates the cGAMP transitions that can be detected by LC-MS. FIG. 10B graphically illustrates quantification of cGAMP in chromosomally unstable urine triple-negative breast cancer cells (4T1) using targeted LC-MS metabolomics. As illustrated, knockdown of cGAS in 4T1 cells reduces the abundance of cGAMP.

### DETAILED DESCRIPTION

As illustrated herein, human metastases are significantly more chromosomally unstable compared with their primary tumor counterparts. More specifically, ongoing chromosome segregation errors, as well as the presence of micronuclei or cytosolic DNA, are predictive of metastasis as increasing chromosome segregation errors enriches for metastasis-initiating tumor cell subpopulations. Conversely, reduction in chromosomal instability leads to durable suppression of metastatic outbreaks even in highly aneuploid—yet stable—cells. The methods and compositions described herein are useful for detecting, monitoring, and treating such chromosomal instabilities and metastatic cancers.

### Detection and Monitoring of Cancer

As illustrated herein, chromosomal instability is a marker indicating that a subject has cancer and chromosomal instability is especially useful for predicting, detecting and monitoring metastatic cancer. A large percentage (60-80%) of human solid tumors contain chromosomal instability. Hence, methods for diagnosing cancer, especially metastatic cancer, are described herein. Such methods are surprisingly effective at predicting, detecting, monitoring and treating cancer, including metastatic cancer. The methods of treatment described herein can be paired with the methods for predicting, detecting and monitoring metastatic cancer.

For example, one method for predicting, detecting and monitoring cancer (including metastatic cancer) can include obtaining a sample from a subject; and detecting and/or quantifying whether cells within the sample exhibit chromosomal instability. The methods can also include treating the subject when chromosomal instability is detected in the subject's sample.

For example, one method includes initiating treatment or modifying treatment of a subject having cells or tissues that have detectable levels of chromosomal instability, where the treatment includes administration of an agent that can reduce the incidence or progression of metastatic cancer.

As used herein, “obtaining a test sample” involves removing a sample of tissue or fluid from a patient, receiving a sample of tissue or fluid from a patient, receiving a patient's tissue or fluid sample from a physician, receiving a patient's tissue or fluid sample via mail delivery and/or removing a patient's tissue or fluid sample from a storage apparatus (e.g., a refrigerator or freezer) or a facility. Thus, obtaining a test sample can involve removal or receipt of the test sample directly from the patient, but obtaining a test sample can also include receipt of a test sample indirectly from a medical worker, from a storage apparatus/facility, from a mail delivery service after transportation from a medical facility, and any combination thereof. The test sample can therefore originate in one location, and be transported to another location where it is received and tested. Any of these activities or combinations of activities involves “obtaining a test sample.” The test sample can be body fluid or a tissue sample. For example, the test sample can be a cell sample that is suspected of containing cancer cells. The sample can include cells and/or tissues from one or more primary tumors, tumor cells derived from primary tumors, tumor cells purified from the circulation, metastatic cell samples, or cells derived from metastatic tumors. Samples can include cells from established metastases, for example because increased chromosomal instability is a marker for a more aggressive disease. For example, the sample can be a tissue biopsy of breast or lung tissues (or of any of the tissue types mentioned herein). In another example, when detecting some cancer markers (e.g. cGAMP levels) to predict, detect, or monitor cancer (especially metastatic cancer), the sample can be a bodily fluid such as blood, serum, plasma, urine, ascites fluid, lymph fluid, or a combination thereof.

As used herein detecting and/or quantifying whether cells within the sample exhibit chromosomal instability can include detecting and/or quantifying micronuclei, chromosomal missegregation, or cytosolic chromosomal DNA in cells of sample. Detecting and/or quantifying micronuclei, chromosomal missegregation, cytosolic DNA, or a combination thereof can be done, for example, by examining cell chromosomes through a microscope, and counting the number(s) of micronuclei, chromosomal missegregations, cytosolic DNA, or a combination thereof.

In some cases, the cell samples can be fixed and/or lysed. Anaphase cells can be selected for analysis. Chromosomes can in some cases be treated with a protease (e.g., trypsin), for example, to improve visualization. In some cases, the chromosomes can be stained with a dye or a labeled antibody that facilitates visualization of chromosomes or DNA. Examples of dyes that can be used include Hematoxylin and Eosin (H&E) stain, 4′,6-diamidino-2-phenylindole (DAPI) stain, quinacrine stain, Giemsa stain, and other chromosomal or DNA stains.

Cancer, especially metastatic cancer, can be predicted, detected, or undergoing progression, for example, when at least 10%, or at least 11%, or at least 12%, or at least 13%, or at least 14%, or at least 15% of chromosomes exhibit missegregations. In some cases, cancer, especially metastatic cancer, can be predicted, detected, or undergoing progression when about 15-20% of chromosomes exhibit missegregations.

Micronuclei can be easier to identify than chromosomal missegregations. Cancer, especially metastatic cancer, can be predicted, detected, or can be undergoing progression, for example, when at least 3%, at least 4% or at least 5% of cells exhibit micronuclei. In some cases, cancer, especially metastatic cancer, can be predicted, detected, or undergoing progression when about 5% to 8% of cells exhibit micronuclei.

In some cases, any amount of cytosolic DNA is indicative of cancer. Cytosolic DNA can be detected by DNA (staining) in the cytosol (rather than in nuclei). To detect cytosolic DNA any convenient DNA stain can be used. For example, a stain for double-stranded DNA can be used for detecting and quantifying cytosolic DNA. Cancer, especially metastatic cancer, can be predicted, can be detected, or can be undergoing progression, for example, when a 1-fold to 2-fold increase in staining intensity within the cytosol is observed compared to a normal non-cancer tissue. The normal, non-cancerous tissue used for comparison can be from the same patient or it can be a reference tissue derived from normal tissue samples.

An assay for detecting and quantifying cyclic guanosine monophosphate-adenosine monophosphate (cGAMP) is described herein and can be used to identify patients with cancer, including metastatic cancer. For example, total cGAMP concentration in a sample can be used as a marker for metastasis, by comparing the cGAMP levels in the sample compared to a reference normal tissue or adjacent normal tissue taken from the same patient. Increases in cGAMP of 10%, or 20%, or 30%, or 50%, or 70%, or 80%, or 90% can identify a patient who has or will develop cancer, including metastatic cancer. In some cases, increases in cGAMP at 1-fold to 2-fold over normal can identify a patient who has or will develop cancer, including metastatic cancer. Increased cGAMP concentrations in pre-therapy and shortly post therapy samples is a marker for tumor response. An increase of an additional 1-fold to 2-fold change in cGAMP levels is an indication of tumor response.

A method is described herein for diagnosing metastatic disease in patients using cGAMP as a novel metabolite biomarker for CIN driven cancers and metastatic disease. Measurements of cGAMP can serve as a clinical modality to accurately and specifically identify patients with metastatic disease. Measurement of cGAMP in patient samples (tumor, non-cancerous tissues, blood, serum, urine, and plasma), and the relative presence or absence of cGAMP therein, may also provide information that clinicians can correlate with a probable diagnosis of cancer aggressiveness or metastatic disease, as well as a negative diagnosis (e.g., normal or lack of disease).

In addition, a method is described herein for monitoring patient response to treatment based on determining the levels of cGAMP over time and establishing a cGAMP profile. Such a method can include generating a cGAMP profile in a subject, comprising of obtaining a sample from the subject; using liquid chromatography and/or mass spectroscopy to measure the level of cGAMP; and based on the comparison, generating a prolife that indicates whether the subject has metastatic disease. The reference profile can be obtained from a population of healthy control subjects without metastatic disease, population of subjects having localized cancerous disease, and a population of subjects having metastatic disease.

The cGAMP concentrations or amounts measured in a sample can be compared to normal reference values from a normal tissue (not necessarily from the same patient) or, if available to cGAMP levels in adjacent normal tissues. For example, in the case of a patient with mastectomy after the diagnosis or breast cancer, measurement of cGAMP levels in a sample of the normal breast (not involved with cancer) can be used as a reference or control value. Alternatively, for patients in which normal tissue is unavailable, a reference banked normal tissue from non-cancerous breasts for example can be used as a reference or control.

Once a profile is established, cGAMP levels can be used as a point of reference to compare and characterize unknown samples and samples for which further information is sought. For example, a decreased level of cGAMP (at least 10% or more, or a decrease of greater than 1-fold, 2-fold or more relative to a baseline) relative to a control (e.g., a sample taken from a subject at an earlier point in time or mean cGAMP levels determined from a population profile mentioned above) may indicate a positive treatment outcome. However, an increased level of cGAMP (at least 10% or more, or an increase greater than 1-fold,) can indicate the presence or likelihood of metastatic disease and poor treatment outcome.

The determination of metastatic disease is based on the measured level of cGAMP as compared to a reference control level or a personalized longitudinal time points. The control level is indicative of the level of the one in a control subject who does not have metastatic disease, or before and after treatment.

In both aforementioned embodiments, measuring the level of cGAMP as a biomarker can include using liquid chromatography-mass spectrometry (LC-MS).

In brief, samples are collected from urine, blood, plasma, serum and cerebrospinal fluid. In certain embodiments, the sample also comprises of tumor cells or normal tissue cells adjacent to a tumor. Once collected, the sample is processed as described herein. Non-limiting, exemplary processing steps for use in embodiments of the invention include extraction of organic acids, column purification (e.g., anion exchange purification), chromatography (e.g., size-exclusion chromatography), centrifugation, and alcohol treatment (e.g. methanol or ethanol).

For example, cells from a cell sample can be washed and then frozen on liquid nitrogen to preserve metabolic state of the cells. Cells can then be collected/scraped into cold methanol (−80° C.). Methanolic metabolite extracts can then purified by Solid Phase Extraction (SPE) using HyperSep aminopropyl solid phase columns as described by Collins et al. (Cell Host & Microbe 17(6): 820-828 (2015)). Effluents can be dried and reconstituted in 70% acetonitrile in ddH₂O. The reconstituted effluents can be analyzed by LC-MS/MS analysis.

In some cases, serum or media can be evaluated for cGAMP concentrations or amounts. To detect/quantify secreted cGAMP in culture media, aliquots of conditioned media can be collected, mixed 80:20 with methanol, and centrifuged at 3,000 rpm for 20 minutes at 4 degrees Celsius. The resulting supernatant can be collected and stored at −80 degrees Celsius prior to LC-MS/MS to assess cGAMP levels.

To measure whole-cell associated metabolites, media can be aspirated and cells can be harvested, e.g., at a non-confluent density.

A variety of different liquid chromatography (LC) separation methods can be used.

Each method can be coupled by negative electrospray ionization (ESI, −3.0 kV) to triple-quadrupole mass spectrometers operating in multiple reaction monitoring (MRM) mode, with MS parameters optimized on infused metabolite standard solutions.

Methods are also described herein that identify ongoing breast cancer metastasis and/or patients who will undergo or survive breast cancer metastasis. Decreased expression of one or more of the following genes in a test sample can identify ongoing breast cancer metastasis and/or patients who will undergo breast cancer metastasis: PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, FGF5 or NTN.

As described herein, elevated expression of these genes PREDICTS increased distant-metastasis free survival in breast cancer. Elevated expression of the following genes is referred to as the chromosomal instability (CIN) signature: PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, FGF5, and NTN4. Hence, methods are also described herein that identify patients who can have metastasis free survival where the method involves quantifying expression of one or more of PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, or FGF5 gene in a patient sample to obtain a measured quantified expression level for one or more of these genes of the patient. In some cases, this method can involve measuring expression levels of these genes but no other genes.

Microarray gene expression datasets deposited in the KM-Plotter database (see website at www.kmplot.com) were evaluated as described herein. The following microarray probes were used for each gene (please note that some genes have multiple names and alternate names could be listed below): 219132_at (PELI2), 205289_at (BMP2), 207586_at (SHH), 230398_at (TNS4), 227123_at (RAB3B), 213194_at (ROBO1), 227911_at (ARHGAP28), 213385_at (CHN2), 206224_at (CST1), 203305_at (F13A1), 208146_s_at (CPVL), 226492_at (SEMA6D), 201431_s_at (DPYSL3), 228640_at (PCDH7), 209781_s_at (etoile), 210972_x_at (TRA@), 220169_at (TMEM156), 206994_at (CST4), 266_s_at (CD24), 210311_at (FGF5), 200948_at (MLF2). A cutoff value of 36 percentile was used such that the patients with cumulative expression of the genes above that which were in the bottom 36-percentile had higher metastasis-free survival.

In the second data set, publicly deposited gene expression data derived from next-gen sequencing was used and the median expression values were used as a cutoff value to identify patients with improved survival. Those having expression values greater than the median expression values of PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, an FGF5 had improved survival. Thus, expression levels of each of these genes can be quantified in a patient sample and these quantified expression level can be compared to median reference expression levels for each of these genes. Such median reference expression levels for each of these genes can be the median expression of each of these genes in samples from a series of patients with metastatic cancer.

The sample tested can be from a patient with breast cancer, for example, a patient without detectable metastatic breast cancer, or one without significant metastatic breast cancer. Similarly, the median reference expression levels can be obtained from a series of samples from patients with ongoing metastatic breast cancer.

In this type of analysis, it is typical to use cutoff values ranging from the 25-percentile to the 75-percentile depending on the patient population and assay used.

Similar results obtained using the first and second methods.

Hence, a method is described herein to identify patients with improved survival. The method can include collecting samples from patients with a primary cancer type (e.g., primary breast cancer); RNA purification and preparation according to standard protocols for NextGen sequencing (see, e.g., website at qiagen.com/us/shop/sample-technologies/ma/total-rna/measy-mini-kit/#orderinginformation); determining the relative or absolute RNA expression levels using RT-PCR, NextGen sequencing or microarray method; summing up the expression values of the 23 genes; determining in this cohort the best cutoff to predict distant metastasis-free survival (DMFS); using this as an absolute cutoff for subsequent patients. Note in some cases a normal tissue reference control can be used for optimal calibration (e.g. breast tissue for breast cancer, normal pancreas for pancreatic cancer etc.).

The measured quantified expression level(s) so obtained can be compared to a control, for example, a median or mean expression level of one or more corresponding PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, or FGF5 gene in a set of patients with ongoing breast cancer metastasis. A patient can have metastasis free survival when the measured quantified expression level(s) are greater than the control level. For example, such a patient with increased metastasis free survival when the measured quantified expression level(s) are greater than the control level, can survive for at least 5 months, at least 10 months, at least 12 months, at least 15 months, at least 20 months, at least 25 months, at least 50 months, or at least 100 months more than a control set of patients with ongoing breast cancer metastasis.

In some cases, the decreased or increased expression can be of two or more, or three or more, or four or more, or five or more, or six or more, or seven or more, or eight or more, or nine or more, ten or more, or eleven or more, or twelve or more, or thirteen or more, or fourteen or more, or fifteen or more, or sixteen or more, or seventeen or more, or eighteen or more, or nineteen or more, or twenty or more, or twenty-one or more, or twenty-two or more of these genes. As used herein, decreased or increased expression of these genes can be at least a 10%, or 20% or 30%, or 40%, or 50%, or 60%, or 75%, or 100% decrease or increase in expression of the foregoing genes compared to a control. Such a decrease or increase of expression of these genes can also be at least a 1.2-fold, or 1.5-fold, or 2-fold, or 3-fold, or 5-fold, or 7-fold, or 10-fold increase compared to a control. Such a control can be healthy or non-cancerous tissue sample. In other cases, the control can be a cancerous or metastatic tissue.

### Treatment Methods

Surprisingly, the pro-metastatic phenotype imparted by chromosomal instability is driven by a tumor cell-intrinsic inflammatory response to cytosolic double-stranded DNA (dsDNA). Sensing of cytosolic DNA by cyclic GMP-AMP synthase (cGAS), and its downstream effector STING, activates the noncanonical NF-κB pathway and drives invasion and metastasis in a tumor cell-autonomous manner. This unexpected link between chromosomal instability and innate cellular inflammation offers new avenues for therapeutic intervention in genomically unstable tumors. Hence, the treatment methods described herein can include methods for identifying whether cells in a patent sample exhibit increased levels of cytosolic DNA, micronuclei, chromosomal missegregation, or a combination thereof. As described herein, increased levels of cGAMP are also indicative of cancer, especially metastatic cancer. Patients with increased levels of cytosolic DNA, micronuclei, chromosomal missegregation, or a combination thereof can then be treated as described herein or by a variety of other treatment methods.

For example, one method can include administering a metastatic chemotherapeutic agent to a patient with a cell sample or bodily fluid sample:
a. having at least 10%, or at least 11%, or at least 12%, or at least 13%, or at least 14%, or at least 15% detectable chromosomal missegregations within one or cells of the cell sample;
b. having at least 3%, at least 4% or at least 5% of cells detectable micronuclei within one or cells of the cell sample;
c. having detectable cytosolic double-stranded DNA within one or cells of the cell sample; or
d. having at least 10%, or 20%, or 30%, or 50%, or 70%, or 80%, or 90% greater concentration or amount of cGAMP in the cell sample or bodily fluid sample;
to thereby treat metastatic cancer in the patient.

A variety of chemotherapeutic agents can be employed. Methods described herein can, for example, include administering kinesin-13 proteins such as Kif2b, MCAK/Kif2c, and/or KIF13A and, optionally, administering ABCC4 and/or ABCG2 proteins. Methods described herein can include expression of kinesin-13 proteins such as Kif2b, MCAK/Kif2c, and/or KIF13A in a transgene or vector, and, optionally, expression of ABCC4 and/or ABCG2 in a transgene or vector. The methods can also include inhibiting STING, cGAS, NF-κB transcription factor p52, NF-κB transcription factor ReIB, ENPP1, LTβR, BAFFR, CD40, RANK, FN14, NIK (MAP3K14), MST1, or any combination thereof.

For example, methods and compositions are described herein that involve increased expression and/or activity of kinesin-13 proteins such as Kif2b, MCAK/Kif2c, or KIF13A in cells. Such methods and compositions are useful for treating cancer. The methods and compositions can include increased expression and/or activity of ABCC4, ABCG2, or a combination thereof. Agonists of such kinesin-13 proteins, ABCC4 proteins, ABCG2 proteins, or a combination thereof ca be used to increase the activity of these proteins.

The methods and compositions described herein can also include inhibiting STING, cGAS, NF-κB transcription factor p52, NF-κB transcription factor ReIB, ENPP1, LTPR, BAFFR, CD40, RANK, FN14, NIK (MAP3K14), MST1, or any combination thereof in a mammalian cell. The cells can be in vitro (e.g., in culture) or in vivo (e.g., within a subject animal).

Compositions and methods described herein can include use of kinesin-13 proteins such as Kif2b, MCAK/Kif2c, and/or KIF13A proteins. The compositions and methods can also include use of kinesin-13 nucleic acids encoding kinesin-13 such as Kif2b, MCAK/Kif2c, KIF13A, or a combination thereof. The compositions and methods can also include one or inhibitors of STING, cGAS, NF-κB transcription factor p52, NF-κB transcription factor ReIB, ENPP1, LTβR, BAFFR, CD40, RANK, FN14, NIK (MAP3K14), MST1, or a combination thereof. Examples of such inhibitors include antibodies or inhibitory nucleic acids (e.g., in a carrier or expressed from an expression vector). Such compositions and methods are useful for treating and inhibiting the development of cancer, including metastatic cancer.

As described herein increased activity and/or levels of kinesin-13 proteins such as Kif2b, MCAK/Kif2c, and/or KIF13A, as well as increased activity and/or levels of ABCC4 and/or ABCG2 can reduce the incidence and/or progression of cancer, including metastatic cancer. Reducing expression of STING, cGAS, NF-κB transcription factor p52, NF-κB transcription factor ReIB, ENPP1, LTβR, BAFFR, CD40, RANK, FN14, NIK (MAP3K14), MST1, or any combination thereof can also reduce the incidence and/or progression of cancer, including metastatic cancer.

Sequences for kinesin-13 proteins and nucleic acids such as Kif2b, MCAK/Kif2c, and KIF13A, as well as ABCC4, ABCG2 proteins and nucleic acids, and sequences for STING, cGAS, NF-κB transcription factor p52, and NF-κB transcription factor ReIB, ENPP1, LTβR, BAFFR, CD40, RANK, FN14, NIK (MAP3K14), and MST1 are available, for example, from the database maintained by the National Center for Biotechnology Information (NCBI) data at ncbi.nlm.nih.gov.

For example, one kinesin-13 protein is the a Kif2b protein, which can have the following human sequence (SEQ ID NOA1; NCBI accession number NP_115948).

| | |
|---|---|
| 1 | MASQFCLPES PCLSPLKPLK PHFGDIQEGI YVAIQRSDKR |
| 41 | IHLAVVTEIN RENYWVTVEW VEKAVKKGKK IDLETILLLN |
| 81 | PALDSAEHPM PPPPLSPLAL APSSAIRDQR TATKWVAMIP |
| 121 | QKNQTASGDS LDVRVPSKPC LMKQKKSPCL WEIQKLQEQR |
| 161 | EKRRRLQQEI RARRALDVNT RNPNYEIMHM IEEYRRHLDS |
| 201 | SKISVLEPPQ EHRICVCVRK RPLNQRETTL KDLDIITVPS |
| 241 | DNVVMVHESK QKVDLTRYLQ NQTFCFDHAF DDKASNELVY |
| 231 | QFTAQPLVES IFRKGMATCF AYGQTGSGKT YTMGGDFSGT |
| 321 | AQDCSKGIYA LVAQDVFLLL RNSTYEKLDL KVYGTFFEIY |
| 361 | GGKVYDLLNW KKKLQVLEDG NQQIQVVGLQ EKEVCCVEEV |
| 401 | LNLVEIGNSC RTSRQTPVNA HSSRSHAVFQ IILKSCRIMH |
| 441 | GKFSLVDLAG NERCADTTKA SRKRQLEGAE INKSLLALKE |
| 481 | CILALCQNKP HTPFRASKLT LVLRDSFIGQ NSSTCMIATI |
| 521 | SPGMTSCENT LNTLRYANRV KKLNVDVRPY HRGHYPIGHE |
| 561 | APRMLKSHIG NSEMSLQRDE FIKIPYVQSE EQKEIEEVET |
| 601 | LPTLLGKDTT ISGKGSSQWL ENIQERAGGV HHDIDFCIAR |
| 641 | SLSILEQKID ALTEIQKKLK LLLADLHVKS KVE |

A cDNA sequence that encodes the SEQ ID NO:1 human Kif2b protein is shown below as SEQ ID NO:2 (NCBI accession number NM_032559).

| | |
|---|---|
| 1 | GTAGTGGCCC CAGTCCGGGC CCCGGCGCGC TAGGCTCACA |
| 41 | AAGGCAGGCA CAGACTGCAA CCCTGCTCAG TGCTCCGGGC |
| 81 | GCTTCAGGCT GGCTTGGGTC CTGCTGCTCC AACCCCAAGG |
| 121 | GCCCTGGAGC GCTCCCTGAT ACCTCCATCA CTCACCATGG |
| 161 | CCAGCCAGTT CTGCCTCCCT GAATCCCCAT GTCTCTCGCC |
| 201 | CCTGAAACCC TTGAAGCCAC ATTTCGGAGA CATCCAAGAG |
| 241 | GGCATCTACG TGGCGATCCA GCGCAGTGAC AAGCGGATCC |
| 281 | ACCTCGCTGT GGTCACGGAG ATCAACAGAG AAAACTATTG |
| 321 | GGTCACGGTA GAGTGGGTGG AGAAAGCAGT CAAAAAAGGC |
| 361 | AAGAAGATTG ACCTGGAGAC CATACTCCTG CTGAATCCAG |
| 401 | CTCTGGACTC TGCTGAACAC CCCATGCCGC CCCCGCCCTT |
| 441 | ATCCCCCTTG GCTCTGGCGC CCTCTTCGGC CATCAGGGAC |
| 481 | CAGCGTACCG CCACGAAATG GGTTGCGATG ATCCCCCAGA |
| 521 | AAAACCAAAC AGCCTCAGGG GACAGCCTGG ATGTGAGGGT |
| 561 | CCCCACCAAA CCTTGTCTGA TGAAGCAGAA AAAGTCTCCC |
| 601 | TGCCTCTGGG AAATCCAGAA ACTGCAGGAC CAGCGCCAAA |
| 641 | AGCGCAGGCC GCTGCAGCAG GAGATCCGAG CTAGACGCGC |
| 681 | CCTCGATGTC AATACCAGAA ACCCCAACTA CGAAATCATC |
| 721 | CACATGATCG AAGAGTATCG CAGGCACCTG GACACCACCA |
| 761 | AGATCTCAGT CCTGGACCCC CCGCAAGAAC ATCGCATCTG |
| 801 | CGTCTGCGTG AGGAAGCGGC CTCTCAACCA GCGAGAGACA |
| 841 | ACCTTAAAGG ACCTGGATAT CATCACCGTC CCCTCGGACA |
| 881 | ATGTGGTTAT GGTGCATGAG TCCAAGCAAA AGGTGGACCT |
| 921 | CACTCGCTAC CTGCAGAACC AGACCTTCTG CTTCGACCAT |
| 961 | GCCTTCGATG ACAAAGCCTC CAACGAGTTG GTGTACCAGT |
| 1001 | TCACCGCCCA GCCACTGGTG GAGTCCATCT TCCGCAAGGG |
| 1041 | CATGGCCACC TGCTTTGCCT ATGGGCAGAC GGGAAGTGGG |
| 1081 | AAGACGTACA CCATGGGTGG AGACTTTTCA GGAACGGCCC |
| 1121 | AAGATTGTTC TAAGGGCATT TATGCTCTGG TGGCACAGGA |
| 1161 | TGTCTTTCTC CTGCTCAGAA ACTCCACATA TGAGAAGCTG |
| 1201 | GACCTCAAAG TCTATGGGAC ATTTTTTGAG ATTTATGGGG |
| 1241 | GCAAGGTGTA TGATTTGTTG AACTGGAAGA AGAAGCTGCA |
| 1281 | AGTCCTTGAG GATGGCAATC AGCAAATCCA AGTGGTCGGG |
| 1321 | CTGCAGGAGA AAGAGGTGTG TTGTGTGGAG GAAGTGCTGA |
| 1361 | ACCTGGTGGA AATAGGGAAT AGCTGTCGGA CTTCCAGGCA |
| 1401 | AACACCTGTC AACGCTCACT CATCCAGGAG CCATGCAGTG |
| 1441 | TTCCAGATCA TCCTGAAGTC AGGACGGATA ATGCATGGCA |
| 1481 | AGTTTTCCCT CCTTGATTTA GCTGGGAATG AAAGAGGAGC |
| 1521 | AGATACAACC AACCCCACCC CGAAAACCCA GCTCGAAGGC |
| 1561 | GCAGAGATTA ACAAGACTCT TCTACCCCTC AAAGAATCTA |
| 1601 | TTCTGGCTTT CGCTCAGAAC AAGCCTCACA CCCCATTCAG |
| 1641 | AGCCAGCAAA CTCACACTGG TGCTCCGGGA CTCCTTTATA |
| 1681 | GGCCAGAACT CCTCCACTTG CATGATTGCT ACCATCTCTC |
| 1721 | CGGGGATGAC CTCTTGTGAA AACACTCTCA ACACTTTAAG |
| 1761 | ATATGCAAAC AGAGTAAAAA AATTAAATGT AGATGTAAGG |
| 1801 | CCCTACCATC GTGGCCACTA TCCGATTGGA CATGAGGCAC |
| 1841 | CAAGGATGTT AAAAAGTCAC ATCGGAAATT CAGAAATGTC |
| 1881 | CCTTCAGAGG GATGAATTTA TTAAAATACC TTATGTACAG |
| 1921 | AGTGAGGAGC AGAAAGAGAT TGAAGAGGTT GAAACATTAC |
| 1961 | CCACTCTGTT AGGGAAGGAT ACCACAATTT CAGGGAAGGG |
| 2001 | ATCTAGCCAA TGGCTGGAAA ACATCCAGGA GAGAGCTGGT |
| 2041 | GGAGTACACC ATGATATTGA TTTTTGCATT GCCCGGTCTT |
| 2081 | TGTCCATTTT GGAGCAGAAA ATTGATGCTC TGACCGAGAT |
| 2121 | CCAAAAGAAA CTGAAATTAT TACTAGCTGA CCTCCACGTG |
| 2161 | AAGAGCAAGG TAGAGTGAAG CCAATGGCGA GAGATCAGGT |
| 2201 | CCGAAATGCT GCATTGCTGC AGTTTCCACC ACTCTTATAC |
| 2241 | AGGAAAACTG TCCAAATTAT CTAAAGATCC TCCTGAGAAG |
| 2281 | CTTAAAACAT CTTAAAATAC ACTGATGGGA AACATGCTCT |
| 2321 | TTCTTCTGCC TCTGT |

A kinesin-13 protein is the MCAK/Kif2c protein, which can have the following human sequence (SEQ ID NO:3; NCBI accession number BAG50306.1).

| | |
|---|---|
| 1 | MAMDSSLQAR LFPGLAIKIQ RSNGLIHSAN VRTVNLEKSC |
| 41 | VSVEWAEGGA TKGKEIDFDD VAAINPELLQ LLPLHPKDNL |
| 81 | PLQENVTIQK QKRRSVNSKI PAPKESLRSR STRMSTVSEL |
| 121 | RITAQENDME VELPAAANSR KQFSVPPAPT RPSCPAVAEI |
| 161 | PLRMVSEEME EQVHSIRGSS SANPVNSVRR KSCLVREVEK |
| 201 | MKNKREEKKA QNSEMRMKRA QEYDSSFPNW EFARMIKEFR |
| 241 | ATLECHPLTM TDPIEEHRIC VCVRKRPLNK QELAKKEIDV |
| 281 | ISIPSKCLLL VHEPKLKVDL TKYLENQAFC FDFAFDETAS |
| 321 | NEVVYRFTAR PLVQTIFEGG KATCFAYGQT GSGKTHTMGG |
| 361 | DLSGKAQNAS KGIYAMASRD VFLLKNOPCY RKLGLEVYVT |
| 401 | FFEIYNGKLF DLLNKKAKLR VLEDGKQQWQ VVGLQEHLVN |
| 441 | SADDVIKMLD MGSACRTSGQ TFANSNSSRS HACFQIILRA |
| 481 | KGRMHGKFSL VDLAGNERGA DTSSADRQTR MEGAEINKSL |
| 521 | LALKECIRAL GQNKAHTPFR ESKLTQVLRD SFIGENSRTC |
| 561 | MIATISPGIS SCEYTLNTLR YADRVKELSP HSGPSGEQLI |
| 601 | QMETEEMEAC SNGALIPGNL SKEEEELSSQ MSSFNEAMTQ |
| 641 | IRELEEKAME ELKEIIQQGP DWLELSEMTE QPDYDLETFV |
| 681 | NKAESALAQQ AKHFSALRDV IKALRLAMQL EEQASRQISS |
| 721 | KKRPQ |

A cDNA sequence that encodes the SEQ ID NO:3 human MCAK/Kif2c protein is shown below as SEQ ID NOA4 (NCBI accession numberAB3264115.1).

| | |
|---|---|
| 1 | ACGCTTGCGC GCGGGATTTA AACTGCGGCG GTTTACGCGG |
| 41 | CGTTAAGACT TCGTAGGGTT AGCGAAATTG AGGTTTCTTG |
| 81 | GTATTGCGCG TTTCTCTTCC TTGCTGACTC TCCGAATGGC |
| 121 | CATGGACTCC TCGCTTCAGG CCCGCCTGTT TCCCGGTCTC |
| 161 | GCTATCAAGA TCCAACGCAG TAATGGTTTA ATTCACAGTG |
| 201 | CCAATGTAAC GACTGTGAAC TTGGAGAAAT CCTGTGTTTC |
| 241 | AGTGGAATGG GCAGAAGGAG GTGCCACAAA GGGCAAAGAG |
| 281 | ATTGATTTTG ATGATGTGGC TGCAATAAAC CCAGAACTCT |
| 321 | TACAGCTTCT TCCCTTACAT CCGAAGGACA ATCTGCCCTT |
| 361 | GCAGGAAAAT GTAACAATCC AGAAACAAAA ACGGAGATCC |
| 401 | GTCAACTCCA AAATTCCTGC TCCAAAAGAA AGTCTTCGAA |
| 441 | GCCGCTCCAC TCGCATGTCC ACTGTCTCAG AGCTTCGCAT |
| 481 | CACGGCTCAG GAGAATGACA TGGAGGTGGA GCTGCCTGCA |
| 521 | GCTGCAAACT CCCGCAAGCA GTTTTCAGTT CCTCCTGCCC |
| 561 | CCACTAGGCC TTCCTGCCCT GCAGTGGCTG AAATACCATT |
| 601 | GAGGATGGTC AGCGAGGAGA TGGAAGAGCA AGTCCATTCC |
| 641 | ATCCGTGGCA GCTCTTCTGC AAACCCTGTG AACTCAGTTC |
| 681 | GGAGGAAATC ATGTCTTGTG AAGGAAGTGG AAAAAATGAA |
| 721 | GAACAAGCGA GAAGAGAAGA AGGCCCAGAA CTCTGAAATG |
| 761 | AGAATGAAGA GAGCTCAGGA GTATGACAGT AGTTTTCCAA |
| 801 | ACTGGGAATT TGCCCCAATC ATTAAAGAAT TTCGGGCTAC |
| 841 | TTTGGAATGT CATCCACTTA CTATGACTGA TCCTATCGAA |
| 881 | GAGCACAGAA TATGTGTCTG TGTTAGGAAA CGCCCACTGA |
| 921 | ATAAGCAAGA ATTGGCCAAG AAAGAAATTG ATGTGATTTC |
| 961 | CATTCCTAGC AAGTGTCTCC TCTTGGTACA TGAACCCAAG |
| 1001 | TTGAAAGTGG ACTTAACAAA GTATCTGGAC AACCAAGCAT |
| 1041 | TCTGCTTTGA CTTTGCATTT GATGAAACAG CTTCGAATGA |
| 1081 | AGTTGTCTAC AGGTTCACAC CAAGGCCACT GGTACAGACA |
| 1121 | ATCTTTGAAG GTGGAAAAGC AACTTGTTTT GCATATGGCC |
| 1161 | AGACAGGAAG TGGCAAGACA CATACTATGG GCGGAGACCT |
| 1201 | CTCTGGGAAA GCCCAGAATG CATCCAAAGG GATCTATGCC |
| 1241 | ATGGCCTCCC GGGACGTCTT CCTCCTGAAG AATCAACCCT |
| 1281 | GCTACCGGAA GTTGGGCCTG GAAGTCTATG TGACATTCTT |
| 1321 | CGAGATCTAC AATGGGAAGC TGTTTGACCT GCTCAACAAG |
| 1361 | AAGGCCAAGC TGCGCGTGCT GGAGGACGCC AAGCAACAGG |
| 1401 | TGCAAGTGGT GGGGCTGCAG GAGCATCTGG TTAACTCTGC |
| 1441 | TGATGATGTC ATCAAGATGC TCGACATGCG CAGCGCCTGC |
| 1481 | AGAACCTCTG GGCAGACATT TGCCAACTCC AATTCCTCCC |
| 1521 | GCTCCCACGC GTGCTTCCAA ATTATTCTTC GAGCTAAAGG |
| 1561 | GAGAATGCAT GGCAAGTTCT CTTTGGTAGA TCTGGCAGGG |
| 1601 | AATGAGCGAG GCGCAGACAC TTCCAGTGCT GACCGCCAGA |
| 1641 | CCCGCATGGA GGGCGCAGAA ATCAACAAGA GTCTCTTAGC |
| 1631 | CCTGAAGGAG TGCATCACCG CCCTGGGACA GAACAAGGCT |
| 1721 | CACACCCCGT TCCGTGAGAG CAAGCTGACA CAGGTGCTGA |
| 1761 | GGGACTCCTT CATTGGGGAG AACTCTAGGA CTTGCATGAT |
| 1801 | TGCCACGATC TCACCAGGCA TAAGCTCCTG TGAATATACT |
| 1841 | TTAAACACCC TGAGATATGC AGACAGGGTC AAGGAGCTGA |
| 1881 | GCCCCCACAG TGGGCCCAGT GGAGAGCAGT TGATTCAAAT |
| 1921 | GGAAACAGAA GAGATGGAAG CCTGCTCTAA CGGGGCGCTG |
| 1961 | ATTCCAGGCA ATTTATCCAA GGAAGAGGAG GAAGTGTCTT |
| 2001 | CCCAGATGTC CAGCTTTAAC GAAGCCATGA CTCAGATCAG |
| 2041 | GGAGCTGGAG GAGAAGGCTA TGGAAGAGCT CAAGGAGATC |
| 2081 | ATACAGCAAG GACCAGACTG GCTTGAGCTC TCTGAGATGA |
| 2121 | CCGAGCAGCC AGACTATGAC CTGGAGACCT TTGTGAACAA |
| 2161 | AGCGGAATCT GCTCTGGCCC AGCAAGCCAA GCATTTCTCA |
| 2201 | GCCCTGCGAG ATGTCATCAA GGCCTTAGGC CTGGCCATGC |
| 2241 | AGCTGGAAGA GCAGGCTAGC AGACAAATAA GCAGCAAGAA |
| 2281 | ACGGCCCCAG TGACGACTGC AAATAAAAAT CTGTTTGGTT |
| 2321 | TGACACCCAG CCTCTTCCCT GGCCCTCCCC AGAGAACTTT |
| 2361 | GGGTACCTGG TGGGTCTAGG CAGGGTCTGA GCTGGGACAG |
| 2401 | GTTCTGGTAA ATGCCAAGTA TGGGGGCATC TGGGCCCAGG |
| 2441 | GCAGCTGGGG AGGGGGTCAG AGTCACATGG GACACTCCTT |
| 2481 | TTCTGTTCCT CAGTTGTCGC CCTCACGAGA GGAAGGAGCT |
| 2521 | CTTAGTTACC CTTTTGTGTT GCCCTTCTTT CCATCAAGGG |
| 2561 | GAATGTTCTC AGCATAGAGC TTTCTCCGCA GCATCCTGCC |
| 2601 | TGCGTGGACT GGCTGCTAAT GGAGAGCTCC CTGGGGTTGT |
| 2641 | CCTGGCTCTG GGGAGAGAGA CGGAGCCTTT AGTACAGCTA |
| 2681 | TCTGCTGGCT CTAAACCTTC TACGCCTTTG GGCCGAGCAC |
| 2721 | TGAATGTCTT GTACTTTAAA AAAATGTTTC TGAGACCTCT |
| 2761 | TTCTACTTTA CTGTCTCCCT AGAGTCCTAG AGGATCCCTA |
| 2801 | CTGTTTTCTG TTTTATGTGT TTATACATTG TATGTAACAA |
| 2841 | TAAAGAGAAA AAATAAAAAA AAAAAAAAAA AAAAAAAAAA |
| 2881 | AAAAAA |

Another kinesin-13 protein is the KIR13A protein, which can have the following human sequence (SEQ ID NO:5; NCBI accession number NP_071396.4).

| | |
|---|---|
| 1 | MSDTKVKVAV RVRPMNRREL ELNTKCVVEM EGNQTVLHPP |
| 41 | PSNTKQGERK PPKVFAFDYC FWSMDESNTT KYAGQEVVFK |
| 81 | CLGEGILEKA FQGYNACIFA YGQTGSGKSF SMMGHAEQLG |
| 121 | LIPRLCCALF KRISLEQNES QTFKVEVSYM EIYNEKVRDL |
| 161 | LDPKGSRQSL KVREHKVLGP YVDGLSQLAV TSFEDIESLM |
| 201 | SEGNKSRTVA ATNMNEESSR SHAVFNIIIT QTLYDLQSGN |
| 241 | SGEKVSKVSL VDLAGSERVS KTGAAGERLK EGSNINKSLT |
| 281 | TLGLVISSLA DQAAGKGKSK FVPYRDSVLT WLLKDNLGGN |
| 321 | SQTSMIATIS PAADNYEETL STLRYADRAK RIVNHAVVNE |
| 361 | DPNAKVIREL REEVEKLREQ LSQAEAMKAP ELKEKLEESE |
| 401 | KLIKELTVTW EEKLRKTEEI AQERQRQLES MGISLEMSGI |
| 441 | KVGDDKCYLV NLNADPALNE LLVYYLKDHT RVGADTSQDI |
| 481 | QLFGIGIQPQ HCEIDIASDG DVILTPKENA RSCVNGTLVC |
| 521 | STTQLWHGDR ILWGNNHFFR INLPKRKRRD WLKDFEKETG |
| 561 | PPEHDLDAAS EASSEPDYNY EFAQMEVIMK TLNSNDPVQN |
| 601 | VVQVLEKQYL EEKRSALEEQ RLMYERELEQ LRQQLSPDRQ |
| 641 | PQSSGPDRLA YSSQTAQQKV TQWAEERDEL FRQSLAKLRE |
| 681 | QLVKANTLVR EANFLAEEMS KLTDYQVTLQ IPAANLSANR |
| 721 | KRGAIVSEPA IQVRRKGKST QVWTIEKLEN KLIDMRDLYQ |
| 761 | EWKEKVPEAK RLYGKRGDPF YEAQENHNLI GVANVFLECL |
| 801 | FCDVKLQYAV PIISQQGEVA GRLHVEVMRV TGAVPERVVE |
| 841 | DDSSENSSES GSLEVVDSSG EIIHRVKKLT CRVKIKEATG |
| 881 | LPINLSNFVF CQYTFWDQCE STVAAPVVDP EVPSPQSKDA |
| 921 | QYTVTFSHCK DYVVNVTEEF LEFISDGALA IEVWGHRCAG |
| 961 | NGSSIWEVDS LHAKTRTLHD RWNEVTRRIE MWISILELNE |
| 1001 | LGEYAAVELH QAKDVNTGGI FQLRQGHSRR VQVTVKPVQH |
| 1041 | SGTLPLMVEA ILSVSIGCVT ARSTKLQRGL DSYQRDDEDG |
| 1081 | DDMDSYQEED LNCVRERWSD ALIKRREYLD EQIKKVSNKT |
| 1121 | EKTEDDVERE AQLVEQWVGLTEERNAVLVP APGSGIPGAP |
| 1161 | ADWIPPPGME THIPVLFLDL NADDLSANEQ LVGPHASCVN |
| 1201 | SILPKEHGSQ FFYLPIIKHS DDEVSATASW DSSVHDSVHL |
| 1241 | NRVTPQNERI YLIVKTTVQL SHPAAMELVL RKRIAANIYN |
| 1281 | KQSFTQSLKR RISLKNIFYS CGVTYEIVSN IPKATEEIED |
| 1321 | RETTALLAAR SENEGTSDGE TYIEKYTRGV LQVENILSLE |
| 1361 | RLRQAVTVKE ALSTKARHIR RSLSTPNVHN VSSSRPDLSG |
| 1401 | FDEDDKGWPE NQLDMSDYSS SYQDVACYGT LPRDSPRRNK |
| 1441 | EGCTSETPHA LTVSPFKAFS PQPPKFFKPL MPVKEEHKKR |
| 1481 | IALEARPLLS QESMPPPQAH NPGCIVPSGS NGSSMPVEHN |
| 1521 | SKREKKIDSE EEENELEAIN RKLISSQPYV PVEFADFSVY |
| 1561 | NASLENREWF SSKVDLSNSR VLEKEVSRSP TTSSITSGYF |
| 1601 | SHSASNATLS DMVVPSSDSS DQLAIQTKDA DSTEHSTPSL |
| 1641 | VHDFRPSSNK ELTEVEKGLV KDKIIVVPLK ENSALAKGSP |
| 1681 | SSQSIPEKNS KSLCRTGSCS ELDACPSKIS QPARGFCPRE |
| 1721 | VTVEHTTNIL EDHSFTEFMG VSEGKDFDGL TDSSAGELSS |
| 1761 | RRSLPNKTGG KTVSDGLHHP SQLHSKLEND QVIIPEAAFW |
| 1801 | VLCCQ |

A cDNA sequence that encodes the SEQ ID NO:5 human KIF13A protein is shown below as SEQ ID NO:6 (NCBI accession number NM_022113.5).

| | |
|---|---|
| 1 | CGGGATGGCC CGCGCGCCTC GGCGCTGCCT CTCGGAGCTC |
| 41 | ACGGCGGAGC GGCGGCGGCC GCGCTCGAGG GGCGCGCGGC |
| 81 | TGCAGCGGCG GCGGCGCCGC GCGTGAGGGG CCGCCTAAGG |
| 121 | CCGAGCGGGC GCGGCGAGCG GCCGGGCGAG CGCAGCCAAC |
| 161 | ATGTCGGATA CCAAGGTAAA AGTTGCCGTC CGGGTCCGGC |
| 201 | CCATGAACCG ACGAGAACTG GAACTGAACA CCAAGTGCGT |
| 241 | GGTGGAGATG GAAGGGAATC AAACGGTCCT GCACCCTCCT |
| 281 | CCTTCTAACA CCAAACAGGG AGAAAGGAAA CCTCCCAAGG |
| 321 | TATTTGCCTT TGATTATTGC TTTTGGTCCA TGGATGAATC |
| 361 | TAACACTACA AAATACGCTG GTCAAGAAGT GGTTTTCAAG |
| 401 | TGCCTTGGGG AAGGAATTCT TGAAAAAGCC TTTCAGGGGT |
| 441 | ATAATGCGTG TATTTTTGCA TATGGACAGA CAGGTTCGGG |
| 481 | AAAATCCTTT TCCATGATGG GCCATGCTGA GCAGGTGGGC |
| 521 | CTTATTCCAA GGCTCTGCTG TGCTTTATTT AAAAGGATCT |
| 561 | CTTTGGAGCA AAATGAGTCA CAGACCTTTA AAGTTGAAGT |
| 601 | GTCCTATATG GAAATTTATA ATGAGAAAGT TCGGGATCTT |
| 641 | TTAGACCCCA AAGGGAGTAG ACAGTCTCTT AAAGTTCGAG |
| 681 | AACATAAAGT TTTGGGACCA TATGTAGATG GTTTATCTCA |
| 721 | ACTAGCTGTC ACTAGTTTTG AGGATATTGA GTCATTGATG |
| 761 | TCTGAGGGAA ATAAGTCTCG AACGGTAGCT GCTACCAACA |
| 801 | TGAACGAAGA AAGCAGCCGC TCCCATGCTG TGTTCAACAT |
| 841 | CATAATCACA CAGACACTTT ATGACCTGCA GTCTGGGAAT |
| 881 | TCCGGGGAGA AAGTCAGTAA GGTCAGCTTG GTAGACCTGG |
| 921 | CGGGTAGCGA AAGAGTATCT AAAACAGGAG CTGCAGGAGA |
| 961 | CCGACTGAAA GAAGGCAGCA ACATTAACAA ATCGCTTACA |
| 1001 | ACCTTCGGGT TGGTTATATC ATCACTGGCT GACCAGGCAG |
| 1041 | CTGGCAAGGG TAAAAGCAAA TTTGTGCCTT ATCGAGATTC |
| 1081 | AGTCCTCACT TGGCTGCTTA AGGACAACTT GGGGGGCAAC |
| 1121 | AGCCAAACCT CTATGATAGC CACAATCAGC CCAGCCGCAG |
| 1161 | ACAACTATGA AGAGAGCCTC TCCACATTAA GATATGCAGA |
| 1201 | CCGAGCCAAA AGGATTGTGA ACCATGCTGT TGTGAATGAG |
| 1241 | GACCCCAACG CAAAAGTGAT CCGAGAACTG CGGGAGGAAG |
| 1281 | TCGAGAAAGT GAGAGAGCAG CTCTCTCAGG CAGAGGCCAT |
| 1321 | GAAGGCCCCT GAACTGAAGG AGAAGCTCGA AGAGTCTGAA |
| 1361 | AAGCTGATAA AAGAACTAAC AGTGACTTGG GAAGAGAAGC |
| 1401 | TGAGAAAAAC AGAAGAGATA GCACAGGAAA GACAACGACA |
| 1441 | AGTTGAAAGC ATGGGGATTT CCCTGGAGAT GTCCGGTATC |
| 1481 | AAGGTGGGGG ATGACAAATG CTACTTAGTC AATCTGAATG |
| 1521 | CAGACCCTGC TCTTAACGAA CTTCTGGTTT ATTATTTAAA |
| 1561 | GGATCACACC AGGGTGGGTG CAGATACCTC TCAAGATATC |
| 1601 | CAGCTTTTTG GCATAGGAAT TCAGCCTCAG CACTGTGAGA |
| 1641 | TTGACATTGC ATCTGATGGA GACGTCACTC TCACTCCAAA |
| 1681 | AGAAAATGCA AGGTCCTGTG TGAACGGCAC CCTTGTGTGC |
| 1721 | AGTACCACCC AGCTGTGGCA TGGTGACCGA ATCCTATGGG |
| 1761 | GAAATAATCA CTTTTTTAGA ATAAACTTAC CTAAGAGGAA |
| 1801 | ACGTCGAGAT TGGTTGAAAG ACTTTGAAAA AGAAACGGGC |
| 1841 | CCGCCAGAGC ATGACCTGGA TGCAGCCAGT GAGGCTTCCT |
| 1881 | CTGAACCAGA CTATAACTAT GAATTTGCAC AGATGGAAGT |
| 1921 | TATCATGAAA ACCCTGAATA GTAATGACCC AGTTCAAAAT |
| 1961 | GTGGTTCAGG TCCTGGAGAA ACAATACCTA GAAGAAAAGA |
| 2001 | GAAGTGCCCT AGAGGAGCAG CGGCTCATGT ATGAGCGGGA |
| 2041 | ACTGGAGCAA CTCCGCCAGC AGCTCTCCCC CGACAGGCAG |
| 2081 | CCACAGAGTA GCGGCCCTGA CCGCCTGGCC TACAGCAGCC |
| 2121 | AGACACCGCA CCAGAAGGTG ACCCAGTGGG CAGAAGAGAG |
| 2161 | GGATGAACTC TTCCGACAAA GCCTGGCAAA ACTGCGAGAG |
| 2201 | CAGCTGGTTA AAGCTAATAC CTTGGTGAGG GAAGCAAACT |
| 2241 | TCCTGGCTGA GGAAATGAGC AAACTCACCG ATTACCAAGT |
| 2281 | GACTCTTCAG ATCCCTGCTG CAAACCTCAG TGCCAATAGG |
| 2321 | AAGAGAGGTG CAATAGTGAG TGAACCAGCT ATCCAAGTGA |
| 2361 | GGAGGAAAGG AAAGAGCACC CAAGTGTGGA CCATTGAGAA |
| 2401 | GCTGGAGAAT AAATTAATTG ACATGAGAGA CCTTTACCAA |
| 2441 | GAATGGAAGG AAAAAGTTCC TGAGGCAAAG AGACTCTACG |
| 2481 | GAAAACGAGG TGACCCTTTC TATGAAGCCC AAGAAAATCA |
| 2521 | CAACCTCATC GGGGTGGCGA ATGTATTCTT GGAATGCCTC |
| 2561 | TTCTGTGATG TGAAACTTCA GTATGCAGTC CCTATCATCA |
| 2601 | GCCAGCAGGG GGAGGTTGCA GGGCGTCTCC ACGTGGAAGT |
| 2641 | GATGCGTGTT ACAGGAGCTG TTCCAGAGCG TGTGGTGGAG |
| 2681 | GATGACTCTT CGGAGAATTC CAGTGAAAGT GGGAGCCTTG |
| 2721 | AAGTCGTAGA CAGCAGCGGG GAAATCATTC ACCGAGTCAA |
| 2761 | AAAGCTGACA TGTCGGGTAA AAATTAAAGA AGCAACGGGG |
| 2801 | CTGCCCTTAA ACCTCTCAAA TTTTGTCTTC TGTCAATACA |
| 2841 | CATTCTGGGA CCAGTGTGAG TCTACGGTGG CTGCCCCGGT |
| 2881 | GGTGGACCCC GAGGTGCCTT CACCACAGTC CAAGGATGCC |
| 2921 | CAGTACACAG TGACCTTCTC CCACTGTAAG GACTATGTGG |
| 2961 | TGAATGTAAC AGAAGAATTT CTGGAGTTCA TTTCAGATGG |
| 3001 | AGCACTGGCC ATTGAAGTAT GGGGCCACCG GTGTGCTGGA |
| 3041 | AATGGCAGCT CCATCTGGGA GGTCGATTCT CTTCATGCTA |
| 3081 | AGACAAGAAC ACTGCATGAC AGGTGGAATG AAGTAACGCG |
| 3121 | AAGAATAGAA ATGTGGATCT CCATATTAGA ATTGAATGAG |
| 3161 | TTAGGAGAGT ATGCTGCAGT GGAACTTCAT CAGGCAAAAG |
| 3201 | ATGTCAACAC AGGAGGCATC TTTCAACTTA GACAGGGTCA |
| 3241 | TTCCCGTAGA GTACAAGTCA CGGTGAAACC TGTGCAGCAT |
| 3281 | TCAGGGACAC TGCCACTTAT GGTTGAAGCC ATCCTGTCAG |
| 3321 | TATCCATCGG CTGTGTAACT GCCAGGTCCA CCAAACTCCA |
| 3361 | AAGAGGGCTG GACAGTTACC AGAGAGATGA TGAGGATGGT |
| 3401 | GATGATATGG ATAGTTATCA GGAAGAAGAC TTAAACTGCG |
| 3441 | TAAGGGAGAG GTGGTCAGAT GCACTCATTA AACGACGAGA |
| 3481 | ATACCTGGAT GAACAGATAA AAAAAGTCAG CAATAAAACA |
| 3521 | GAGAAAACAG AGGACGATGT GGAGCGGGAA GCCCAGCTTG |
| 3561 | TGGAGCAGTG GGTAGGGCTG ACTGAGGAAA GGAATGCTGT |
| 3601 | GCTGGTGCCA GCCCCAGGCA GTGGGATTCC TGGGGCACCT |
| 3641 | GCCGACTGGA TCCCACCTCC TGGAATGGAA ACCCACATAC |
| 3681 | CAGTTCTCTT CCTCGATTTG AATGCGGATG ACCTCAGTGC |
| 3721 | CAATGAGCAG CTTGTTGGCC CCCATGCATC CGGCGTGAAC |
| 3761 | TCCATCCTGC CCAAGGAGCA TGGCAGCCAG TTTTTCTACC |
| 3801 | TGCCCATCAT AAAGCACAGT GATGATGAGG TTTCAGCCAC |
| 3841 | AGCCTCTTGG GATTCCTCGG TGCATGATTC TGTTCACTTG |
| 3881 | AATAGGGTCA CACCACAGAA TGAAAGGATT TACCTAATTG |
| 3921 | TGAAAACCAC AGTTCAACTC AGCCACCCTG CTGCTATGGA |
| 3961 | GTTAGTATTA CGAAAACGAA TTGCAGCCAA TATTTACAAC |
| 4001 | AAACAGAGTT TCACGCAGAG TTTGAAGAGG AGAATATCCC |
| 4041 | TGAAAAATAT ATTTTATTCC TGTGGTGTAA CCTATGAAAT |
| 4081 | AGTATCCAAT ATACCAAAGG CAACTGAGGA GATAGAGGAC |
| 4121 | CGGGAAACGC TGGCTCTCCT GGCAGCAAGG AGTGAAAACG |
| 4161 | AAGGCACATC AGATGGGGAG ACGTACATTG AGAAGTACAC |
| 4201 | TCGAGGCGTC CTGCAGGTGG AAAACATTCT GAGTCTTGAA |
| 4241 | CGGCTCCGGC AGGCCGTCAC AGTCAAAGAA GCACTTTCCA |
| 4281 | CCAAAGCCCG GCACATTCGG AGGAGCCTCA GTACACCAAA |
| 4321 | TGTTCATAAT GTCTCTTCCA GCCGACCGGA CCTTTCTGGC |
| 4361 | TTTGATGAAG ATGACAAGGG TTGGCCAGAG AACCAGTTGG |
| 4401 | ACATGTCTGA CTATAGCTCC AGTTACCAAG ATGTAGCATG |
| 4441 | TTATGGAACT TTACCCAGGG ATTCTCCTCG AAGGAATAAA |
| 4481 | GAAGGTTGTA CATCAGAGAC TCCTCATGCC TTAACCGTCA |
| 4521 | GCCCTTTTAA AGCATTCTCT CCTCAGCCAG CAAAGTTTTT |
| 4561 | CAAGCCCCTA ATGCCTGTAA AAGAGGAGCA TAAGAAAAGG |
| 4601 | ATAGCCCTGG AAGCAAGGCC TCTTCTAAGC CAGGAGAGCA |
| 4641 | TGCCTCCACC TCAGGCACAT AACCCTGGCT GCATTGTACC |
| 4681 | CTCAGGAAGC AATGGCAGCA GCATGCCAGT AGAACACAAT |
| 4721 | AGCAAACGTG AGAAGAAGAT TGACTCTGAG GAGGAAGAAA |
| 4761 | ATGAGCTGGA AGCTATTAAC AGGAAGCTAA TAAGTTCACA |
| 4801 | GCCTTATGTA CCTGTGGAGT TTGCTGACTT CAGTGTTTAC |
| 4841 | AATGCCAGCT TGGAGAACAG GGAATGGTTT TCCTCTAAAG |
| 4881 | TAGATCTGTC AAACTCACGG GTCTTGGAGA AAGAAGTGTC |
| 4921 | CCGTAGCCCT ACCACCAGCA GTATTACCAG TGGCTACTTT |
| 4961 | TCCCACAGTG CCTCCAATGC CACCCTGTCT GACATGGTGG |
| 5001 | TCCCTTCTAG TGACAGCTCA GACCAGCTGG CCATTCAGAC |
| 5041 | GAAGGATGCA GACTCCACCG AGCACTCCAC ACCATCGCTT |
| 5081 | GTGCATGATT TCAGGCCGTC CTCAAACAAA GAGTTGACAG |
| 5121 | AAGTCGAAAA AGGCTTGGTA AAGGACAAGA TAATTGTGGT |
| 5161 | GCCACTCAAG GAAAACAGTG CCTTAGCCAA AGGGAGCCCA |
| 5201 | TCATCCCAGA GCATCCCTGA GAAAAACTCC AAATCACTGT |
| 5241 | GCAGGACTGG CTCATGTTCA GAACTAGATG CCTGCCCCAG |
| 5281 | CAAAATTAGC CAGCCAGCCA GGGGATTCTG CCCCAGGGAG |
| 5321 | GTGACGGTAG AACACACCAC CAACATCCTT GAAGACCATT |
| 5361 | CTTTCACAGA ATTTATGGGA GTGTCAGAGG GAAAAGATTT |
| 5401 | TGATGGTTTG ACAGATTCTT CTGCTGGAGA GCTTTCCAGT |
| 5441 | AGGAGGAGTC TACCAAATAA AACAGGCGGC AAGACTGTCT |
| 5481 | CCGATGGGCT CCACCACCCC AGCCAGCTGC ATTCCAAGTT |
| 5521 | AGAGAATGAC CAGGTAATAA TTCCAGAGGC AGCCTTTTGG |
| 5561 | GTTCTYTGCT GTCAATGAGT ATGTCTAACT GTATGTCAAC |
| 5601 | CCCAGAGGCC CTTCACCGCA ACAACTTGGT AGGAAAGATT |
| 5641 | CATCCAGTTG TTTGTGACAG CAAAGATGAG CCCACAGAGA |
| 5681 | AGGAGGCTCA CTTCCTGCAC AGCTGTCTCT GTCGGAGAGC |
| 5721 | AAGTCTGTTT TGGGAACTAG AACGCAATTG TGAAATTATA |
| 5761 | AGACCAGTGG ATTTTTTTAC CTGGCACATG GGTTGGTGTT |
| 5801 | GAATGAAGTG TTCAGATGGA TAAGGATCAA TCTCATATTC |
| 5841 | ATTCCCTGGG ATGTTTAGTT ACCAGTTTTC CCAAAGTGTT |
| 5881 | CTGGTAGCAT CTACCATATT TCATCAAATC TGTGATTCCT |
| 5921 | TTGATTATTA TATGAACCAT TATTTTATGT ATCATTAAGA |
| 5961 | AAAAATACTG CCAATTAAAC TCTGTCATAT CAACAAAAAA |
| 6001 | AAAAA |

An example sequence for a human MCAK protein is shown below as SEQ 25 ID NO:7; NCBI accession number NP 006836.2).

| | |
|---|---|
| 1 | MAMDSSLQAR LFPGLAIKIQ RSNGLIHSAN VRTVNLEKSC |
| 41 | VSVEWAEGGA TKGKEIDFDD VAAINPELLQ LLPLHPKDNL |
| 81 | PLQENVTIQK QKRRSVNSKI PAPKESLRSR STRMSTVSEL |
| 121 | RITAQENDME VELPAAANSR KQFSVPPAPT RPSCPAVAEI |
| 161 | PLRMVSEEME EQVHSIRGSS SANPVNSVRR KSCLVKEVEK |
| 201 | MKNKREEKKA QNSEMRMKRA QEYDSSFPNW EFARMIKEFR |
| 241 | ATLECHPLTM TDPIEEHRIC VCVRKRPLNK QELAKKEIDV |
| 281 | ISIPSKCLLL VHEPKLKVDL TKYLENQAFC FDFAFDETAS |
| 321 | NEVVYRFTAR PLVQTIFEGG KATCFAYGQT GSGKTHTMGG |
| 361 | DLSGKAQNAS KGIYAMASRD VFLLKNQPCY RKLGLEVYVT |
| 401 | FFEIYNGKLF DLLNKKAKLR VLEDGKQQVQ VVGLQEHLVN |
| 441 | SADDVIKMID MGSACRTSGQ TFANSNSSRS HACFQIILRA |
| 431 | KGRMHGKFSL VDLAGNERGA DTSSADRQTR MEGAEINKSL |
| 521 | LALKECIRAL GQNKAHTPFR ESKLTQVLRD SFIGENSRTC |
| 561 | MIATISPGIS SCEYTLNTLR YADRVKELSP HSGPSGEQLI |
| 601 | QMETEEMEAC SNGALIPGNL SKEEEELSSQ MSSFNEAMTQ |
| 641 | IRELEEKAME ELKEIIQQGP DWLELSEMTE QPDYDLETFV |
| 681 | NKAESALAQQ AKHFSALRDV IKALRLAMQL EEQASRQISS |
| 721 | KKRPQ |

A cDNA sequence that encodes the SEQ ID NO:7 human MCAK protein is shown below as SEQ ID NO:8 (NCBI accession number NM_006845.3).

| | |
|---|---|
| 1 | ACGCTTGCGC GCGGGATTTA AACTGCGGCG GTTTACGCGG |
| 41 | CGTTAAGACT TCGTAGGGTT AGCGAAATTG AGGTTTCTTG |
| 81 | GTATTGCGCG TTTCTCTTCC TTGCTGACTC TCCGAATGGC |
| 121 | CATGGACTCG TCGCTTCAGG CCCGCCTGTT TCCCGGTCTC |
| 161 | GCTATCAAGA TCCAACGCAG TAATGGTTTA ATTCACAGTG |
| 201 | CCAATGTAAG GACTGTGAAC TTGGAGAAAT CCTGTGTTTC |
| 241 | AGTGGAATGG GCAGAAGGAG GTGCCACAAA GGGCAAAGAG |
| 281 | ATTGATTTTG ATGATGTGGC TGCAATAAAC CCAGAACTCT |
| 321 | TACAGCTTCT TCCCTTACAT CCGAAGGACA ATCTGCCCTT |
| 361 | GCAGGAAAAT GTAACAATCC AGAAACAAAA ACGGAGATCC |
| 401 | GTCAACTCCA AAATTCCTGC TCCAAAAGAA AGTCTTCGAA |
| 441 | GCCGCTCCAC TCGCATGTCC ACTGTCTCAG AGCTTCGCAT |
| 481 | CACGGCTCAG GAGAATGACA TGGAGGTGGA GCTGCCTGCA |
| 521 | GCTGCAAACT CCCGCAAGCA GTTTTCAGTT CCTCCTGCCC |
| 561 | CCACTAGGCC TTCCTGCCCT GCAGTGGCTG AAATACCATT |
| 601 | GAGGATGGTC AGCGAGGAGA TGGAAGAGCA AGTCCATTCC |
| 641 | ATCCGAGGCA GCTCTTCTGC AAACCCTGTG AACTCAGTTC |
| 681 | GGAGGAAATC ATGTCTTGTG AAGGAAGTGG AAAAAATGAA |
| 721 | GAACAAGCGA GAAGAGAAGA AGGCCCAGAA CTCTGAAATG |
| 761 | AGAATGAAGA GAGCTCAGGA GTATGACAGT AGTTTTCCAA |
| 801 | ACTGGGAATT TGCCCGAATG ATTAAAGAAT TTCGGGCTAC |
| 841 | TTTGGAATGT CATCCACTTA CTATGACTGA TCCTATCGAA |
| 881 | GAGCACAGAA TATGTGTCTG TGTTAGGAAA CGCCCACTGA |
| 921 | ATAAGCAAGA ATTGGCCAAG AAAGAAATTG ATGTGATTTC |
| 961 | CATTCCTAGC AAGTGTCTCC TCTTGGTACA TGAACCCAAG |
| 1001 | TTGAAAGTGG ACTTAACAAA GTATCTGGAG AACCAAGCAT |
| 1041 | TCTGCTTTGA CTTTGCATTT GATGAAACAG CTTCGAATGA |
| 1081 | AGTTGTCTAC AGGTTCACAG CAAGGCCACT GGTACAGACA |
| 1121 | ATCTTTGAAG GTGGAAAAGC AACTTGTTTT GCATATGGCC |
| 1161 | AGACAGGAAG TGGCAAGACA CATACTATGG GCGGAGACCT |
| 1201 | CTCTGGGAAA GCCCAGAATG CATCCAAAGG GATCTATGCC |
| 1241 | ATGGCCTCCC GGGACGTCTT CCTCCTGAAG AATCAACCCT |
| 1281 | GCTACCGGAA GTTGGGCCTG GAAGTCTATG TGACATTCTT |
| 1321 | CGAGATCTAC AATGGGAAGC TGTTTGACCT GCTCAACAAG |
| 1361 | AAGCCCAAGC TGCGCGTGCT GGAGGACGGC AAGCAACAGG |
| 1401 | TGCAAGTGGT GGGGCTGCAG GAGCATCTGG TTAACTCTGC |
| 1441 | TGATGATGTC ATCAAGATGA TCGACATGGG CAGCGCCTGC |
| 1481 | AGAACCTCTG GGCAGACATT TGCCAACTCC AATTCCTCCC |
| 1521 | GCTCCCACGC GTGCTTCCAA ATTATTCTTC GAGCTAAAGG |
| 1561 | GAGAATGCAT GGCAAGTTCT CTTTGGTAGA TCTGGCAGGG |
| 1601 | AATGAGCGAG GCGCGGACAC TTCCAGTGCT GACCGGCAGA |
| 1641 | CCCGCATGGA GGGCGCAGAA ATCAACAAGA GTCTCTTAGC |
| 1681 | CCTGAAGGAG TGCATCAGGG CCCTGGGACA GAACAAGGCT |
| 1721 | CACACCCCGT TCCGTGAGAG CAAGCTGACA CAGGTGCTGA |
| 1761 | GGGACTCCTT CATTGGGGAG AACTCTAGGA CTTGCATGAT |
| 1801 | TGCCACGATC TCACCAGGCA TAAGCTCCTG TGAATATACT |
| 1841 | TTAAACACCC TGAGATATGC AGACAGGGTC AAGGAGCTGA |
| 1881 | GCCCCCACAG TGGGCCCAGT GGAGAGCAGT TGATTCAAAT |
| 1921 | GGAAACAGAA GAGATGGAAG CCTGCTCTAA CGGGGCGCTG |
| 1961 | ATTCCAGGCA ATTTATCCAA GGAAGAGGAG GAACTGTCTT |
| 2001 | CCCAGATGTC CAGCTTTAAC GAAGCCATGA CTCAGATCAG |
| 2041 | GGAGCTGGAG GAGAAGGCTA TGGAAGAGCT CAAGGAGATC |
| 2081 | ATACAGCAAG GACCAGACTG GCTTGAGCTC TCTGAGATGA |
| 2121 | CCGAGCAGCC AGACTATGAC CTGGAGACCT TTGTGAACAA |
| 2161 | AGCGGAATCT GCTCTGGCCC AGCAAGCCAA GCATTTCTCA |
| 2201 | GCCCTGCGAG ATGTCATCAA GGCCTTGCGC CTGGCCATGC |
| 2241 | AGCTGGAAGA GCAGGCTAGC AGACAAATAA GCAGCAAGAA |
| 2281 | ACGGCCCCAG TGACGACTGC AAATAAAAAT CTGTTTGGTT |
| 2321 | TGACACCCAG CCTCTTCCCT GGCCCTCCCC AGAGAACTTT |
| 2361 | GGGTACCTGG TGGGTCTAGG CAGGGTCTGA GCTGGGACAG |
| 2401 | GTTCTGGTAA ATGCCAAGTA TGGGGGCATC TGGGCCCAGG |
| 2441 | GCAGCTGGGG AGGGGGTCAG AGTGACATGG GACACTCCTT |
| 2481 | TTCTGTTCCT CAGTTGTCGC CCTCACGAGA GGAAGGAGCT |
| 2521 | CTTAGTTACC CTTTTGTGTT GCCCTTCTTT CCATCAAGGG |
| 2561 | GAATGTTCTC AGCATAGACC TTTCTCCGCA GCATCCTGCC |
| 2601 | TGCGTGGACT GGCTGCTAAT GGAGAGCTCC CTGGGGTTGT |
| 2641 | CCTGGCTCTG GGGAGAGAGA CGGAGCCTTT AGTACAGCTA |
| 2681 | TCTGCTGGCT CTAAACCTTC TACGCCTTTG GGCCGAGCAC |
| 2721 | TGAATGTCTT GTACTTTAAA AAAATGTTTC TGAGACCTCT |
| 2761 | TTCTACTTTA CTGTCTCCCT AGAGATCCTA GAGGATCCCT |
| 2801 | ACTGTTTTCT GTTTTATGTG TTTATACATT GTATGTAACA |
| 2841 | ATAAAGAGAA AAAATAAATC AGCTGTTTAA GTGTGTGGAA |
| 2881 | AAAAAAAAAA AAAAAA |

An example sequence for a human ABCC4 protein is shown below as SEQ ID NO:9; NCBI accession number AAH41560.1).

| | |
|---|---|
| 1 | MLPVYQEVKP NPLQDANLCS RVFFWWLNPL FKIGHKRRLE |
| 41 | EDDMYSVLPE DRSQHLGEEL QGFWDKEVLR AENDAQKPSL |
| 81 | TRAIIKCYWK SYLVLGIFTL IEESAKVIQP IFLGKIINYF |
| 121 | ENYDPMDSVA LNTAYAYATV LTFCTLILAI LHHLYFYHVQ |
| 161 | CAGMRLRVAM CHMIYRKALR LSNMAMGKTT TGQIVNLLSN |
| 201 | DVNKFDQVTV FLHFLWAGPL QAIAVTALLW MEIGISCLAG |
| 241 | MAVLIILLPL QSCFGKLFSS LRSKTATFTD ARIRTMNEVI |
| 281 | TGIRIIKMYA WEKSFSNLIT NLRKKEISKI LRSSCLRGMN |
| 321 | LASFFSASKI IVFVTFTTYV LLGSVITASR VFVAVTLYGA |
| 361 | VRLTVKLFFP SAIERVSEAI VSIRRIQTFL LLDEISQRNR |
| 401 | QLPSDGKKMV HVQDFTAFWD KASETPTLQG LSFTVRPGEL |
| 441 | LAVVGPVGAG KSSLLSAVLG ELAPSHGLVS VHGRIAYVSQ |
| 481 | QPWVFSGTLR SNILFGKKYE KERYEKVIKA CALKKDLQLL |
| 521 | EDGDLTVIGD RGTTLSGGQK ARVNLARAVY QDADIYLLDD |
| 561 | PLSAVDAEVS RHLFELCICQ ILHEKITILV THQLQYLKAA |
| 601 | SQILILKDGK MVQKGTYTEF LKSGIDFGSL LKKDNEESEQ |
| 641 | PPVPGTPTLR NRTFSESSVW SQQSSRPSLK DGALESQDTE |
| 681 | NVPVTLSEEN RSEGKVGFQA YKNYFRAGAH WIVFIFLILL |
| 721 | NTAAQVAYVL QDWWLSYWAN KQSMLNVTVN GGGNVTEKLD |
| 761 | LNWYLGIYSG LTVATVLFGI ARSLLVFYVL VNSSQTLHNK |
| 801 | MFESILKAPV LFFDRNPIGR ILNRFSKDIG HLDDLLPLTF |
| 841 | LDFIQRWDLA VLSWLVSNS |

A cDNA sequence that encodes the SEQ ID NO:9 human ABCC4 protein is shown below as SEQ ID NO:10 (NCBI accession number BC041560.1).

| | |
|---|---|
| 1 | GGCCGGAGCC CCAGCATCCC TGCTTGAGGT CCAGGAGCGG |
| 41 | AGCCCGCGGC CACCGCCGCC TGATCAGCGC GACCCCGGCC |
| 81 | CGCGCCCGCC CCGCCCGGCA AGATGCTGCC CGTGTACCAG |
| 121 | GAGGTGAAGC CCAACCCGCT GCAGGACGCG AACCTCTGCT |
| 161 | CACGCGTGTT CTTCTGGTGG CTCAATCCCT TGTTTAAAAT |
| 201 | TGGCCATAAA CGGAGATTAG AGGAAGATGA TATGTATTCA |
| 241 | GTGCTGCCAG AAGACCGCTC ACAGCACCTT GGAGAGGAGT |
| 281 | TGCAAGGGTT CTGGGATAAA GAAGTTTTAA GAGCTGAGAA |
| 321 | TGACGCACAG AAGCCTTCTT TAACAAGAGC AATCATAAAG |
| 361 | TGTTACTGGA AATCTTATTT AGTTTTGGGA ATTTTTACGT |
| 401 | TAATTGAGGA AAGTGCCAAA GTAATCCAGC CCATATTTTT |
| 441 | GGGAAAAATT ATTAATTATT TTGAAAATTA TGATCCCATG |
| 481 | GATTCTGTGG CTTTGAACAC AGCGTACGCC TATGCCACGG |
| 521 | TGCTGACTTT TTGCACGCTC ATTTTGGCTA TACTGCATCA |
| 561 | CTTATATTTT TATCACGTTC AGTGTGCTGG GATGAGGTTA |
| 601 | CGAGTAGCCA TGTGCCATAT GATTTATCGG AAGGCACTTC |
| 641 | GTCTTAGTAA CATGGCCATG GGGAAGACAA CCACAGGCCA |
| 681 | GATAGTCAAT CTGCTGTCCA ATGATGTGAA CAAGTTTGAT |
| 721 | CAGGTGACAG TGTTCTTACA CTTCCTGTGG GCAGGACCAC |
| 761 | TGCAGGCGAT CGCACTGACT GCCCTACTCT GGATGGAGAT |
| 801 | AGGAATATCG TGCCTTGCTG GGATGGCAGT TCTAATCATT |
| 841 | CTCCTGCCCT TGCAAAGCTG TTTTGGGAAG TTGTTCTCAT |
| 881 | CACTGAGGAG TAAAACTGCA ACTTTCACGG ATGCCAGGAT |
| 921 | CAGGACCATG AATGAAGTTA TAACTGGTAT AAGGATAATA |
| 961 | AAAATGTACG CCTGGGAAAA GTCATTTTCA AATCTTATTA |
| 1001 | CCAATTTGAG AAAGAAGGAG ATTTCCAAGA TTCTGAGAAG |
| 1041 | TTCCTGCCTC AGGGGGATGA ATTTGGCTTC GTTTTTCAGT |
| 1081 | GCAAGCAAAA TCATCGTGTT TGTGACCTTC ACCACCTACG |
| 1121 | TGCTCCTCGG CAGTGTGATC ACAGCCAGCC GCGTGTTCGT |
| 1161 | GGCAGTGACG CTGTATGGGG CTGTGCGGCT GACGGTTACC |
| 1201 | CTCTTCTTCC CCTCAGCCAT TGAGAGGGTG TCAGAGGCAA |
| 1241 | TCGTCAGCAT CCGAAGAATC CAGACCTTTT TGCTACTTGA |
| 1281 | TGAGATATCA CAGCGCAACC GTCAGCTGCC GTCAGATGGT |
| 1321 | AAAAAGATGG TGCATGTGCA GGATTTTACT GCTTTTTGGG |
| 1361 | ATAAGGCATC AGAGACCCCA ACTCTACAAG GCCTTTCCTT |
| 1401 | TACTGTCAGA CCTGGCGAAT TGTTAGCTGT GGTCGGCCCC |
| 1441 | GTGGGAGCAG GGAAGTCATC ACTGTTAAGT GCCGTGCTCG |
| 1481 | GGGAATTGGC CCCAAGTCAC GGGCTGGTCA GCGTGCATGG |
| 1521 | AAGAATTGCC TATGTGTCTC AGCAGCCCTG GGTGTTCTCG |
| 1561 | GGAACTCTGA GGAGTAATAT TTTATTTGGG AAGAAATACG |
| 1601 | AAAAGGAACG ATATGAAAAA GTCATAAAGG CTTGTGCTCT |
| 1641 | GAAAAAGGAT TTACAGCTGT TGGAGGATGG TGATCTGACT |
| 1681 | GTGATAGGAG ATCGGGGAAC CACGCTGAGT GGAGGGCAGA |
| 1721 | AAGCACGGGT AAACCTTGCA AGAGCAGTGT ATCAAGATGC |
| 1761 | TGACATCTAT CTCCTGGACG ATCCTCTCAG TGCAGTAGAT |
| 1801 | GCGGAAGTTA GCAGACACTT GTTCGAACTG TGTATTTGTC |
| 1841 | AAATTTTGCA TGAGAAGATC ACAATTTTAG TGACTCATCA |
| 1881 | GTTGCAGTAC CTCAAAGCTG CAAGTCAGAT TCTGATATTG |
| 1921 | AAAGATGGTA AAATGGTGCA GAAGGGGACT TACACTGAGT |
| 1961 | TCCTAAAATC TGGTATAGAT TTTGGCTCCC TTTTAAAGAA |
| 2001 | GGATAATGAG GAAAGTGAAC AACCTCCAGT TCCAGGAACT |
| 2041 | CCCACACTAA GGAATCGTAC CTTCTCAGAG TCTTCGGTTT |
| 2081 | GGTCTCAACA ATCTTCTAGA CCCTCCTTGA AAGATGGTGC |
| 2121 | TCTGGAGAGC CAAGATACAG AGAATGTCCC AGTTACACTA |
| 2161 | TCAGAGGAGA ACCGTTCTGA AGGAAAAGTT GGTTTTCAGG |
| 2201 | CCTATAAGAA TTACTTCAGA GCTGGTGCTC ACTGGATTGT |
| 2241 | CTTCATTTTC CTTATTCTCC TAAACACTGC AGCTCAGGTT |
| 2281 | GCCTATGTGC TTCAAGATTG GTGGCTTTCA TACTGGGCAA |
| 2321 | ACAAACAAAG TATGCTAAAT GTCACTGTAA ATGGAGGAGG |
| 2361 | AAATGTAACC GAGAAGCTAG ATCTTAACTG GTACTTAGGA |
| 2401 | ATTTATTCAG CTTTAACTGT AGCTACCGTT CTTTTTGGCA |
| 2441 | TAGCAAGATC TCTATTGGTA TTCTACGTCC TTGTTAACTC |
| 2481 | TTCACAAACT TTGCACAACA AAATGTTTGA GTCAATTCTG |
| 2521 | AAAGCTCCGG TATTATTCTT TGATAGAAAT CCAATAGGAA |
| 2561 | GAATTTTAAA TCGTTTCTCC AAAGACATTG GACACTTGGA |
| 2601 | TGATTTGCTG CCGCTGACCT TTTTAGATTT CATCCAGAGA |
| 2641 | TGGGATCTCG CTGTGTTGTC CTGGCTGGTC TCAAACTCCT |
| 2681 | AGGCTCAAGC AATCCTCCTC CCTCCTCAAG CAAACCTCAG |
| 2721 | TGCTGGGATT ATAGGCATGA GCCACTGTAC CTGGCTAAAT |
| 2761 | GTTGTTTTTT TGATATTCAA TTTTTGTTTA TAGAATTTTC |
| 2801 | ATTTGTTTTG CTCTTATACT TTTCATCTTT TTATGTTTAT |
| 2841 | TGACCAATTA AATATCATTT GGGTAACCAC CTAAAAAAAA |
| 2881 | AAAAAAAAAA |

An example sequence for a human ABCG2 protein is shown below as SEQ ID NO: 11; NCBI accession number AAG52982.1).

| | |
|---|---|
| 1 | MSSSNVEVFI PVSQGNTNGF PATASNDLKA FTEGAVLSFH |
| 41 | NICYRVKLKS GFLPCRKPVE KEILSNINGI MKPGLNAILG |
| 81 | PTGGGKSSLL DVLAARKDPS GLSGDVLING APRPANFKCN |
| 121 | SGYVVQDDVV MGTLTVRENL QFSAALRLAT TMTNHEKNER |
| 161 | INRVIQELGL DKVADSKVGT QFIRGVSGGE RKRTSIGMEL |
| 201 | ITDPSILFLD EPTTGLDSST ANAVLLLLKR MSKQCRTIIF |
| 241 | SIHQPRYSIF KLFDSLTLLA SGRLMFHGPA QEALGYFESA |
| 281 | GYHCEAYNNP ADFFLDIING DSTAVALNRE EDFKATEIIE |
| 321 | PSKQDKPLIE KLAEIYVNSS FYKETKAELH QLSGGEKKKK |
| 361 | ITVFKEISYT TSFCHQLRWV SKRSFKNLLG NPQASIAQII |
| 401 | VTVVLGLVIG AIYFCLKNDS TGIQNRAGVL FFLTTNQCFS |
| 441 | SVSAVELFVV EKKLFIHEYI SGYYRVSSYF LGKLLSDLLP |
| 481 | MRMLPSIIFT CIVYFMLGLK AKADAFFVMM FTLMMVAYSA |
| 521 | SSMALAIAAG QSVVSVATLL MTICFVFMMI FSGLLVNLTT |
| 561 | IASWLSWLQY FSIPRYGFTA LQHNEFLGQN FCPGLNATGN |
| 601 | NPCNYATCTG EEYLVKQGID LSPWGLWKNH VALACMIVIF |
| 641 | LTIAYLKLLF LKKYS |

A cDNA sequence that encodes the SEQ ID NO:11 human ABCG2 protein is shown below as SEQ ID NO:12 (NCBI accession number AY017168.1).

| | |
|---|---|
| 1 | ACCGTGCACA TGCTTGGTGG TCTTGTTAAG TGGAAACTGC |
| 41 | TGCTTTAGAG TTTGTTTGGA AGGTCCGGGT GACTCATCCC |
| 81 | AACATTTACA TCCTTAATTG TTAAAGCGCT GCCTCCGAGC |
| 121 | GCACGCATCC TGAGATCCTG AGCCTTTGGT TAAGACCGAG |
| 161 | CTCTATTAAG CTGAAAAGAT AAAAACTCTC CAGATGTCTT |
| 201 | CCAGTAATGT CGAAGTTTTT ATCCCAGTGT CACAAGGAAA |
| 241 | CACCAATGGC TTCCCCGCGA CAGCTTCCAA TGACCTGAAG |
| 281 | GCATTTACTG AAGGAGCTGT GTTAAGTTTT CATAACATCT |
| 321 | GCTATCGAGT AAAACTGAAG AGTGGCTTTC TACCTTGTCG |
| 361 | AAAACCAGTT GAGAAAGAAA TATTATCGAA TATCAATGGG |
| 401 | ATCATGAAAC CTGGTCTCAA CGCCATCCTG GGACCCACAG |
| 441 | GTGGAGGCAA ATCTTCGTTA TTAGATGTCT TAGCTGCAAG |
| 481 | GAAAGATCCA AGTGGATTAT CTGGAGATGT TCTGATAAAT |
| 521 | GGAGCACCGC GACCTGCCAA TTTCAAATGT AATTCAGGTT |
| 561 | ACGTGGTACA AGATGATGTT GTGATGGGCA CTCTGACGGT |
| 601 | GAGAGAAAAC TTACAGTTCT CAGCAGCTCT TCGGCTTGCA |
| 641 | ACAACTATGA CGAATCATGA AAAAAACGAA CGGATTAACA |
| 681 | GGGTCATTCA AGAGTTAGGT CTGGATAAAG TGGCAGACTC |
| 721 | CAAGGTTGGA ACTCAGTTTA TCCGTGGTGT GTCTGGAGGA |
| 761 | GAAAGAAAAA GGACTAGTAT AGGAATGGAG CTTATCACTG |
| 801 | ATCCTTCCAT CTTGTTCTTG GATGAGCCTA CAACTGGCTT |
| 841 | AGACTCAAGC ACAGCAAATG CTGTCCTTTT GCTCCTGAAA |
| 881 | AGGATGTCTA AGCAGGGACG AACAATCATC TTCTCCATTC |
| 921 | ATCAGCCTCG ATATTCCATC TTCAAGTTGT TTGATAGCCT |
| 961 | CACCTTATTG GCCTCAGGAA GACTTATGTT CCACGGGCCT |
| 1001 | GCTCAGGAGG CCTTGGGATA CTTTGAATCA GCTGGTTATC |
| 1041 | ACTGTGAGGC CTATAATAAC CCTGCAGACT TCTTCTTGGA |
| 1081 | CATCATTAAT GGAGATTCCA CTGCTGTGGC ATTAAACAGA |
| 1121 | GAAGAAGACT TTAAAGCCAC AGAGATCATA GAGCCTTCCA |
| 1161 | AGCAGGATAA GCCACTCATA GAAAAATTAG CGGAGATTTA |
| 1201 | TGTCAACTCC TCCTTCTACA AAGAGACAAA AGCTGAATTA |
| 1241 | CATCAACTTT CCGGGGGTGA GAAGAAGAAG AAGATCACAG |
| 1281 | TCTTCAAGGA GATCAGCTAC ACCACCTCCT TCTGTCATCA |
| 1321 | ACTCAGATGG GTTTCCAAGC GTTCATTCAA AAACTTGCTG |
| 1361 | GGTAATCCCC AGGCCTCTAT AGCTCAGATC ATTGTCACAG |
| 1401 | TCGTACTGGG ACTGGTTATA GGTGCCATTT ACTTTGGGCT |
| 1441 | AAAAAATGAT TCTACTGGAA TCCAGAACAG AGCTGGGGTT |
| 1481 | CTCTTCTTCC TGACGACCAA CCAGTGTTTC AGCAGTGTTT |
| 1521 | CAGCCGTGGA ACTCTTTGTG GTAGAGAAGA AGCTCTTCAT |
| 1561 | ACATGAATAC ATCAGCGGAT ACTACAGAGT GTCATCTTAT |
| 1601 | TTCCTTGGAA AACTGTTATC TGATTTATTA CCCATGAGGA |
| 1641 | TGTTACCAAG TATTATATTT ACCTGTATAG TGTACTTCAT |
| 1681 | GTTAGGATTG AAGGCAAAGG CAGATGCCTT CTTCGTTATG |
| 1721 | ATGTTTACCC TTATGATGGT GGCTTATTCA GCCAGTTCCA |
| 1761 | TGGCACTGGC CATAGCAGCA GGTCAGAGTG TGGTTTCTGT |
| 1801 | AGCAACACTT CTCATGACCA TCTGTTTTGT GTTTATGATG |
| 1841 | ATTTTTTCAG GTCTGTTGGT CAATCTCACA ACCATTGCAT |
| 1881 | CTTGGCTGTC ATGGCTTCAG TACTTCAGCA TTCCACGATA |
| 1921 | TGGATTTACG GCTTTGCAGC ATAATGAATT TTTGGGACAA |
| 1961 | AACTTCTGCC CAGGACTCAA TGCAACAGGA AACAATCCTT |
| 2001 | GTAACTATGC AACATGTACT GGCGAAGAAT ATTTGGTAAA |
| 2041 | GCAGGGCATC GATCTCTCAC CCTGGGGCTT GTGGAAGAAT |
| 2081 | CACGTGGCCT TGGCTTGTAT GATTGTTATT TTCCTCACAA |
| 2121 | TTGCCTACCT GAAATTGTTA TTTCTTAAAA AATATTCTTA |
| 2161 | AATTTCCCCT TAATTCAGTA TGATTTATCC TCACATAAAA |
| 2201 | AAGAAGCACT TTGATTGAAG TATTCAAAAA AAAAAAAAAA |
| 2241 | AAAAAAA |

Kinsin-13, MCAK, ABCC4, and/or ABCG2 proteins and nucleic acids can exhibit sequence variation. However, variants with less than 100% sequence identity to the amino acid and nucleic acid sequences shown herein can still have similar activities. For example, Kinsin-13, MCAK, ABCC4, and/or ABCG2 proteins and nucleic acid with at least 90%, at least 92%, at least 95%, at least 96%, at least 97%, at least 98%, at least 99%, or at least 99.5% sequence identity to any of SEQ ID NOs: 1-12 can still be used in the compositions and methods described herein.

The kinsin-13, MCAK, ABCC4, and/or ABCG2 proteins can be administered to subjects who may exhibit chromosomal instability, or who may be suffering from cancer or be suspected of developing cancer. Similarly, expression cassettes and/or expression vectors encoding kinsin-13, MCAK, ABCC4, and/or ABCG2 proteins can be administered to subjects who may exhibit chromosomal instability, or who may be suffering from cancer or be suspected of developing cancer.

In addition, kinsin-13, MCAK, ABCC4, and/or ABCG2 agonists can be administered to enhance kinesin-13 protein activities. For example, the Kinesin 13 agonist referred to as UMK57, which is specific for Kif2c/MCAK, can be administered to subjects who may exhibit chromosomal instability, or who may be suffering from cancer or be suspected of developing cancer. The structure of UMK57 is shown below, where X is a methyl (CH₃) group.

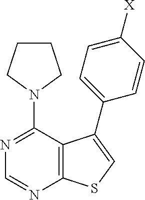

In some cases, the expression of various endogenous nucleic acids (mRNAs) and proteins can be inhibited. For example, the expression of the following can be inhibited STING, cGAS, NF-κB transcription factor p52, NF-κB transcription factor ReIB, ENPP1, LTβR, BAFFR, CD40, RANK, FN14, NIK (MAP3K14), MST1, or any combination thereof.

One example of a human STING protein sequence (SEQ ID NO:13; NCBI accession number NP 938023 XP 291127) is shown below.

| | |
|---|---|
| 1 | MPHSSLHPSI PCPRGHGAQK AALVLLSACL VTLWGLGEPP |
| 41 | EHTLRYLVLH LASLQLGLLL NGVCSLAEEL RHIHSRYRGS |
| 81 | YWRTVRACLG CPLRRGALLL LSIYFYYSLP NAVGPPFTWM |
| 121 | LALLGLSQAL NILLGLKGLA PAEISAVCEK GNFNVAHGLA |
| 161 | WSYYIGYLRL ILPELQARIR TYNQHYNNLL RGAVSQRLYI |
| 201 | LLPLDCGVPD NLSMADPNIR FLDKLPQQTG DHAGIKDRVY |
| 241 | SNSIYELLEN GQRAGTCVLE YATPLQTLFA MSQYSQAGFS |
| 281 | REDRLEQAKL FCRTLEDILA DAPESQNNCR LIAYQEPADD |
| 321 | SSFSLSQEVL RHLRQEEKEE VTVGSLKTSA VPSTSTMSQE |
| 361 | PELLISGMEK PLPLRTDFS |

A cDNA sequence that encodes the SEQ ID NO: 13 human STING protein is shown below as SEQ ID NO:14 (NCBI accession number NM_198282 XM_291127).

| | |
|---|---|
| 1 | TATAAAAATA GCTCTTGTTA CCGGAAATAA CTGTTCATTT |
| 41 | TTCACTCCTC CCTCCTAGGT CACACTTTTC AGAAAAAGAA |
| 81 | TCTGCATCCT GGAAACCAGA AGAAAAATAT GAGACGGGCA |
| 121 | ATCATCGTGT GATGTGTGTG CTGCCTTTGG CTGAGTGTGT |
| 161 | GGAGTCCTGC TCAGGTGTTA GGTACAGTGT GTTTGATCGT |
| 201 | GGTGGCTTGA GGGGAACCCG CTGTTCAGAG CTGTGACTGC |
| 241 | GGCTGCACTC AGAGAAGCTG CCCTTGGCTG CTCGTAGCGC |
| 281 | CGGGCCTTCT CTCCTCGTCA TCATCCAGAG CAGCCAGTGT |
| 321 | CCGGGAGGCA GAAGATGCCC CACTCCAGCC TGCATCCATC |
| 361 | CATCCCGTGT CCCAGGGGTC ACGGGGCCCA GAAGGCAGCC |
| 401 | TTGGTTCTGC TGAGTGCCTG CCTGGTGACC CTTTGGGGGC |
| 441 | TAGGAGAGCC ACCAGAGCAC ACTCTCCGGT ACCTGGTGCT |
| 481 | CCACCTAGCC TCCCTGCAGC TGGGACTGCT GTTAAACGGG |
| 521 | GTCTGCAGCC TGGCTGAGGA GCTGCGCCAC ATCCACTCCA |
| 561 | GGTACCGGGG CAGCTACTGG AGGACTGTGC GGGCCTGCCT |
| 601 | GGGCTGCCCC CTCCGCCGTG GGGCCCTGTT GCTGCTGTCC |
| 641 | ATCTATTTCT ACTACTCCCT CCCAAATGCG GTCGGCCCGC |
| 681 | CCTTCACTTG GATGCTTGCC CTCCTGGGCC TCTCGCAGGC |
| 721 | ACTGAACATC CTCCTGGGCC TCAAGGGCCT GGCCCCAGCT |
| 761 | GAGATCTCTG CAGTGTGTGA AAAAGGGAAT TTCAACGTGG |
| 801 | CCCATGGGCT GGCATGGTCA TATTACATCG GATATCTGCG |
| 841 | GCTGATCCTG CCAGAGCTCC AGGCCCGGAT TCGAACTTAC |
| 881 | AATCAGCATT ACAACAACCT GCTACGGGGT GCAGTGAGCC |
| 921 | AGCGGCTGTA TATTCTCCTC CCATTGGACT GTGGGGTGCC |
| 961 | TGATAACCTG AGTATGGCTG ACCCCAACAT TCGCTTCCTG |
| 1001 | GATAAACTGC CCCAGCAGAC CGGTGACCAT GCTGGCATCA |
| 1041 | AGGATCGGGT TTACAGCAAC AGCATCTATG AGCTTCTGGA |
| 1081 | GAACGGGCAG CGGGCGGGCA CCTGTGTCCT GGAGTACGCC |
| 1121 | ACCCCCTTGC AGACTTTGTT TGCCATGTCA CAATACAGTC |
| 1161 | AAGCTGGCTT TAGCCGGGAG GATAGGCTTG AGCAGGCCAA |
| 1201 | ACTCTTCTGC CCGACACTTG AGGACATCCT GGCAGATGCC |
| 1241 | CCTGAGTCTC AGAACAACTG CCGCCTCATT GCCTACCAGG |
| 1281 | AACCTGCAGA TGACAGCAGC TTCTCGCTGT CCCAGGAGGT |
| 1321 | TCTCCGGCAC CTGCGGCAGG AGGAAAAGGA AGAGGTTACT |
| 1361 | GTGGGCAGCT TGAAGACCTC AGCGGTGCCC AGTACCTCCA |
| 1401 | CGATGTCCCA AGAGCCTGAG CTCCTCATCA GTGGAATGGA |
| 1441 | AAAGCCCCTC CCTCTCCGCA CGGATTTCTC TTGAGACCCA |
| 1481 | GGGTCACCAG GCCAGAGCCT CCAGTGGTCT CCAAGCCTCT |
| 1521 | GGACTGGGGG CTCTCTTCAG TGGCTGAATG TCCAGCAGAG |
| 1561 | CTATTTCCTT CCACAGGGGG CCTTGCAGGG AAGGGTCCAG |
| 1601 | GACTTGACAT CTTAAGATGC GTCTTGTCCC CTTGGGCCAG |
| 1641 | TCATTTCCCC TCTCTGAGCC TCGGTGTCTT CAACCTGTGA |
| 1681 | AATGGGATCA TAATCACTGC CTTACCTCCC TCACGGTTGT |
| 1721 | TGTGAGGACT GAGTGTGTGG AAGTTTTTCA TAAACTTTGG |
| 1761 | ATGCTAGTGT ACTTAGGGGG TGTGCCAGGT GTCTTTCATG |
| 1801 | GCGCCTTCCA CACCCACTCC CCACCCTTCT CCCCTTCCTT |
| 1841 | TGCCCCGGGA CGCCGAACTC TCTCAATGGT ATCAACAGGC |
| 1881 | TCCTTCGCCC TCTGGCTCCT GGTCATGTTC CATTATTGGG |
| 1921 | GAGCCCCAGC AGAAGAATGG AGAGGAGGAG GAGGCTGAGT |
| 1961 | TTGGGGTATT GAATCCCCCG GCTCCCACCC TGCAGCATCA |
| 2001 | AGGTTGCTAT GGACTCTCCT GCCGGGCAAC TCTTGCGTAA |
| 2041 | TCATGACTAT CTCTAGGATT CTGGCACCAC TTCCTTCCCT |
| 2081 | GGCCCCTTAA GCCTAGCTGT GTATCGGCAC CCCCACCCCA |
| 2121 | CTAGAGTACT CCCTCTCACT TGCGGTTTCC TTATACTCCA |
| 2161 | CCCCTTTCTC AACGGTCCTT TTTTAAAGCA CATCTCAGAT |
| 2201 | TACCCAAAAA AAAAAAAAAA AAA |

A cGAS (cyclic GMP-AMP synthase) protein can include the following human sequence (SEQ ID NO:15; NCBI accession number NP_612450).

| | |
|---|---|
| 1 | MQPWHGKAMQ RASEAGATAP KASARNARGA PMDPTESPAA |
| 41 | PEAALPKAGK FGPARKSGSR QKKSAPDTQE RPPVRATGAR |
| 81 | AKKAPQRAQD TQPSDATSAP GAEGLEPPAA REPALSRAGS |
| 121 | CRQRGARCST KPRPPPGPWD VPSPGLPVSA PILVRRDAAP |
| 161 | GASKLRAVLE KLKLSRDDIS TAAGMVKGVV DHLLLRLKCD |
| 201 | SAFRGVGLLN TGSYYEHVKI SAPNEFDVMF KLEVPRIQLE |
| 241 | EYSNTRAYYF VKFKRNPKEN PLSQFLEGEI LSASKMLSKF |
| 281 | RKIIKEEIND IKDTDVIMKR KRGGSPAVTL LISEKISVDI |
| 321 | TLALESKSSW PASTQEGLRI QNWLSAKVRK QLRLKPFYLV |
| 361 | PKHAKEGNGF QEETWRLSFS HIEKEILNNH GKSKTCCENK |
| 401 | EEKCCRKDCL KLMKYLLEQL KERFKDKKHL DKFSSYHVKT |
| 441 | AFFHVCTQNP QDSQWDRKDL GLCFDNCVTY FLQCLRTEKL |
| 481 | ENYFIPEFNL FSSNLIDKRS KEFLTKQIEY ERNNEFPVFD |
| 521 | EF |

A cDNA sequence that encodes the SEQ ID NO: 15 human cGAS protein is shown below as SEQ ID NO: 16 (N CBI accession number NM 13841).

| | |
|---|---|
| 1 | AGCCTGGGGT TCCCCTTCGG GTCGCAGACT CTTGTGTGCC |
| 41 | CGCCAGTAGT GCTTGGTTTC CAACAGCTGC TGCTGGCTCT |
| 81 | TCCTCTTGCG GCCTTTTCCT GAAACGGATT CTTCTTTCGG |
| 121 | GGAACAGAAA GCGCCAGCCA TGCAGCCTTG GCACGGAAAG |
| 161 | GCCATGCAGA GAGCTTCCGA GGCCGGAGCC ACTGCCCCCA |
| 201 | AGGCTTCCGC ACGGAATGCC AGGGGCGCCC CGATGGATCC |
| 241 | CACCCAGTCT CCGGCTGCCC CCGAGGCCGC CCTGCCTAAG |
| 281 | GCGGGAAAGT TCGGCCCCGC CAGGAAGTCG GGATCCCGGC |
| 321 | AGAAAAAGAG CGCCCCGGAC ACCCAGGAGA GGCCGCCCGT |
| 361 | CCGCGCAACT GGGGCCCGCG CCAAAAAGGC CCCTCAGCGC |
| 401 | GCCCAGGACA CCCAGCCGTC TGACGCCACC AGCGCCCCTG |
| 441 | GGGCAGAGGG GCTGGAGCCT CCTGCGGCTC GGGAGCCGGC |
| 481 | TCTTTCCAGG GCTGGTTCTT GCCGCCAGAG GGGCGCGCGC |
| 521 | TGCTCCACGA AGCCAAGACC TCCGCCCGGG CCCTGGGACG |
| 561 | TGCCCAGCCC CGGCCTGCCG GTCTCGGCCC CCATTCTCGT |
| 601 | ACGGAGGGAT GCGGCGCCTG GGGCCTCGAA GCTCCGGGCG |
| 641 | GTTTTGGAGA AGTTGAAGCT CAGCCGCGAT GATATCTCCA |
| 681 | CGGCGGCGGG GATGGTGAAA GGGGTTGTGG ACCACCTGCT |
| 721 | GCTCAGACTG AAGTGCGACT CCGCGTTCAG AGGCGTCGGG |
| 761 | CTGCTGAACA CCGGGAGCTA CTATGAGCAC GTGAAGATTT |
| 801 | CTGCACCTAA TGAATTTGAT GTCATGTTTA AACTGGAAGT |
| 841 | CCCCAGAATT CAACTAGAAG AATATTCCAA CACTCGTGCA |
| 881 | TATTACTTTG TGAAATTTAA AAGAAATCCG AAAGAAAATC |
| 921 | CTCTGAGTCA GTTTTTAGAA GGTGAAATAT TATCAGCTTC |
| 961 | TAAGATGCTG TCAAAGTTTA GGAAAATCAT TAAGGAAGAA |
| 1001 | ATTAACGACA TTAAAGATAC AGATGTCATC ATGAAGAGGA |
| 1041 | AAAGAGGAGG GAGCCCTGCT GTAACACTTC TTATTAGTGA |
| 1081 | AAAAATATCT GTGGATATAA CCCTGGCTTT GGAATCAAAA |
| 1121 | AGTAGCTGGC CTGCTAGCAC CCAAGAAGGC CTGCGCATTC |
| 1161 | AAAACTGGCT TTCAGCAAAA GTTAGGAAGC AACTACGACT |
| 1201 | AAAGCCATTT TACCTTGTAC CCAAGCATGC AAAGGAAGGA |
| 1241 | AATGGTTTCC AAGAAGAAAC ATGGCGGCTA TCCTTCTCTC |
| 1281 | ACATCGAAAA GGAAATTTTG AACAATCATG GAAAATCTAA |
| 1321 | AACGTGCTGT GAAAACAAAG AAGAGAAATG TTGCAGGAAA |
| 1361 | GATTGTTTAA AACTAATGAA ATACCTTTTA GAACAGCTGA |
| 1401 | AAGAAAGGTT TAAAGACAAA AAACATCTGG ATAAATTCTC |
| 1441 | TTCTTATCAT GTGAAAACTG CCTTCTTTCA CGTATGTACC |
| 1481 | CAGAACCCTC AAGACAGTCA GTGGGACCGC AAAGACCTGG |
| 1521 | GCCTCTGCTT TGATAACTGC GTGACATACT TTCTTCAGTG |
| 1561 | CCTCAGGACA GAAAAACTTG AGAATTATTT TATTCCTGAA |
| 1601 | TTCAATCTAT TCTCTAGCAA CTTAATTGAC AAAAGAAGTA |
| 1641 | AGGAATTTCT GACAAAGCAA ATTGAATATG AAAGAAACAA |
| 1681 | TGAGTTTCCA GTTTTTGATG AATTTTGAGA TTGTATTTTT |
| 1721 | AGAAAGATCT AAGAACTAGA GTCACCCTAA ATCCTGGAGA |
| 1761 | ATACAAGAAA AATTTGAAAA GGGGCCAGAC GCTGTGGCTC |
| 1801 | AC |

An NF-κB transcription factor p52 protein can include the following human sequence (SEQ ID NO: 17; NCBI accession number NP_001309863

| | |
|---|---|
| 1 | MESCYNPGLD GIIEYDDFKL NSSIVEPKEP APETADGPYL |
| 41 | VIVEQPKQRG FRFRYGCEGP SHGGLPGASS EKGRKTYPTV |
| 81 | KICNYEGPAK IEVDLVTHSD PPRAHAHSLV GKQCSELGIC |
| 121 | AVSVGPKDMT AQFNNLGVLH VTKKNMMGTM IQKLQRQRLR |
| 161 | SRPQGLTEAE QRELEQEAKE LKKVMDLSIV RLRFSAFLRA |
| 201 | SDGSFSLPLK PVISQPIHDS KSPGASNLKI SRMDKTAGSV |
| 241 | RGGDEVYLLC DKVQKDDIEV RFYEDDENGW QAFGDFSPTD |
| 281 | VHKQYAIVFR TPPYHKMKIE RPVTVFLQLK RKRGGDVSDS |
| 321 | KQFTYYPLVE DKEEVQRKRR KALPTESQPF GGGSHMGGGS |
| 361 | GGAAGGYGGA GGGGSLGFFP SSLAYSPYQS GAGPMGCYPG |
| 401 | GGGGAQMAAT VPSRDSGEEA AEPSAPSRTP QCEPQAPEML |
| 441 | QRAREYNARL FGLAQRSARA LLDYGVTADA RALLAGQRHL |
| 481 | LTAQDENGDT PLHLAIIHGQ TSVIEQIVYV IHHAQDLGVV |
| 521 | NLTNHLHQTP LHLAVITGQT SVVSFLLRVG ADPALLDRHG |
| 561 | DSAMHLALRA GAGAPELLRA LLQSGAPAVP QLLHMPDFEG |
| 601 | LYPVHLAVRA RSPECLDLLV DSGAEVEATE RQGGRTALHL |
| 641 | ATEMEELGLV THLVTKLRAN VNARTFAGNT PLHLAAGLGY |
| 681 | PTLTRLLLKA GADIHAENEE PLCPLPSPPT SDSDSDSEGP |
| 721 | EKDTRSSFRG HTPLDLTCST KVKTLLLNAA QNTMEPPLTP |
| 761 | PSPAGPGLSL GDTALQNLEQ LLDGPEAQGS WAELAERLGL |
| 801 | RSLVDTYRQT TSPSGSLLRS YELAGGDLAG LLEALSDMGI |
| 841 | EEGVRLLRGP ETRDKLPSTA EVKEDSAYGS QSVEQEAEKL |
| 881 | GPPPEPPGGL CHGHPQPQVH |

A cDNA sequence that encodes the SEQ ID NO: 17 human NF-κB transcription factor p52 protein is shown below as SEQ ID NO: 18 (NCBI accession number NM_001322934 XM_005269860).

| | |
|---|---|
| 1 | GCCTCCCGCC CCTCCCGTCG CGAGGGCGGG GCCAGTGGCG |
| 41 | TCATTTCCAG GCCCGCCCCC TCCGGCCCCG CCTCCCCTTG |
| 81 | GTATTTTCGG GACTTTCCTA AGCTGCTCTA ACTTTCCTGC |
| 121 | CCCTTCCCCG GCCAAGCCCA ACTCCGGATC TCGCTCTCCA |
| 161 | CCGGATCTCA CCCGCCACAC CCGGACAGGC GGCTGGAGGA |
| 201 | GGCGGGCGTC TAAAATTCTG GGAAGCAGAA CCTGGCCGGA |
| 241 | GCCACTAGAC AGAGCCGGGC CTAGCCCAGA GACATGGAGA |
| 281 | GTTGCTACAA CCCAGGTCTG GATGGTATTA TTGAATATGA |
| 321 | TGATTTCAAA TTGAACTCCT CCATTGTGGA ACCCAAGGAG |
| 361 | CCAGCCCCAG AAACAGCTGA TGGCCCCTAC CTGGTGATCG |
| 401 | TGGAACAGCC TAAGCAGAGA GGCTTCCGAT TTCGATATGG |
| 441 | CTGTGAAGGC CCCTCCCATG GAGGACTGCC CGGTGCCTCC |
| 481 | AGTGAGAAGG GCCGAAAGAC CTATCCCACT GTCAAGATCT |
| 521 | GTAACTACGA GGGACCAGCC AAGATCGAGG TGGACCTGGT |
| 561 | AACACACAGT GACCCACCTC GTGCTCATGC CCACAGTCTG |
| 601 | GTGGGCAAGC AATGCTCGGA GCTGGGGATC TGCGCCGTTT |
| 641 | CTGTGGGGCC CAAGGACATG ACTGCCCAAT TTAACAACCT |
| 681 | GGGTGTCCTG CATGTGACTA AGAAGAACAT GATGGGGACT |
| 721 | ATGATACAAA AACTTCAGAG GCAGCGGCTC CGCTCTAGGC |
| 761 | CCCAGGGCCT TACGGAGGCC GAGCAGCGGG AGCTGGAGCA |
| 801 | AGAGGCCAAA GAACTGAAGA AGGTGATGGA TCTGAGTATA |
| 841 | GTGCGGCTGC GCTTCTCTGC CTTCCTTAGA GCCAGTGATG |
| 881 | GCTCCTTCTC CCTGCCCCTG AAGCCAGTCA TCTCCCAGCC |
| 921 | CATCCATGAC AGCAAATCTC CGGGGGCATC AAACCTGAAG |
| 961 | ATTTCTCGAA TGGACAAGAC AGCAGGCTCT GTGCGGGGTG |
| 1001 | GAGATGAAGT TTATCTGCTT TGTGACAAGG TGCAGAAAGA |
| 1041 | TGACATTGAG GTTCGGTTCT ATGAGGATGA TGAGAATGGA |
| 1081 | TGGCAGGCCT TTGGGGACTT CTCTCCCACA GATGTGCATA |
| 1121 | AACAGTATGC CATTGTGTTC CGGACACCCC CCTATCACAA |
| 1161 | GATGAAGATT GAGCGGCCTG TAACAGTGTT TCTGCAACTG |
| 1201 | AAACGCAAGC GAGGAGGGGA CGTGTCTGAT TCCAAACAGT |
| 1241 | TCACCTATTA CCCTCTGGTG GAAGACAAGG AAGAGGTGCA |
| 1281 | GCGGAAGCGG AGGAAGGCCT TGCCCACCTT CTCCCAGCCC |
| 1321 | TTCGGGGGTG GCTCCCACAT GGGTGGAGGC TCTGGGGGTG |
| 1361 | CAGCCGGGGG CTACGGAGGA GCTGGAGGAG GTGGCAGCCT |
| 1401 | CGGTTTCTTC CCCTCCTCCC TGGCCTACAG CCCCTACCAG |
| 1441 | TCCGGCGCGG GCCCCATGGG CTGCTACCCG GGAGGCGGGG |
| 1481 | GCGGGGCGCA GATGGCCGCC ACGGTGCCCA GCAGGGACTC |
| 1521 | CGGGGAGGAA GCCGCGGAGC CGAGCGCCCC CTCCAGGACC |
| 1561 | CCCCAGTGCG AGCCGCAGGC CCCGGAGATG CTGCAGCGAG |
| 1601 | CTCGAGAGTA CAACGCGCGC CTGTTCGGCC TGGCGCAGCG |
| 1641 | CAGCGCCCGA GCCCTACTCG ACTACGGCGT CACCGCGGAC |
| 1681 | GCGCGCGCGC TGCTGGCGGG ACAGCGCCAC CTGCTGACGG |
| 1721 | CGCAGGACGA GAACGGAGAC ACACCACTGC ACCTAGCCAT |
| 1761 | CATCCACGGG CAGACCAGTG TCATTGAGCA GATAGTCTAT |
| 1801 | GTCATCCACC ACGCCCAGGA CCTCGGCGTT GTCAACCTCA |
| 1841 | CCAACCACCT GCACCAGACG CCCCTGCACC TGGCGGTGAT |
| 1881 | CACGGGGCAG ACGAGTGTGG TGAGCTTTCT GCTGCGGGTA |
| 1921 | GGTGCAGACC CAGCTCTGCT GGATCGGCAT GGAGACTCAG |
| 1961 | CCATGCATCT GGCGCTGCGG GCAGGCGCTG GTGCTCCTGA |
| 2001 | GCTGCTGCGT GCACTGCTTC AGAGTGGAGC TCCTGCTGTG |
| 2041 | CCCCAGCTGT TGCATATGCC TGACTTTGAG GGACTGTATC |
| 2081 | CAGTACACCT GGCGGTCCGA GCCCGAAGCC CTGAGTGCCT |
| 2121 | GGATCTGCTG GTGGACACTG GGGCTGAAGT GGAGGCCACA |
| 2161 | GAGCGCCAGG GGGGACGAAC AGCCTTGCAT CTAGCCACAG |
| 2201 | AGATGGAGGA GCTGGGGTTG GTCACCCATC TGGTCACCAA |
| 2241 | GCTCCGGGCC AACGTGAACG CTCGCACCTT TGCGGGAAAC |
| 2281 | ACACCCCTGC ACCTGGCAGC TGGACTGGGG TACCCGACCC |
| 2321 | TCACCCGCCT CCTTCTGAAG GCTGGTGCTG ACATCCATGC |
| 2361 | TGAAAACGAG GAGCCCCTGT GCCCACTGCC TTCACCCCCT |
| 2401 | ACCTCTGATA GCGACTCGGA CTCTGAAGGG CCTGAGAAGG |
| 2441 | ACACCCGAAG CAGCTTCCGG GGCCACACGC CTCTTGACCT |
| 2481 | CACTTGCAGC ACCAAGGTGA AGACCTTGCT GCTAAATGCT |
| 2521 | GCTCAGAACA CCATGGAGCC ACCCCTGACC CCGCCCAGCC |
| 2561 | CAGCAGGGCC GGGACTGTCA CTTGGTGATA CAGCTCTGCA |
| 2601 | GAACCTGGAG CAGCTGCTAG ACGGGCCAGA AGCCCAGGGC |
| 2641 | AGCTGGGCAG AGCTGGCAGA GCGTCTGGGG CTGCGCAGCC |
| 2681 | TGGTAGACAC GTACCGACAG ACAACCTCAC CCAGTGGCAG |
| 2721 | CCTCCTGCGC AGCTACGAGC TGGCTGGCGG GGACCTGGCA |
| 2761 | GGTCTACTGG AGGCCCTGTC TGACATGGGC CTAGAGGAGG |
| 2801 | GAGTGAGGCT CCTGAGGCCT CCAGAAACCC GAGACAAGCT |
| 2841 | GCCCAGCACA GCAGAGGTGA AGGAAGACAG TGCGTACGGG |
| 2881 | AGCCAGTCAG TGGAGCAGGA GGCAGAGAAG CTGGGCCCAC |
| 2921 | CCCCTGAGCC ACCAGGAGGG CTCTGCCACG GGCACCCCCA |
| 2961 | GCCTCAGGTG CACTGACCTG CTGCCTGCCC CCAGCCCCCT |
| 3001 | TCCCGGACCC CCTGTACAGC GTCCCCACCT ATTTCAAATC |
| 3041 | TTATTTAACA CCCCACACCC ACCCCTCAGT TGGGACAAAT |
| 3081 | AAAGGATTCT CATGGGAAGG GGAGGACCCC TCCTTCCCAA |
| 3121 | CTTATGGCA |

An NF-κB transcription factor ReIB protein can include the following human sequence (SEQ ID NO: 19; NCBI accession number NP 006500).

| | |
|---|---|
| 1 | MLRSGPASGP SVPTGRAMPS RRVARPPAAP ELGALGSPDL |
| 41 | SSLSLAVSRS TDELEIIDEY IKENGFGLDG GQPGPGEGLP |
| 81 | RLVSRGAASL STVTLGPVAP PATPPPWGCP LGRLVSPAPG |
| 121 | PGPQPHLVIT EQPKQRGMRF RYECEGRSAG SILGESSTEA |
| 161 | SKTLPAIELR DCGGLREVEV TACLVWKDWP HRVHPHSLVG |
| 201 | KDCTDGICRV RLRPHVSPRH SFNNLGIQCV RKKEIEAAIE |
| 241 | RKIQLGIDPY NAGSLKNHQE VDMNVVRICF QASYRDQQGQ |
| 281 | MRRMDPVLSE PVYDKKSTNT SELRICRINK ESGPCTGGEE |
| 321 | LYLLCDKVQK EDISVVFSRA SWEGRADFSQ ADVHRQIAIV |
| 361 | FKTPPYEDLE IVEPVTVNVF LQRLTDGVCS EPLPFTYLPR |
| 401 | DHDSYGVDKK RKRGMPDVLG ELNSSDPHGI ESKRRKKKPA |
| 441 | ILDHFLPNHG SGPFLPPSAL LPDPDFFSGT VSLPGLEPPG |
| 481 | GPDLLDDGFA YDPTAPTLFT MLDLLPPAPP HASAVVCSGG |
| 521 | AGAVVGETPG PEPLTTDSYQ APGPGDGGTA SLVGSNMFPN |
| 541 | HYREAAFGGG LLSPGPEAT |

A cDNA sequence that encodes the SEQ ID NO: 19 human NF-κB transcription factor ReIB protein is shown below as SEQ ID NO:20 (NCBI accession number NM 006509).

| | |
|---|---|
| 1 | GGCCCCGCGC CCCGCGCAGC CCCGGGCGCC GCGCGTCCTG |
| 41 | CCCGGCCTGC GGCCCCAGCC CTTGCGCCGC TCGTCCGACC |
| 81 | CGCGATCGTC CACCAGACCG TGCCTCCCGG CCGCCCGGCC |
| 121 | GGCCCGCGTG CATGCTTCGG TCTGGGCCAG CCTCTGGGCC |
| 161 | GTCCGTCCCC ACTGGCCGGG CCATGCCGAG TCGCCGCGTC |
| 201 | GCCAGACCGC CGGCTGCGCC GGAGCTGGGG GCCTTAGGGT |
| 241 | CCCCCGACCT CTCCTCACTC TCGCTCGCCG TTTCCAGGAG |
| 281 | CACAGATGAA TTGGAGATCA TCGACGAGTA CATCAAGGAG |
| 321 | AACGGCTTCG GCCTGGACGG GGGACAGCCG GGCCCGGGCG |
| 361 | AGGGCCTGCC ACGCCTGGTG TCTCGCCGGG CTCCGTCCCT |
| 401 | GAGCACGGTC ACCCTGGGCC CTGTGGCGCC CCCAGCCACG |
| 441 | CCGCCGCCTT GGGGCTGCCC CCTGGGCCGA CTAGTGTCCC |
| 481 | CAGCGCCGGG CCCGGGCCCG CAGCCGCACC TGGTCATCAC |
| 521 | GGAGCAGCCC AAGCAGCGCG GCATGCGCTT CCGCTACGAG |
| 561 | TGCGAGGGCC GCTCGGCCGG CAGCATCCTT GGGGAGAGCA |
| 601 | GCACCGAGGC CAGCAAGACG CTGCCCGCCA TCGAGCTCCG |
| 641 | GGATTGTGGA GGGCTGCGGG AGGTGGAGGT GACTGCCTGC |
| 681 | CTGGTGTGGA AGGACTGGCC TCACCGAGTC CACCCCCACA |
| 721 | GCCTCGTGGG GAAAGACTGC ACCGACGGCA TCTGCAGGGT |
| 761 | GCGGCTCCGG CCTCACGTCA GCCCCCGGCA CAGTTTTAAC |
| 801 | AACCTGGGCA TCCAGTGTGT GAGGAAGAAG GAGATTGAGG |
| 841 | CTGCCATTGA GCGGAAGATT CAACTGGGCA TTGACCCCTA |
| 881 | CAACGCTGGG TCCCTGAAGA ACCATCAGGA AGTAGACATG |
| 921 | AATGTGGTGA GGATCTGCTT CCAGGCCTCA TATCGGGACC |
| 961 | AGCACCGACA GATGCGCCGG ATGGATCCTG TGCTTTCCGA |
| 1001 | GCCCGTCTAT GACAAGAAAT CCACAAACAC ATCAGAGCTG |
| 1041 | CGGATTTGCC CAATTAACAA GGAAAGCGGG CCGTGCACCG |
| 1081 | GTGGCGAGGA GCTCTACTTG CTCTGCGACA AGGTGCAGAA |
| 1121 | AGAGGACATA TCAGTGGTGT TCAGCAGGGC CTCCTGGGAA |
| 1161 | GGTCGGGCTG ACTTCTCCCA GGCCGACGTG CACCGCCAGA |
| 1201 | TTGCCATTGT GTTCAAGACG CCGCCCTACG AGGAGCTGGA |
| 1241 | GATTGTCGAG CCCGTGACAG TCAACGTCTT CCTGCAGCGG |
| 1281 | CTCACCGATG GGGTCTGCAG CGAGCCATTG CCTTTCACGT |
| 1321 | ACCTGCCTCG CGACCATGAC AGCTACGGCG TGGACAAGAA |
| 1361 | GCGGAAACGG GGGATGCCCG ACGTCCTTGG GGAGCTGAAC |
| 1401 | AGCTCTGACC CCCATGGCAT CGAGAGCAAA CGGCGGAAGA |
| 1441 | AAAAGCCGGC CATCCTGGAC CACTTCCTGC CCAACCACGG |
| 1481 | CTCAGGCCCG TTCCTCCCGC CGTCAGCCCT GCTGCCAGAC |
| 1521 | CCTGACTTCT TCTCTGGCAC CGTGTCCCTG CCCGGCCTGG |
| 1561 | AGGCCCCTGG CGGGCCTGAC CTCCTGGACG ATGGGTTTGC |
| 1601 | CTACGACCCT ACGGGCCCCA CACTCTTCAC CATGCTGGAC |
| 1641 | CTGCTGCCCC CGGCACCGCC ACACGCTAGC GCTGTTGTGT |
| 1681 | GCAGCGGAGG TGCCGGGGCC GTGGTTGGGG AGACCCCCGG |
| 1721 | CCCTCAACCA CTGACACTCG ACTCCTACCA GGCCCCGGGC |
| 1761 | CCCGGGGATG GAGGCACCGC CAGCCTTGTG GGCAGCAACA |
| 1801 | TGTTCCCCAA TCATTACCGC GAGGCGGCCT TTGGGGGCGG |
| 1841 | CCTCCTATCC CCGGGGCCTG AAGCCACGTA GCCCCGCGAT |
| 1881 | GCCAGAGGAG GGGCACTGGG TGGGGAGGGA GGTGGAGGAG |
| 1921 | CCGTGCAATC CCAACCACCA TGTCTAGCAC CCCCATCCCC |
| 1961 | TTGGCCCTTC CTCATGCTTC TGAAGTGGAC ATATTCAGCC |
| 2001 | TTGGCGAGAA GCTCCGTTGC ACGGGTTTCC CCTTGAGCCC |
| 2041 | ATTTTACAGA TGAGGAAACT GAGTCCGGAG AGGAAAAGGG |
| 2081 | AGATGGCTCC CGTGCAGTAG CTTGTTAGAG CTGCCTCTGT |
| 2121 | CCCCACATGT GGGGGCACCT TCTCCAGTAG GATTCGGAAA |
| 2161 | AGATTCTAGA TATGGGAGGA GGGGGCAGAT TCCTGGCCCT |
| 2201 | CCCTCCCCAG ACTTGAAGGT GGGGGGTAGG TTGGTTGTTC |
| 2241 | AGAGTCTTCC CAATAAAGAT GAGTTTTTGA GCCTCCGGGA |
| 2281 | AAAAAAAAAA AAAAAAA |

For example, a ENPP1 protein can include the following human sequence (SEQ ID NO:21; NCBI accession number NP_006199.2).

| | |
|---|---|
| 1 | MERDGCAGGG SRGGEGGRAP REGPAGNGRD RGRSHAAEAP |
| 41 | GDPQAAASLL APMDVGEEPL EKAARARTAK DPNTYKVLSL |
| 81 | VLSVCVLTTI LGCIFGLKPS CAKEVKSCKG RCFERTFGNC |
| 121 | RCDAACVELG NCCLDYQETC IEPEHIWTCN KFRCGEKRLT |
| 161 | RSLCACSDDC KDKGDCCINY SSVCQGEKSW VEEPCESINE |
| 201 | PQCPAGFETP PTLLFSLDGF RAEYLHTWGG LLPVISKLKK |
| 241 | CGTYTKNMRP VYPTKTFPNH YSIVTGLYPE SHGIIDNKMY |
| 281 | DPKMNASFSL KSKEKFNPEW YKGEPIWVTA KYQGLKSGTF |
| 321 | FWPGSDVEIN GIFPDIYKMY NGSVPFEERI LAVLQWLQLP |
| 361 | KDERPHFYTL YLEEPDSSGH SYGPVSSEVI KALQRVDGMV |
| 401 | GMLMDGLKEL NLHRCLNLIL ISDHGMEQGS CKKYIYLNKY |
| 441 | LGDVKNIKVI YGPAARLRPS DVPDKYYSFN YEGIARNLSC |
| 481 | REPNQHFKPY LKHFLPKRLH FAKSDRIEPL TFYLDPQWQL |
| 521 | ALNPSERKYC GSGFHGSDNV FSNMQALFVG YGPGFKHGIE |
| 561 | ADTFENIEVY NLMCDLLNLT PAPNNGTHGS LNHLLKNPVY |
| 601 | TPKHPKEVHP LVQCPFTRNP RDNLGCSCNP SILPIEDFQT |
| 641 | QFNLTVAEEK IIKHETLPYG RPRVLQKENT ICLLSQHQFM |
| 681 | SGYSQDILMP LWTSYTVDRN DSFSTEDFSN CLYQDFRIPL |
| 721 | SPVHKCSFYK NNTKVSYGFL SPPQLNKNSS GIYSEALLTT |
| 761 | NIVPMYQSFQ VIWRYFHDTL LRKYAEERNG VNVVSGPVFD |
| 801 | FDYDGRCDSL ENLRQKRRVI RNQEILIPTH FFIVLTSCKD |
| 841 | TSQTPLHCEN LDTLAFILPH RTDNSESCVH GKHDSSWVEE |
| 881 | LLMLHRARIT DVEHITGLSF YQQRKEPVSD ILKLKTHLPT |
| 921 | FSQED |

A cDNA sequence that encodes the SEQ ID NO:21 human ENPP1 protein is shown below as SEQ ID NO:22 (NCBI accession number NM 006208.2).

| | |
|---|---|
| 1 | CCGGAGCGGC CGGGGCCACG ATGGAGCGCG ACGGCTGCGC |
| 41 | GGGGGGCGGG AGCCGCGGCG GCGAGGGCGG GCGCGCTCCC |
| 81 | CGGGAGGGCC CGGCGGGGAA CGGCCGCGAT CGGGGCCGCA |
| 121 | GCCACGCTGC CGAGGCGCCC GGGGACCCGC AGGCGGCCGC |
| 161 | GTCCTTGCTG GCCCCTATGC ACGTGGGGGA GGAGCCGCTG |
| 201 | GAGAAGGCGG CGCGCGCCCG CACTGCCAAG GACCCCAACA |
| 241 | CCTATAAACT ACTCTCGCTG GTATTGTCAG TATGTGTCTT |
| 281 | AACAACAATA CTTGGTTCTA TATTTGGGTT GAAACCAAGC |
| 321 | TGTGCCAAAG AAGTTAAAAG TTGCAAAGGT CGCTGTTTCG |
| 361 | AGAGAACATT TGGGAACTCT CGCTGTGATG CTGCCTGTGT |
| 401 | TGAGCTTGGA AACTGCTCTT TAGATTACCA GGAGACGTGC |
| 441 | ATAGAACCAG AACATATATG GACTTGCAAC AAATTCAGGT |
| 481 | GTGGTGAGAA AAGGTTGACC AGAAGCCTCT GTGCCTGTTC |
| 521 | AGATGACTGC AAGGACAAGG GCGACTGCTG CATCAACTAC |
| 561 | AGTTCTGTGT GTCAAGGTGA GAAAAGTTGG GTAGAAGAAC |
| 601 | CATGTGAGAG CATTAATGAG CCACAGTGCC CAGCAGGGTT |
| 641 | TGAAACGCCT CCTACCCTCT TATTTTCTTT GGATGGATTC |
| 681 | AGGGCAGAAT ATTTACACAC TTGGGGTGGA CTTCTTCCTG |
| 721 | TTATTAGCAA ACTAAAAAAA TGTGGAACAT ATACTAAAAA |
| 761 | CATGAGACCG GTATATCCAA CAAAAACTTT CCCCAATCAC |
| 801 | TACAGCATTG TCACCGGATT GTATCCAGAA TCTCATGGCA |
| 841 | TAATCGACAA TAAAATGTAT GATCCCAAAA TGAATGCTTC |
| 881 | CTTTTCACTT AAAAGTAAAG AGAAATTTAA TCCTGAGTGG |
| 921 | TACAAAGGAG AACCAATTTC GGTCACAGCT AAGTATCAAG |
| 961 | GCCTCAAGTC TGGCACATTT TTCTGGCCAG GATCAGATGT |
| 1001 | GGAAATTAAC GGAATTTTCC CAGACATCTA TAAAATGTAT |
| 1041 | AATGGTTCAG TACCATTTCA AGAAAGGATT TTAGCTGTTC |
| 1081 | TTCAGTGGCT ACAGCTTCCT AAAGATGAAA GACCACACTT |
| 1121 | TTACACTCTG TATTTAGAAG AACCAGATTC TTCAGGTCAT |
| 1161 | TCATATGGAC CAGTCAGCAG TGAAGTCATC AAAGCCTTGC |
| 1201 | ACACGGTTCA TGCTATGGTT GGTATGCTGA TGGATGGTCT |
| 1241 | GAAAGAGCTC AACTTGCACA GATGCCTGAA CCTCATCCTT |
| 1281 | ATTTCAGATC ATGGCATGGA ACAAGGCAGT TGTAAGAAAT |
| 1321 | ACATATATCT GAATAAATAT TTGGGGGATG TTAAAAATAT |
| 1361 | TAAAGTTATC TATGGACCTG CAGCTCGATT GAGACCCTCT |
| 1401 | GATGTCCCAG ATAAATACTA TTCATTTAAC TATGAAGGCA |
| 1441 | TTGCCCGAAA TCTTTCTTGC CGGGAACCAA ACCAGCACTT |
| 1481 | CAAACCTTAC CTGAAACATT TCTTACCTAA GCGTTTGCAC |
| 1521 | TTTGCTAAGA GTGATAGAAT TGAGCCCTTG ACATTCTATT |
| 1561 | TGGACCCTCA GTGGCAACTT GCATTGAATC CCTCAGAAAG |
| 1601 | GAAATATTGT GGAAGTGGAT TTCATGGCTC TGACAATGTA |
| 1641 | TTTTCAAATA TGCAAGCCCT CTTTGTTGGC TATGGACCTG |
| 1681 | GATTCAAGCA TGGCATTGAG GCTGACACCT TTGAAAACAT |
| 1721 | TGAAGTCTAT AACTTAATGT GTGATTTACT GAATTTGACA |
| 1761 | CCGGCTCCTA ATAACGGAAC TCATGGAAGT CTTAACCACC |
| 1801 | TTCTAAAGAA TCCTGTTTAT ACGCCAAAGC ATCCCAAAGA |
| 1841 | AGTCCACCCC CTGGTACAGT GCCCCTTCAC AAGAAACCCC |
| 1881 | AGAGATAACC TTGGCTGCTC ATGTAACCCT TCGATTTTGC |
| 1921 | CGATTGAGGA TTTTCAAACA CAGTTCAATC TGACTGTGGC |
| 1961 | AGAAGAGAAG ATTATTAAGC ATGAAACTTT ACCCTATGGA |
| 2001 | AGACCTAGAG TTCTCCAGAA GGAAAACACC ATCTGTCTTC |
| 2041 | TTTCCCAGCA CCAGTTTATG AGTGGATACA GCCAAGACAT |
| 2081 | CTTAATGCCC CTTTGGACAT CCTATACCGT GGACAGAAAT |
| 2121 | GACAGTTTCT CTACGGAAGA CTTCTCCAAC TGTCTGTACC |
| 2161 | AGGACTTTAG AATTCCTCTT AGTCCTGTCC ATAAATGTTC |
| 2201 | ATTTTATAAA AATAACACCA AAGTGAGTTA CGGGTTCCTC |
| 2241 | TCCCCACCAC AACTAAATAA AAATTCAAGT GGAATATATT |
| 2281 | CTGAAGCTTT GCTTACTACA AATATAGTGC CAATGTACCA |
| 2321 | GAGTTTTCAA GTTATATGGC GCTACTTTCA TGACACCCTA |
| 2361 | CTGCGAAAGT ATGCTGAAGA AAGAAATGGT GTCAATGTCG |
| 2401 | TCAGTGGTCC TGTGTTTGAC TTTGATTATG ATGGACGTTG |
| 2441 | TGATTCCTTA GAGAATCTGA GGCAAAAAAG AAGAGTCATC |
| 2481 | CGTAACCAAG AAATTTTGAT TCCAACTCAC TTCTTTATTG |
| 2521 | TGCTAACAAG CTGTAAAGAT ACATCTCAGA CGCCTTTGCA |
| 2561 | CTGTGAAAAC CTAGACACCT TAGCTTTCAT TTTGCCTCAC |
| 2601 | AGGACTGATA ACAGCGAGAG CTGTGTGCAT GGGAAGCATG |
| 2641 | ACTCCTCATG GGTTGAAGAA TTGTTAATGT TACACAGAGC |
| 2681 | ACGGATCACA GATGTTGAGC ACATCACTGG ACTCAGCTTC |
| 2721 | TATCAACAAA GAAAAGAGCC AGTTTCAGAC ATTTTAAAGT |
| 2761 | TGAAAACACA TTTGCCAACC TTTAGCCAAG AAGACTGATA |
| 2801 | TGTTTTTTAT CCCCAAACAC CATGAATCTT TTTGAGAGAA |
| 2841 | CCTTATATTT TATATAGTCC TCTAGCTACA CTATTGCATT |
| 2881 | GTTCAGAAAC TGTCGACCAG AGTTAGAACG GAGCCCTCGG |
| 2921 | TGATGCGGAC ATCTCAGGGA AACTTGCGTA CTCAGCACAG |
| 2961 | CAGTGGAGAG TGTTCCTGTT GAATCTTGCA CATATTTGAA |
| 3001 | TGTGTAAGCA TTGTATACAT TGATCAAGTT CGGGGGAATA |
| 3041 | AAGACAGACC ACACCTAAAA CTGCCTTTCT GCTTCTCTTA |
| 3081 | AAGGAGAAGT AGCTGTGAAC ATTGTCTGGA TACCAGATAT |
| 3121 | TTGAATCTTT CTTACTATTG GTAATAAACC TTGATGGCAT |
| 3161 | TGGGCAAACA GTAGACTTAT AGTAGGGTTG GGGTAGCCCA |
| 3201 | TGTTATGTGA CTATCTTTAT GAGAATTTTA AAGTGGTTCT |
| 3241 | GGATATCTTT TAACTTGGAG TTTCATTTCT TTTCATTGTA |
| 3281 | ATCAAAAAAA AAATTAACAG AAGCCAAAAT ACTTCTGAGA |
| 3321 | CCTTGTTTCA ATCTTTGCTG TATATCCCCT CAAAATCCAA |
| 3361 | GTTATTAATC TTATGTGTTT TCTTTTTAAT TTTTTGATTG |
| 3401 | GATTTCTTTA GATTTAATGG TTCAAATGAG TTCAACTTTG |
| 3441 | AGGGACGATC TTTGAATATA CTTACCTATT ATAAAATCTT |
| 3481 | ACTTTGTATT TGTATTTAAA AAAGAAAAAT ATTCCTATCC |
| 3521 | TGCTCACTGG TAATTAACAT AGGTTTAAAA TGGCTTCAAA |
| 3561 | TGTGGCCCTA TAGACGGTTA AAATTGTACC TTATCTTGGC |
| 3601 | AAAACTTCAG AGCACCAGTC AGTGCATGCA AGGTGCCATT |
| 3641 | TTTTATTGAG ATGCTTAGAA TGTTTCTTTC TGTGCACAAG |
| 3681 | ACTTACCCTA CCAGCAGCAG AGCCATTCTC TGTTGAGTGG |
| 3721 | TTCATTTTGA AGTTCCACAG ATTGAAGAGA ACATGCCACC |
| 3761 | AATCACCTCA CATCTTCTTG GTGGACATGA TAAATGACAC |
| 3801 | AATGAACTTG ATTTCTTTAC TACCTTGACT GTACCTTTTT |
| 3841 | ATCCCTACCT GTGAACCTTC AAAGACTGCA TTAACTTTTA |
| 3881 | GGCTACATAG GTCCAATTGA GGTATAATAT CAGTACACCA |
| 3921 | AAGATTTTTA TATGTCCTTC GTGTGACCAT TCTTCAACGG |
| 3961 | CCTAAGGGCC AGCTGCAAAG ACTTTTGGAA AATACAATTT |
| 4001 | ACAACTCAAA ATTATTTAAT AATTTAGGAA GTTGCTTTTT |
| 4041 | TTTTTTTTTT TTTTCAGTCC TGCAGTTTCC TGAAGCTCTG |
| 4081 | TATATGATAT TTTTTTCAGC CTGCTTCTCT CTGTTGTTCA |
| 4121 | GATTAGGTAA TTTTATTCTT CTGTCTCGAA GCTCACTGAT |
| 4161 | TCTTTATTCT GTCTAATCTG TTCTGCTGTT GAGCCCATTT |
| 4201 | ATTCCTGATT TTTATATTTT AGTTATTGTA TGTTTTATTT |
| 4241 | CTAAAATTTC CATTCAGTTT TTCTTTATAT CTTCTATTTG |
| 4281 | CTGAGAATTT CTGTCTCTTT GCTGAGACTT TCTACGTTTT |
| 4321 | CATTTGTTTC AAGTGCATTT ATACTTGCTT GTTGAAGAAT |
| 4361 | TTTTATGATG GCTGCTGTAA AATCCTTATC AGATAATTCC |
| 4441 | AACATCTGTC ACCTCATTGT TTGCATCTAC TGATGGTCTT |
| 4441 | TTTTCCATTC GGAAACATTT TCCTGTTTCT TGGTGTGTGG |
| 4481 | AATGATTTTT TATTGAAACC TGGATATTTT TAGGTATTAT |
| 4521 | GTTATGAGAC TATGGGTCTT ATTTAAACCT TCTGCTTTAG |
| 4561 | CCAACTTTCT CAGATACCAC CACAGCAGGG GAATTGGGAG |
| 4601 | CACTGCTTCA TTATTACCAG GTGTCGCTAG GAGTCCAGGT |
| 4641 | TCCCCAGTCA GCCTCCCTTT ATACTGAGTA ACAGGGTCCC |
| 4681 | CTCATTACTA CTGGGCAAGG TGAGAATTCA GTTTCCCATT |
| 4721 | AGGTCTTTAT TGATTCTTCC CTGGCTGGAA TGTGCAGCGG |
| 4761 | CACCTTTTGG TGCACCCTGG GAATCTCCAC TAATGCTATG |
| 4801 | GGACAGAGTG ACCAGGAAGA GCTTCATTAC ACCAGGTGGG |
| 4841 | AATGAAATTC CCAGTAGCCT ACACAGCCTT CTCCGACACC |
| 4831 | ACTCTGGAGT TGTATTCTTC CAGCACACAA ACATACACAA |
| 4921 | TTTAACTCAA AGCATCTTAG CAGAGCTTAA TTAAATGGAT |
| 4961 | AGATGCCTGT TCCCTTTGCT GGATACCAAG AATACAAAAG |
| 5001 | TCAGGGAGTT GGGGCACCTC TTTACAGCTT GGTGAGAGTG |
| 5041 | TAAGTCTGGA CTCCCCACTC AGCATTTGCT GGTATGGGTC |
| 5081 | GGGCCATGGT GTTTTTCCAT GGTGTTTGGT TGGAGTACAG |
| 5121 | CCTTTTTTAC CCTTGCTTGG CTACCCTTTT CTGGTCCTTT |
| 5161 | GGCAGGAGAG AGCAGGACTC TCTTAGGGCT TTTTTTTCCC |
| 5201 | CTGCATTTAT TGACATTTCC AGGTTGCTGA CTTTTTCAGC |
| 5241 | TCCAAGTTGG AAATATATGA GCTGAAAAGA AAATGTAGGG |
| 5281 | AACTCATCAC AGTGTTGTTA CTTGGGCCCC AATGTTCCTA |
| 5321 | GCCTATTTTC TGTCTACTAT TCAGAGTCTT GCTGTGTTTT |
| 5361 | AATATAATAT CCAGGATTTT TATATGCATT TAGCAGAAGG |
| 5401 | ATGTCTACTC TGCCTTTGTA GAAGTGTCTC ACTGATTTTT |
| 5441 | ACATATTTTT CCAGCACACA AACATACACA ATTTAACTCA |
| 5481 | AAGCATCTTA GCAGAGCTTA ATTAAATGGA TAGATGTCTG |
| 5521 | TTCCCTTTGC TGGACGCCAA GAATACAAAA AAGAACAAGT |
| 5561 | GACAATTTTC TCTGTCTTAG GGAGAAGAGA CAGCAGAAGT |
| 5601 | GTAAATGATC CCTAAAGAGT GATAGATGTT ATCATGAAGC |
| 5641 | CACAGGAGGG GTGCCAGGCT GCACAAAAGA GACACTGGAT |
| 5681 | GCTTCTTGGT AGTAGAGGCA GTGGCTTCCC AGCCTTGGGG |
| 5721 | CTAAGGCTTG TAGGGTGAAT TGGAACTTTT CAGATGAGCA |
| 5761 | AGGCAAAGAA GGGACCTTCT AACATTCCTT GGATGGAACA |
| 5801 | TTTTTGACAT TTTCCCATTT ACAGCTACTT ATATTTTCTA |
| 5841 | CAAGTGTCAC TGTGACCAAC TTATGTACAC ATACTTTTTC |
| 5881 | TTGCTTAGTT ATAATAATCT GTTCTTAAAG AAAATGTCAG |
| 5921 | TCTCTACATT CTATGCTGAC TGTTAAGGAA AGAGCACCCA |
| 5961 | CATCTGCTCC TACTTAGCTT TTTTTCTGTG GTTCTTACAC |
| 6001 | AGTATTCCTT TTTTTCTTTT CTTGAAAGAG ACTCCTCCTT |
| 6041 | TCTTTTCTTT TCTTGAAAGA GTTTTAAACA GATAAGATGG |
| 6081 | CAAAAGTGAC TGATCTCTAC TCCCCCAGTT TGAATGGTAA |
| 6121 | ATTTGAATGG TAAATTCCCA TGAACATATA TGGAAATGTC |
| 6161 | TTTATCCTAC TTTCTCCAAT AAAGGCTGTT CTTAGCTTTT |
| 6201 | CAAATGCAAA GTGAAACCTT TATTTATCTT GATTTCTTTT |
| 6241 | TTTTTTTTTT TTTTTTTTTT TTTTTTGAGA TGCTCTGTCA |
| 6281 | CCCAGGCTGG AGTGCAGTGG CAAGATCTTG GCTCACTGCA |
| 6321 | AGCTCCGCCT CCCAGGTTCA CGCCATTCTC CTGGCTCAGC |
| 6361 | CTCCCGAGTA ACTGGGACTA CAGGCACCTG CCGTCACGCC |
| 6401 | TGGCTAATTT TTTGTATTTT TAGTAGAGAA TGGAGTTTCA |
| 6441 | CCGTGTTAGC CAGGATGGTC TCGATCTCCT GACCTTGTGA |
| 6481 | TCTGCCCGCC TCGGCCTCCC AAAGTGCTGG GATTACAGGC |
| 6521 | TCGAGCCACT GCCTCCAGCC TATCCTGATT TCTACTGTCA |
| 6561 | TGCCTCACAT CAGTCCTTTT TTTTTTTTTT GAGACAGAGT |
| 6601 | CTCGCTCTGT GGCCCAGGCT AGACTGCAGT GGCATGATCT |
| 6641 | CGGCTCACTG CAACCTCCAC CTCCGGGGTT CTAGCAATTC |
| 6681 | TCCTGCCTCA GCCTCCTGAG TAGCTGGGAT TATAGGCGCA |
| 6721 | TGCCACACCT GGCTTTTTGT ATTTTAGTGG AGATGGGGTT |
| 6761 | TCACTGTGTT GCTCAGGCTG GTCTTGATCT CCTGAGCTCA |
| 6801 | GACAATCCCC CCGCCTTGGC CTCCCAAAGT GCTAGGATTA |
| 6841 | TAGGCGAGAG CTGCTGTGTG CTTCTTAAGT GAGGTAAGTA |
| 6881 | AGTTCCATAG AAAATTTCCA TCAGTTCATT CATGAAAGAA |
| 6921 | CAAAGAACCT GGCAAAACTT AAAAAAACGT TTCCAAGAAT |
| 6961 | CAGATAAAAG AGGACAAACC TTAGGGAGAA GAAGGCAGCT |
| 7001 | GCTCATTTCC AGCAGGGGAA GTAGCTGCAT AGAGTACAAG |
| 7041 | GACTGGTAGG CCTGTTGGCT GTTCCTGTTT AAGGAGACAA |
| 7081 | GATGGGCATG GAACAGGGAC CACCCCCTCC TCTGGGAGAA |
| 7121 | GCTGTTACCC CCTTCACTTT TCCTCCTCTG TCATTACCCA |
| 7161 | CAATCACTCT CCTTCTTTGC GCTATGGTAG GTGTTTACCC |
| 7201 | ATCATAGGAA TGGGCATTTG AACTTTGAAA CTGAATGTGG |
| 7241 | TGATTACACT TCATGCTGAA GCTTTTCACA TGAGTGCTTT |
| 7281 | CATAAGCATT AAGTAAAATT TTATAATGAC TGCAGTCCAA |
| 7321 | GGACATTTTC CCTGGTTTTT GGCCAGTCTA AATATTGTAA |
| 7361 | GAGAGAGAGA AGAAAAGTGT ACGGAATATA ATTGTCTCTA |
| 7401 | AGCTAAGAAA TGTGGATGTT CAAATAAAAC ATACGTACAG |
| 7441 | AA |

For example, a LTPR protein can include the following human sequence (SEQ ID NO:23; NCBI accession number P36941.1).

| | |
|---|---|
| 1 | MLLPWATSAP GLAWGPLVLG LFGLLAASQP QAVPPYASEN |
| 41 | QTCRDQEKEY YEPQHRICCS RCPPGTYVSA KCSRIRDTVC |
| 81 | ATCAENSYNE HWNYLTICQL CRPCDPVMGL EEIAPCTSKR |
| 121 | KTQCRCQPGM FCAAWALECT HCELLSDCPP GTEAELKDEV |
| 161 | GKGNNHCVPC KAGHFQNTSS PSARCQPHTR CENQGLVEAA |
| 201 | PGTAQSDTTC KNPLEPLPPE MSGTMLMLAV LLPLAFFLLL |
| 241 | ATVFSCIWKS HPSLCRKLGS LLKRRPQGEG PNPVAGSWEP |
| 281 | PKAHPYFPDL VQPLLPISGD VSPVSIGLPA APVLEAGVPQ |
| 321 | QQSPLDLTRE PQLEPGEQSQ VAHGTNGIHV TGGSMTITGN |
| 361 | IYIYNGPVLG GPPGPGDLPA TPEPPYPIPE EGDPGPPGLS |
| 401 | TPHQEDGKAW HLAETEHCGA TPSNRGPRNQ FITHD |

A cDNA sequence that encodes the SEQ ID NO:23 human LTγR protein is shown below as SEQ ID NO:24 (NCBI accession number NM 002342.2).

| | |
|---|---|
| 1 | GCTTTCCCGG CCGCCCCTCC CGCCCCGCAT CGAGGCAGAC |
| 41 | AAGCCTGTTC CTCTTCCCTG GGCTGCGATT GCGACAGGCC |
| 81 | GGCCTGGCTC CCAGCGCTCC CTGTCCCCGC CCCGCGGCCA |
| 121 | GCTCGCTCCA CTCCCACTTC CTGAGCTCCG CCATGGGAGC |
| 161 | CCTGGAGGCC CGGCCTGGCC GCTCCCGGCC CTGGGGTGCA |
| 201 | CATCGGCCCT GAGTCCCGTC CCAGGCTCTG GGCTCGGGCA |
| 241 | GCCGCCGCCA CCGCTGCCCA GGACGTCGGG CCTCCTGCCT |
| 281 | TCCTCCCAGG CCCCCACGTT GCTGGCCGCC TGGCCGAGTG |
| 321 | GCCGCCATGC TCCTGCCTTG GGCCACCTCT GCCCCCGGCC |
| 361 | TGGCCTGGGG GCCTCTGGTC CTGGGCCTCT TCGGGCTCCT |
| 401 | GGCAGCATCG CAGCCCCAGG CGGTGCCTCC ATATGCGTCG |
| 441 | GAGAACCAGA CCTGCAGGGA CCAGGAAAAG GAATACTATG |
| 481 | AGCCCCAGCA CCGCATCTGC TGCTCCCGCT GCCCGCCAGG |
| 521 | CACCTATGTC TCAGCTAAAT GTAGCCGCAT CCGGGACACA |
| 561 | GTTTGTGCCA CATGTGCCGA GAATTCCTAC AACGAGCACT |
| 601 | GGAACTACCT GACCATCTGC CAGCTGTGCC GCCCCTGTGA |
| 641 | CCCAGTGATG GGCCTCGAGG AGATTGCCCC CTGCACAAGC |
| 681 | AAACGGAAGA CCCAGTGCCG CTGCCAGCCG GGAATGTTCT |
| 721 | GTGCTGCCTG GGCCCTCGAG TGTACACACT GCGAGCTACT |
| 761 | TTCTGACTGC CCGCCTGGCA CTGAAGCCGA GCTCAAAGAT |
| 801 | GAAGTTGGGA AGGGTAACAA CCACTGCGTC CCCTGCAAGG |
| 841 | CCGGGCACTT CCAGAATACC TCCTCCCCCA GCGCCCGCTG |
| 881 | CCAGCCCCAC ACCAGGTGTG AGAACCAAGG TCTGGTGGAG |
| 921 | GCAGCTCCAG GCACTGCCCA GTCCGACACA ACCTGCAAAA |
| 961 | ATCCATTAGA GCCACTGCCC CCAGAGATGT CAGGAACCAT |
| 1001 | GCTGATGCTG GCCGTTCTGC TGCCACTGGC CTTCTTTCTG |
| 1041 | CTCCTTGCCA CCGTCTTCTC CTGCATCTGG AAGAGCCACC |
| 1081 | CTTCTCTCTG CAGGAAACTG GGATCGCTGC TCAAGAGGCG |
| 1121 | TCCGCAGGGA GAGGGACCCA ATCCTGTAGC TGGAAGCTGG |
| 1161 | GAGCCTCCGA AGGCCCATCC ATACTTCCCT GACTTGGTAC |
| 1201 | AGCCACTGCT ACCCATTTCT GGAGATGTTT CCCCAGTATC |
| 1241 | CACTGGGCTC CCCGCAGCCC CAGTTTTGGA GGCAGGGGTG |
| 1281 | CCGCAACAGC AGAGTCCTCT GGACCTGACC AGGGAGCCGC |
| 1321 | AGTTGGAACC CGGGGAGCAG AGCCAGGTGG CCCACGGTAC |
| 1361 | CAATGGCATT CATGTCACCG GCGGGTCTAT GACTATCACT |
| 1401 | GGCAACATCT ACATCTACAA TGGACCAGTA CTGGGGGGAC |
| 1441 | CACCGGGTCC TGGAGACCTC CCAGCTACCC CCGAACCTCC |
| 1481 | ATACCCCATT CCCGAAGAGG GGGACCCTGG CCCTCCCGGG |
| 1521 | CTCTCTACAC CCCACCAGGA AGATGGCAAG GCTTGGCACC |
| 1561 | TAGCGGAGAC AGAGCACTGT GGTGCCACAC CCTCTAACAG |
| 1601 | GGGCCCAAGG AACCAATTTA TCACCCATGA CTGACTGAGT |
| 1641 | CTGAGAAAAG GCAGAAGAAG GGGGGCACAA GGGCACCTTC |
| 1681 | TCCCTTGAGG CTGCCCTGCC CACGTGGGAT TCACAGGGGC |
| 1721 | CTGAGTAGGG CCCGGGGAAG CAGAGCCCTA AGGGATTAAG |
| 1761 | GCTCAGACAC CTCTGAGAGC AGGTGGGCAC TGGCTGGGTA |
| 1801 | CGGTGCCCTC CACAGGACTC TCCCTACTGC CTGAGCAAAC |
| 1841 | CTGAGGCCTC CCGGCAGACC CACCCACCCC CTGGGGCTGC |
| 1881 | TCAGCCTCAG GCACGGACAG GGCACATGAT ACCAACTGCT |
| 1921 | GCCCACTACG GCACGCCGCA CCGGAGCACG GCACCGAGGC |
| 1961 | AGCCGCCACA CGGTCACCTG CAAGGACGTC ACGGGCCCCT |
| 2001 | CTAAAGGATT CGTGGTGCTC ATCCCCAAGC TTCAGAGACC |
| 2041 | CTTTGGGGTT CCACACTTCA CGTGGACTGA GGTAGACCCT |
| 2081 | GCATGAAGAT GAAATTATAG GGAGGACGCT CCTTCCCTCC |
| 2121 | CCTCCTAGAG GAGAGGAAAG GGAGTGATTA ACAACTAGGG |
| 2161 | GGTTGGGTAG GATTCCTAGG TATGGGGAAG AGTTTTGGAA |
| 2201 | GGGGAGGAAA ATGGCAAGTG TATTTATATT GTAACCACAT |
| 2241 | CCAAATAAAA ACAATGGGAC CTAGATAAAA AAAAAAAAAA |
| 2281 | AAA |

STING, cGAS, NF-κB transcription factor p52, NF-κB transcription factor ReIB, ENPP1, LT3R, BAFFR, CD40, RANK, FN14, NIK (MAP3K14), and MST1 proteins and nucleic acids can exhibit sequence variation. However, variants with less than 100% sequence identity to the amino acid and nucleic acid sequences shown herein can still have similar activities. For example, STING, cGAS, NF-κB transcription factor p52, NF-κB transcription factor ReIB, ENPP1, LTPR, BAFFR, CD40, RANK, FN14, NIK (MAP3K14), and MST1 proteins and nucleic acid with at least 90%, at least 92%, at least 95%, at least 96%, at least 97%, at least 98%, at least 99%, or at least 99.5% sequence identity to any of SEQ ID NOs: 13-24 can still be used in the compositions and methods described herein.

### Expression Systems

Nucleic acid segments encoding any kinsin-13, MCAK, ABCC4, and/or ABCG2 protein, as well as nucleic acids encoding STING, cGAS, NF-κB transcription factor p52, NF-κB transcription factor ReIB proteins, ENPP1, LTβR, BAFFR, CD40, RANK, FN14, NIK (MAP3K14), MST1, or nucleic acid segments including any STING, cGAS, NF-κB transcription factor p52, NF-κB transcription factor ReIB, ENPP1, LT3R, BAFFR, CD40, RANK, FN14, NIK (MAP3K14), MST1 inhibitory nucleic acid can be inserted into or employed with any suitable expression system. A therapeutically effective quantity of kinsin-13, MCAK, ABCC4, and/or ABCG2 protein can be generated from such expression systems. A therapeutically effective STING, cGAS, NF-κB transcription factor p52, NF-κB transcription factor ReIB, ENPP1, LTPR, BAFFR, CD40, RANK, FN14, NIK (MAP3K14), and/or MST1 inhibitory nucleic acid can also be generated from such expression systems.

Recombinant expression of nucleic acids (or inhibitory nucleic acids) is usefully accomplished using a vector, such as a plasmid. The vector can include a promoter operably linked to nucleic acid segment encoding a kinsin-13, MCAK, ABCC4, and/or ABCG2 protein, or a protein such as a STING, cGAS, NF-κB transcription factor p52, and/or NF-κB transcription factor ReIB, ENPP1, LTβR, BAFFR, CD40, RANK, FN14, NIK (MAP3K14), and/or MST1. In another example, a vector can include a promoter operably linked to nucleic acid segment that encodes a STING, cGAS, NF-κB transcription factor p52, and/or NF-κB transcription factor ReIB, ENPP1, LTPR, BAFFR, CD40, RANK, FN14, NIK (MAP3K14), MST1 inhibitory nucleic acid.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing kinsin-13, KIF13A, MCAK, ABCC4, and/or ABCG2. A variety of prokaryotic and eukaryotic expression vectors suitable for carrying, encoding and/or expressing STING, cGAS, NF-κB transcription factor p52, and/or NF-κB transcription factor ReIB, ENPP1, LTPR, BAFFR, CD40, RANK, FN14, NIK (MAP3K14), and/or MST1 inhibitory nucleic acids can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms; the cDNA sequences may be expressed in either a sense (to produce mRNA) or anti-sense orientation (to produce an anti-sense RNA transcript that is complementary to the mRNA transcript). Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding a kinsin-13, MCAK, ABCC4, and/or ABCG2 protein. In another example, the promoter can be upstream of a STING, cGAS, NF-κB transcription factor p52, and/or NF-κB transcription factor ReIB, ENPP1, LT3R, BAFFR, CD40, RANK, FN14, NIK (MAP3K14), and/or MST1 inhibitory nucleic acid segment.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of a kinsin-13, KIF13A, MCAK, ABCC4, and/or ABCG2 protein, or of STING, cGAS, NF-κB transcription factor p52, and/or NF-κB transcription factor ReIB, ENPP1, LTβR, BAFFR, CD40, RANK, FN14, NIK (MAP3K14), and/or MST1 inhibitory nucleic acids from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pClneo-CMV.

The expression cassette or vector can include nucleic acid sequence encoding a marker product. This marker product is used to determine if the gene has been delivered to the cell and once delivered is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the kinesin-13-related (e.g., kinsin-13, MCAK, ABCC4, ABCG2, or KIF13A), nucleic acid molecule, expression cassette and/or vector, and/or the STING, cGAS, NF-κB transcription factor p52, NF-κB transcription factor ReIB, ENPP1, LT3R, BAFFR, CD40, RANK, FN14, NIK (MAP3K14), and/or MST1 inhibitory nucleic acid molecule, expression cassette and/or vector can be introduced to a cell by any method including, but not limited to, calcium-mediated transformation, electroporation, microinjection, lipofection, particle bombardment and the like. The cells can be expanded in culture and then administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain kinesin-13-related (e.g., kinsin-13, MCAK, ABCC4, ABCG2, or KIF13A) nucleic acid molecules, expression cassettes and/or vectors, and/or that produce STING, cGAS, NF-κB transcription factor p52, NF-κB transcription factor ReIB, ENPP1, LTβR, BAFFR, CD40, RANK, FN14, NIK (MAP3K14), and/or MST1 inhibitory nucleic acids. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem. 286(16): 14383-14395 (2011); Hu et al., Frontiers in Genetics 3 (April 2012); Pegtel et al., Proc. Nat'l Acad Sci 107(14): 6328-6333 (2010); WO/2013/084000; each of which is incorporated herein by reference in its entirety. Cells producing such microvesicles can be used to express the STING, cGAS, NF-κB transcription factor p52, and/or NF-κB transcription factor kinesin-13-related (e.g., kinsin-13, MCAK, ABCC4, ABCG2, or KIF13A), ReIB, ENPP1, LTPR, BAFFR, CD40, RANK, FN14, NIK (MAP3K14), and/or MST1 proteins and/or inhibitory nucleic acids.

Transgenic vectors or cells with a heterologous expression cassette or expression vector that expresses the kinesin-13 protein(s) (e.g., Kif2b, MCAK/Kif2c, kinsin-13, MCAK, ABCC4, ABCG2, or KIF13A) that can optionally also express STING, cGAS, NF-κB transcription factor p52, NF-κB transcription factor ReIB, ENPP1, LTPR, BAFFR, CD40, RANK, FN14, and/or NIK (MAP3K14), and/or MST1 inhibitory nucleic acids can be administered to a subject. Transgenic vectors or cells with a heterologous expression cassette or expression vector can also optionally express ENPP1. Exosomes produced by transgenic cells can be used to deliver kinesin-13/MCAK nucleic acids or protein(s) (e.g., Kif2b, MCAK/Kif2c, ABCC4, ABCG2, and/or KIF13A nucleic acids or protein(s)) to tumor and cancer cells in the subject. Exosomes produced by transgenic cells can be used to deliver STING, cGAS, NF-κB transcription factor p52, NF-κB transcription factor ReIB, ENPP1, LTPR, BAFFR, CD40, RANK, FN14, NIK (MAP3K14), and/or MST1 inhibitory nucleic acids to tumor and cancer cells in the subject.

Methods and compositions that include inhibitors of STING, cGAS, NF-κB transcription factor p52, NF-κB transcription factor ReIB, ENPP1, LTβR, BAFFR, CD40, RANK, FN14, NIK (MAP3K14), MST1, or any combination thereof can involve use of antibodies or inhibitory nucleic acids directed against STING, cGAS, NF-κB transcription factor p52, NF-κB transcription factor ReIB, ENPP1, LTβR, BAFFR, CD40, RANK, FN14, NIK (MAP3K14), MST1, or any combination thereof.

### Inhibitory Nucleic Acids

The expression of the following can be inhibited STING, cGAS, NF-κB transcription factor p52, NF-κB transcription factor ReIB, ENPP1, LTβR, BAFFR, CD40, RANK, FN14, NIK (MAP3K14), MST1, or any combination thereof, for example by use of an inhibitory nucleic acid that specifically recognizes a nucleic acid that encodes STING, cGAS, NF-κB transcription factor p52, NF-κB transcription factor ReIB, ENPP1, LTβR, BAFFR, CD40, RANK, FN14, NIK (MAP3K14), or MST1.

An inhibitory nucleic acid can have at least one segment that will hybridize to a STING, cGAS, NF-κB transcription factor p52, NF-κB transcription factor ReIB, ENPP1, LTPR, BAFFR, CD40, RANK, FN14, NIK (MAP3K14), or MST1 nucleic acid under intracellular or stringent conditions. The inhibitory nucleic acid can reduce expression of a nucleic acid encoding STING, cGAS, NF-κB transcription factor p52, NF-κB transcription factor ReIB, ENPP1, LTβR, BAFFR, CD40, RANK, FN14, NIK (MAP3K14), or MST1. A nucleic acid may hybridize to a genomic DNA, a messenger RNA, or a combination thereof. An inhibitory nucleic acid may be incorporated into a plasmid vector or viral DNA. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally-occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a STING, cGAS, NF-κB transcription factor p52, NF-κB transcription factor ReIB, ENPP1, LTβR, BAFFR, CD40, RANK, FN14, NIK (MAP3K14), or MST1 nucleic acid. Such an inhibitory nucleic acid may be completely complementary to a segment of the STING, cGAS, NF-κB transcription factor p52, NF-κB transcription factor ReIB, ENPP1, LTβR, BAFFR, CD40, RANK, FN14, NIK (MAP3K14), or MST1 nucleic acid. Alternatively, some variability is permitted in the inhibitory nucleic acid sequences relative to STING, cGAS, NF-κB transcription factor p52, NF-κB transcription factor ReIB, ENPP1, LTβR, BAFFR, CD40, RANK, FN14, NIK (MAP3K14), or MST1 sequences. An inhibitory nucleic acid can hybridize to a STING, cGAS, NF-κB transcription factor p52, NF-κB transcription factor ReIB, ENPP1, LTPR, BAFFR, CD40, RANK, FN14, NIK (MAP3K14), or MST1 nucleic acid under intracellular conditions or under stringent hybridization conditions, and is sufficiently complementary to inhibit expression of a STING, cGAS, NF-κB transcription factor p52, NF-κB transcription factor ReIB, ENPP1, LTβR, BAFFR, CD40, RANK, FN14, NIK (MAP3K14), or MST1 nucleic acid. Intracellular conditions refer to conditions such as temperature, pH and salt concentrations typically found inside a cell, e.g. an animal or mammalian cell. One example of such an animal or mammalian cell is a myeloid progenitor cell. Another example of such an animal or mammalian cell is a more differentiated cell derived from a myeloid progenitor cell. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a STING, cGAS, NF-κB transcription factor p52, NF-κB transcription factor ReIB, ENPP1, LT3R, BAFFR, CD40, RANK, FN14, NIK (MAP3K14), or MST1 coding sequence, each separated by a stretch of contiguous nucleotides that are not complementary to adjacent coding sequences, can inhibit the function of a STING, cGAS, NF-κB transcription factor p52, NF-κB transcription factor ReIB, ENPP1, LTβR, BAFFR, CD40, RANK, FN14, NIK (MAP3K14), or MST1 nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

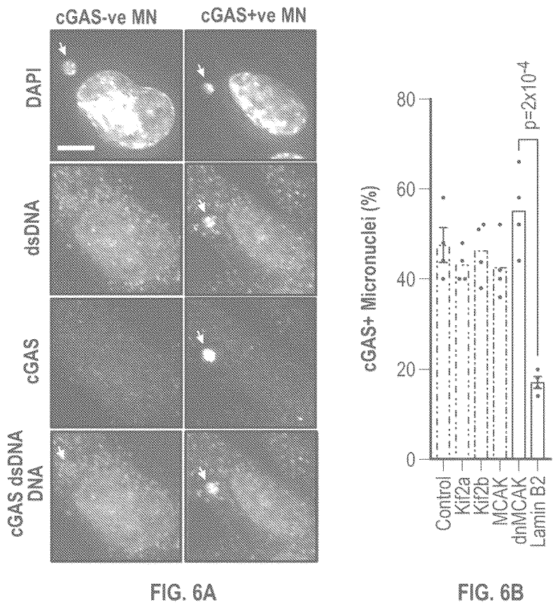

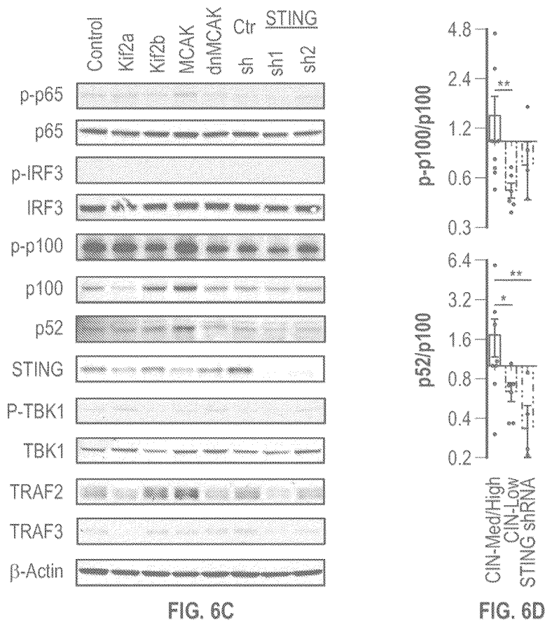

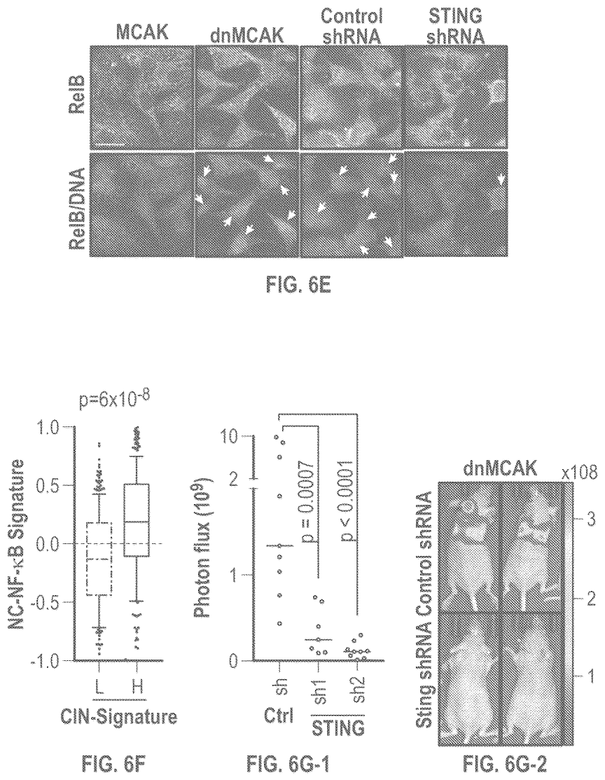

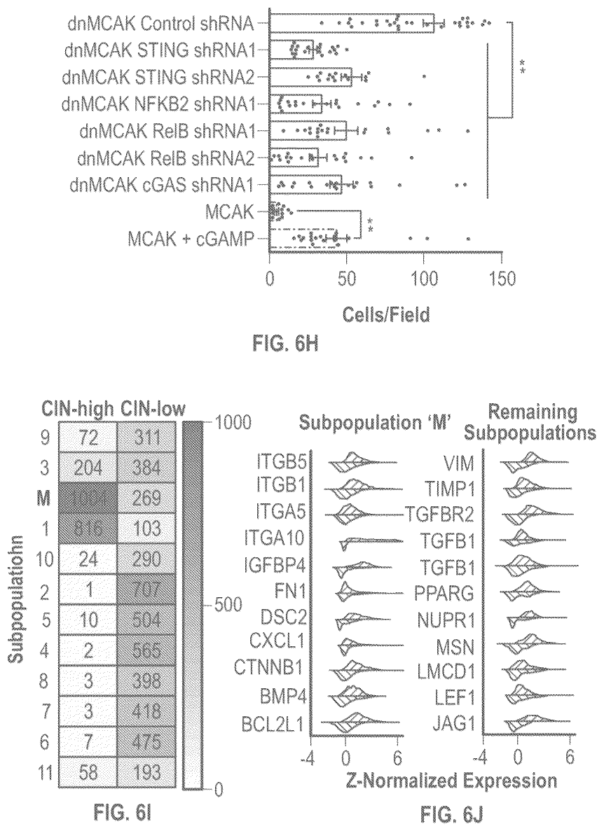

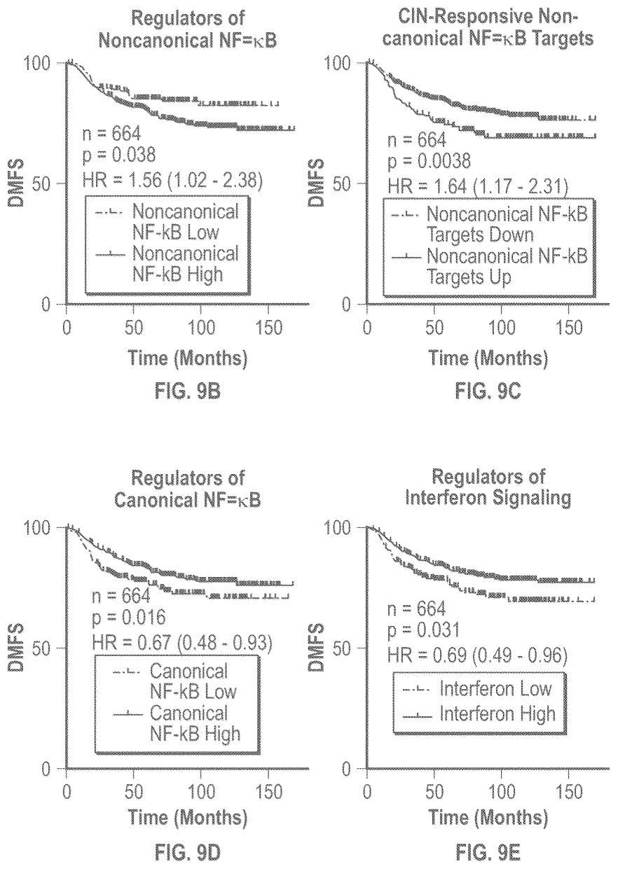

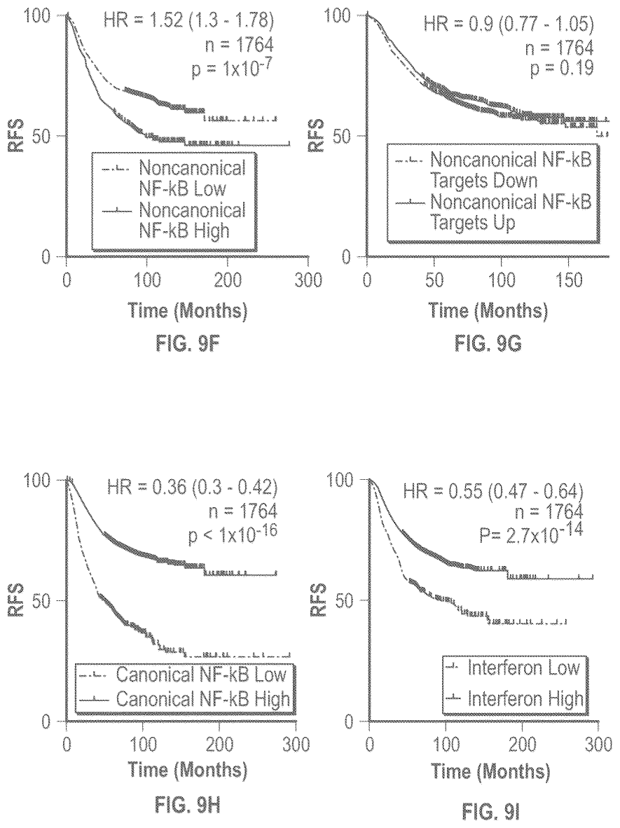

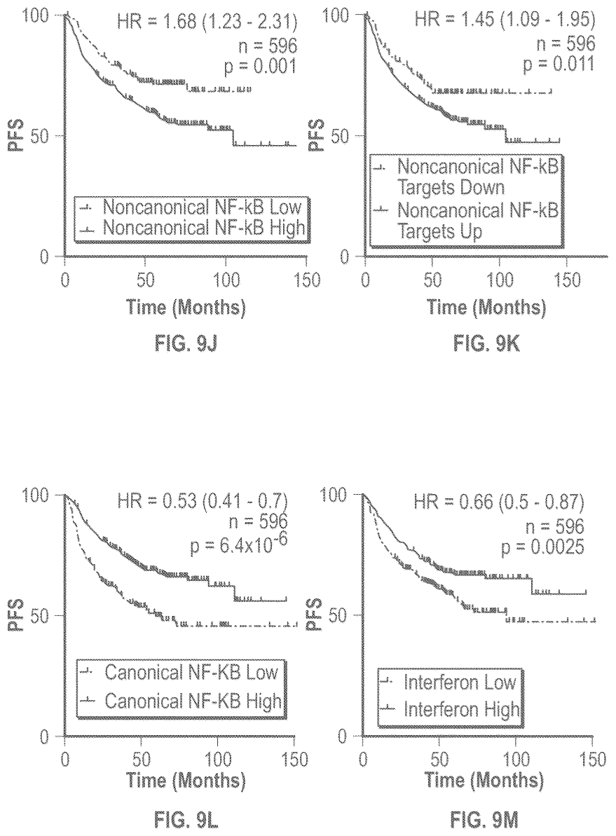

Examples of a nucleic acid encoding STING, cGAS, NF-κB transcription factor p52, NF-κB transcription factor ReIB, ENPP1, LTβR, BAFFR, CD40, RANK, FN14, NIK (MAP3K14), or MST1 are shown herein. Example 1 provides examples of inhibitory nucleic acid sequences, including SEQ ID NOs:25-36. See also FIGS. 6 and 9.

The inhibitory nucleic acid molecule may be single or double stranded (e.g. a small interfering RNA (siRNA)), and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex.

Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-0 alkyl (usually in chimeras with RNase-H dependent antisense), peptide nucleic acid (PNA), locked nucleic acid (LNA) and morpholino antisense.

Small interfering RNAs, for example, may be used to specifically reduce STING, cGAS, NF-κB transcription factor p52, NF-κB transcription factor ReIB, ENPP1, LTPR, BAFFR, CD40, RANK, FN14, NIK (MAP3K14), or MST1 translation such that translation of the encoded polypeptide is reduced. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the STING, cGAS, NF-κB transcription factor p52, NF-κB transcription factor ReIB, ENPP1, LTPR, BAFFR, CD40, RANK, FN14, NIK (MAP3K14), or MST1 mRNA transcript. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting STING, cGAS, NF-κB transcription factor p52, NF-κB transcription factor ReIB, ENPP1, LTβR, BAFFR, CD40, RANK, FN14, NIK (MAP3K14), or MST1 expression. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/mai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO:60). SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target STING, cGAS, NF-κB transcription factor p52, NF-κB transcription factor ReIB, ENPP1, LTβR, BAFFR, CD40, RANK, FN14, NIK (MAP3K14), or MST1 nucleic acid.

An inhibitory nucleic acid may be prepared using available methods, for example, by expression from an expression vector encoding the sequence of the STING, cGAS, NF-κB transcription factor p52, NF-κB transcription factor ReIB, ENPP1, LTβR, BAFFR, CD40, RANK, FN14, NIK (MAP3K14), and/or MST1 nucleic acid, or a complement thereof. Alternatively, it may be prepared by chemical synthesis using naturally-occurring nucleotides, modified nucleotides or any combinations thereof. In some embodiments, the STING, cGAS, NF-κB transcription factor p52, NF-κB transcription factor ReIB, and ENPP1, LTβR, BAFFR, CD40, RANK, FN14, NIK (MAP3K14), and/or MST1 nucleic acids are made from modified nucleotides or non-phosphodiester bonds, for example, that are designed to increase biological stability of the STING, cGAS, NF-κB transcription factor p52, NF-κB transcription factor ReIB, ENPP1, LTPR, BAFFR, CD40, RANK, FN14, NIK (MAP3K14), and/or MST1 nucleic acids or to increase intracellular stability of the duplex formed between the inhibitory nucleic acids and other (e.g., endogenous) nucleic acids.

For example, the STING, cGAS, NF-κB transcription factor p52, NF-κB transcription factor ReIB, ENPP1, LTβR, BAFFR, CD40, RANK, FN14, NIK (MAP3K14), and/or MST1 nucleic acids can be peptide nucleic acids that have peptide bonds rather than phosphodiester bonds.

Naturally-occurring nucleotides that can be employed in STING, cGAS, NF-κB transcription factor p52, NF-κB transcription factor ReIB, ENPP1, LTβR, BAFFR, CD40, RANK, FN14, NIK (MAP3K14), and/or MST1 nucleic acids include the ribose or deoxyribose nucleotides adenosine, guanine, cytosine, thymine and uracil. Examples of modified nucleotides that can be employed in STING, cGAS, NF-κB transcription factor p52, NF-κB transcription factor ReIB, ENPP1, LTβR, BAFFR, CD40, RANK, FN14, NIK (MAP3K14), and/or MST1 nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, STING, cGAS, NF-κB transcription factor p52, NF-κB transcription factor, ReIB nucleic acids as well as the ENPP1, LTβR, BAFFR, CD40, RANK, FN14, NIK (MAP3K14), and/or MST1 inhibitory nucleic acids may include modified nucleotides, as well as natural nucleotides such as combinations of ribose and deoxyribose nucleotides. The inhibitory nucleic acids and may be of same length as wild type (e.g., SEQ ID NO:14, 16, 18, 20, 22 or 24). The STING, cGAS, NF-κB transcription factor p52, and NF-κB transcription factor ReIB nucleic acids as well as the ENPP1, LTPR, BAFFR, CD40, RANK, FN14, NIK (MAP3K14), and/or MST1 inhibitory nucleic acids can also be longer and include other useful sequences. In some embodiments, the STING, cGAS, NF-κB transcription factor p52, NF-κB transcription factor ReIB, ENPP1, LTβR, BAFFR, CD40, RANK, FN14, NIK (MAP3K14), and/or MST1 nucleic acids are somewhat shorter. For example, the STING, cGAS, NF-κB transcription factor p52, NF-κB transcription factor ReIB, ENPP1, LTPR, BAFFR, CD40, RANK, FN14, NIK (MAP3K14), and/or MST1 inhibitory nucleic acids can include a segment that has nucleic acid sequence (e.g., SEQ ID NO:14, 16, 18, 20, 22, or 24) that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

### Antibodies

Antibodies can be used as inhibitors of STING, cGAS, NF-κB transcription factor p52, NF-κB transcription factor ReIB, ENPP1, LTβR, BAFFR, CD40, RANK, FN14, and NIK (MAP3K14), MST1. Antibodies can be raised against various epitopes of the STING, cGAS, NF-κB transcription factor p52, NF-κB transcription factor ReIB, ENPP1, LTβR, BAFFR, CD40, RANK, FN14, and NIK (MAP3K14), MST1 proteins. Some antibodies for STING, cGAS, NF-κB transcription factor p52, NF-κB transcription factor ReIB, ENPP1, LTβR, BAFFR, CD40, RANK, FN14, and NIK (MAP3K14), MST1 proteins may also be available commercially. However, the antibodies contemplated for treatment pursuant to the methods and compositions described herein are preferably human or humanized antibodies, and are highly specific for their targets.

In one aspect, the present disclosure relates to use of isolated antibodies that bind specifically to STING, cGAS, NF-κB transcription factor p52, NF-κB transcription factor ReIB proteins, ENPP1, LTβR, BAFFR, CD40, RANK, FN14, NIK (MAP3K14), or MST1. Such antibodies may be monoclonal antibodies. Such antibodies may also be humanized or fully human monoclonal antibodies. The antibodies can exhibit one or more desirable functional properties, such as high affinity binding to STING, cGAS, NF-κB transcription factor p52, NF-κB transcription factor ReIB proteins, ENPP1, LTβR, BAFFR, CD40, RANK, FN14, NIK (MAP3K14), or MST1, or the ability to inhibit binding of STING, cGAS, NF-κB transcription factor p52, NF-κB transcription factor ReIB proteins, ENPP1, LTβR, BAFFR, CD40, RANK, FN14, NIK (MAP3K14), or MST1 receptor.

Methods and compositions described herein can include STING, cGAS, NF-κB transcription factor p52, NF-κB transcription factor ReIB proteins, ENPP1, LTβR, BAFFR, CD40, RANK, FN14, NIK (MAP3K14), or MST1 antibodies, or a combination of STING, cGAS, NF-κB transcription factor p52, NF-κB transcription factor ReIB proteins, ENPP1, LTβR, BAFFR, CD40, RANK, FN14, NIK (MAP3K14), MST1 antibodies.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. a domain of STING, cGAS, NF-κB transcription factor p52, NF-κB transcription factor ReIB proteins, ENPP1, LTPR, BAFFR, CD40, RANK, FN14, NIK (MAP3K14), or MST1). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CHI domains; (ii) a F(ab′)2 fragment, a bivalent fragment comprising two Fab fragments linked by a disulfide bridge at the hinge region; (iii) a Fd fragment consisting of the V_{H} and CHI domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds STING, cGAS, NF-κB transcription factor p52, NF-κB transcription factor ReIB proteins, ENPP1, LTβR, BAFFR, CD40, RANK, FN14, NIK (MAP3K14), or MST1 is substantially free of antibodies that specifically bind antigens other than STING, cGAS, NF-κB transcription factor p52, NF-κB transcription factor ReIB proteins, ENPP1, LTβR, BAFFR, CD40, RANK, FN14, NIK (MAP3K14), or MST1). An isolated antibody that specifically binds STING, cGAS, NF-κB transcription factor p52, NF-κB transcription factor ReIB proteins, ENPP1, LTβR, BAFFR, CD40, RANK, FN14, NIK (MAP3K14), or MST1 may, however, have cross-reactivity to other antigens, such as STING, cGAS, NF-κB transcription factor p52, NF-κB transcription factor ReIB proteins, ENPP1, LTβR, BAFFR, CD40, RANK, FN14, NIK (MAP3K14), or MST1-family molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.” The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human STING, cGAS, NF-κB transcription factor p52, NF-κB transcription factor ReIB proteins, ENPP1, LT3R, BAFFR, CD40, RANK, FN14, NIK (MAP3K14), or MST1” is intended to refer to an antibody that binds to human STING, cGAS, NF-κB transcription factor p52, NF-κB transcription factor ReIB proteins, ENPP1, LTβR, BAFFR, CD40, RANK, FN14, NIK (MAP3K14), or MST1 with a K_{D} of 1×10⁻⁷ M or less, more preferably 5×10⁻⁸ or less, more preferably 1×10⁻⁸ or less, more preferably 5×10⁻⁹ M or less, even more preferably between 1×10⁻⁸ and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human STING, cGAS, NF-κB transcription factor p52, NF-κB transcription factor ReIB proteins, ENPP1, LTβR, BAFFR, CD40, RANK, FN14, NIK (MAP3K14), or MST1. Preferably, an antibody of the invention binds to STING, cGAS, NF-κB transcription factor p52, NF-κB transcription factor ReIB proteins, ENPP1, LTβR, BAFFR, CD40, RANK, FN14, NIK (MAP3K14), or MST1 with high affinity, for example with a K_{D} of 1×10⁻⁷ M or less. The antibodies can exhibit one or more of the following characteristics:
(a) binds to human STING, cGAS, NF-κB transcription factor p52, NF-κB transcription factor ReIB proteins, ENPP1, LTβR, BAFFR, CD40, RANK, FN14, NIK (MAP3K14), or MST1 with a K_{D} of 1×10⁻⁷ M or less;
(b) inhibits the function or activity of STING, cGAS, NF-κB transcription factor p52, NF-κB transcription factor ReIB proteins, ENPP1, LTβR, BAFFR, CD40, RANK, FN14, NIK (MAP3K14), or MST1;
(c) inhibits cancer (e.g., metastatic cancer); or
(d) a combination thereof.

Assays to evaluate the binding ability of the antibodies toward STING, cGAS, NF-κB transcription factor p52, NF-κB transcription factor ReIB proteins, ENPP1, LTβR, BAFFR, CD40, RANK, FN14, NIK (MAP3K14), or MST1 can be used, including for example, ELISAs, Western blots and RIAs. The binding kinetics (e.g., binding affinity) of the antibodies also can be assessed by standard assays known in the art, such as by Biacore™. analysis.

Given that each of the subject antibodies can bind to STING, cGAS, NF-κB transcription factor p52, NF-κB transcription factor ReIB proteins, ENPP1, LTPR, BAFFR, CD40, RANK, FN14, NIK (MAP3K14), or MST1, the V_{L} and V_{H} sequences can be “mixed and matched” to create other binding molecules that bind to STING, cGAS, NF-κB transcription factor p52, NF-κB transcription factor ReIB proteins, ENPP1, LTPR, BAFFR, CD40, RANK, FN14, NIK (MAP3K14), or MST1. The binding properties of such “mixed and matched” antibodies can be tested using the binding assays described above and assessed in assays described in the examples. When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds STING, cGAS, NF-κB transcription factor p52, NF-κB transcription factor ReIB proteins, ENPP1, LTPR, BAFFR, CD40, RANK, FN14, NIK (MAP3K14), or MST1.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphavbeta3 antibodies using a heavy and light chain variable CDR3 domain. Hence, in some cases a mixed and matched antibody or a humanized antibody contains a CDR3 antigen binding domain that is specific for STING, cGAS, NF-κB transcription factor p52, NF-κB transcription factor ReIB proteins, ENPP1, LTPR, BAFFR, CD40, RANK, FN14, NIK (MAP3K14), or MST1.

### Small Molecules

Small molecule modulators of STING, cGAS, NF-κB transcription factor p52, and NF-κB transcription factor ReIB, ENPP1, LTβR, BAFFR, CD40, RANK, FN14, NIK (MAP3K14), and/or MST1 are also available. For example, the SK4A compound is a specific inhibitor of ENNP1 (Arad et al., SAT0037An ENPP1-Specific Inhibitor Attenuates Extracellular Ecto-Pyrophosphatase Activity in Human Osteoarthftic Cartilage, see website at ard.bmj.com/content/74/Suppl_2/662.1 (2015)).

In addition, the following compound (L524-0366) is an FN14 antagonist.

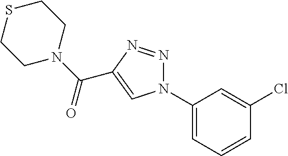 [SMILES: O=C(c1cn(-c2cccc(Cl)c2)nn1)N1CCSCC1]

### Assays for Drug Development

Methods are also described herein for screening metastatic tumor samples for susceptibility to treatment with candidate compounds. Specifically, the methods can include assay steps for identifying a candidate compound that selectively interferes with proliferation or viability of cells exhibiting increased chromosomal instability (e.g., CIN-mutant cells) or metastatic cells that have elevated levels of cGAMP.

If proliferation or viability of cells exhibiting increased chromosomal instability (e.g., CIN-mutant cells) is decreased in the presence of a test compound as compared to a normal control cell then that test compound has utility for reducing the growth and/or metastasis of cells exhibiting such increased chromosomal instability.

Similarly, if a cell or population of cells has elevated levels cGAMP then that cell or cell population is cancerous or will develop cancer. When cGAMP levels of such a cell or population of cells exhibits decreased levels of cGAMP as compared to previous levels for the cGAMP secreting cells, then that test compound has utility for reducing the growth and/or metastasis of cells that have elevated levels of cGAMP.

An assay can include determining whether a compound can specifically cause decreased levels of cGAMP from metastatic or CIN cancer cells, or cell lines.

If the compound does cause decreased levels, then the compound can be selected/identified for further study, such as for its suitability as a therapeutic agent to treat a cancer. For example: the candidate compounds identified by the selection methods featured in the invention can be further examined for their ability to target a tumor or to treat cancer by, for example, administering the compound to an animal model.

The cells that are evaluated can include cells from a patient with cancer (including a patient with metastatic cancer), or cells from a known cancer type or cancer cell line, or cells exhibiting an overproduction of cGAMP. A compound that can reduce the production of cGAMP from any of these cell types can be administered to a patient.

For example, one method can include (a) obtaining a cell or tissue sample from a patient: (b) measuring the amount or concentration of cGAMP produced from a known number or weight of cells or tissues from the sample to generate a reference cGAMP value; (c) mixing the same known number or weight of cells or tissues from the sample with a test compound to generate a test assay; (d) measuring the cGAMP amount or concentration in the test assay (either in the cell medium or in the cells or tissues) to generate a test assay cGAMP value, (e) optionally repeating steps (c) and (d); and selecting a test compound with a lower test assay cGAMP value than the reference cGAMP value. The method can further include administering a test compound to an animal model, for example, to further evaluate the toxicity and/or efficacy of the test compound. In some cases, the method can further include administering the test compound to the patent from whom the cell or tissue sample as obtained.

For example, another method can include assays useful for identifying KIF2B and KIF2C/MCAK agonists or activators. KIF2B and KIF2C/MCAK are related molecular kinesin motor proteins that utilize the energy of ATP hydrolysis to regulate microtubule dynamics and chromosome-kinetochore attachments. The central role of KIF2B and MCAK over expression or hyper activation is suppressing chromosomal instability (CIN) makes them attractive targets for cancer therapy. An in vitro assay and imaging method are described below that can be used to identify and assess potent activators of KIF2B and MCAK.

Measuring the kinetics of ATP hydrolysis can be used to screen for compounds that activate KIF2B and MCAK and that suppress CIN This assay is based upon an absorbance shift (330 to 360 nm) that occurs when 2-amino-6-mercapto-7-methylpurine ribonucleoside (MESG) is converted to 2-amino-6-mercapto-7-methyl purine in the presence of inorganic phosphate. The reaction is catalyzed by purine nucleoside phosphorylase (PNP). One molecule of inorganic phosphate (Pi) will yield one molecule of 2-amino-6-mercapto-7-methyl purine in a irreversible reaction. Thus, the absorbance at 360 nm is directly proportional to the amount of Pi generated in the ATPase reaction: and can be used as a proxy for MCAK activity.

Alternatively, ADP production can also be monitored as a readout for MCAK activity using the Transcreener ADP assay from BellBrook Labs. This assay is based on the ability of ADP to displace a fluorescent tracer (633 nm) bound to an antibody the specifically recognizes ADP Displacement of the tracer causes a decrease in fluorescence measured by laser excitation at 633 nm. Thus, activity of MCAK can be calculated by plotting the concentration of drug used and the amount of ADP produced/decrease in fluorescent intensity.

The following is another example of a method for identifying and assessing the potency of MCAK activators. MCAK negatively regulates microtubule length by binding microtubule tips and promoting microtubule depolymerization. Therefore, distance between γ-tubulin-labeled centrosomes can be measured as an indirect readout for MCAK activity in cells. Spindle length is inversely proportional to MCAK activity and can serve as proxy to evaluate potential compounds that promote MCAK activity. This method can be adapted for screening compounds by using a high-throughput imaging microscope.

Compounds (e.g, top hits identified by any method described herein) can be used in a cell-based assay using lagging chromosomes, micronuclei, or chromosome missegregation with Fluorescent in situ hybridization (FISH) as a readout of their efficacy Cells having chromosomes with labeled γ-tubulin centromeres can be used. Alternatively, labeled antibodies that bind to γ-tubulin in centrosomes can be used in the assays

Assay methods are also described herein for identifying and assessing the potency of inhibitors of NF-kB Inducing Kinase (NIK). NF-kB Inducing Kinase (NIK) mediates non-canonical NF-kB signaling and is associated with metastasis. Therefore, the inhibition of NIK may suppress CIN induced inflammatory responses and metastasis. Specific inhibition of the kinase function of NIK provides an approach to assess the potency of various compounds. Two methods are described below to identify and assess NIK inhibition.

ADP production can be monitored as a readout for MCAK activity using the Transcreener ADP assay from BellBrook Labs. This assay is based on the ability of ADP to displace a fluorescent tracer (633 nm) bound to an antibody the specifically recognizes ADP. Competitive displacement of the tracer causes a decrease in fluorescence, as measured by laser excitation at 633 nm. Thus, the activity of MCAK can be calculated by plotting the concentration of drug used and the amount of ADP produced/decrease in fluorescent intensity.

Inhibition of NIK provides an approach to directly inhibit the non-canonical NF-κB pathway. This assay relies on quantification of the nuclear translocation of p52 (RELB; non-canonical NF-kB signaling) using high content cellular imaging. For RELB nuclear translocation assay, cells are treated with different concentrations of compounds and stimulated with 100 ng/mL of an antagonistic antilymphotoxin beta receptor (LT-PR) antibody, a potent activator of non-canonical NF-kB signaling. The RELB translocation into the nucleus is quantified by the ratio of the nuclear over cytoplasmic signal intensity Potent compounds are discovered that selectively inhibit the nuclear translocation of RELB.

The compounds so identified can be useful for selectively targeting tumors or treating cancers characterized by CIN. For example, the compounds are useful for treating tumors or cancer types that exhibit overproduction of cGAMP.

“Treatment” or “treating” refers to both therapeutic treatment, and prophylactic or preventative measures. Those in need of treatment include those already with the disorder as well as those prone to have the disorder, or those in whom the disorder is to be prevented.

“Subject” for purposes of treatment refers to any animal classified as a mammal or bird, including humans, domestic and farm animals, and zoo, sports, or pet animals, such as dogs, horses, cats, cows, etc. Preferably, the subject is human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, lung, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

The term “hematological malignancies” includes adult or childhood leukemia and lymphomas, Hodgkin's disease, lymphomas of lymphocytic and cutaneous origin, acute and chronic leukemia, plasma cell neoplasm and cancers associated with AIDS.

The inventive methods and compositions can also be used to treat cancer of the breast, cancer of the lung, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. In some cases, metastatic cancers are treated but primary cancers are not treated. Information regarding numerous types of cancer can be found, e.g., from the American Cancer Society (cancer.org), or from, e.g., Wilson et al. (1991) Harrison's Principles of Internal Medicine, 12th Edition, McGraw-Hill, Inc.

In some embodiments, the cancer and/or tumors to be treated are those that originate as breast or lung cancers.

Treatment of, or treating, metastatic cancer can include the reduction in cancer cell migration or the reduction in establishment of at least one metastatic tumor. The treatment also includes alleviation or diminishment of more than one symptom of metastatic cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent metastatic cancer, it may substantially eliminate metastatic tumor formation and growth, and/or it may arrest or inhibit the migration of metastatic cancer cells.

Anti-cancer activity can reduce the progression of a variety of cancers (e.g., breast, lung, or prostate cancer) using methods available to one of skill in the art. Anti-cancer activity, for example, can determined by identifying the lethal dose (LD₁₀₀) or the 50% effective dose (ED50) or the dose that inhibits growth at 50% (GI₅₀) of an agent of the present invention that prevents the migration of cancer cells. In one aspect, anti-cancer activity is the amount of the agent that reduces 50%, 60%, 70%, 80%, 90%, 95%, 97%, 98%, 99% or 100% of cancer cell migration, for example, when measured by detecting expression of a cancer cell marker at sites proximal or distal from a primary tumor site, or when assessed using available methods for detecting metastases.

In another example, agents that promote chromosomal instability can be administered to sensitize tumor cells to immune therapies. Chromosomal instability promotes a viral-like response that synergizes with immune checkpoint blockades. Hence, by administering an agent that promotes chromosomal instability, tumor cells can become more sensitive to the immune system and to various immune therapies.

### Compositions

The invention also relates to compositions containing chemotherapeutic agents. Such an agent can be a polypeptide, a nucleic acid encoding a polypeptide (e.g., within an expression cassette or expression vector), a small molecule, a compound identified by a method described herein, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include a Kinsin-13, MCAK, ABCC4, and/or ABCG2 protein or polypeptide having at least 90% amino acid sequence identity to SEQ ID NO:1, 3, 5, 7, 9, 11, or a combination of such Kinsin-13, MCAK, ABCC4, and/or ABCG2 proteins or polypeptides. In other embodiments, the compositions can include a Kinsin-13, MCAK, ABCC4, and/or ABCG2 nucleic acid or expression cassette that includes a nucleic acid segment encoding a Kinsin-13, MCAK, ABCC4, and/or ABCG2 protein. For example, the nucleic acid or expression cassette can have a nucleic acid sequence with at least 90% sequence identity to any of SEQ ID NO: 2, 4, 6, 8, 10, 12.

In some embodiments, the chemotherapeutic agents of the invention (e.g., polypeptide, a nucleic acid encoding a polypeptide (e.g., within an expression cassette or expression vector), a small molecule, a compound identified by a method described herein, or a combination thereof), are administered in a “therapeutically effective amount.” Such a therapeutically effective amount is an amount sufficient to obtain the desired physiological effect, such a reduction of at least one symptom of cancer. For example, chemotherapeutic agents can reduce cell metastasis by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%. Symptoms of cancer can also include tumor cachexia, tumor-induced pain conditions, tumor-induced fatigue, tumor growth, and metastatic spread. Hence, the chemotherapeutic agents may also reduce tumor cachexia, tumor-induced pain conditions, tumor-induced fatigue, tumor growth, or a combination thereof by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the chemotherapeutic agents may be administered as single or divided dosages. For example, chemotherapeutic agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, peptides, or nucleic acid chosen for administration, the disease, the weight, the physical condition, the health, and the age of the mammal. Such factors can be readily determined by the clinician employing animal models or other test systems that are available in the art.

Administration of the chemotherapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the chemotherapeutic agents and compositions of the invention may be essentially continuous over a preselected period of time or may be in a series of spaced doses. Both local and systemic administration is contemplated.

To prepare the composition, small molecules, compounds, polypeptides, nucleic acids, expression cassettes, and other agents are synthesized or otherwise obtained, purified as necessary or desired. These small molecules, compounds, polypeptides, nucleic acids, expression cassettes, and other agents can be suspended in a pharmaceutically acceptable carrier and/or lyophilized or otherwise stabilized. The small molecules, compounds, polypeptides, nucleic acids, expression cassettes, other agents, and combinations thereof can be adjusted to an appropriate concentration, and optionally combined with other agents. The absolute weight of a given small molecule, compound, polypeptide, nucleic acid, and/or other agents included in a unit dose can vary widely. For example, about 0.01 to about 2 g, or about 0.1 to about 500 mg, of at least one molecule, compound, polypeptide, nucleic acid, and/or other agent, or a plurality of molecules, compounds, polypeptides, nucleic acids, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the chemotherapeutic agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of chemotherapeutic agent for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the chemotherapeutic agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The chemotherapeutic agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the chemotherapeutic agent with liquid carriers, solid matrices, semi-solid carriers, finely divided solid carriers or combinations thereof, and then, if necessary, introducing or shaping the product into the desired delivery system. For example, the chemotherapeutic agent(s) can be linked to a convenient carrier such as a nanoparticle, albumin, polyalkylene glycol, or be supplied in prodrug form. The chemotherapeutic agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the chemotherapeutic agent(s) and/or other agents can sometimes be administered in an oral dosage form, that oral dosage form can be formulated so as to protect the small molecules, compounds, polypeptides, nucleic acids, expression cassettes, and combinations thereof from degradation or breakdown before the small molecules, compounds, polypeptides, nucleic acids encoding such polypeptides, and combinations thereof provide therapeutic utility. For example, in some cases the small molecules, compounds, polypeptides, nucleic acids encoding such polypeptide, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The chemotherapeutic agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

A chemotherapeutic agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as chemotherapeutic agents, anti-viral agents, antibacterial agents, antimicrobial agents and/or preservatives. Examples of additional therapeutic agents that may be used include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives: microtubule-stabilizing agents such as paclitaxel (Taxol®), docetaxel (Taxotere™), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compositions can also be used in conjunction with radiation therapy.

The present description is further illustrated by the following examples, which should not be construed as limiting in any way. The contents of all cited references (including literature references, issued patents, published patent applications as cited throughout this application) are hereby expressly incorporated by reference.

### Example 1: Materials and Methods

This Examples describes some of the materials and methods employed in the development of the invention.

Genomic analysis of Primary-metastasis matched pairs.

Whole exome DNA sequence data from 61 brain metastases with matched primary tumor and normal (Brastianos et al. Cancer Discovery 5, 1164-1177 (2015)) was downloaded from the database of Genotypes and Phenotypes (dbGAP) and processed as described (McGranahan et al. Science 351, 1463-1469 (2016)) to derive allele specific segmented DNA copy number data for each sample. The weighted Genome Instability Index (wGII), describing the proportion of the genome that was classified as aberrant relative to tumor ploidy, was determined as described (Burrell et al., Nature 494, 492-496 (2013)).

### Mitelman Database Analysis.

All available breast adenocarcinoma cases in the Mitelman database (Mitelman et al. Database of Chromosome Aberrations and Gene Fusions in Cancer. cgap.nci.nih.gov Available at: cgap.nci.nih.gov/Chromosomes/Mitelman) were analyzed. Primary literature was reviewed to determine the source of the sample (primary tumor or metastasis). When clonal karyotype was reported as a range, the average value was used for this clone. Karyotype aberrations included structural aberrations as well as numerical deviations from the overall karyotype of the clone.

### Analysis of Chromosome Segregation in HNSCC.

Primary tumor specimens were analyzed from 60 patients with head and neck squamous cell carcinoma (HNSCC) (Chung et al. Cancer Cell 5, 489-500 (2004)). Forty patients had Hematoxylin and Eosin-stained (H&E) primary tumor samples of sufficient quality for high-resolution microscopy analysis. Analysis was restricted to cells fixed while undergoing anaphase as previously described (Bakhoun et al. Clin. Cancer Res. 17, 7704-7711 (2011); Zaki et al. Cancer 120, 1733-1742 (2014)). Chromosome missegregation was defined by hematoxylin staining presence in between the remaining segregating chromosomes during anaphase and it was reported as the percentage of cells undergoing anaphase with evidence of chromosome missegregation. Clinical lymph node status was defined by clinical examination or radiographic evidence of lymph node tumor involvement (Chung et al. Cancer Cell 5, 489-500 (2004)).

### Single-Cell Karyotyping.

Cultures were treated with colcemid at a final concentration of 0.1 μg ml⁻¹. Following 45 min incubation at 37° C., the cultures were trypsinized, resuspended in pre-warmed 0.075M KCl, incubated for an additional 10 minutes at 37° C. and fixed in methanol-acetic acid (3:1). The fixed cell suspension was then dropped onto slides, stained in 0.08 μg/ml DAPI in 2×SSC for 5 minutes and mounted in antifade solution (Vectashield, Vector Labs). Metaphase spreads were captured using the Nikon Eclipse E800 epifluorescence microscope equipped with GenASI Cytogenetic suite (Applied Spectral Imaging, Carlsbad). For each sample a minimum of 20 inverted DAPI-stained metaphases were fully karyotyped and analyzed according to the International System of Human Cytogenetic Nomenclature (ISCN) 2013.

### Cell Culture.

Cell lines were purchased from the American Type Culture Collection (ATCC). Tumor (MDA-MB-231 and H2030) and 293T cells were cultured in DMEM supplemented with 10% FBS and 2 mM of L-Glutamine in the presence of penicillin (50 Uml⁻¹) and streptavidin (50 μgml⁻¹). All cells tested negative for mycoplasma. Cell confluence was measured using IncuCyte live-cell analysis system (Essen Bioscience).

### Immunofluorescence Microscopy.

Cell fixation and antibody staining were performed as described (Bakhoun et al. Nat Commun 6, 5990 (2015)). Briefly, cells were fixed with ice-cold (−30 C) methanol for 15 minutes—when staining for centromeres, centrosomes, cGAS, Vimentin, β-actin, or α-tubulin—or 4% paraformaldehyde—when staining for ReIB, p65, IRF3, ssDNA, dsDNA, CoxIV, or β-catenin. Subsequently, cells were permeabilized using 1% triton for 4 minutes. See Table 1 for antibody information.

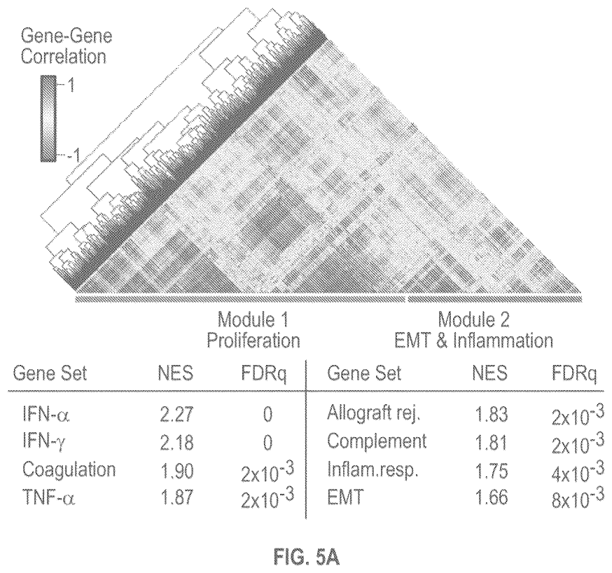

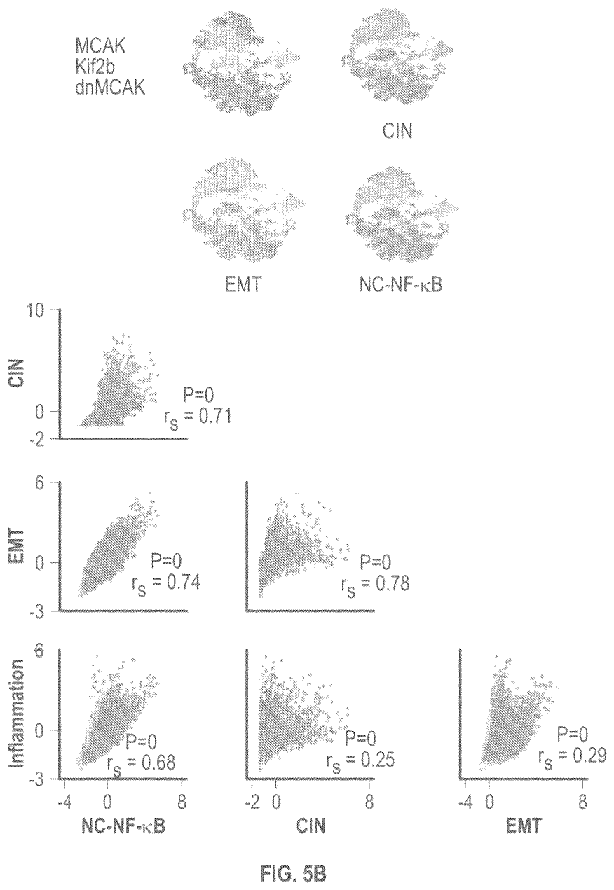

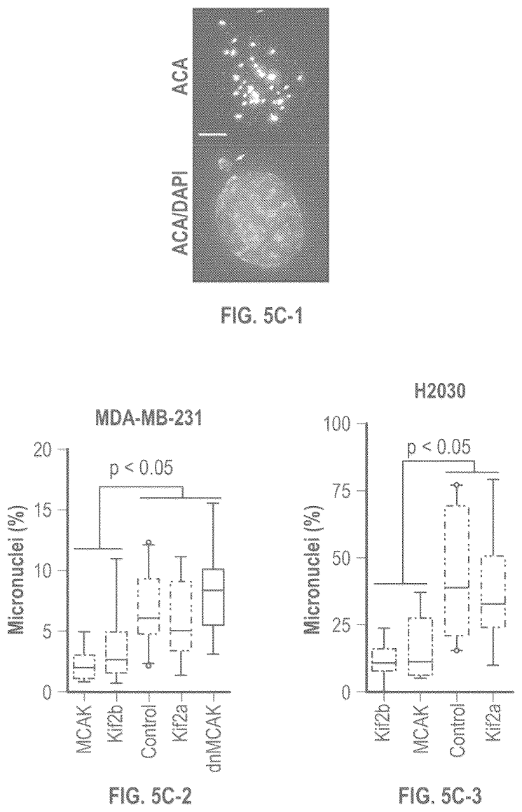

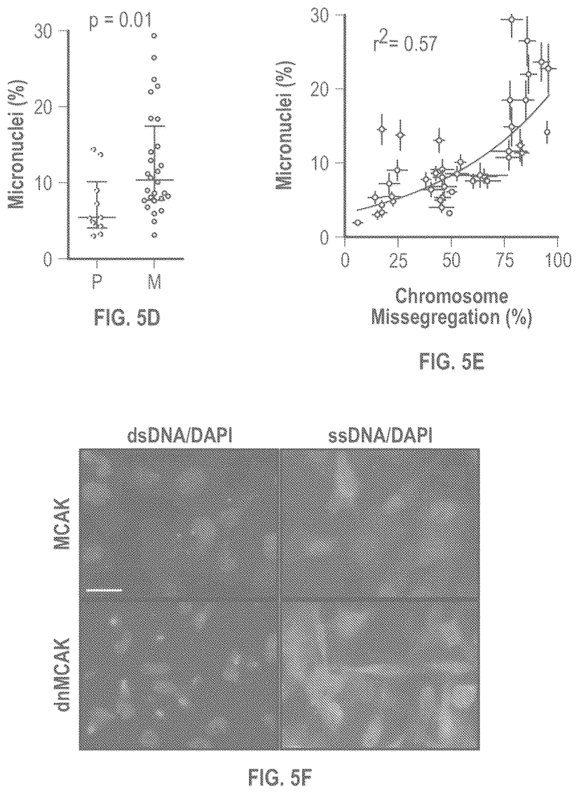

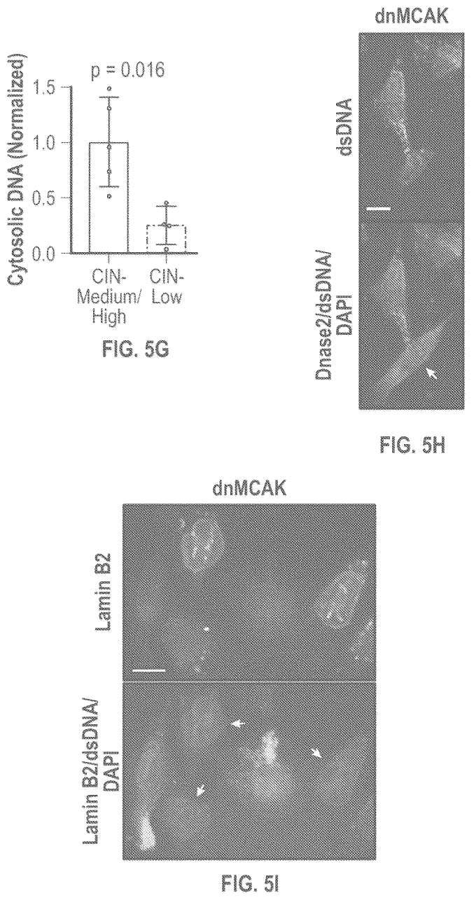

TABLE 1
Antibodies used for immunofluorescence

| Antibody Target | Source | Catalog No. |
|---|---|---|
| α-tubulin | Sigma Aldrich | T9026 |
| β-actin | Abcam | ab8227 |
| β-catenin | Abcam | ab16051 |
| cGAS | Sigma Aldrich | HPA031700 |
| Cox IV | Abcam | ab16056 |
| dsDNA | Abcam | AB27156 |
| dsDNA | Thermo Fisher | MAB1293MI |
| (FIG. 5f) | Scientific | |
| Human centromere | Antibodies | 15-234-0001 |
| proteins | Incorporated | |
| IRF3 | Abcam | ab68481 |
| p65 | Abcam | ab16502 |
| Pericentrin | Abcam | ab4448 |
| RelB | Cell Signaling | 4922 |
| | Technology | |
| ssDNA | Thermo Fisher | MAB3299MI |
| | Scientific | |
| Vimentin | Abcam | ab201637 |

For selective plasma membrane permeabilization used for cytosolic dsDNA and ssDNA staining, cells were treated with 0.02% saponin for 5 minutes after fixation. For single—stranded (Thermo Fisher FEREN321) and double stranded (Life Technologies—EN0771)-specific nuclease treatment, cells were also permeabilized with 0.02% saponin for 2 minutes and treated with either nucleases for 10 minutes before fixation using 4% paraformaldehyde. TBS-BSA was used as a blocking agent during antibody staining. DAC was added together with secondary antibodies. Cells were mounted with Prolong Diamond Antifade Mountant (Life Technologies—P36961).

Immunoblotting.

Cells were pelleted and lysed using RIPA buffer. Protein concentration was determined using BCA protein assay and 20-30 mg of total protein were loaded in each lane. Proteins were separated by gradient SOS-PAGE and transferred to PVDF membranes. See Table 2 for antibody information.

TABLE 2
Antibodies used for immunoblots

| Antibody Target | Company | Catalog No. |
|---|---|---|
| β-actin | Abcam | ab8227 |
| cGAS | Sigma Aldrich | HPA031700 |
| GFP | Life Technologies | A11122 |
| IRF3 | Abcam | ab68481 |
| p100/p52 | Cell Signaling | 4882 |
| p65 | Abcam | ab16502 |
| phospho-IRF3 | Cell Signaling | 4947 |
| phospho-p100 | Abcam | 194919 |
| phospho-p65 | Cell Signaling | 3033 |
| phospho-TBK1 | Cell Signaling | 5483 |
| RelB | Cell Signaling | 4922 |
| STING | Cell Signaling | 13647 |
| TBK1 | Cell Signaling | 3013 |
| TRAF2 | Cell Signaling | 4712 |
| TRAF3 | Cell Signaling | 4729 |

For quantitative comparisons shown in FIG. 6D, immunoblots from three biological replicates were used. Band intensities were obtained using ImageJ (see website at imagej.nih.gov/ij), normalized to β-actin (loading control) and background was subtracted. Ratios were normalized to control cells.

### Knockdown and Overexpression Constructs.

Luciferase expression was achieved using pLVX plasmid (expressing tdTomato) and cells stably expressing luciferase were sorted for tdTomato expression. Kinesin-13 expression was achieved using plasmid (pEGFP) transfection or lentiviral (pLenti-GIII-CMV-GFP-2A-Puro) expression where cells were selected using G418 (0.5 mgml⁻¹) or puromycin (5 μgml⁻¹), respectively. Dnase2 overexpression was achieved using a pLenti-GIII-CMV-RFP-2A-Puro plasmid with puromycin used for selection. Plasmids containing kinesin-13 or Lamin B2 (pQCXIB-mCherry-Imnb2) constructs were kindly offered by the Compton and Hetzer Laboratories, respectively. Blasticidin was used to select for Imnb2 expressing cells at 10 μgml⁻¹. All other plasmids were purchased from Applied Biological Materials Inc. (www.abmgood.com). Stable knockdown of STING, NFKB2, ReIB, and cGAS were achieved using shRNAs in pRRL (SGEP or SGEN) plasmids and were obtained from the MSKCC RNA Interference Core. Two to four distinct shRNA hairpins were screened per target. Targeted shRNA sequences are listed in Table 3.

TABLE 3
Anti-sense shRNA sequences

| | Entrez | shRNA | shRNA |
|---|---|---|---|
| Gene Name | ID | ID | anti-sense sequence |
| cGAS | 115004 | 2 | TTCATATTCAATTTGCTTTGTC |
| | | | (SEQ ID NO: 25) |
| | | 1 | TTAGTTTTAAACAATCTTTCCT |
| | | | (SEQ ID NO: 26) |
| | | 3 | TTCTAAAAACTGACTCAGAGGA |
| | | | (SEQ ID NO: 27) |
| NFKB2 | 4791 | 1 | TTCAGTTGCAGAAACACTGTTA |
| | | | (SEQ ID NO:28) |
| | | 3 | TCATCATATTCAATAATACCAT |
| | | | (SEQ ID NO: 29) |
| | | 2 | TGAAGTTTTTGTATCATAGTCC |
| | | | (SEQ ID NO: 30) |
| RelB | 5971 | 3 | TTCCTCATCTGTAAAATGGGCT |
| | | | (SEQ ID NO: 31) |
| | | 1 | TAATGATTGGGGAACATGTTGC |
| | | | (SEQ ID NO: 32) |
| | | 4 | TTTCTTGTCATAGACGGGCTCG |
| | | | (SEQ ID NO: 33) |
| | | 2 | TCAAAAACTCATCTTTATTGGG |
| | | | (SEQ ID NO: 34) |
| STING | 340061 | 2 | TTATGATCCCATTTCACAGGTT |
| | | | (SEQ ID NO: 35) |
| | | 1 | TCTCAAGAGAAATCCGTGCGGA |
| | | | (SEQ ID NO: 36) |

### Animal Studies.

Animal experiments were performed in accordance with protocols approved by the Weill Cornell Medicine Institutional Animal Care and Use Committee. For disease-specific survival, power analysis indicated that 10 mice per group will be sufficient to detect a difference at relative hazard ratios of <0.2 or >5 with 80% power and 95% confidence, given a median disease-specific survival of 3 months in the control group and a total follow up period of 250 days. There was no need to randomize animals. Investigators were not blinded to group allocation. Intracardiac injection was performed as previously described (Chen et al. Nature 533, 493-498 (2016)). Briefly, cells were trypsinized and washed with PBS and a 1×10⁵ cells (in 100 μl of PBS) were injected into the left cardiac ventricle of female athymic 6-7-week-old athymic nude (nu/nu) mice (Jackson Laboratory strain 002019). Mice were then immediately injected with D-luciferin (150 mgkg⁻¹) and subjected to bioluminescence imaging (BLI) using tan IVIS Spectrum Xenogen instrument (Caliper Life Sciences) to ensure systemic dissemination of tumor cells. Metastatic burden was measured at week 5 after injection using BLI and in the case of MDA-MB-231 mice BLI images were taken every 1-2 weeks for up to 17 weeks. BLI images were analyzed using Living Image Software v.2.50. Disease-specific survival endpoint was met when the mice died or met the criteria for euthanasia under the IACUC protocol and had radiographic evidence of metastatic disease. For Orthotopic tumor implantation, 2.5×10⁵ cells in 50 μl of PBS were mixed 1:1 with Matrigel (BD Biosciences) and injected into the fourth mammary fat pad. Only one tumor was implanted per animal. Primary tumors were surgically excited when they reached ˜1.5 cm in the largest dimension and metastatic dissemination was assessed using BLI imaging at 1-week to 3-week intervals for up to 30 weeks. Distant metastasis-free survival endpoint was met when BLI signal was seen outside of site of primary tumor transplantation. To derive short-term culture from primary tumors and metastases, anesthetized animals (isofluorane) were imaged then sacrificed. Ex-vivo BLI was subsequently performed on harvested organs to define the precise location of the metastatic lesion. Primary tumors and metastases were subsequently mechanically dissociated and cultured in DMEM with selection media to select for tumor cells. All subsequent assays were performed after one passage.

### Patient-Derived Xenografts (PDX) Assays.

PDX models of human metastatic breast cancers were successfully generated by transplanting the freshly obtained surgically excised tumor specimens from patients consented under the IRB approved protocol (MSKCC IRB #97-094) in female NOD-scid IL2Rgamma^{null} (NSG) (Jackson Laboratories strain 005557). The estrogen receptor-positive PDX was derived from breast cancer metastatic to the bone. The triple-negative PDX was established out of an axillary lymph node metastasis from a patient with inflammatory breast cancer. PDXs were maintained for a maximum of three serial passages. Briefly, freshly obtained tumor tissue specimens were either directly transplanted in the mammary fat-pad of the mice or minced into 1-2 mm pieces in serum free MEM medium with nonessential amino acids (Cat #41500018, Thermofisher) transduced with lentiviral vectors expressing either GFP-luciferase or pUltra-Chili-Luc plasmid (Addgene plasmid: 48688) followed by transplantation into mice. Typically, PDX tumor growth became evident during the first 1-3 weeks post engrafting and tumor continued to grow for additional 4-8 weeks. Primary tumor growth and metastases were followed using BLI or spectrum CT imaging. At the time of harvesting of primary tumors and metastases, we derived primary cell cultures directly from primary tumors as well as lung and liver metastases. Briefly, 500 mg of fresh bulk tumor tissues were chopped into 1-2 mm³ sized pieces and incubated in Accutase (AT104; Innovative Cell Technologies) for cell detachment and separation over 1-2 hours. The dissociated tissues were sieved through 100-μm cell strainers and pelleted the cells by centrifugation at 1200 RPM. The pellets are washed and resuspended in the above MEM buffer with 3% FBS. Cells were analyzed for chromosome missegregation after one passage.

RNA sequencing and analysis. Bulk RNA was extracted from cells using the QIAShredder (Qiagen—79654) and the RNA extraction kit (Qiagen—74106) and sequenced using HiSeq2500 or HiSeq4000 (Illumina Inc.). The quality of the raw FASTQ files were checked with FastQC (see website at bioinformatics.babraham.ac.uk/projects/fastqc/), then mapped to human reference GRCh38 using STAR (v2.4.1d, 2-pass mode) (Dobin et al. Bioinformatics 29, 15-21 (2013)). Gene expression was estimated using cufflinks (v2.2.1, default parameters) and HTSeq (v0.6.1) (Trapnell et al. Nat Biotechnol 28, 511-515 (2010); Anders et al. Bioinformatics 31, 166-169 (2015)). Differential expression analyses were performed using DESeq2 (v1.14.1) (Love et al. Genome Biol. 15, 550 (2014)). Prior to any unsupervised analyses, expression counts were transformed using variance-stabilizing transformation using the DESeq2 R package. All custom code, statistical analysis, and visualizations were performed in Python or R. We used Nextflow to manage some of the computational pipelines (see website at nextflow.io).

### Single-Cell RNA Sequencing.

Cells were trypsinized and resuspended in PBS. 21 ul of a cellular suspension at 400 cells/ul, >95% viability, were loaded onto to the 10X Genomics Chromium platform to generate barcoded single-cell GEMs. Single-cell RNA sequencing (scRNA-seq) libraries were prepared according to 10X Genomics specifications (Single Cell 3′ Reagent Kits User Guide PN-120233, 10x Genomics, Pleasanton, CA, USA). GEM-Reverse Transcription (RT) (55° C. for 2 h, 85° C. for 5 min; held at 4° C.) was performed in a C1000 Touch Thermal cycler with 96-Deep Well Reaction Module (Bio-Rad, Hercules). After RT, GEMs were broken and the single-strand cDNA was cleaned up with DynaBeads MyOne Silane Beads (Thermo Fisher Scientific, Waltham, MA) and SPRIselect Reagent Kit (0.6×SPRI; Beckman Coulter). cDNA was amplified for 14 cycles using the C1000 Touch Thermal cycler with 96-Deep Well Reaction Module (98° C. for 3 min; 98° C. for 15 s, 67° C. for 20 s, and 72° C. for 1 min×14 cycles; 72° C. for 1 min; held at 4° C.). Quality of the cDNA was analyzed using an Agilent Bioanalyzer 2100 (Santa Clara, CA). The resulting cDNA was sheared to −200 bp using a Covaris S220 instrument (Covaris, Wobum, MA) and cleaned using 0.6× SPRI beads. The products were end-, ‘A’-tailed and ligated to adaptors provided in the kit. A unique sample index for each library was introduced through 10 cycles of PCR amplification using the indexes provided by in the kit (98° C. for 45 s; 98° C. for 20 s, 60° C. for 30 s, and 72° C. for 20 s×14 cycles; 72° C. for 1 min; held at 4° C.). After two SPRI cleanups, libraries were quantified using Qubit fluorometric quantification (Thermo Fisher Scientific, Waltham, MA) and the quality assessed on an Agilent Bioanalyzer 2100. Four libraries were pooled and clustered on a HiSeq2500 rapid mode at 10 μM on a pair end read flow cell and sequenced for 98 cycles R1, followed by 14 bp 17 Index (10X Barcode), 8 bp 15 Index (sample Index) and 10 bp on R2 (UMI). Primary processing of sequencing images was done using Illumina's Real Time Analysis software (RTA). Demultiplexing and post processing was done using the 10X Genomics Cell Ranger pipeline as per the manufacturer recommendations. Single cell RNA sequencing data (scRNA-seq) was processed from raw reads to a molecule count array using the Cell Ranger pipeline (Zheng et al. Nat Commun 8, 14049 (2017)). Additionally, to minimize the effects of experimental artifacts on the analysis, data was pre-processed to filter out cells with low total molecule counts (library size), low complexity and high mitochondrial content, identified by a bimodal fit. Remaining cells were normalized by dividing the expression level of each gene in a cell by its total library size and then scaling by the median library size of all cells). After normalizing by library size; principal component analysis (PCA) was performed to improve robustness of the constructed Markov Matrix generated when computing diffusion eigenvalues for imputation of dropout noise (van Dijk et al. bioRxiv (2017)). The number of principle components was chosen to retain approximately 80% of variance in the data and excluded the first principal component, which was highly correlated with library size. Imputation of both he normalized and unnormalized count matrix was performed using a Markov matrix raised to the power of 3 (power corresponds the approximate number of weighted nearest neighbors) and with a gene expression distribution computed according to 21 nearest neighboring cells as described (van Dijk et al. bioRxiv (2017)). Subpopulations were identified using Phenograph (Levine et al. Cell 162, 184-197 (2015)) and genes differentially expressed in at least one subpopulation were identified by the Kruskal-Wallis rank statistic using a bootstrapping method for random down-sampling of matched molecule and cell counts from each subpopulation. t-Distributed Stochastic Neighbor Embedding (t-SNE) was used to visualize subpopulation structure based on the first 20 principle components of the imputed count matrix, subsetted by the top 5,150 differentially expressed genes (False Discovery Rate (FDR) q of Kruskal Wallis rank statistic <0.05). Mean expression of key gene signatures in population M versus other subpopulations were z-normalized and visualized by violin plots. All gene signatures are annotated near the end of Example 1. The correlation between gene signatures was computed using the Spearman Rank Correlation Coefficient according to mean expression of all genes per signature per cell. Ward's minimum variance method was applied to hierarchically cluster cells by their normalized expression of differentially expressed epithelial-to-mesenchymal transition (EMT) genes.

### Patient Survival Analysis.

Genes used for survival analysis include PELI2, BMP2, SHH, TNS4, RAB3B, ROBO1, ARHGAP28, CHN2, CST1, F13A1, CPVL, SEMA6D, NHSL2, GTF21P7, DPYSL3, PCDH7, KHDRBS3, TRAC, TMEM156, CST4, CD24, FGF5, (optionally NTN4) (see Table 5).

Two independent datasets were used to evaluate survival markers. The first was a meta-analysis (Györffy et al., Breast Cancer Res. Treat. 123, 725-731 (2010)) and a validation cohort (Hatzis et al. J. Am. Med. Assoc. 305, 1873-1881 (2011)). For the meta analysis, publicly available microarray gene expression datasets deposited in the KM-Plotter database (www.kmplot.com) were used, with the following microarray probes for each gene (note that some genes have multiple names and alternate names could be listed below): 219132_at (PELI2), 205289_at (BMP2), 207586_at (SHH), 230398_at (TNS4), 227123_at (RAB3B), 213194_at (ROBO1), 227911_at (ARHGAP28), 213385_at (CHN2), 206224_at (CST1), 203305_at (F13A1), 208146_s_at (CPVL), 226492_at, (SEMA6D), 201431_s_at (DPYSL3), 228640_at (PCDH7), 209781_s_at (etoile), 210972_x_at (TRA@), 220169_at (TMEM156), 206994_at (CST4), 266_s_at (CD24), 210311_at (FGF5), 200948_at (MLF2). For the meta-analysis cohort, the JetSet best probe set was used and auto-selection was used for best cutoff between the 25^{th} and 75^{th} percentile. For the validation cohort in which DMFS data was available (Hatzis et al. JAMA 305, 1873-1881 (2011)), the z-normalized expression data for a dataset and the median value was used as a cutoff. DMFS curves were compared using the log-rank test. For the first dataset, the best cutoff value was determined to be the 36-percentile was then used such that the patients with cumulative expression of the genes above that were in the bottom 36-percentile had higher metastasis-free survival. In the second data set, publicly deposited gene expression data was used that was derived from next-gen sequencing and the median expression values were used as a cutoff and obtained similar results. In this type of analysis, it is typical to use cutoff values ranging from the 25-percentile to the 75-percentile depending on the patient population and assay used thus we should include that.

### In Vitro Invasion and Migration Assays.

For the invasion and migration/chemotaxis assays the CytoSelect cell invasion (CBA-110) and cell migration (CBA-100) kits, respectively, were used. Briefly, 3×10⁵ cells were suspended in serum-free media and placed on top of the membrane. Media containing serum was placed at the bottom and cells, which have invaded to the inferior surface of the collagen membrane, were stained and counted 18-24 hours later. For the chemotaxis assay, we used a colorimetric approach (OD 560 nm) for quantification. For the scratch assay, cells were treated with mitomycin C (10 μgml⁻¹) for 1 hour when they reached >90% confluence and then placed in DMEM containing 1% FBS. Wounds were applied using p200 pipette tip and images of the wound were taken immediately and at subsequent regular intervals. ImageJ was used for quantification of wound surface area.

### Quantification of Cytosolic DNA.

Approximately 1×10⁷ cells were lysed and the nuclear, cytosolic, and mitochondrial fractions were obtained using the mitochondrial isolation kit (Thermo Fisher—89874). Protease inhibitors were not used to enable subsequent DNA purification. Mitochondria were purified at 12,000×g to minimize their contamination in the cytosolic fraction. DNA was subsequently isolated from the nuclear, cytosolic, mitochondrial fractions using the Qiagen DNeasy blood and tissue kit (Qiagen—69506) and dsDNA was quantified using Qubit 2.0 (Invitrogen) using Qubit dsDNA HS Reagent.

### Data Availability.

All RNA sequencing data was deposited in the Sequence Read Archive (SRA, www.ncbi.nlm.nih.gov/sra). Single-cell RNAseq data was deposited under the following accession number: SRP104750. Bulk RNAseq data was deposited under the following accession number: SRP104476. Access link at website ftp://ftp-trace.ncbi.nlm.nih.gov/sra/review/SRP104476_20170424_100917_3d522deaf85 577451c01974654b36ad3 CIN gene expression signature for assessing survival: PELI-2, BMP2, SHH, TNS4, RAB3B, ROBO1, ARHGAP28, CHN2, CST1, F13A1, CPVL, SEMA6D, NHSL2, GTF2IP7, DPYSL3, PCDH7, KHDRBS3, TRAC, TMEM156, CST4, CD24, FGF5, (optionally NTN4). Examples of sequences for the proteins and nucleic acids encoding these proteins, are illustrated in Table 5.

TABLE 5
CIN Gene Expression Signature Genes

| Gene | Example CIN |
|---|---|
| Name | Gene Expression Signature Human Sequence |
| PELI2 | mfspgqeehc apnkepvkyg elvvlgynga lpngdrqrrk |
| | srfalykrpk angvkpstvh vistpqaska isckgqhsis |
| | ytlsrnqtvv veythdkdtd mfqvgrstes pidfvvtdti |
| | sgsqntdeaq itqstisrfa crivcdrnep ytarifaagf |
| | dsskniflge kaakwknpdg hmdglttngv lvmhprggft |
| | eesqpgvwre isvcgdvytl retrsaqqrg klvesetnvl |
| | qdgslidlcg atllwrtadg lfhtptqkhi ealrqeinaa |
| | rpgcpvglnt lafpsinrke vveekqpway lscghvhgyh |
| | nwghrsdtea nerecpmcrt vgpyvplwlg ceagfyvdag |
| | ppthaftpcg hvcseksaky wsqiplphgt hafhaacpfc |
| | atqlvgeqnc iklifqgpid (SEQ ID NO: 37; cDNA |
| | sequence NCBI accession no. NM_021255.2) |
| BMP2 | mvagtrclla lllpqvllgg aaglvpelgr rkfaaassgr |
| | pssqpsdevl sefelrllsm fglkqrptps rdavvppyml |
| | dlyrrhsgqp gspapdhrle raasrantvr sfhheeslee |
| | lpetsgkttr rfffnlssip teefitsael qvfreqmqda |
| | lqnnssfhhr iniyeiikpa tanskfpvtr lldtrivnqn |
| | asrwesfdvt pavmrwtaqg hanhgfvvev ahleekqgvs |
| | krhvrisrsl hqdehswsqi rpllvtfghd gkghplhkre |
| | krqakhkqrk rlkssckrhp lyvdfsdvgw ndwivappgy |
| | hafychgecp fpladhlnst nhaivqtlvn svnskipkac |
| | cvptelsais mlyldenekv vlknyqdmvv egcgcr |
| | (SEQ ID NO: 38; cDNA sequence NCBI |
| | accession no. NM_001200.3) |
| SHH | mlllarclll vlvssllvcs glacgpgrgf gkrrhpkklt |
| | playkqfipn vaektlgasg ryegkisrns erfkeltpny |
| | npdiifkdee ntgadrlmtq rckdklnala isvmnqwpgv |
| | klrvtegwde dghhseeslh yegravditt sdrdrskygm |
| | larlaveagf dwvyyeskah ihcsvkaens vaaksggcfp |
| | gsatvhleqg gtklvkdlsp gdrvlaaddq grllvsdflt |
| | fldrddgakk vfyvietrep rerllltaah llfvaphnds |
| | atgepeassg sgppsggalg pralfasrvr pgqrvyvvae |
| | rdgdrrllpa avhsvtlsee aagayaplta qgtilinrvl |
| | ascyavieeh swahrafapf rlahallaal apartdrggd |
| | sqggdrgggg grvaltapga adapgagata gihwysqlly |
| | qigtwlldse alhplgmavk ss (SEQ ID NO: 39; |
| | cDNA sequence NCBI accession no. |
| | NM_000193.3) |
| TNS4 | mgskassphg lgsplvaspr lekrlgglap qrgsrisvls |
| | aspvsdvsym fgssgsllhs snsshqsssr slespansss |
| | slhslgsysl ctrpsdfqap rnptltmgqp rtphspplak |
| | ehasscppsi tnsmvdipiv lingcpepgs sppqrtpghq |
| | nsvqpgaasp snpcpatrsn sqtlsdapft tcpegpardm |
| | qptmkfvmdt skywfkpnit reqaiellrk eepgafvird |
| | sssyrgsfgl alkvqevpas aqsrpqedsn dlirhflies |
| | sakgvhlkga deepyfgsls afvcqhsima lalpckitip |
| | grelggadga sdstdspasc qkksagchtl ylssysvetl |
| | tgalavqkai sttferdilp tptvvhfkvt eqgitltdvq |
| | rkvffrrhyp lttlrfcgmd peqrkwqkvc kpswifgfva |
| | ksqtepqenv chlfaevdmv qpasqviglv tallqdaerm |
| | (SEQ ID NO: 40; cDNA sequence NCBI |
| | accession no. BC013706.1) |
| RAB3B | masvtdgktg vkdasdqnfd ymfklliign ssvgktsflf |
| | ryaddtftpa fvstvgidfk vktvyrhekr vklqiwdtag |
| | qervrtitta yyrgamgfil myditneesf navqdwatqi |
| | ktyswdnaqv ilvgnkcdme eervvptekg qllaeqlgfd |
| | ffeasakeni svrqaferlv daicdkmsds ldtdpsmigs |
| | skntrlsdtp pllqqncsc (SEQ ID NO: 41; cDNA |
| | sequence NCBI accession no. NM_002867.3) |
| ROBO1 | miaepahfyl fgliclcsgs rlrqedfppr ivehpsdliv |
| | skgepatlnc kaegrptpti ewykggerve tdkddprshr |
| | mllpsgslff lrivhgrksr pdegvyvcva rnylgeavsh |
| | naslevailr ddfrqnpsdv mvavgepavm ecqpprghpe |
| | ptiswkkdgs plddkderit irggkimity trksdagkyv |
| | cvgtnmvger esevaeltvl erpsfvkrps nlavtvddsa |
| | efkceargdp vptvrwrkdd gelpksryei rddhtlkirk |
| | vtagdmgsyt cvaenmvgka easatltvqv gsepphfvvk |
| | prdqvvalgr tvtfqceatq npqpaifwrr egsqnllfsy |
| | qppqsssrfs vsqtgdltit nvqrsdvgyy icqtinvags |
| | iitkaylevt dviadrpppv irqgpvnqtv avdgtfvlsc |
| | vatgspvpti lwrkdgvlvs tqdsrikqle ngvlqiryak |
| | lgdtgrytci astpsgeatw sayievqefg vpvqpprptd |
| | pnlipsapsk pevtdvsrnt vtlswqpnln sgatptsyii |
| | eafshasgss wqtvaenvkt etsaikglkp naiylflvra |
| | anavgisdps qisdpvktqd vlptsqgvdh kqvqrelgna |
| | vlhlhnptvl ssssievhwt vdqqsqyiqg ykilvrpsga |
| | nhgesdwlvf evrtpaknsv vipdlrkgvn yeikarpffn |
| | efqgadseik faktleeaps appqgvlvsk ndgngtailv |
| | swqpppedtq ngmvqevkvw clgnetryhi nktvdgstfs |
| | vvipflvpgi rysvevaast gagsgyksep gfigldahgn |
| | pvspedqvsl aqqisdvvkq pafiagigaa cwiilmvfsi |
| | wlyrhrkkrn gltstyagir kvtyqrggea vssggrpgll |
| | nisepaaqpw ladtwpntgn nhndcsiscc tagngnsdsn |
| | lttysrpadc ianynnqldn kqtnlmlpes tvygdvdlsn |
| | kinemktfns pnlkdgrfvn psgqptpyat tqliqsnlsn |
| | nmnngsgdsg ekhwkplgqq kqevapvqyn iveqnklnkd |
| | yrandtvppt ipyngsydqn tggsynssdr gsstsgsqqh |
| | kkggartpkv pkqggmawad llppppahpp phsnseeyni |
| | svdesydqem pcpvpparmy lqqdeleeee dergptppvr |
| | gaasspaays yshqstatlt pspqeelqpm lqdcpeetgh |
| | mqhqpdrrrq pvsppppprp ispphtygyi sqplvsdmdt |
| | dapeeeedea dmevakmqtr rlllrglegt passvgdles |
| | svtgsmingw qsaseednis sgrssysssd gsfftdadfa |
| | qavaaaaeya glkvarrqmq daagrrhfha sqcprptspv |
| | stdsnmsaav mqktrpakkl khqpghlrre tytddlpppp |
| | vpppaikspt aqsktglevr pvvvpklpsm dartdrssdr |
| | kgssvkgrev ldgrqvvdmr tnpgdpreaq eqqndgkgrg |
| | nkaakrdlpp akthliqedi lpvcrptfpt snnprdpsss |
| | ssmssrgsgs rqreqanvgr rniaemqvlg gyergednne |
| | eleetes (SEQ ID NO: 42; cDNA sequence NCBI |
| | accession no. BC112336.1) |
| ARHGAP28 | mnelprdtcg nhtnqldgtk eerelprvik tsgsmpddas |
| | lnsttlsdas qdkegsfavp rsdsvailet ipvlpvhsng |
| | spepgqpvqn aisdddflek nippeaeels fevsysemvt |
| | ealkrnklkk seikkedyvl tkfnvqktrf glteagdlsa |
| | edmkkirhls lieltaffda fgiqlkrnkt ekvkgrdngi |
| | fgvpltvlld gdrkkdpgvk vplvlqkffe kveesglese |
| | gifrlsgcta kvkqyreeld akfnadkfkw dkmchreaav |
| | mlkaffrelp tslfpveyip afislmergp hvkvqfqalh |
| | lmvmalpdan rdaaqalmtf fnkvianesk nrmslwnist |
| | vmapnlffsr skhsdyeell lantaahiir lmlkyqkilw |
| | kvpsflitqv rrmneatmll kkqlpsvrkl lrrktleret |
| | aspktskvlq kspsarrmsd vpegvirvha pllskvsmai |
| | qlnnqtkakd ilakfqyenr ilhwqraals flngkwvkke |
| | reestetnrs pkhvflftig ldist (SEQ ID NO: 43; |
| | cDNA sequence NCBI accession no. |
| | BC065274.1) |
| CHN2 | maassnssls gssyssdaee yqppiwksyl yqlqqeaprp |
| | kriicpreve nrpkyvgref hgiisreqad ellggvegay |
| | ilresqrqpg cytlalrfgn qtlnyrlfhd gkhfvgekrf |
| | esihdlvtdg litiyietka aeyiskmttn piyehiqyat |
| | llrekvsrri srskneprkt nvtheehtav ekisslvrra |
| | althndnhfn yekthnfkvh tfrgphwcey canfmwglia |
| | ggvrcsdcgl nvhkqcskhv pndcqpdlkr ikkvyccdlt |
| | tlvkahntgr pmvvdicire iearglkseg lyrvsgfteh |
| | iedvkmafdr dgekadisan vypdiniitg alklyfrdlp |
| | ipvitydtys kfidaakisn aderleavhe vlmllppahy |
| | etlrylmihl kkvtmnekdn fmnaenlgiv fgptimrppe |
| | dstittlhdm ryqklivqil ienedvif SEQ ID NO: |
| | 44; nucleotide sequence NCBI accession no. |
| | LS482359.1) |
| CST1 | maqylstlll llatlavala wspkeedrii pggiynadln |
| | dewvqralhf aiseynkatk ddyyrrplrv lrarqqtvgg |
| | vnyffdvevg rtictksqpn idtcafheqp elqkkqlcsf |
| | eiyevpwenr rslvksrcqe s (SEQ ID NO: 45; |
| | nucleotide sequence NCBI accession no. |
| | NM_001898.2) |
| F13A1 | msetsrtafg grravppnns naaeddlptv elqgvvprgv |
| | nlqeflnvts vhlfkerwdt nkvdhhtdky ennklivrrg |
| | gsfyvqidfs rpydprrdlf rveyvigryp genkgtyipv |
| | pivselqsgk wgakivmred rsvrlsiqss pkcivgkfrm |
| | yvavwtpygv lrtsrnpetd tyilfnpwce ddavyldnek |
| | ereeyvlndi gvifygevnd iktrswsygq fedgiidtcl |
| | yvmdraqmdl sgrgnpikvs rvgsamvnak ddegvlvgsw |
| | dniyaygvpp sawtgsvdil levrssenpv rygqcwvfag |
| | vfntflrclg iparivtnyf sahdndanlq mdifleedgn |
| | vnskltkdsv wnyhcwneaw mtrpdlpvgf gqwqavdstp |
| | qensdgmyrc gpasvgaikh ghvcfqfdap fvfaevnsdl |
| | iyitakkdgt hvvenvdath igklivtkqi gqdgmmditd |
| | tykfqegqee erlaletalm ygakkplnte gvmksrsnvd |
| | mdfevenavl gkdfklsitf rnnshnryti taylsanitf |
| | ytgvpkaefk ketfdvtlep lsfkkeavli qageymgqll |
| | eqaslhffvt arinetrdvl akqkstvlti peiiikvrgt |
| | qvvgsdmtvt veftnplket lrnvwvhldg pgvtrpmkkm |
| | freirpnstv qweevcrpwv sghrkliasm ssdslrhvyg |
| | elavqiqrrp sm (SEQ ID NO: 46; nucleotide |
| | sequence NCBI accession no. NM_000129.3) |
| CPVL | mvgamwkviv slvllmpgpc dglfrslyrs vsmppkgdsg |
| | qplfltpyie agkiqkgrel slvgpfpgln mksyagfltv |
| | nktynsnlff wffpaqiqpe dapvvlwlqg gpggssmfql |
| | fvehgpyvvt snmtlrdrdf pwtttlsmly idnpvgtqfs |
| | ftddthgyav neddvardly saliqffqif peyknndfyv |
| | tgesyagkyv paiahlihsl npvrevkinl ngiaigdgys |
| | dpesiiggya eflyqiglld ekqkkyfqkq checiehirk |
| | qnwfeafeil dklldgdlts dpsyfqnvtg csnyynflrc |
| | tepedqlyyv kflslpevrq aihvgnqtfn dgtivekylr |
| | edtvqsvkpw lteimnnykv liyngqldii vaaaltersl |
| | mgmdwkgsqe ykkaekkvwk ifksdsevag yirqagdfhq |
| | viirggghil pydqplrafd minrfiygkg wdpyvg |
| | (SEQ ID NO: 47; nucleotide sequence NCBI |
| | accession no. AY358549.2) |
| SEMA6D | mrvfllcayi lllmvsqlra vsfpeddepl ntvdyhysrq |
| | ypvfrgrpsg nesqhrldfq lmlkirdtly iagrdqvytv |
| | nlnempktev ipnkkltwrs rqqdrencam kgkhkdechn |
| | fikvfvprnd emvfvcgtna fnpmcryyrl stleydgeei |
| | sglarcpfda rqtnvalfad gklysatvad flasdaviyr |
| | smgdgsalrt ikydskwike phflhaieyg nyvyfffrei |
| | avehnnlgka vysrvarick ndmggsqrvl ekhwtsflka |
| | rlncsvpgds ffyfdvlqsi tdiiqingip tvvgvfttql |
| | nsipqsavca fsmddiekvf kgrfkeqktp dsvwtavped |
| | kvpkprpgcc akhglaeayk tsidfpdetl sfikshplmd |
| | savppiadep wftktrvryr ltaisvdhsa gpyqnytvif |
| | vgseagmvik vlaktspfsl ndsvlleeie aynhakcsae |
| | needkkvisl qldkdhhaly vafssciiri plsrcerygs |
| | ckksciasrd pycgwlsqgs cgrvtpgmll ltedffafhn |
| | hsaegyeqdt efgntahlgd chgvrwevqs qesnqmvhmn |
| | vlitcvfaaf vlgafiagva vycyrdmfvr knrkihkdae |
| | saqsctdssg sfaklnglfd spvkeyqqni dspklysnll |
| | tsrkelppng dtksmvmdhr gqppelaalp tpestpvlhq |
| | ktlqamkshs ekahghgasr ketpqffpss ppphsplshg |
| | hipsaivlpn athdyntsfs nsnahkaekk lqnidhpltk |
| | ssskrdhrrs vdsrntlndl lkhlndpnsn pkaimgdiqm |
| | ahqnlmldpm gsmsevppkv pnreaslysp pstlprnspt |
| | krvdvpttpg vpmtslerqr gyhknssqrh sisampknln |
| | spngvllsrq psmnrggymp tptgakvdyi qgtpvsvhlq |
| | pslsrqssyt sngtlprtql krtpslkpdv ppkpsfvpqt |
| | psvrplnkyt v (SEQ ID NO: 48; nucleotide |
| | sequence NCBI accession no. BC150253.1) |
| C9orf152 | maegsrtqap gkgpplsiqf lraqyeglkr qqrtqahllv |
| | lpkgqntpap aesmvnavwi nkerrsslsl eeadsevegr |
| | leeaaqgclq apkspwhthl emhclvqtsp qdtshqvhhr |
| | gklvgsdqrl ppegdthlfe tnqmtqqgtg ipeaaqlpcq |
| | vgntqtkave sglkfstqcp lsiknphrsg kpayypfpqr |
| | ktprisqaar nlglygsa (SEQ ID NO: 49; |
| | nucleotide sequence NCBI accession no. |
| | NM_001012993.2) |
| NHSL2 | mesmgmvysv psscngptes tfstswkgda ftymtpsats |
| | qsnqvnengk npscgnswvs lnkvpplvpk eaatllvard |
| | npagcsgsag yperliqqrh mperpskigl ltsgtsrlet |
| | gpggasrfre rslsvptdsg ttdvdydeeq kaneacalpf |
| | astssegsns adniaslsaq qeaqhrrqrs ksislrkakk |
| | kpspptrsvs lvkdepgllp eggsalpkdq rpkslclsle |
| | hqghhsshpd aqghpaipnh kdpestqfsh hwyltdwksg |
| | dtyqslssss tatgttviec tqvqgssesl aspstsratt |
| | psqlsievea reisspgrpp glmspssgvs sqsetptptv |
| | smsltlghlp ppsssvrvrp vvperksslp ptspmekfpk |
| | srlsfdlplt sspnldlsgm sisirsktkv srhhsetnfg |
| | vklaqktnpn qpimpmvtqs dlrsvrlrsv sksepeddie |
| | speyaeepra eevftlperk tkppvaekpp varrppslvh |
| | kppsvpeeya ltsptlampp rssigharpl pqdsytvvrk |
| | pkpssfpdgr spgestapss lvftpfasss daffsgtqqp |
| | pqgsvedegp kvrvlperis lqsqeeaekk kgkipppvpk |
| | kpsvlylplt sptaqmeayv aeprlplspi itleedtkcp |
| | atgddlqsig qrvtstpqad sereasplg (SEQ ID NO: |
| | 50; nucleotide sequence NCBI accession no. |
| | BC136756.1) |
| GTF21P7 | TGCCTCCAGA AAGGGTTGAG AAGATAATGG ATCAGATTGA |
| | AAAGTACATC ATGACTCATC TCTGTAAATA TGCGTTCTGT |
| | CCAGAACCCC AGTGAGCCTG GAAGACTGGG TGCTATGGGA |
| | AATGTCATCA ATCCAATGCT AGTGAAAGAT GTGACTGGGG |
| | AATGCTGAAA AATGCGCACC CCTGGGAGGA ATGAGGAAAG |
| | ATGACATCCA CTGACTIGTT ATTTTTTTGA GAAGGAGTCT |
| | TGCTCTGTTG CCCAGGCTGG AGTGTGGTGG CACGATCTCG |
| | GCTCACTGAT GATGAGAAGA AAGATCTTGC CATTCAAAAG |
| | AGGATCACAG IGCAACCITC TCTCTCCTCT CACAAACACC |
| | ACGAATGTCG TCACCTCACC TATCCATCTC CCTCAAGCCA |
| | GCTTTTGACC TGAACTGGTT ATTTCCTACT TGCCTCCTGG |
| | ACTTGCTAAT AAAATAAACA CTAAAGCTTC CCACTTTCTA |
| | AAAACACCAT CAACCCCTGA GAGTAATCAA AACCITCCTC |
| | AAATTGAGGT CACTGTGGAA GGAGAATCTA ATGCCTGATG |
| | ATCTGTCACT ATCTCCCATC ACCCCCAGAT GGGACCATCT |
| | AGTTGCAGGA AAAGAAGGTC AAGACTCCCA GTCATTCTAC |
| | ATTATGCCTC AGCCAAGATG TCTCACCCCA CTCTCTCTGA |
| | TGCAACAAGA AGCCCCTGGA GAACGTTTCA GTCCCATTTT |
| | GTACTTCTGT CATGTGCTCA TCACAGTCTG |
| DPYSL3 | masgrrgwds sheddlpvyl arpgttdqvp rqkyggmfcn |
| | vegafesktl dfdalsvgqr gaktprsgqg sdrgsgsrpg |
| | iegdtprrgq greesrepap aspapagvei rsatgkevlq |
| | nlgpkdksdr llikggrivn ddqsfyadiy medglikqig |
| | dnlivpggvk tieangkmvi pggidvhthf qmpykgmttv |
| | ddffqgtkaa laggttmiid hvypepessl teayekwrew |
| | adgksccdya lhvdithwnd svkqevqnli kdkgvnsfmv |
| | ymaykdlyqv sntelyeift clgelgaiaq vhaengdiia |
| | qeqtrmlkmg itgpeghvls rpeeleaeav fraitiasqt |
| | naplyvtkvm sksaadlisq arkkgnvvfg epitaslgid |
| | gthvwsknwa kaaafvtspp lspdpttpdy insllasgdl |
| | qlsgsahctf staqkaigkd nftaipegtn gveermsviw |
| | dkavatgkmd enqfvavtst naakifnlyp rkgrisvgsd |
| | sdlviwdpda vkivsaknhq saaeynifeg melrgaplvv |
| | icqgkimled gnlhvtqgag rfipcspfsd yvykrikarr |
| | kmadlhavpr gmydgpvfdl tttpkggtpa gsargsptrp |
| | nppyrnlhqs gfslsgtqvd egvrsaskri vappggrsni |
| | tsls (SEQ ID NO: 51; nucleotide sequence |
| | NCBI accession no. BC077077.1) |
| PCDH7 | mlrmrtagwa rgwclgccll lplslslaaa kqllryrlae |
| | egpadvrign vasdlgivtg sgevtfsles gseylkidnl |
| | tgelstserr idreklpqcq mifdenecfl dfevsvigps |
| | qswvdlfegq vivldindnt ptfpspvltl tveenrpvgt |
| | lyllptatdr dfgrngiery ellqepgggg sggesrraga |
| | adsapypggg gngasgggsg gskrrldase ggggtnpggr |
| | ssvfelqvad tpdgekqpql ivkqaldreq rdsyeltlrv |
| | rdggdpprss qailrvlitd vndnsprfek svyeadlaen |
| | sapgtpilql raadldvgvn gqieyvfgaa tesvrrllrl |
| | detsgwlsvl hridreevnq lrftvmardr qqppktdkat |
| | vvlnikdend nvpsieirki griplkdgva nvaedvlvdt |
| | pialvqvsdr dqgengvvtc tvvgdvpfql kpasdteqdq |
| | nkkkvflhts tpldyeatre fnvvivavds gspslssnns |
| | livkvgdtnd nppmfgqsvv evvfpennip gervatvlat |
| | dadsgknaei aysldssvmg ifaidpdsgd ilvntvldre |
| | gtdryefkvn akdkgipvlq gsttvivqva dkndndpkfm |
| | qdvftfyvke nlqpnspvgm vtvmdadkgr naemslyiee |
| | nnnifsiend tgtiystmsf drehqttytf rvkavdggdp |
| | prsatatvsl fvmdendnap tvtlpknisy tllppssnvr |
| | tvvatvlatd sddginadln ysivggnpfk lfeidptsqv |
| | vslvgkltqk hyglhrlvvq vndsgqpsqs tttlvhvfvn |
| | esvsnataid sqiarslhip ltqdiagdps yeiskqrlsi |
| | vigvvagimt viliilivvm arycrsknkn gyeagkkdhe |
| | dfftpqqhdk skkpkkdkkn kkskqplyss ivtveaskpn |
| | gqrydsvnek lsdspsmgry rsvnggpgsp dlarhyksss |
| | plptvqlhpq sptagkkhqa vqdlppantf vgagdnisig |
| | sdhcseyscq tnnkyskqmr lhpyitvfg (SEQ ID |
| | NO: 52; nucleotide sequence NCBI accession |
| | no. NM_002589.2) |
| KHDRBS3 | meekylpelm aekdsidpsf thalrlvnQe iekfqkgegk |
| | eekyidvvin khmklgqkvl ipvkqfpkfn fvgkllgprg |
| | nslkrlgeet ltkmsilgkg smrdkakeee lrksgeakyf |
| | hlnddlhvli evfappaeay armghaleei kkflipdynd |
| | eirqaqlqel tylnggsena dvpvvrgkpt lrtrgvpapa |
| | itrgrgqvta rpvgvvvprq tptprqvlst rqpvsrgrql |
| | itprargvpp tqyrpppppp tqetygeydy ddgygtayde |
| | qsydsydnsy stpaqsgady ydyghqlsee tydsygqeew |
| | tnsrhkapsa rtakgvyrdq pygry (SEQ ID NO: 53; |
| | nucleotide sequence NCBI accession no. |
| | BC063536.1) |
| TRAC | pniqnpdpav yqlrdskssd ksvclftdfd sqtnvsgskd |
| | sdvyitdktv ldmrsmdfks nsavawsnks dfacanafnn |
| | siipedtffp spesscdvkl veksfetdtn lnfgnlsvig |
| | frilllkvag fnllmtlrlw ss (SEQ ID NO: 54; |
| | nucleotide sequence NCBI accession no. |
| | X02592.1) |
| TMEM156 | mtktallklf vaivitfili lpeyfktpke rtlelsclev |
| | clgsnftysl sslnfsfvtf lqpvretqii mriflnpsnf |
| | rnftrtcqdi tgefkmcssc lvcepkgnmd fisqeqtskv |
| | lirrgsmevk andfhspcqh fnfsvaplvd hleeynttch |
| | lknhtgrsti medepskeks inytcrimey pndcihislh |
| | lemdiknitc smkitwyilv llvfifliil tirkilegqr |
| | rvqkwqshrd kptsvllrgs dseklralnv qvlsaettqr |
| | lpldqvqevl ppipel (SEQ ID NO: 55; |
| | nucleotide sequence NCBI accession no. |
| | BC030803.1) |
| CST4 | marplctlll lmatlagala ssskeenrii pggiydadln |
| | dewygralhf aiseynkate deyyrrplqv lrareqtfgg |
| | vnyffdvevg rtictksqpn ldtcafheqp elqkkqlcsf |
| | eiyevpwedr mslvnsrcqe a (SEQ ID NO: 56; |
| | nucleotide sequence NCBI accession no. |
| | NM001899.2) |
| CD24 | mgramvarlg lgllllalll ptqiyssett tqtssnssqs |
| | tsnsglapnp tnattkaaqg algstaslfv vslsllhlys |
| | (SEQ ID NO: 57; nucleotide sequence NCBI |
| | accession no. FJ226006.1) |
| FGF5 | mslsfllllf fshlilsawa hgekrlapkg qpgpaatdrn |
| | prgsssrqss ssamssssas sspaaslgsq gsgleqssfq |
| | wspsgrrtgs lycrvgigfh lqiypdgkvn gsheanmlsv |
| | leifaysqgi vgirgvfsnk flamskkgkl hasakftddc |
| | kfrerfqens yntyasaihr tektgrewyv alnkrgkakr |
| | gcsprvkpqh isthflprfk gseqpelsft vtvpekkkpp |
| | spikpkipls aprkntnsvk yrlkfrfg (SEQ ID NO: |
| | 58; nucleotide sequence NCBI accession no. |
| | NM_004464.3) |

### CIN-Responsive Noncanonical NF-kB Signature:

### PPARG, DDIT3, NUPR1, RAB3B, IGFBP4, LRRC8C, TCP11L2, MAFK, NRG1, F2R, KRT19, CTGF, ZFC3H1, MACROD1, GSTA4, SCN9A, BDNF, LACTB

* Genes in bold were suppressed (negative values were used in survival and TCGA analyses)
Noncanonical NF-kB regulatory genes:

### NFKB2, ReIB, MAP3K14, TRAF2, TRAF3, BIRC2, BIRC3

* Genes in bold were suppressed (negative values were used in survival analysis)

### Canonical NF-kB Regulatory Genes:

### NFKB1, ReIA, TRAF1, TRAF4, TRAF5, TRAF6

Interferon regulatory genes

### IRF1, IRF3, IRF7, TBK1

Regulators of epithelial-to-mesenchymal transition (EMT): VIM, ZEB2, SNAI2, ZEB1
Inflammation genes:
RGS16, DENND5A, BTG2, STAT3, IFITM3, CD47, SLAMF7, REL, BCL6, IL18BP, NAMPT, PDE4B, IL8, PSME2, P2RX4, IFI44, CCR7, KLF10, ADRM1, KLF9, NFIL3, CNP, LDLR, HES1, HLA-A, PARP9, NUB1, STAT2, VIP, TGIF1, PVR, MOV10, PSMA2, EIF4E3, IER3, PLA2G4A, TRAFD1, MYD88, VAMP5, TRIM14, TUBB2A, BPGM, B2M, HRH1, PSMB9, LATS2, PTPN6, DCBLD2, PSMB8, ILiR1, PSMB2, SQSTM1, PTX3, ITGA5, EDN1, SLC31A1, SAMHD1, PNPT1, CSF1, TNFRSF9, SOCS1, RELB, VEGFA, ARL4A, DUSP5, CMKLR1, CD38, SLC4A4, SP110, PLAU, DDX58, PSME1, TRAF1, SPSB1, TDRD7, F2RL1, EPSTI1, SAMD9L, NINJ1, RNF19B, LIF, RIPK1, SLC2A6, IRF7, PTAFR, IRAK2, CD14, ITGB8, SCARF1, KIF1B, FOSL2, SOCS3, DUSP1, IRF1, SLC2A3, HBEGF, CXCL3, TNIP1, AHR, SGMS2, FZD5, GCH1, SLC25A28, OSMR, RSAD2, APOL6, ICOSLG, JAG1, GOS2, GEM, KLF4, NFKB1, STAT1, HLA-C, IFIH1, LY6E, EFNA1, SLC16A6, BHLHE40, TRIM26, CD82, CYBB, IL15RA, GABBR1, RELA, PHLDA2, MAP3K8, NUP93, IL7R, PTPRE, IFI27, SNN, NR4A2, SPPL2A, RHOG, SAT1, SLC7A1, IL6, IL15, RAF1, CCL20, ACVR1B, BIRC2, RBCK1, LAP3, ID2, TNFSF10, SIK1, BST2, PANX1, GADD45A, PML, CD40, TRIM21, SECTM1, SSPN, TXNIP, BTG1, AREG, KYNU, PTGS2, IRS2, C3AR1, STAT4, ATP2A2, BIRC3, MAP2K3, CXCL1, NFKBIA, IFNAR1, MET, NR4A1, CXCL2, EBI3, CD83, DNAJB4, CASP7, PHLDA1, NLRC5, IL1B, TRIM25, IERS, RNF213, IL10, NFAT5, ADAR, PNP, MMP14, ICAM4, PPAP2B, SDC4, ABCA1, DUSP2, EIF2AK2, IER2, HERC6, BMP2, IL7, ISG20, GMPR, PSEN1, XAF1, SERPINB8, MTHFD2, EREG, TNFAIP3, TMEM140, KDM6B, CXCL11, CASP1, CYR61, IRF9, GBP2, ADM, TRIP10, PTGER2, METTL7B, SOD2, OAS2, CSF3, SERPINE1, MXD1, ICAM1, ZC3H12A, BCL3, PFKFB3, OGFR, SRI, IFNAR2, FUT4, IL6ST, TNIP2, DUSP4, PROCR, TLR2, OASL, JAK2, C1S, NMI, UBE2L6, LAMP3, TRIB1, TIPARP, IFIT3, GFPT2, IFI30, PPP1R15A, FAM46A, ELF1, UPP1, NOD1, CCL5, FOS, VAMP8, RTP4, TPBG, IL23A, BEST1, CEBPB, TNFSF15, SCN1B, P2RY2, STAT5A, CHST2, HIF1A, ZFP36, KLF2, LPAR1, EHD1, PLSCR1, PDLIM5, OAS1, CXCL10, JUNB, PFKP, CD274, CD55, TNFSF9, ADORA2B, ETS2, OAS3, CASP8, ISG15, WARS, SLC7A2, TNFRSF1B, PARP14, FAS, SAMD9, EIF1, CD74, TOR1B, PTPN2, MARCKS, ST8SIA4, SEMA4D, LYSMD2, ATF3, FOSB, PSMB10, ISOC1, PSMA3, IFNGR2, SMAD3, RIPK2, MARCH1, DHX58, IL4R, TRIM5, LITAF, B4GALT5, NLRP3, ITGB3, CIITA, IFITM1, PIM1, BTG3, CD44, PLK2, DRAM1, FPR1, RHOB, EGR1, GNAI3, C1R, NCOA3, PARP12, AB11, RCAN1, EMP3, IRF2, HLA-DMA, LAMB3, MYC, ATP2B1, YRDC, HLA-DRB1, NDP, MCL1, F3, MT2A, IFI44L, SERPINB2, MAFF, FJX1, LGALS3BP, IL18, GADD45B, TLR1, CEBPD, GNA15, CSF2, SPHK1, IFI35, LYN, PNRC1, IRF5, IFITM2, BANK1, AXL, KLF6, PTGER4, CASP3, PMEPA1, TNC, ZBTB10, PCDH7, CCRL2, CDKN1A, CCNL1, PER1, TLR3, B4GALT1, CLCF1, MVP, CFB, NFKBIE, PTPN1, USP18, NFKB2, CASP4, TNFAIP2, ACVR2A, CX3CL1, IFIT1, EMR1, CFLAR, DDX60, IDO1, CFH, IFIT2, NCOA7, INHBA, TIMP1, RNF144B, MX1, ATP2C1, TSC22D1, PELI1, TAPBP, GBP4, CCND1, SLC31A2, SGK1, ZNFX1, RAPGEF6, CCL2, HLA-B, NFE2L2, UBA7, HAS2, JUN, SLC11A2, FOSL1, SELL, PLAUR, BATF2, TNFAIP8, ST3GAL5, TANK, ARID5B, MX2, TAP1.

Migration and motility genes: CALD1, CAV2, EGFR, FN1, ITGB1, JAG1, MSN, MST1R, NODAL, PDGFRB, RAC1, STAT3, TGFB1, VIM.

### Example 2: Increased Chromosomal Instability in Human Metastases

This Example describes experiments illustrating that chromosomal instability is associated with human metastases.

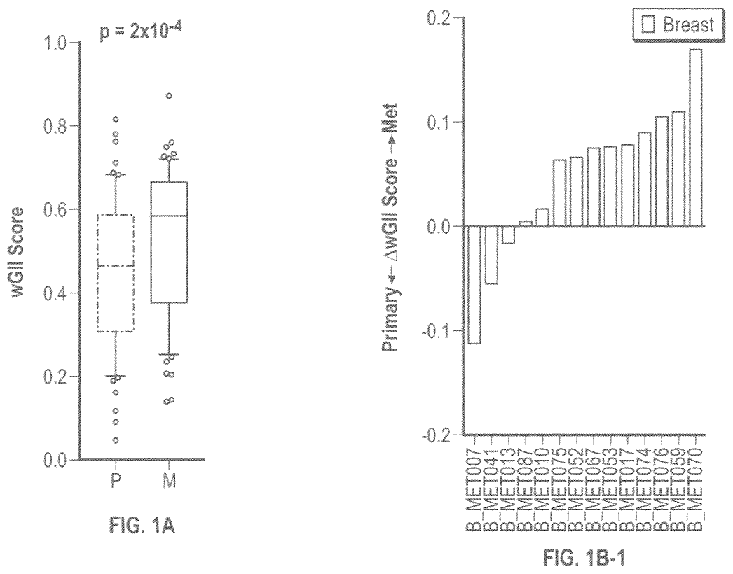

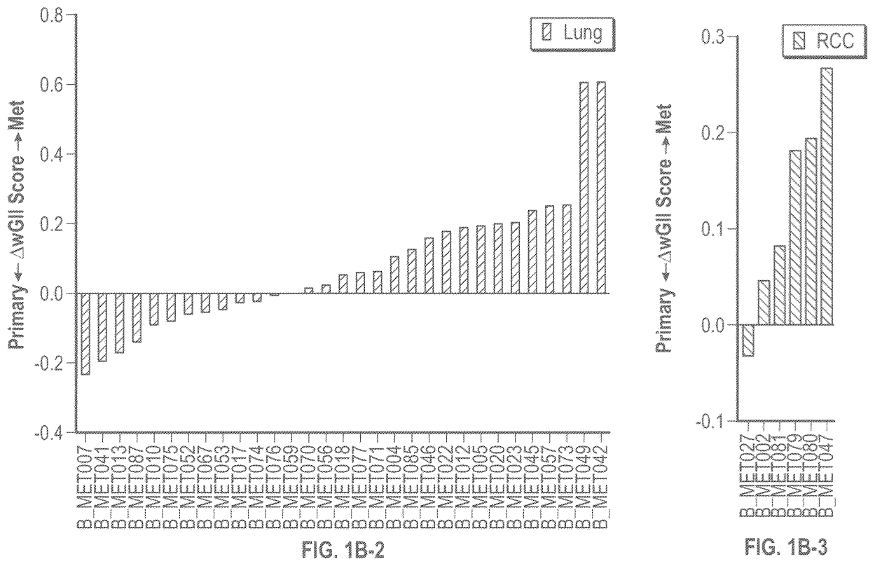

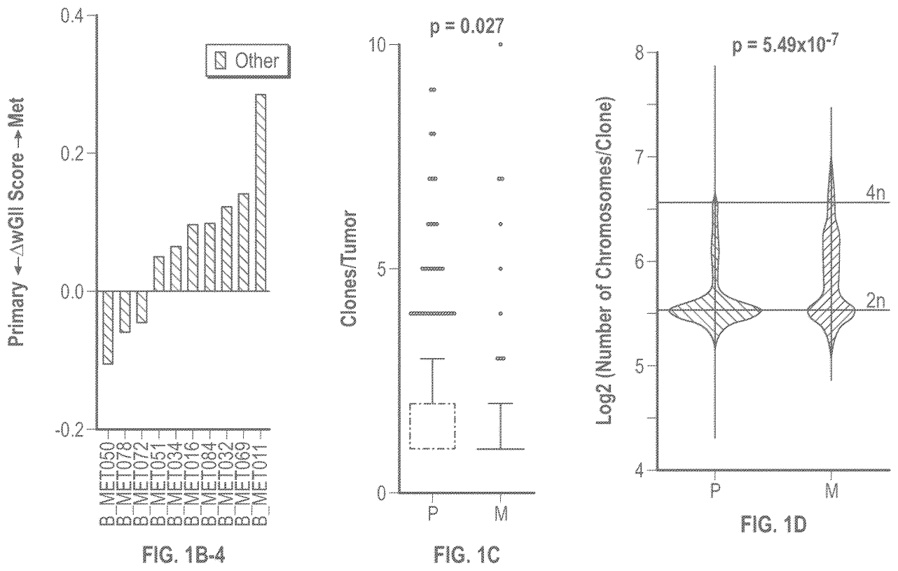

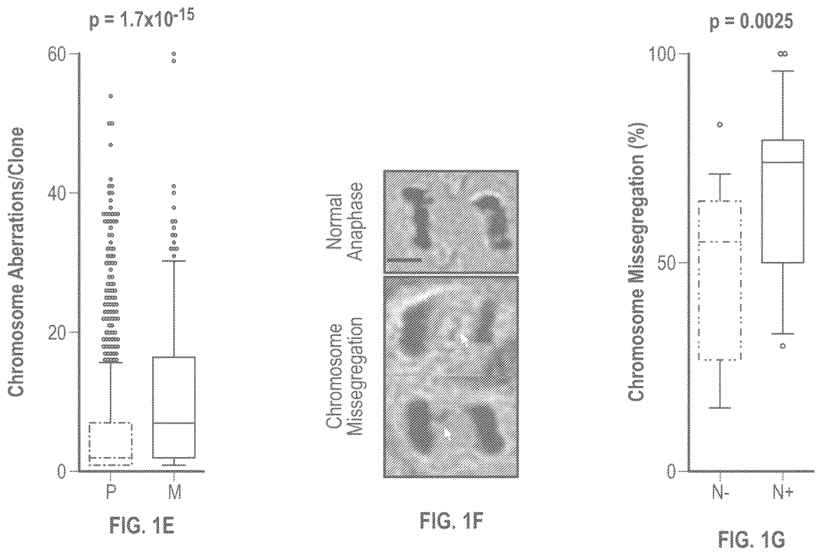

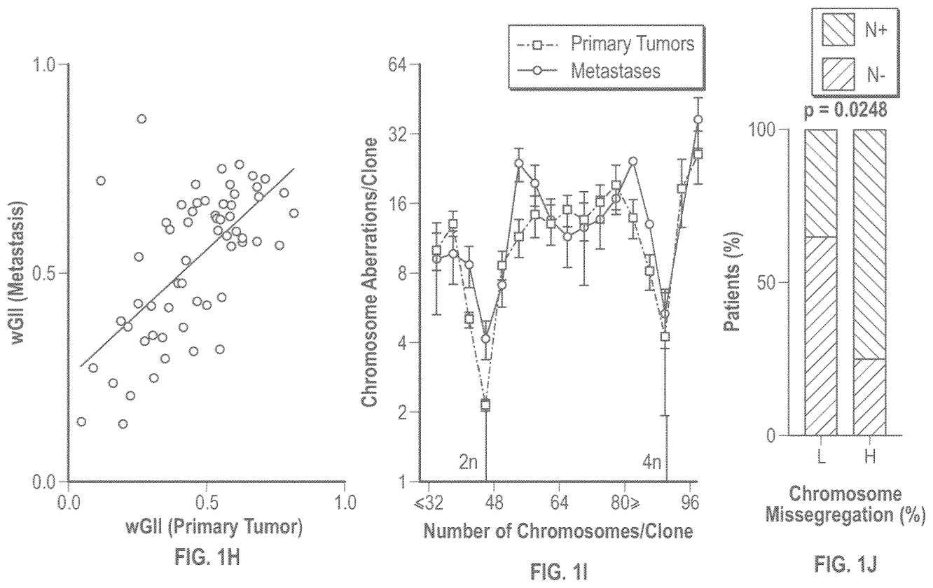

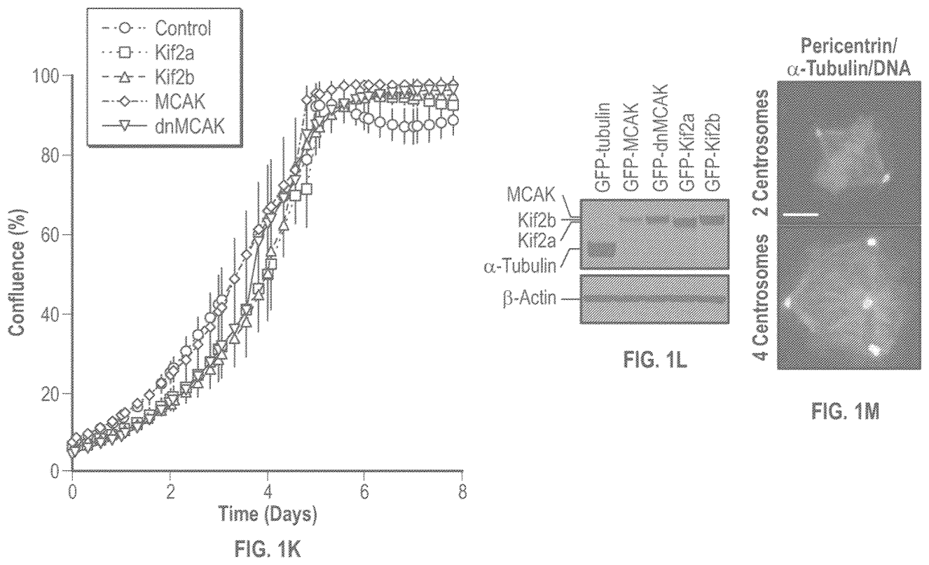

To investigate whether chromosomal instability is associated with human metastases, whole-exome sequence data was compared from 61 primary tumors, comprising 13 tumor types, and matched with brain metastases using data from a recently published cohort (Brastianos et al. Cancer Discovery 5, 1164-1177 (2015)). These data were reanalyzed using the weighted-genomic integrity index (wGII) as a genomic proxy for chromosomal instability. wGII assesses copy number heterogeneity by measuring the percentage of the genome that deviates from the average tumor ploidy (Burrell et al. Nature 494, 492-496 (2013)). There was a significant bias whereby metastases were more likely to have higher wGII scores compared to their matched primary tumors (FIG. 1A-1B-1 to 1B-4, 1H).

Using a second approach, karyotype information was analyzed from 637 primary breast tumors and 131 breast cancer metastases archived in the Mitelman Database of chromosomal translocations (Mitelman et al. website at cgap.nci.nih.gov/Chromosomes/Mitelman). Primary breast tumors contained more clones, as defined by single-cell karyotype analysis, yet they exhibited a strong predilection for normal, near-diploid (2n), karyotypes. On the other hand, samples derived from breast cancer metastases showed significant enrichment for near-triploid (3n) karyotypes and had, on average, twice as many chromosomal aberrations per clone as compared to primary tumors (FIG. 1C-1E). It has been postulated that near-triploid karyotypes represent a convergent optimized evolutionary state where chromosomal instability is maximized (Carter et al. Nat Biotechnol 30, 413-421 (2012); Laughney et al. Cell Rep 12, 809-820 (2015); Storchova et al. J Cell Sci 121, 3859-3866 (2008)). Accordingly, the number of chromosomal aberrations was highest in tumor samples with karyotypes ranging between the diploid and tetraploid (4n) range (FIG. 1I).

Using a third approach, we analyzed data from primary tumor samples taken from patients with locally advanced head and neck squamous cell carcinoma (SCC) for which clinical data on lymph node metastasis at the time of diagnosis was available (Chung et al. Cancer Cell 5, 489-500 (2004)). As a measure of the dynamic nature of chromosomal instability, we directly assessed chromosome segregation integrity in cells fixed while undergoing anaphase (Bakhoun et al. Clin. Cancer Res. 17, 7704-7711 (2011)). The presence of chromatin between normally segregating chromosomes was taken as evidence for chromosome missegregation (FIG. 1F). Primary tumors with associated lymph node metastases had higher rates of chromosome missegregation compared with tumors without lymph node spread. Similarly, patients, whose tumors demonstrated high chromosome missegregation rates, were more likely to present with clinically involved lymph node metastases (FIG. 1F, 1J). Using these three orthogonal approaches, we conclude that chromosomal instability is enriched in human metastases and when present in primary tumors, it is associated with a higher predilection for spread.

### Example 3: Chromosomal Instability Drives Metastasis

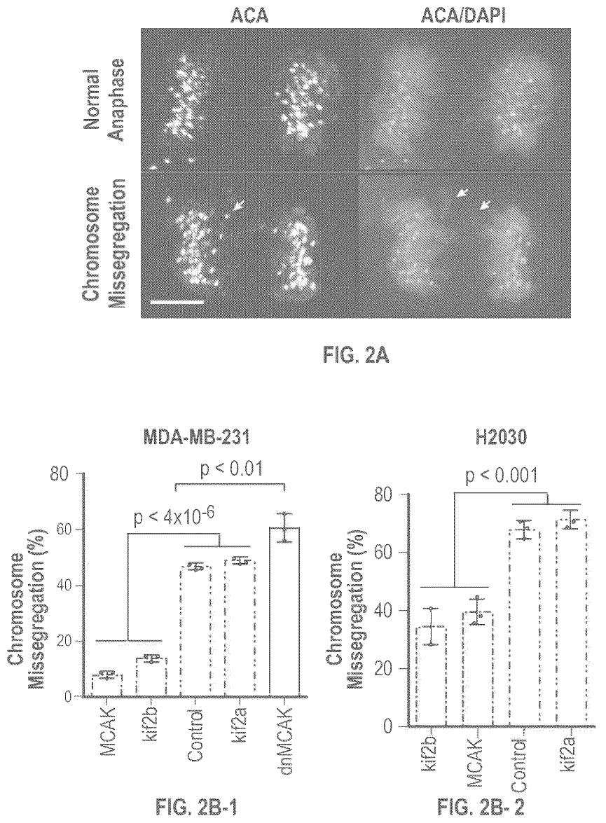

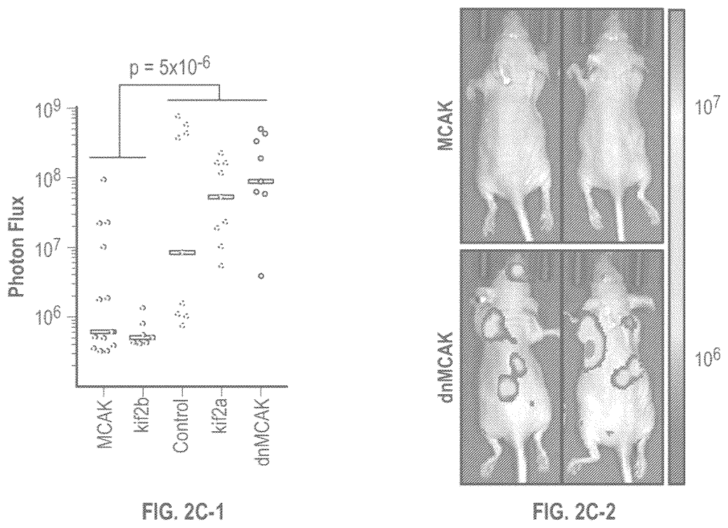

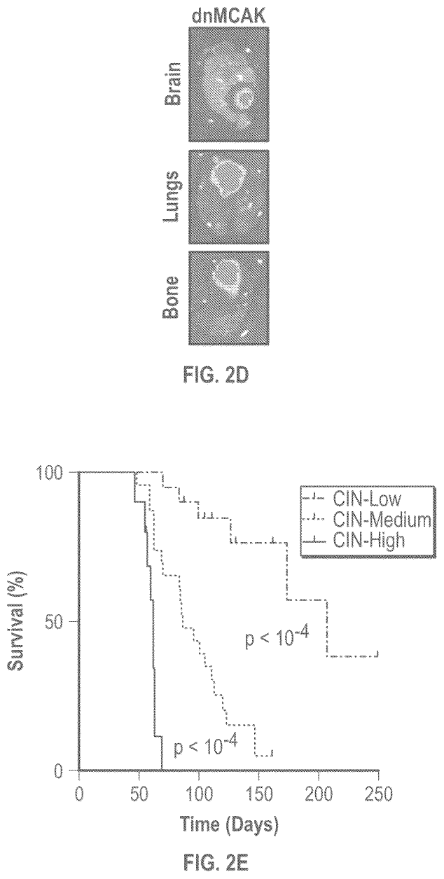

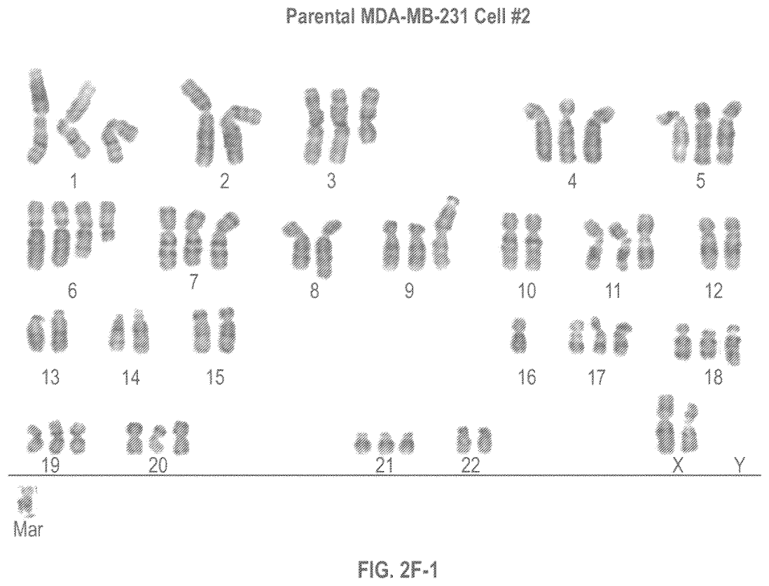

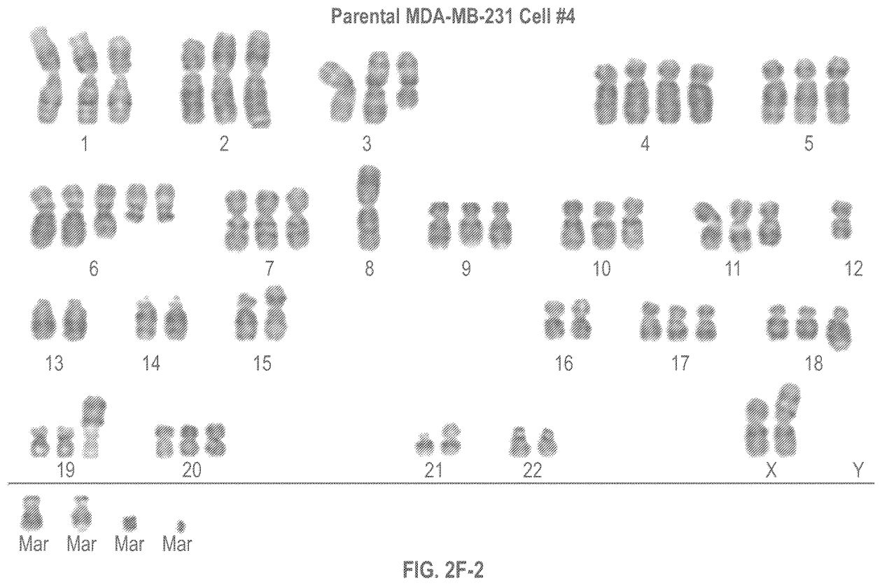

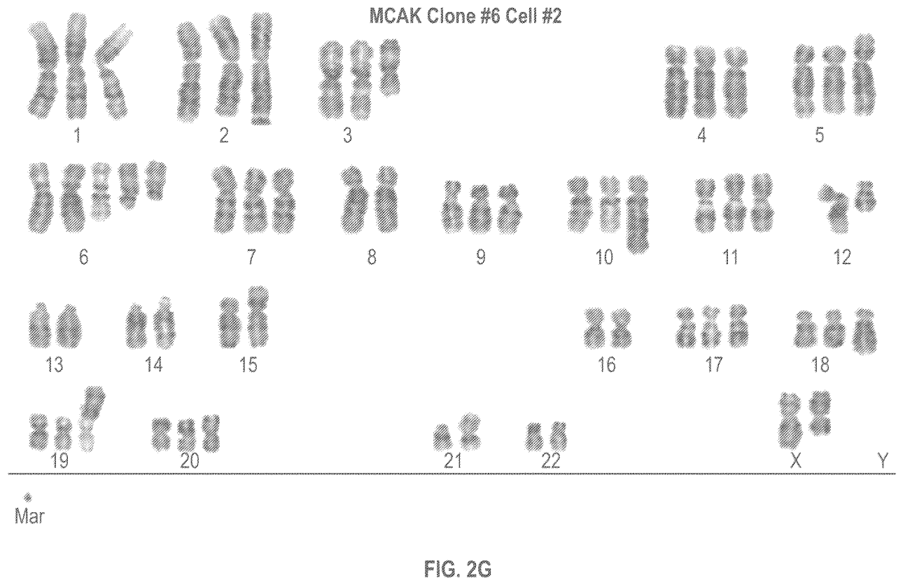

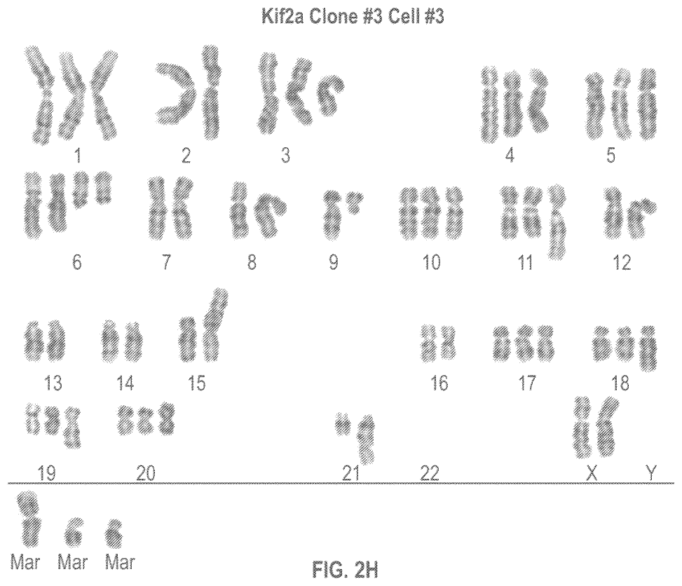

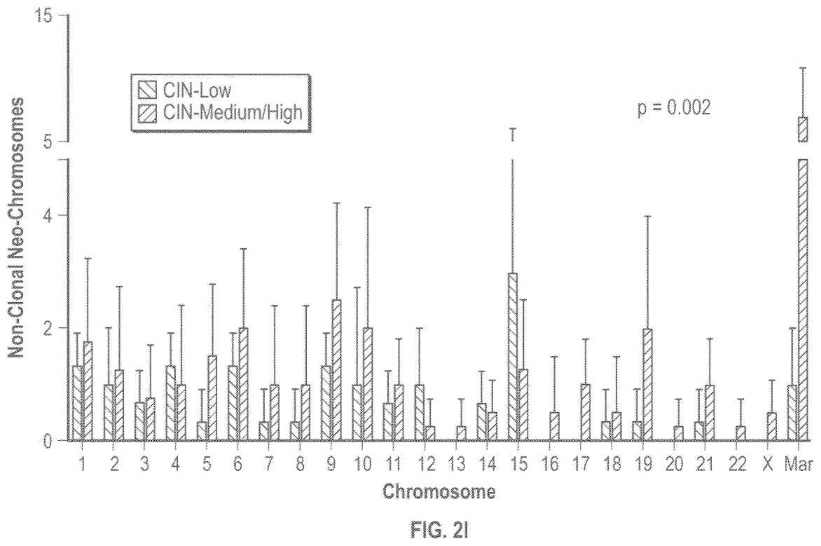

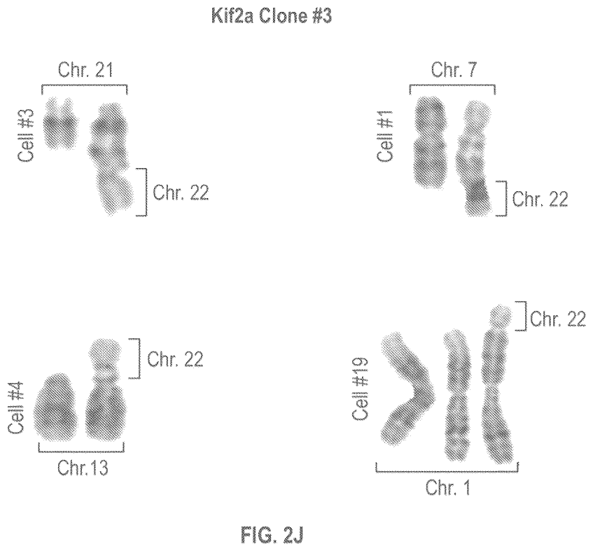

To determine whether chromosomal instability is causally involved in metastasis, we devised a genetic approach (Bakhoun et al., Nat. Cell Biol. 11, 27-35 (2009); Bakhoun et al., Nat Commun 6, 5990 (2015)) to alter the rate of chromosome missegregation in transplantable tumor models of human TNBC (MDA-MB-231) and lung adenocarcinoma (H2030). Cells from these highly metastatic tumor models exhibit elevated basal rates of chromosomal instability with 47% and 67% of anaphase cells, respectively, showing evidence of chromosome segregation errors during anaphase (FIG. 2A, 2B-1 to 2B-2). These cells, with unperturbed chromosome segregation rates, are referred to a as CIN-medium cells. Overexpression of either Kif2b or MCAK/Kif2c in these cells led to significant suppression of chromosome segregation errors (referred to as CIN-low cells). Conversely, overexpression of a dominant negative form of MCAK²⁴ (dnMCAK) led to a further increase in chromosome segregation errors in MDA-MB-231 cells—referred to as C/N-high (FIG. 2B-1 to 2B-2, FIG. 1L).

Overexpression of Kinesin-13 proteins did not alter cellular proliferation rates in culture or the number of centrosomes per cell (FIG. 1K, 1M). As an important control, Kif2a was overexpressed, Kif2a is a third member of the microtubule-depolymerizing kinesin-13 proteins that lacks any kinetochore or centromere localization domains (Ems-McClung et al. Semin. Cell Dev. Biol. 21, 276-282 (2010)). Kif2a overexpression had no effect on chromosomal instability despite exhibiting microtubule-depolymerizing activity on interphase microtubules similar to that of Kif2b and MCAK (FIGS. 2B-1 and 2B-2).

Karyotyping of the parental MDA-MB-231 cell line revealed widely aneuploid (near-triploid) chromosome content and demonstrated significant karyotypic heterogeneity as well as chromosomal abnormalities, as expected from a chromosomally unstable cell line (FIG. 2F-1 to 2F-2). Suppression of chromosomal instability in these cells led to a reduction in karyotypic heterogeneity in single-cell derived clones, as evidenced by the presence of fewer neo-chromosomes (chromosomes exhibiting non-clonal structural abnormalities) in CIN-low cells as compared to CIN-medium or CIN-high (FIG. 2G-2I). For instance, chromosome 22 was fused with other chromosomes leading to unique chromosomal combinations in different cells within the same Kif2a-expressing clonal population (FIG. 2J), indicating convergent karyotypic evolution conferred by chromosomal instability. Conversely, such events were uncommon in CIN-low clones. Nonetheless, CIN-low cells maintained highly aneuploid karyotypes, yet they faithfully propagated these abnormal karyotypes in a stable manner (FIG. 2G, 2I). By comparing chromosomally stable aneuploid cells to their chromosomally unstable aneuploid counterparts, we can experimentally examine the role of chromosomal instability, independently of aneuploidy, in metastasis.

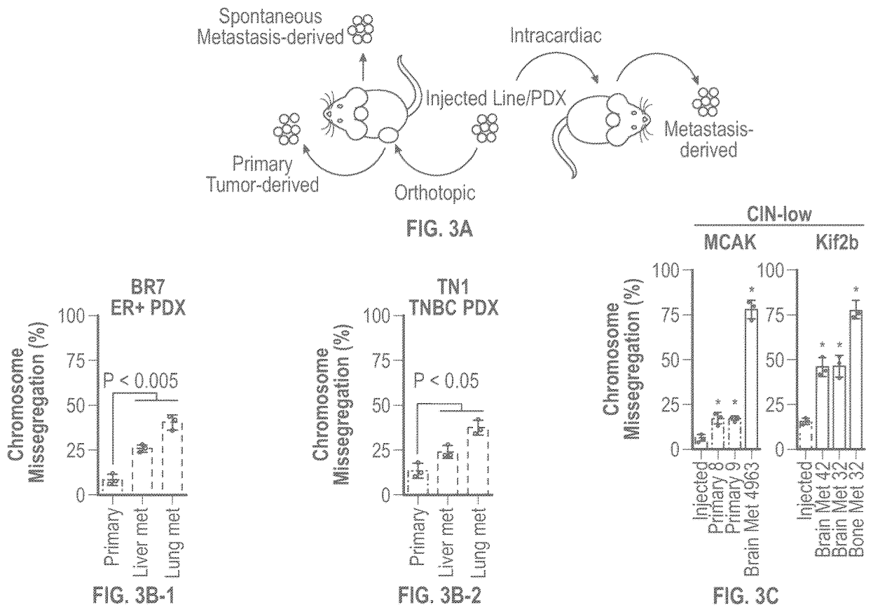

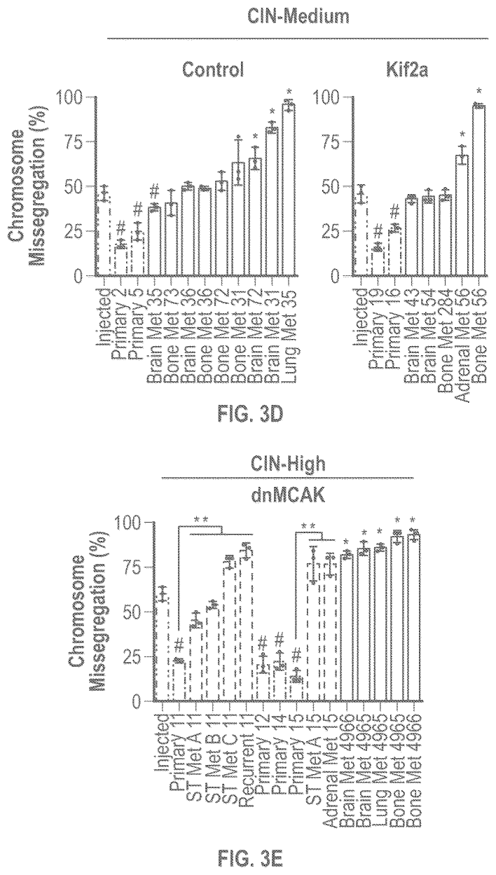

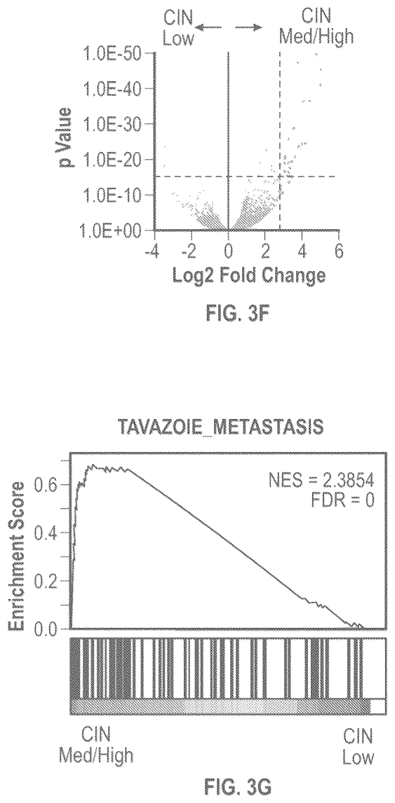

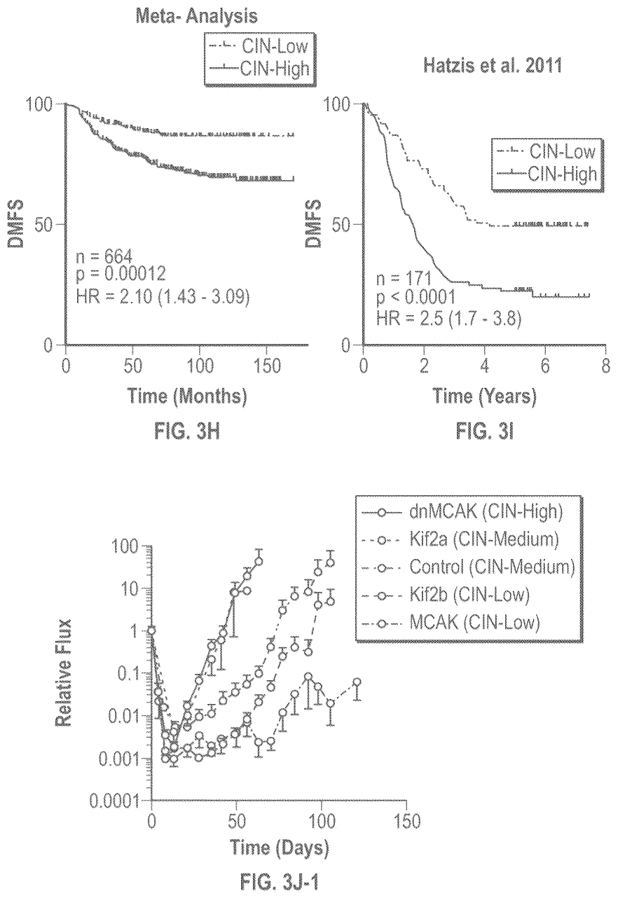

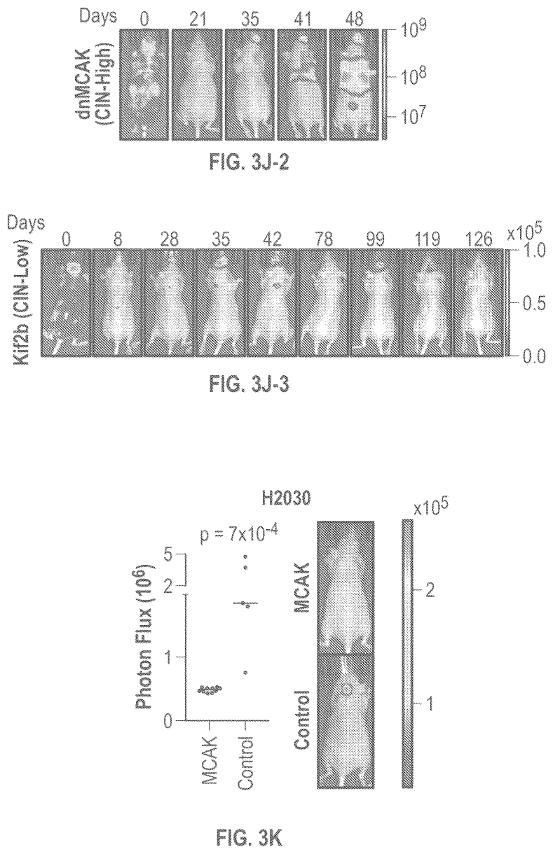

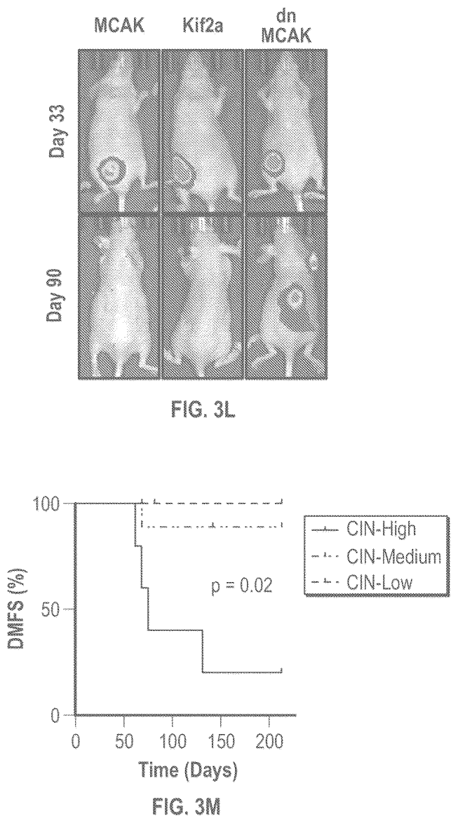

MDA-MB-231 cells were directly injected in the left cardiac ventricles of athymic mice to enable systemic dissemination (FIGS. 3J-1 and 3J-2, Day 0). Metastatic colonization was then tracked using a bioluminescence reporter assay. Experimentally altering chromosome missegregation rates had a dramatic effect on metastatic colonization, whereby mice harboring CIN-high cells rapidly succumbed to widespread disease within 60 days of injection with metastases present in the brain, bone, lungs, adrenal glands, and soft tissues. Conversely, mice injected with CIN-low cells exhibited a strikingly lower metastatic tumor burden and had a median survival of 207 days with some living over 290 days (FIG. 2C-2E, 3J). In some animals, CIN-low metastases waxed-and-waned and, at times, spontaneously resolved, whereas CIN-high metastases involved multiple organs and rapidly progressed leading to death (FIGS. 3J-1 and 3J-2), indicating a potential role for chromosomal instability in the initiation as well as maintenance of metastases. Similar results were obtained after intraventricular injection of lung adenocarcinoma H2030 cells (FIG. 3K).

To assess the role of chromosomal instability in metastasis starting from the primary tumor setting, we performed orthotopic injections of MDA-MB-231 in the mammary fat pad followed by surgical excision of the primary tumor to enable time for metastatic dissemination (FIG. 3L, see methods described in Example 1). Chromosomal instability status did not noticeably alter primary tumor implantation efficiency as both CIN-low, CIN-medium, and CIN-high tumors were capable of forming palpable tumors at similar rates (not shown), however mice orthotopically injected with CIN-high cells exhibited a significantly shorter distant metastasis-free survival (DMFS) compared to animals injected with CIN-low tumor cells, which had no metastatic events (FIG. 3M). Collectively, these results show that chromosomal instability is a critical factor in tumor metastasis and that suppressing chromosomal instability reduces metastatic potential even in highly abnormal and aneuploid cells.

To evaluate the selection dynamics with respect to chromosomal instability during tumor dissemination, we assessed chromosome missegregation in the injected cells as well as cells (passage 1) derived from primary tumors or metastatic colonies (FIGS. 3J-1 and 3J-2). This analysis was first performed in two metastasis-competent patient-derived xenografts (PDX) belonging to two breast cancer subtypes: ER+ and TNBC (see Example 1). In both PDX tumor models, cells derived from orthotopically transplanted primary tumors had lower chromosome missegregation rates compared to matched metastases derived from the same animal (FIG. 3B). This analysis was then repeated using MDA-MB-231 cells and found that regardless of the chromosomal instability status of the injected cells, the majority of metastases enriched for cells that had significantly higher rates of chromosome missegregation compared to the injected cells (FIG. 3C-3E). Conversely, cells derived from most primary tumors had significantly lower rates of chromosome missegregation compared to the injected cells (FIG. 3D-3E). When CIN-high cells were injected (FIG. 3e, left-most bar) in the mammary fat pad, chromosome missegregation rates significantly decreased in the primary tumors (FIG. 3E, bars labeled ‘primary’) before increasing once more in the metastases spontaneously arising in the same animal (FIG. 3E, corresponding bars labeled ‘met’). These results reveal the potential for rapid genomic plasticity arising from chromosomal instability and demonstrate a strong selective pressure for high rates of chromosome missegregation during the evolution of metastasis.

### Example 4: Chromosomal Instability Enriches Mesenchymal Traits

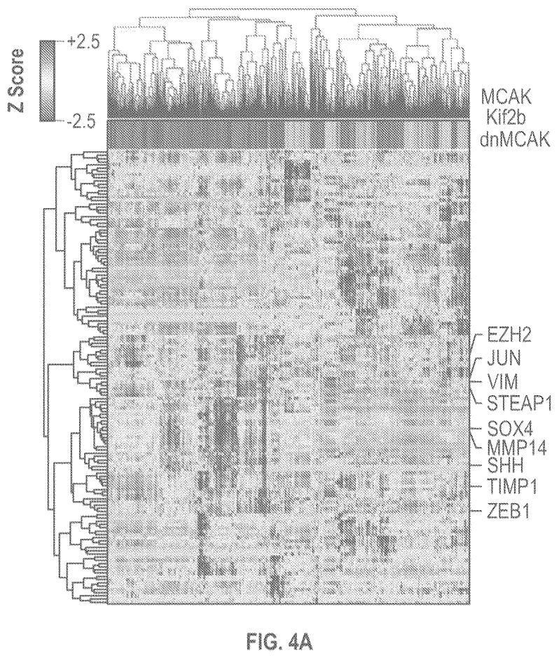

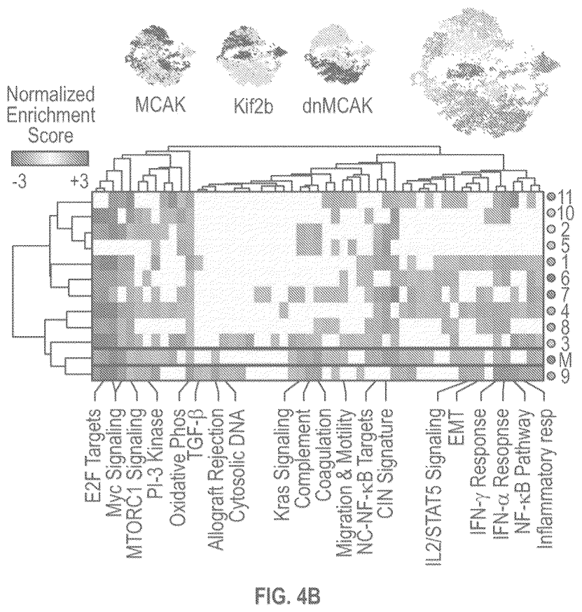

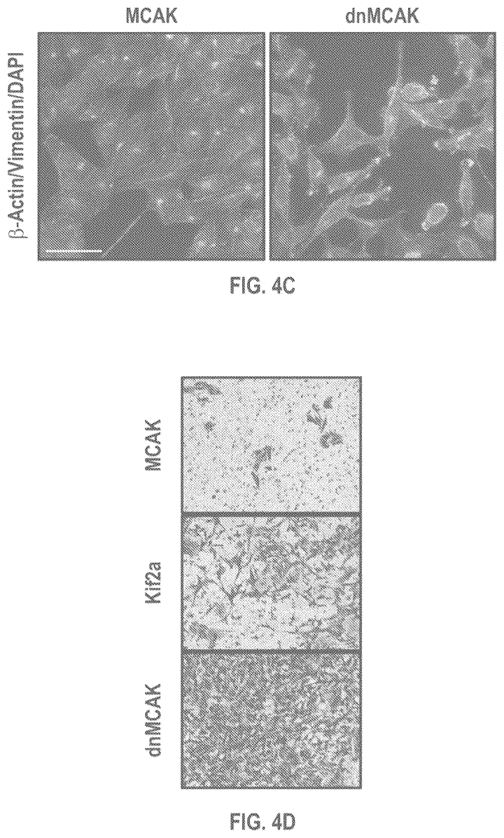

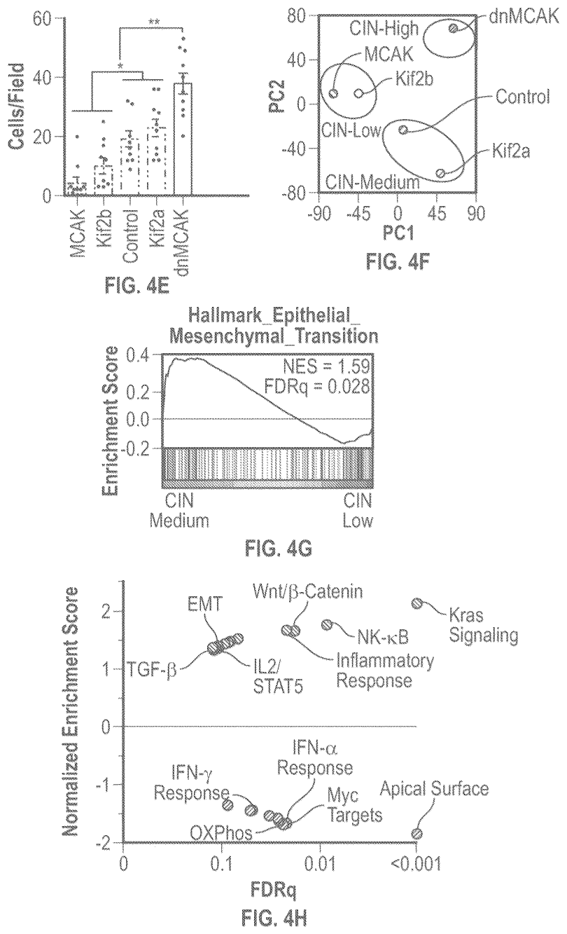

To examine the cellular changes in response to chromosomal instability, we performed bulk RNA sequencing (RNA-seq) of CIN-low, CIN-medium, and CIN-high MDA-MB-231 cells and found 1,584 differentially expressed genes when comparing CIN-low to CIN-medium/high (FIG. 3F). Principle component analysis (PCA) on gene-expression accurately separated samples according to their chromosomal instability status (FIG. 4F). Gene set enrichment analysis (GSEA) revealed that metastasis-related gene sets were amongst the most highly enriched in CIN-medium/high cells compared with CIN-low (FIG. 3G), indicating that chromosome missegregation induces a transcriptional change similar to that observed in metastasis. Indeed, the top 23 differentially expressed genes in CIN-medium/high compared with CIN-low were highly prognostic in human breast cancer patients as they predicted distant-metastasis-free survival (DMFS) in a meta-analysis (Györffy et al. Breast Cancer Res. Treat. 123, 725-731 (2010)) as well as a validation cohort (Hatzis et al., JAMA 305, 1873-1881 (2011)) (FIG. 3H-31).

This list of 23 genes whose elevated expression PREDICTS increased distant-metastasis free survival in breast cancer is referred to as the chromosomal instability (CIN) signature and includes elevated expression of: PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, FGF5, and NTN4. Such predictive power was largely preserved across tumor subtypes, grades, and lymph node status. For example, the 23-gene chromosomal instability (CIN) signature accurately identified that CIN-low patients had increased distant-metastasis free survival compared to CIN-high patients with a variety of breast cancers including node-negative, node-positive, grade 2, grade 3, grade 1/2, grade 3, ER+, ER−, and Her2+ breast cancers.

Epithelial-to-mesenchymal (EMT) transcriptional programs were also highly enriched in CIN-medium/high cells (FIG. 4G). To further understand how chromosomal instability influences cellular heterogeneity, single-cell RNA sequencing (scRNA-seq) was performed using a bead-based molecular barcoding technology (Klein et al. Cell 161, 1187-1201 (2015)) on two CIN-low MDA-MB-231 cell lines (Kif2b and MCAK) and one CIN-high cell line (dnMCAK) comprising a total of 6,821 cells. Single-cell library size was consistent across samples. Clustering of single cells using key EMT genes successfully classified most cells based on their CIN-status and it revealed a fraction of cells that was highly enriched in mesenchymal markers including key EMT regulators such as vimentin and ZEB1. This fraction was primarily comprised of dnMCAK expressing CIN-high cells (FIG. 4A). Conversely, CIN-low cells were highly enriched in epithelial markers.

Unsupervised graph-based clustering (Levine et al. Cell 162, 184-197 (2015)) based on all genes was then employed to identify intrinsic subpopulations in an unbiased manner. A subpopulation (referred to as subpopulation ‘M’) was identified that exhibited increased expression of genes involved in epithelial-to-mesenchymal transition (EMT) and metastasis and it was concomitantly enriched for the chromosomal instability (CIN) gene signature. Subpopulation M included 45% of the total dnMCAK expressing cells compared to only 6% of the CIN-low cells, respectively (FIG. 4B, FIG. 6I-6J).

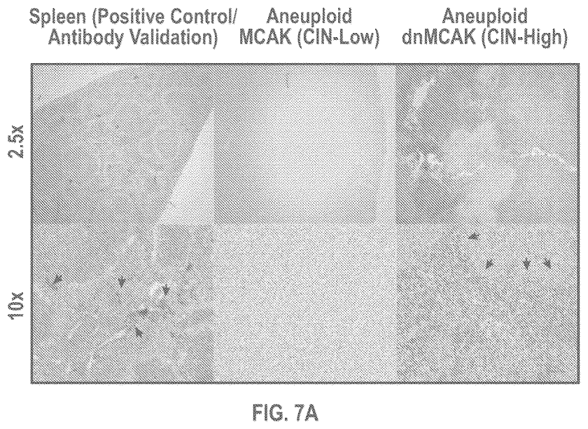

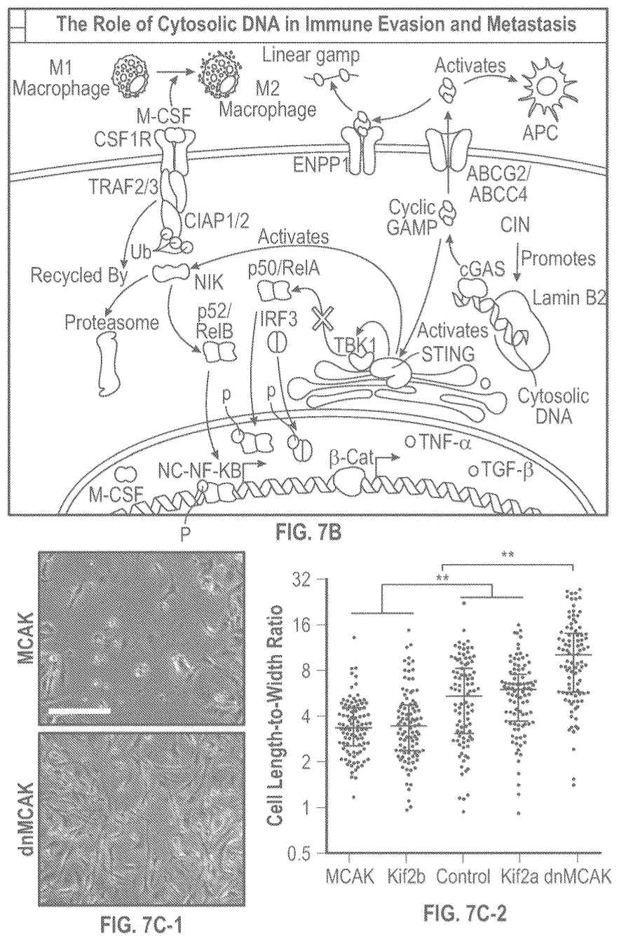

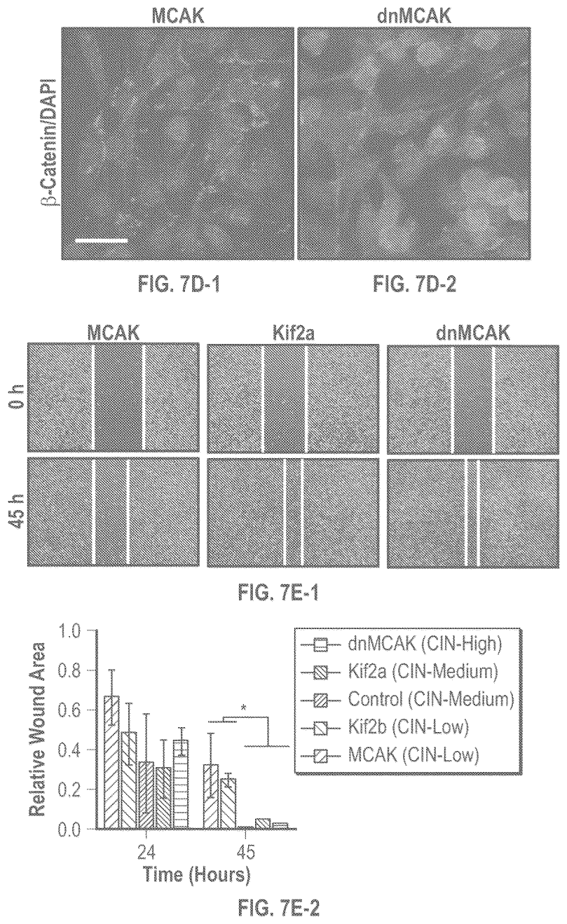

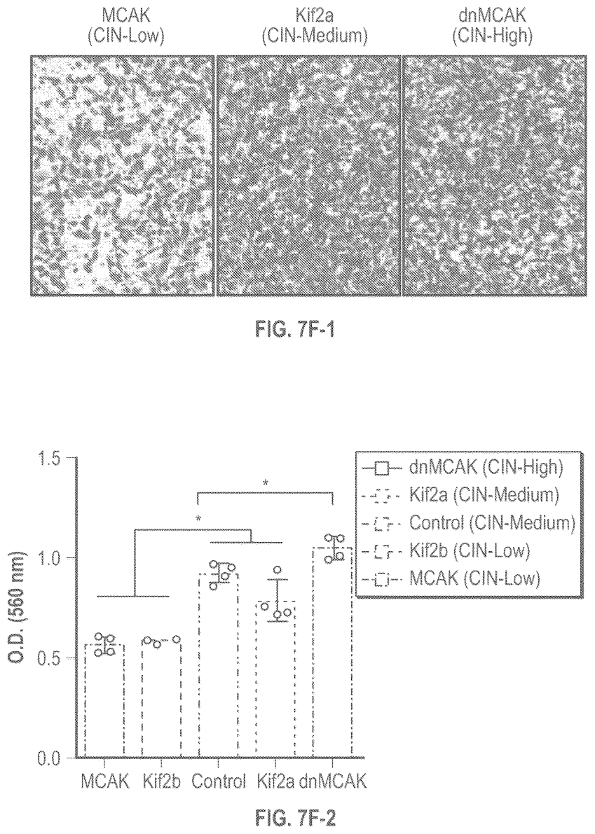

These results were validated experimentally using high-resolution fluorescence microscopy whereby we found cells expressing dnMCAK to have more elongated features (defined by length-to-width ratio) exhibiting actin cytoskeletal reorganization. They also exhibited mesenchymal characteristics such as diffuse vimentin staining and changes in localization of β-catenin: from cell-to-cell junctions in MCAK expressing cells to the cytoplasm and nucleus of dnMCAK expressing cells (FIG. 4C, FIG. 7C-7D). Accordingly, cells with high levels of chromosomal instability exhibited increased migratory capacity and were significantly more invasive through collagen basement membranes in vitro (FIG. 4D, FIG. 7E-7F). Collectively, these results demonstrate that chromosomal instability promotes a cell-autonomous invasive program that facilitates the metastatic process.

### Example 5: Chromosomal Instability-Induced Cell-Intrinsic Inflammation

This Example illustrates that chromosomal instability induces intrinsic inflammation.

To further define chromosomal instability-responsive pathways, a gene-gene Pearson correlation analysis was performed using scRNA-seq data and identified two large gene modules. Module 2 contained genes involved in epithelial-to-mesenchymal transition (EMT) as well as a large number of inflammatory pathways (FIG. 5A).

As described in Example 1, the EMT genes include VIM, ZEB2, SNAI2, and ZEB1. The inflammatory pathway genes include RGS16, DENND5A, BTG2, STAT3, IFITM3, CD47, SLAMF7, REL, BCL6, IL18BP, NAMPT, PDE4B, IL8, PSME2, P2RX4, IFI44, CCR7, KLF10, ADRM1, KLF9, NFIL3, CNP, LDLR, HES1, HLA-A, PARP9, NUB1, STAT2, VIP, TGIF1, PVR, MOV10, PSMA2, EIF4E3, IER3, PLA2G4A, TRAFD1, MYD88, VAMP5, TRIM14, TUBB2A, BPGM, B2M, HRH1, PSMB9, LATS2, PTPN6, DCBLD2, PSMB8, IL1R1, PSMB2, SQSTM1, PTX3, ITGA5, EDN1, SLC31A1, SAMHD1, PNPT1, CSF1, TNFRSF9, SOCS1, RELB, VEGFA, ARL4A, DUSP5, CMKLR1, CD38, SLC4A4, SP110, PLAU, DDX58, PSME1, TRAF1, SPSB1, TDRD7, F2RL1, EPSTI1, SAMD9L, NINJ1, RNF19B, LIF, RIPK1, SLC2A6, IRF7, PTAFR, IRAK2, CD14, ITGB8, SCARF1, KIF1B, FOSL2, SOCS3, DUSP1, IRF1, SLC2A3, HBEGF, CXCL3, TNIP1, AHR, SGMS2, FZD5, GCH1, SLC25A28, OSMR, RSAD2, APOL6, ICOSLG, JAG1, GOS2, GEM, KLF4, NFKB1, STAT1, HLA-C, IFIH1, LY6E, EFNA1, SLC16A6, BHLHE40, TRIM26, CD82, CYBB, IL15RA, GABBR1, RELA, PHLDA2, MAP3K8, NUP93, IL7R, PTPRE, IFI27, SNN, NR4A2, SPPL2A, RHOG, SAT1, SLC7A1, IL6, IL15, RAF1, CCL20, ACVR1B, BIRC2, RBCK1, LAP3, ID2, TNFSF10, SIK1, BST2, PANX1, GADD45A, PML, CD40, TRIM21, SECTM1, SSPN, TXNIP, BTG1, AREG, KYNU, PTGS2, IRS2, C3AR1, STAT4, ATP2A2, BIRC3, MAP2K3, CXCL1, NFKBIA, IFNAR1, MET, NR4A1, CXCL2, EB13, CD83, DNAJB4, CASP7, PHLDA1, NLRC5, IL1B, TRIM25, IERS, RNF213, I10, NFAT5, ADAR, PNP, MMP14, ICAM4, PPAP2B, SDC4, ABCA1, DUSP2, EIF2AK2, IER2, HERC6, BMP2, IL7, ISG20, GMPR, PSEN1, XAF1, SERPINB8, MTHFD2, EREG, TNFAIP3, TMEM140, KDM6B, CXCL11, CASP1, CYR61, IRF9, GBP2, ADM, TRIP10, PTGER2, METTL7B, SOD2, OAS2, CSF3, SERPINE1, MXD1, ICAM1, ZC3H12A, BCL3, PFKFB3, OGFR, SRI, IFNAR2, FUT4, IL6ST, TNIP2, DUSP4, PROCR, TLR2, OASL, JAK2, C1S, NMI, UBE2L6, LAMP3, TRIB1, TIPARP, IFIT3, GFPT2, IFI30, PPP1R15A, FAM46A, ELF1, UPP1, NOD1, CCL5, FOS, VAMP8, RTP4, TPBG, IL23A, BEST1, CEBPB, TNFSF15, SCN1B, P2RY2, STAT5A, CHST2, HIF1A, ZFP36, KLF2, LPAR1, EHD1, PLSCR1, PDLIM5, OAS1, CXCL10, JUNB, PFKP, CD274, CD55, TNFSF9, ADORA2B, ETS2, OAS3, CASP8, ISG15, WARS, SLC7A2, TNFRSFIB, PARP14, FAS, SAMD9, EIF1, CD74, TOR1B, PTPN2, MARCKS, ST8SIA4, SEMA4D, LYSMD2, ATF3, FOSB, PSMB10, ISOC1, PSMA3, IFNGR2, SMAD3, RIPK2, MARCH1, DHX58, IL4R, TRIM5, LITAF, B4GALT5, NLRP3, ITGB3, CIITA, IFITM1, PIM1, BTG3, CD44, PLK2, DRAM1, FPR1, RHOB, EGR1, GNAI3, C1R, NCOA3, PARP12, AB11, RCAN1, EMP3, IRF2, HLA-DMA, LAMB3, MYC, ATP2B1, YRDC, HLA-DRB1, NDP, MCL1, F3, MT2A, IFI44L, SERPINB2, MAFF, FJX1, LGALS3BP, IL18, GADD45B, TLR1, CEBPD, GNA15, CSF2, SPHK1, IFI35, LYN, PNRC1, IRF5, IFITM2, BANK1, AXL, KLF6, PTGER4, CASP3, PMEPA1, TNC, ZBTB10, PCDH7, CCRL2, CDKN1A, CCNL1, PER1, TLR3, B4GALT1, CLCF1, MVP, CFB, NFKBIE, PTPN1, USP18, NFKB2, CASP4, TNFAIP2, ACVR2A, CX3CL1, IFIT1, EMR1, CFLAR, DDX60, IDO1, CFH, IFIT2, NCOA7, INHBA, TIMP1, RNF144B, MX1, ATP2C1, TSC22D1, PELI1, TAPBP, GBP4, CCND1, SLC31A2, SGK1, ZNFX1, RAPGEF6, CCL2, HLA-B, NFE2L2, UBA7, HAS2, JUN, SLC11A2, FOSL1, SELL, PLAUR, BATF2, TNFAIP8, ST3GAL5, TANK, ARID5B, MX2, and TAP1.

The chromosomal instability signature genes include PELI2, BMP2, SHH, TNS4, RAB3B, ROBO1, ARHGAP28, CHN2, CST1, F13A1, CPVL, SEMA6D, NHSL2, GTF21P7, DPYSL3, PCDH7, KHDRBS3, TRAC, TMEM156, CST4, CD24, FGF5, and NTN4. This list of 23 genes whose elevated expression predicts increased distant-metastasis free survival in breast cancer is referred to as the chromosomal instability (CIN) signature when elevated expression of these genes is detected.

There was a significant correlation between inflammation-related genes, the chromosomal instability signature genes, and EMT genes, all of which were highly enriched in subpopulation M (FIG. 48, black box; FIG. 5B). Bulk RNA-seq data also revealed significant enrichment for genes involved in the inflammatory response and TNF-α/NF-κB pathways in chromosomal instability-medium/high cells (FIG. 4H). These data indicate that a relationship may exist between chromosomal instability and tumor cell-intrinsic inflammation.

Induction of cell-intrinsic inflammation in response to chromosomal instability, even prior to in vivo transplantation, is unexpected and is reminiscent of a viral infection. We then asked whether chromosomal instability might induce cellular inflammation by introducing genomic DNA into the cytosol, thus eliciting intrinsic cellular inflammation normally reserved for anti-viral immunity.

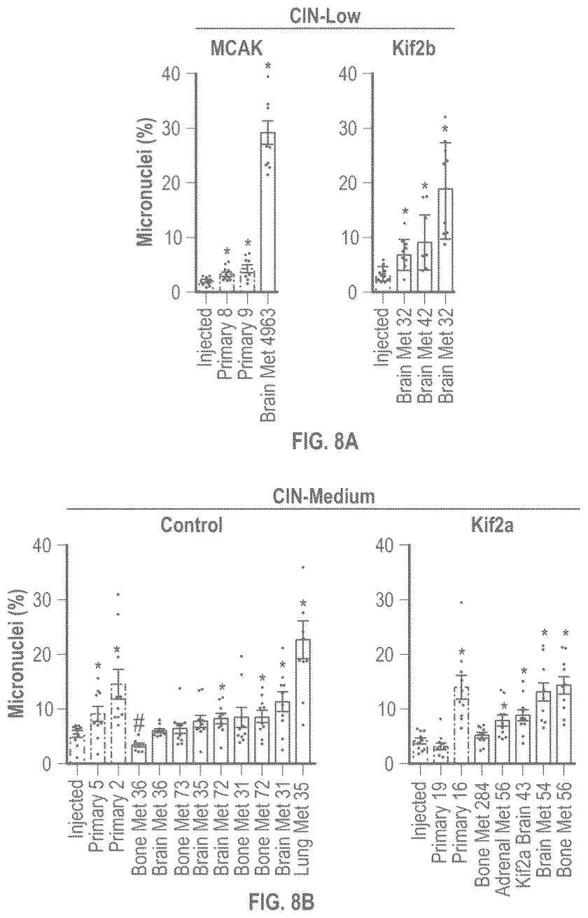

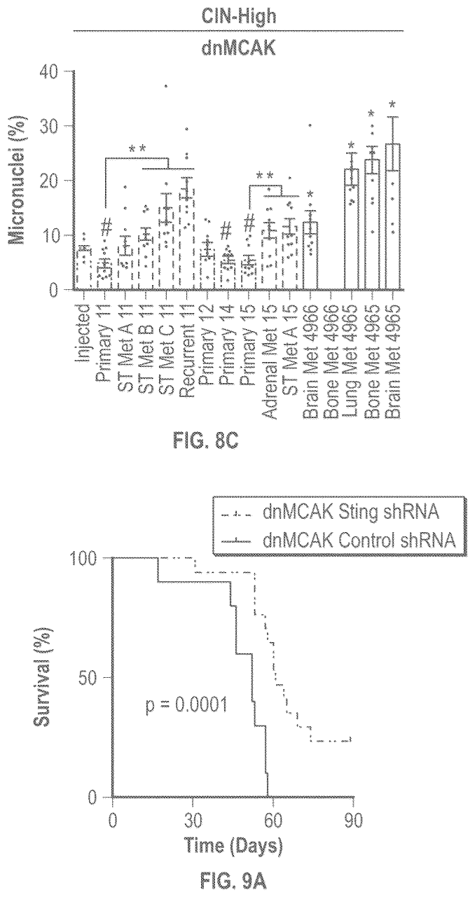

Chromosomal instability-medium/high exhibited a higher preponderance for micronuclei, as seen when comparing cells derived from metastatic lesions as compared to primary tumors. There was an overall significant correlation between chromosome missegregation rates and the frequency of micronuclei (FIG. 5C-5E, FIG. 8A-8C).

To determine if the presence of rupture-prone micronuclei contributed to the generation of cytosolic DNA, cells were stained using two different anti-dsDNA antibodies after selective plasma membrane permeabilization. In each case, cells expressing dnMCAK exhibited significantly increased levels of cytosolic dsDNA and single-stranded DNA (ssDNA) compared to cells exhibiting low levels of chromosomal instability (FIG. 5G). The dsDNA signal, which was distinct from mitochondrial staining, disappeared after treatment with double-strand-specific—but not single-strand-specific—nuclease and after overexpression of Dnase2, confirming the specificity of these antibodies (FIG. 5H).

Direct quantification of dsDNA levels after subcellular fractionation revealed a four-fold reduction in cytosolic DNA in cells exhibiting low levels of chromosomal instability compared to cells exhibiting medium to high levels of chromosomal instability (CIN-medium/high cells; FIG. 5G). Finally, whole-genome sequencing at 30× coverage of subcellular fractions confirmed the genomic origin of cytosolic DNA (not shown). To further ascertain that cytosolic dsDNA arises from micronuclear rupture, mCherry-Lamin B2 was overexpressed as a means to stabilize micronuclear envelopes (Hatch et al. Cell 154, 47-60 (2013)) and cells were observed to ascertain whether there was selective reduction in cytosolic dsDNA staining in Lamin B2 overexpressing cells (FIG. 5I). Collectively, these results demonstrate that chromosomal instability induces cytosolic DNA of genomic origin through micronuclear rupture.

### Example 6: Metastasis from Cytosolic DNA Response

This Example illustrates that exposure of DNA to cytosol can lead to cancer cell metastasis.

Cytosolic dsDNA elicits a distinct signaling pathway leading to the induction of type I interferon signaling used to combat viral infection. To explore the downstream consequences of cytosolic dsDNA in chromosomally unstable cells, cells were stained for cyclic GMP-AMP synthase (cGAS), a key sensor of cytosolic DNA (Sun et al. Science 339, 786-791 (2013)). cGAS exhibited a striking localization to approximately half of all micronuclei that were present regardless of the level of chromosomal instability (FIG. 6A-6B). cGAS-plus micronuclei were positively stained using anti-dsDNA antibody after selective plasma membrane permeabilization whereas cGAS-minus micronuclei did not (FIG. 6A). Furthermore, stabilizing micronuclear envelopes through Lamin B2 overexpression (Hatch et al., Cell 154, 47-60 (2013)), significantly diminished the relative fraction of micronuclei with cGAS staining (FIG. 6B). Collectively, these results demonstrate that micronuclear rupture is required for cytosolic DNA sensing by cGAS. And, although chromosomal instability does not influence micronuclear integrity per se, it increases the overall number of micronuclei per cell and consequently the probability of cGAS activation (FIG. 5C-5E, FIG. 6A-6B).

cGAS catalyzes the formation of 2′3′-cyclic GMP-AMP (cGAMP), which in turn activates stimulator of interferon genes (STING, also known as TMEM173) to induce Type I interferon production. Increased STING protein levels were observed in CIN-high cells (FIG. 6C). However, there was no evidence for activation of downstream interferon-regulatory factors or the canonical NF-κB pathway as evidenced by the lack of significant changes in p65 or IRF phosphorylation as well as absence of their nuclear translocation (FIG. 6C). This is consistent with observations that cancer cells suppress interferon production downstream of cytosolic DNA sensing (Stetson et al., Cell 134, 587-598 (2008); Lau et al. Science 350, 568-571 (2015)). Cytosolic DNA, however, can activate the noncanonical NF-κB pathway in a STING-dependent and a TBK1-independent manner (Abe et al. J. Virol. 88, 5328-5341 (2014)).

Evidence was observed for noncanonical NF-κB pathway activation in cells exhibiting medium to high levels of chromosomal instability (CIN-medium/high cells). These cells had lower levels of the noncanonical NF-κB precursor protein, p100, as well as increased quantities of phosphorylated p100 and its cleaved product, p52, relative to the total p100 pool, in line with activation of the noncanonical pathway (FIG. 6C-6D). There was also significant reduction in the levels of the noncanonical NF-κB pathway inhibitor, TRAF2 (FIG. 6C). Nuclear translocation was observed of ReIB, the binding partner of p52, in CIN-medium/high cells cells exhibiting medium to high levels of chromosomal instability (FIG. 6E).

Interestingly, STING depletion abolished noncanonical NF-κB activation and ReIB nuclear translocation and it was associated with negative enrichment in the TNF-α/NF-κB as well as other inflammatory and EMT pathways (FIG. 6D-6E).

Bulk RNA-seq data revealed a number of noncanonical NF-κB target genes, which were upregulated in response to chromosomal instability (hence referred to as CIN-responsive NC-NF-κB genes, which include PPARG, DDIT3, NUPR1, RAB3B, IGFBP4, LRRC8C, TCP11L2, MAFK, NRG1, F2R, KRT19, CTGF, ZFC3H1, MACROD1, GSTA4, SCN9A, BDNF, LACTB). Similarly, the single-cell analysis showed that there was a significant correlation between the chromosomal instability-signature genes and the CIN-responsive NC-NF-κB genes (FIG. 4B and FIG. 5B).

To validate the relationship between chromosomal instability-signature genes and the CIN-responsive NC-NF-κB genes in an independent dataset, RNA-seq data were analyzed from the TCGA breast cancer database. Significant upregulation of CIN-responsive NC-NF-κB genes was observed in tumors with higher levels of the CIN-signature genes (FIG. 6F). Furthermore, higher expression of key regulators of the noncanonical NF-κB pathway or its CIN-responsive target genes was associated with shorter DMFS and disease-free survival in breast and lung cancers. Conversely, upregulation of canonical NF-κB pathway (NFKB1, ReIA, TRAF1, TRAF4, TRAF5, TRAF6) or interferon-regulatory factors (IRF1, IRF3, IRF7, TBK1) were associated with improved prognosis (FIG. 9).

Collectively, these data show that chromosomal instability induces a cytosolic dsDNA response manifested in the selective activation of the noncanonical NF-κB pathway and these features are associated with poor prognosis.

To test whether STING activity is important for metastasis in a tumor cell-autonomous manner, intracardiac injection of STING-depleted cells that exhibit high levels of chromosomal instability was performed. There was significant reduction in metastatic dissemination and lifespan extension in mice injected with STING-depleted cells compared to mice injected with their STING-replete counterparts (FIGS. 6G-1 and 6G-2, FIG. 9A).

Similarly, depletion of STING, cGAS, or the noncanonical NF-κB transcription factors p52 and ReIB led to a significant decrease in the invasive potential of cells exhibiting high levels of chromosomal instability (CIN-high cells; FIG. 6H).

On the other hand, addition of cGAMP increased the ability of MCAK (CIN-low) cells to migrate and invade through a collagen membrane (FIG. 6H).

Therefore, tumor-cell autonomous STING activation in response to cytosolic DNA promotes invasion and metastasis, in part, through the noncanonical NF-κB pathway.

### Example 7: Chromosomal Instability is Also Correlated with Immune Infiltrate

The data provided herein shown that a novel pathway exists that links chromosomal instability (CIN) to metastasis and formation of tumor immune infiltrate through tumor-cell intrinsic inflammatory response to cytosolic DNA. The pathway identified by the inventors is summarized in FIG. 7B. Briefly, the inventors found that CIN promotes the formation of chromosome-containing micronuclei, which often rupture exposing their DNA content to the cellular cytoplasm (or cytosol). This unusual situation—which does not occur in normal cells—is reminiscent of a viral infection. After sensing cytosolic DNA through cGAS, cancer cells promote the formation of cGAMP (a small molecule) that in turn activates STING. Instead of upregulating the canonical pathways cancer cells activate the noncanonical NF-kB pathway (NIK and ReIB/p52) which leads to upregulation of pro-metastasis programs. In the meantime, cGAMP can exit tumor cells and activate neighboring stroma, in particular antigen presenting cells by directly engaging with their STING protein.

There are currently pre-clinical efforts underway exploring the use of intratumoral cGAMP injection in activating the immune system to attack tumor cells. The inventors think this effort might not be without its own risk as they have found that cGAMP in tumor cells themselves promotes metastasis—as opposed to its anti-tumor role in activating the immune cells.

The finding that chromosomal instability promotes a viral-like immune response that promotes metastasis yet at the same time recruits a large amount of an immune infiltrate (FIG. 7A) is significant, showing that chromosomally unstable cells are able to survive, thrive and metastasize in the presence of this immune activation.

Cells exhibiting chromosomal instability appear to be proficient at preserving the cytosolic DNA signal (and its byproducts) as much as possible within their own cytoplasm. In other words, they down regulate putative cGAMP transporters ABCG2 and ABCC4. Furthermore, these cells produce significantly higher amounts of ENPP1, a hydrolase that efficiently breaks down cGAMP and is only present on the extracellular leaflet of the plasma membrane. Therefore, these chromosomally unstable tumor cells preserve cGAMP in the intracellular milieu, reduce its export and, if necessary, degrade it when it leaks out. Furthermore, these tumor cells also produce large amounts of M-CSF, which is a cytokine that promotes the generation of pro-tumor M2 macrophages.

Such immune activation can be mobilized to facilitate treatment of cancers associated with chromosomal infiltration.

For example, instead of injecting tumors with cGAMP directly (and risking activating metastasis in tumor cells), the cGAMP produced by chromosomally unstable tumor cells can be against them: by inhibiting ENPP1, which underlies their ability to destroy it once it exists the cells. Another approach would be to use agonists to the ABC transporters to increase cGAMP export to the extracellular space and to activate neighboring immune cells.

### Example 8: cGAMP Detection and Quantification Using Liquid Chromatography-Mass Spectrometry (LC-MS)

This Example illustrates that liquid chromatography-tandem mass spectrometry (LC-MS/MS) is a viable technology for the determination of cGAMP due to its specificity, reproducibility and sensitivity. LC-MS/MS is highly specific, thus minimizing interferences from other nucleotides. The greater specificity of LC-MS/MS is derived from analyte specific precursor to product ion mass-to-charge (m/z) values and/or analyte specific retention time.

### Materials & Methods:

A cGAMP solution was used as a standard. The cGAMP standard solution was prepared in 70% acetonitrile in ddH₂O for LC-MS/MS analysis.

For cell culture, cells were grown in 10 cm plates.

### Collection and Sample Preparation:

Cells were washed twice with PBS and once with LC/MS grade water (to remove salts). Plates were then flash frozen on liquid nitrogen to preserve metabolic state of the cells. Cells were then collected/scraped into 2 ml of cold 80% LC-MS grade methanol (−80C). Methanolic metabolite extracts were then purified by Solid Phase Extraction (SPE) using HyperSep aminopropyl solid phase columns as previously described by Collins, A. C. et al. 2015. Effluents were dried to completeness in a vacuum centrifuge and reconstituted in 70% acetonitrile in ddH₂O at a concentration of 100 μg protein/μL. 15 μL were subjected to LC-MS/MS analysis.

Serum/Media Sample Preparation:

To detect secreted cGAMP in culture media, 500 μl aliquots of conditioned media can be collected, mixed 80.20 with methanol, and centrifuged at 3,000 rpm for 20 minutes at 4 degrees Celsius. The resulting supernatant can be collected and stored at −80 degrees Celsius prior to LC-MS/MS to assess cGAMP levels. To measure whole-cell associated metabolites, media can be aspirated and cells can be harvested. e.g., at a non-confluent density. A variety of different liquid chromatography (LC) separation methods can be used. Each method can be coupled by negative electrospray ionization (ESI, −3.0 kV) to triple-quadrupole mass spectrometers operating in multiple reaction monitoring (MRM) mode, with MS parameters optimized on infused metabolite standard solutions.

Analysis of cGAMP.

After Solid Phase Extraction (SPE), the samples were dried using a vacuum centrifuge (Eppendorf Vacufuge, Eppendorf, Germany) and reconstituted in 70% acetonitrile in ddH₂O. To remove unsolubilized particles, samples were centrifuged at 21,130 g for 10 min at 4° C. The supernatant was injected into an LC/MS-system comprised of an Agilent 1260 HPLC and an Agilent 6460 triple quadrupole mass spectrometer (Agilent Technologies, Santa Clara, CA) equipped with a JetStream electrospray ionization source, using positive ion-monitoring in dynamic multiple reaction monitoring (dMRM). The analyte cGAMP was resolved from interfering signals on an aqueous neutral phase column (Cogent™ Diamond Hydride, 4 μm particle size, 150 mm×2.1 mm; Microsolv Technology Corporation, NJ), at a column compartment temperature of 40° C. The samples were maintained at 4° C. and the injection volume was 15 μL. The gradient-chromatography previously described by Chen et al. (PLoS One 7(6): p. e37149 (2012)) was optimized to achieve chromatographic separation from interfering peaks. The aqueous mobile phase (A) was 50% isopropanol with 0.025% acetic acid, the organic mobile phase (B) was 90% acetonitrile containing 5 mM ammonium acetate. To eliminate the interference of metal ions on the chromatographic peak integrity and ESI ionization, EDTA was added to the mobile phase in a final concentration of 6 uM. The final gradient applied was: 0-1.0 min 99% B, 1.0-10.0 min to 60% B, 10.1-20 min 0% B and 20.1 min 99% B for 10 min to regenerate the column. The flow rate was 0.4 mL/min. Data was saved in centroid mode using Agilent Masshunter workstation acquisition software (B.06.00 Build 6.0.6025.4 SP4). Acquired raw data files were processed with Agilent MassHunter Qualitative Analysis Software (B.07.00 Build 7.0.7024.0, Agilent Technologies) and Quantitative Analysis Software (B.07.01 Build 7.1.524.0). The operating source parameters for MS-analysis were: gas temperature 280° C.; gas flow 11 L/min; nebulizer pressure 35 psi; sheath gas temperature 350° C.; sheath gas flow 11 L/min; capillary voltage 4000 V; nozzle voltage 300 V; fragmentor voltage 145V; cell accelerator voltage 2 V. dMRM data was acquired starting at a run time of 4 min in when the LC-flow was directed to the MS.

Compound specific parameters were optimized using Agilent Optimizer Software (for 6400 Series Triple Quadupole Version B.06.00 Build 6.0.6025.4 SP4).

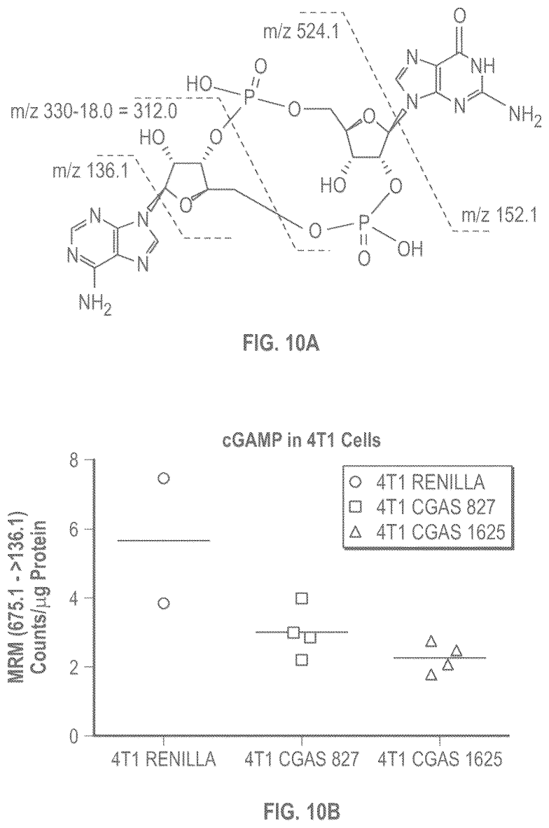

Optimized dMRM transitions resulted in the deglycosylated base ions: for cGAMP the transition 675.1→136.1* (CE 65 eV) represented the formation of adenine and 675.1→152.1** (CE 65 eV) the formation of guanine. Additionally, the dMRM transitions of 675.1→312.0 (CE 61 eV) and 675.1→524.1 (CE 35 eV) were recorded. * indicate quantifier transitions, ** indicate the qualifier transitions (see FIG. 10A). Because all the cGAMP transitions were derived from the same parent ion, all four transitions were summed into a final TIC (total ion current) to increase signal abundances and signal-to-noise ratios.

### Results

FIG. 10B graphically illustrates quantification of cGAMP in chromosomally unstable urine triple-negative breast cancer cells (4T1) using targeted LC-MS metabolomics. As shown, knockdown of cGAS in 4T1 cells reduced the abundance of cGAMP. These results show that cGAMP can be quantified in a variety of samples, and that cGAMP can be a marker for detecting and monitoring metastatic disease in patients.

### Example 9: ATPase Assays for Identifying/Assessing KIF2B and MCAK Agonists

KIF2B and KIF2C/MCAK are related molecular kinesin motor proteins that utilize the energy of ATP hydrolysis to regulate microtubule dynamics and chromosome-kinetochore attachments. The central role of KIF2B and MCAK over expression or hyper activation is to suppress chromosomal instability (CIN), which makes them attractive targets for cancer therapy. Here, two methods (an in vitro assay and an imaging method) are outlined in this Example to identify and assess potent activators of KIF2B and MCAK.

### Method 1 In Vitro Assay for KIF2B or MCAK Activity:

Measuring the kinetics of ATP hydrolysis is a strategy to screen for compounds that activate KIF2B and MCAK and suppress CIN. This assay is based upon an absorbance shift (330 to 360 nm) that occurs when 2-amino-6-mercapto-7-methylpurine ribonucleoside (MESG) is converted to 2-amino-6-mercapto-7-methyl purine in the presence of inorganic phosphate (Pi) (see, e.g., Webb, M. R. 1992. A continuous spectrophotometric assay for inorganic phosphate and for measuring phosphate release kinetics in biological systems. Proc. Natl. Acad. Sci. USA 89: 4884-4887). The reaction is catalyzed by purine nucleoside phosphorylase (PNP). One molecule of inorganic phosphate will yield one molecule of 2-amino-6-mercapto-7-methyl purine in an irreversible reaction. Thus, the absorbance at 360 nm is directly proportional to the amount of Pi generated in the ATPase reaction, and can be used as a proxy for MCAK activity.

Alternatively, ADP production can also be monitored as a readout for MCAK activity using the Transcreener ADP assay from BellBrook Labs. This assay is based on the ability of ADP to displace a fluorescent tracer (633 nm) bound to an antibody the specifically recognizes ADP. Displacement of the tracer causes a decrease in fluorescence measured by laser excitation at 633 nm. Thus, activity of MCAK can be calculated by plotting the concentration of drug used and the amount of ADP produced/decrease in fluorescent intensity.

### Method 2 Cell-Based Assay for KIF2B or MCAK Activity:

MCAK negatively regulates microtubule length by binding microtubule tips and promoting microtubule depolymerization. Therefore, distance between γ-tubulin-labeled centrosomes can be measured as an indirect readout for MCAK activity in cells. Spindle length would be inversely proportional to MCAK activity and can serve as proxy to evaluate potential compounds that promote MCAK activity (see, e.g., Lockhart, A & Cross, R. A. 1996. Kinetics and Motility of the Eg5 Microtubule Motor. Biochemistry 35: 2365-2373). This method can be adapted for screening compounds by using a high-throughput imaging microscope.

Compounds (e.g., top hits identified via any of the methods described herein) can subsequently be used in a cell-based assay using lagging chromosomes, micronuclei, or chromosome missegregation using FISH as a readout of their efficacy. Fluorescent in situ hybridization (FISH) is a molecular cytogenetic technique that uses fluorescent probes that bind to only those parts of the chromosome with a high degree of sequence complementarity. Probes can include a portion of sequence of any of the chromosomes or genes described herein.

### Example 10: ATPase Assays for Identifying/Assessing NF-kB Inducing Kinase (NIK) Inhibitors

NF-kB Inducing Kinase (NIK) mediates non-canonical NF-kB signaling and is associated with metastasis. Therefore, the inhibition of NIK may suppress CIN-induced inflammatory responses and metastasis. This Example outlines two methods that can be used to identify and assess NIK inhibition.

### Method 1:

Specific inhibition of the kinase function of NIK provides an approach to assess the potency of various compounds. Therefore, ADP production can be monitored as a readout for NIK activity using the Transcreener ADP assay from BellBrook Labs. This assay is based on the ability of ADP to displace a fluorescent tracer (633 nm) bound to an antibody the specifically recognizes ADP. Competitive displacement of the tracer causes a decrease in fluorescence, as measured by laser excitation at 633 nm. Thus, the activity of NIK can be calculated by plotting the concentration of drug used and the amount of ADP produced/decrease in fluorescent intensity.

### Method 2:

Inhibition of NIK provides an approach to directly inhibit the non-canonical NF-κB pathway. This assay relies on quantification of the nuclear translocation of p52 (RELB; non-canonical NF-kB signaling) using high content cellular imaging.

An example of a sequence for human RELB is shown below as SEQ ID NO:59.

| | |
|---|---|
| 1 | MLRSGPASGP SVPTGRAMPS RRVARPPAAP ELGALGSPDL |
| 41 | SSLSLAVSRS TDELEIIDEY IKENGFGLDG GQPGPGEGLP |
| 81 | RLVSRGAASL STVTLGPVAP PATPPPWGCP LGRLVSPAPG |
| 121 | PGPQPHLVIT EQPKQRGMRF RYECEGRSAG SILGESSTEA |
| 161 | SKTLPAIELR DCGGLREVEV TACLVWKDWP HRVHPHSLVG |
| 201 | KDCTDGICRV RLRPHVSPRH SFNNLGIQCV RKKEIEAAIE |
| 241 | RKIQLGIDPY NAGSLKNHQE VDMNVVRICF QASYRDQQGQ |
| 281 | MRRMDPVLSE PVYDKKSTNT SELRICRINK ESGPCTGGEE |
| 321 | LYLLCDKVQK EDISVVFSRA SWEGRADFSQ ADVHRQIAIV |
| 361 | FKTPPYEDLE IVEPVTVNVF LQRLTDGVCS EPLPFTYLPR |
| 401 | DHDSYGVDKK RKRGMPDVLG ELNSSDPHGI ESKRRKKKPA |
| 441 | ILDHFLPNHG SGPFLPPSAL LPDPDFFSGT VSLPGLFPPG |
| 481 | GPDLLDDGFA YDPTAPTLFT MLDLLPPAPP HASAVVCSGG |
| 521 | AGAVVGETPG PEPLITDSYQ APGPGDGGTA SLVGSNMFPN |
| 561 | HYREAAFGGG LLSPGPEAT |

For RELB nuclear translocation assay, cells are treated with different concentrations of compounds and stimulated with 100 ng/mL of an antagonistic anti-lymphotoxin beta receptor (LT-βR) antibody (e.g., from Sigma Aldrich), a potent activator of non-canonical NF-kB signaling. RELB translocation into the nucleus is quantified by the ratio of the nuclear over cytoplasmic signal intensity. Potent compounds are discovered that selectively inhibit the nuclear translocation of RELB.

### REFERENCES

1. Nowell, P. C. The clonal evolution of tumor cell populations. Science 194, 23-28(1976).
2. Turajlic, S., Turajlic, S., Swanton, C. & Swanton, C. Metastasis as an evolutionary process. Science 352, 169-175 (2016).
3. Burrell, R. A., McGranahan, N., Bartek, J. & Swanton, C. The causes and consequences of genetic heterogeneity in cancer evolution. Nature 501, 338-345 (2013).
4. Makohon-Moore, A. P. et al. Limited heterogeneity of known driver gene mutations among the metastases of individual patients with pancreatic cancer. Nat. Genet. (2017). doi:10.1038/ng.3764
5. Campbell, P. J. et al. The patterns and dynamics of genomic instability in metastatic pancreatic cancer. Nature 467, 1109-1113 (2010).
6. Notta, F. et at A renewed model of pancreatic cancer evolution based on genomic rearrangement patterns. Nature 538, 378-382 (2016).
7. Crasta, K. K. et al. DNA breaks and chromosome pulverization from errors in mitosis. Nature 482, 53-58 (2012).
8. Burrell, R. A. et al. Replication stress links structural and numerical cancer chromosomal instability. Nature 494, 492-496 (2013).
9. Bakhoum, S. F., Kabeche, L., Mumane, J. P., Zaki, B. I. & Compton, D. A. DNA-Damage Response during Mitosis Induces Whole-Chromosome Missegregation. Cancer Discovery 4, 1281-1289 (2014).
10. Maciejowski, J., Li, Y., Bosco, N., Campbell, P. J. & de Lange, T. Chromothripsis and Kataegis Induced by Telomere Crisis. Cell 163, 1641-1654 (2015).
11. Thompson, S. L., Thompson, S. L., Compton, D. A. & Compton, D. A. Examining the link between chromosomal instability and aneuploidy in human cells. J Cell Biol 180, 665-672 (2008).
12. Cimini, D. et al. Merotelic kinetochore orientation is a major mechanism of aneuploidy in mitotic mammalian tissue cells. J Cell Biol 153, 517-527 (2001).
13. Bakhoum, S. F., Thompson, S. L., Manning, A. L. & Compton, D. A. Genome stability is ensured by temporal control of kinetochore-microtubule dynamics. Nat. Cell Biol. 11, 27-35 (2009).
14. Bakhoum, S. F. et al. Numerical chromosomal instability mediates susceptibility to radiation treatment. Nat Commun 6, 5990 (2015).
15. Bakhoum, S. F. et al. The mitotic origin of chromosomal instability. Curr. Biol. 24, R148-9 (2014).
16. Lengauer, C., Kinzler, K. W. & Vogelstein, B. Genetic instabilities in human cancers. Nature 396, 643-649 (1998).
17. Brastianos, P. K. et al. Genomic Characterization of Brain Metastases Reveals Branched Evolution and Potential Therapeutic Targets. Cancer Discovery 5, 1164-1177 (2015).
18. Mitelman, F., Johansson, B. & Mertens, F. Mitelman Database of Chromosome Aberrations and Gene Fusions in Cancer. cgap.nci.nih.gov Available at: http://cgap.nci.nih.gov/Chromosomes/Mitelman. (Accessed: 24 Oct. 2014)
19. Carter, S. L. S. et al. Absolute quantification of somatic DNA alterations in human cancer. Nat Biotechnol 30, 413-421 (2012).
20. Laughney, A. M., Elizalde, S., Genovese, G. & Bakhoum, S. F. Dynamics of Tumor Heterogeneity Derived from Clonal Karyotypic Evolution. Cell Rep 12, 809-820 (2015).
21. Storchova, Z., Storchova, Z., Kuffer, C. & Kuffer, C. The consequences of tetraploidy and aneuploidy. J Cell Sci 121, 3859-3866 (2008).
22. Chung, C. H. et al. Molecular classification of head and neck squamous cell carcinomas using patterns of gene expression. Cancer Cell 5, 489-500 (2004).
23. Bakhoum, S. F., Danilova, O. V., Kaur, P., Levy, N. B. & Compton, D. A. Chromosomal instability substantiates poor prognosis in patients with diffuse large B-cell lymphoma. Clin. Cancer Res. 17, 7704-7711 (2011).
24. Moore, A. T. et al. MCAK associates with the tips of polymerizing microtubules. J Cell Biol 169, 391-397 (2005).
25. Ems-McClung, S. C. & Walczak, C. E. Kinesin-13s in mitosis: Key players in the spatial and temporal organization of spindle microtubules. Semin. Cell Dev. Biol. 21, 276-282 (2010).
26. Györffy, B. et al. An online survival analysis tool to rapidly assess the effect of 22,277 genes on breast cancer prognosis using microarray data of 1,809 patients. Breast Cancer Res. Treat. 123, 725-731 (2010).
27. Hatzis, C. et al. A genomic predictor of response and survival following taxane-anthracycline chemotherapy for invasive breast cancer. JAMA 305, 1873-1881 (2011).
28. Klein, A. M. et at. Droplet barcoding for single-cell transcriptomics applied to embryonic stem cells. Cell 161, 1187-1201 (2015).
29. Levine, J. H. et al. Data-Driven Phenotypic Dissection of AML Reveals Progenitor-like Cells that Correlate with Prognosis. Cell 162, 184-197 (2015).
30. Abe, T., Abe, T., Barber, G. N. & Barber, G. N. Cytosolic-DNA-mediated, STING-dependent proinflammatory gene induction necessitates canonical NF-κB activation through TBK1. J. Virol. 88, 5328-5341 (2014).
31. Hatch, E. M., Fischer, A. H., Deerinck, T. J. & Hetzer, M. W. Catastrophic nuclear envelope collapse in cancer cell micronuclei. Cell 154, 47-60 (2013).
32. Cai, X., Chiu, Y.-H. & Chen, Z. J. The cGAS-cGAMP-STING pathway of cytosolic DNA sensing and signaling. Mol. Cell 54, 289-296 (2014).
33. Sun, L. et al. Cyclic GMP-AMP synthase is a cytosolic DNA sensor that activates the type I interferon pathway. Science 339, 786-791 (2013).
34. Stetson, D. B., Ko, J. S., Heidmann, T. & Medzhitov, R. Trex1 Prevents Cell-Intrinsic Initiation of Autoimmunity. Cell 134, 587-598 (2008).
35. Lau, L., Gray, E. E., Brunette, R. L. & Stetson, D. B. DNA tumor virus oncogenes antagonize the cGAS-STING DNA-sensing pathway. Science 350, 568-571 (2015).
36. Sun, S.-C. Controlling the fate of NIK: a central stage in noncanonical NF-kappaB signaling. Sci Signal 3, pe18 (2010).
37. Chen, Q. et al. Carcinoma-astrocyte gap junctions promote brain metastasis by cGAMP transfer. Nature 533, 493-498 (2016).
38. Femandes-Alnemri, T., Yu, J.-W., Datta, P., Wu, J. & Alnemri, E. S. AIM2 activates the inflammasome and cell death in response to cytoplasmic DNA. Nature 458, 509-513 (2009).
39. Vitak, N., Hume, D. A., Chappell, K. J., Sester, D. P. & Stacey, K. J. Induction of interferon and cell death in response to cytosolic DNA in chicken macrophages. Dev. Comp. Immunol. 59, 145-152 (2016).
40. Wang, J., Yi, S., Zhou, J., Zhang, Y. & Guo, F. The NF-κB subunit ReIB regulates the migration and invasion abilities and the radio-sensitivity of prostate cancer cells. Int. J. Oncol. 49, 381-392 (2016).
41. Demirci, H., Reed, D. & Elner, V. M. Tissue-based microarray expression of genes predictive of metastasis in uveal melanoma and differentially expressed in metastatic uveal melanoma. J Ophthalmic Vis Res 8, 303-307 (2013).
42. Fritz, R. D. & Radziwill, G. CNK1 promotes invasion of cancer cells through NF-kappaB-dependent signaling. Mol. Cancer Res. 8, 395-406 (2010).
43. Qin, H. et al. Prognostic significance of ReIB overexpression in non-small cell lung cancer patients. Thorac Cancer 7, 415-421 (2016).
44. Yakubov, B. et al. Extracellular tissue transglutaminase activates noncanonical NF-κB signaling and promotes metastasis in ovarian cancer. Neoplasia 15, 609-619 (2013).
45. Rangaswami, H. & Kundu, G. C. Osteopontin stimulates melanoma growth and lung metastasis through NIK/MEKK1-dependent MMP-9 activation pathways. Oncol. Rep. 18, 909-915 (2007).
46. Qu, L. L., He, L., Zhao, X. & Xu, W. Downregulation of miR-518a-3p activates the NIK-dependent NF-κB pathway in colorectal cancer. Int. J. Mol. Med. 35, 1266-1272 (2015).
47. Thu, Y. M. et al. NF-κB inducing kinase (NIK) modulates melanoma tumorigenesis by regulating expression of pro-survival factors through the β-catenin pathway. Oncogene 31, 2580-2592 (2012).
48. Zhang, X. et al. Expression of NF-κB-inducing kinase in breast carcinoma tissue and its clinical significance. Int J Clin Exp Pathol 8, 14824-14829 (2015).
49. Tchoghandjian, A., Jennewein, C., Eckhardt, I., Rajalingam, K. & Fulda, S. Identification of non-canonical NF-κB signaling as a critical mediator of Smac mimetic-stimulated migration and invasion of glioblastoma cells. Cell Death Dis 4, e564 (2013).
50. Yu, J. et al. Noncanonical NF-κB activation mediates STAT3-stimulated IDO upregulation in myeloid-derived suppressor cells in breast cancer. J. Immunol. 193, 2574-2586 (2014).
51. Yang, C. et al. Antagonism of inhibitor of apoptosis proteins increases bone metastasis via unexpected osteoclast activation. Cancer Discovery 3, 212-223 (2013).
52. McGranahan, N. et al. Clonal neoantigens elicit T cell immunoreactivity and sensitivity to immune checkpoint blockade. Science 351, 1463-1469 (2016).
53. Zaki, B. I. et al. Chromosomal instability portends superior response of rectal adenocarcinoma to chemoradiation therapy. Cancer 120, 1733-1742 (2014).
54. Dobin, A. et al. STAR: ultrafast universal RNA-seq aligner. Bioinformatics 29, 15-21 (2013).
55. Trapnell, C. et al. Transcript assembly and quantification by RNA-Seq reveals unannotated transcripts and isoform switching during cell differentiation. Nat Biotechnol 28, 511-515 (2010).
56. Anders, S., Pyl, P. T. & Huber, W. HTSeq-a Python framework to work with high-throughput sequencing data. Bioinformatics 31, 166-169 (2015).
57. Love, M. I., Huber, W. & Anders, S. Moderated estimation of fold change and dispersion for RNA-seq data with DESeq2. Genome Biol. 15, 550 (2014).
58. Zheng, G. X. Y. et al. Massively parallel digital transcriptional profiling of single cells. Nat Commun 8, 14049 (2017).
59. van Dijk, D., Nainys, J., Sharma, R., Kathail, P. & Carr, A. J. MAGIC: A diffusion-based imputation method reveals gene-gene interactions in single-cell RNA-sequencing data. bioRxiv (2017).
60. Györffy, B., Surowiak, P., Budczies, J. & Lanczky, A. Online survival analysis software to assess the prognostic value of biomarkers using transcriptomic data in non-small-cell lung cancer. PLoS ONE 8, e82241 (2013).
61. Chen, Q., et al., Untargeted plasma metabolite profiling reveals the broad systemic consequences of xanthine oxidoreductase inactivation in mice. PLoS One, 2012. 7(6): p. e37149.
62. Collis, A. C., et al., Cyclic GMP-AMP Synthase Is an Innate Immune DNA Sensor for Mycobacterium tuberculosis. Cell Host & Microbe, 2015. 17(6): p. 820-828.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

Statements:

2) A method comprising administering a metastatic chemotherapeutic agent to a patient with a cell sample or bodily fluid sample:
a. having at least 10%, or at least 11%, or at least 12%, or at least 13%, or at least 14%, or at least 15% detectable chromosomal missegregations within one or cells of the cell sample;
b. having at least 3%, at least 4% or at least 5% of cells detectable micronuclei within one or cells of the cell sample;
c. having detectable cytosolic double-stranded DNA within one or cells of the cell sample; or
d. having at least 10%, or 20%, or 30%, or 50%, or 70%, or 80%, or 90% greater concentration or amount of cGAMP in the cell sample or bodily fluid sample;
to thereby treat metastatic cancer in the patient.

3) The method of statement 1, comprising administering a metastatic chemotherapeutic agent to a patient with 15-20% of chromosomes in anaphase cells of the cell sample exhibiting missegregations.

4) The method of statement 1 or 2, comprising administering a metastatic chemotherapeutic agent to a patient with 5-8% of cells in the cell sample exhibiting micronuclei.

5) The method of statement 1, 2, or 3, comprising administering a metastatic chemotherapeutic agent to a patient with 1-fold to 2-fold increase in staining intensity within the cytosol compared to a normal non-cancer tissue.

6) The method of statement 1, 2, 3, or 4, comprising administering a metastatic chemotherapeutic agent to a patient with 1-fold to 2-fold greater concentration or amount of cGAMP in the bodily fluid sample than a non-cancerous bodily fluid sample.

7) The method of statement 1-4 or 5, further comprising monitoring samples from the patient over time to quantify chromosomal missegregations, micronuclei, cytosolic double-stranded DNA, or cGAMP within cells or bodily fluids of the patient.

8) The method of statement 1-5 or 6, wherein the metastatic chemotherapeutic agent is a composition comprising kinesin-13 protein(s) with at least 90%, at least 91%, at least 92%, at least 93%, at least 94%, at least 95%, at least 96%, at least 97%, at least 98%, at least 99% sequence identity to any of SEQ ID NO:1, 3, or 5.

9) The method of statement 1-6 or 7, wherein the metastatic chemotherapeutic agent is a composition comprising a kinesin-13 nucleic acid comprising a sequence with at least 90%, at least 91%, at least 92%, at least 93%, at least 94%, at least 95%, at least 96%, at least 97%, at least 98%, at least 99% sequence identity to any of SEQ ID NO:2, 4, or 6.

10) The method of statement 1-7 or 8, wherein the metastatic chemotherapeutic agent is a composition comprising a MCAK protein with at least 90%, at least 91%, at least 92%, at least 93%, at least 94%, at least 95%, at least 96%, at least 97%, at least 98%, at least 99% sequence identity to any of SEQ ID NO: 7, or a MCAK nucleic acid with at least 90%, at least 91%, at least 92%, at least 93%, at least 94%, at least 95%, at least 96%, at least 97%, at least 98%, at least 99% sequence identity to SEQ ID NO:8.

11) The method of statement 1-8 or 9, wherein the metastatic chemotherapeutic agent is a composition comprising at least one STING, cGAS, NF-κB transcription factor p52, NF-κB transcription factor ReIB, ENPP1, LTβR, BAFFR, CD40, RANK, FN14, NIK (MAP3K14), or MST1 inhibitory nucleic acid.

12) The method of statement 1-9 or 10, wherein the metastatic chemotherapeutic agent is a composition comprising at least one inhibitory nucleic acid having at least 90%, at least 91%, at least 92%, at least 93%, at least 94%, at least 95%, at least 96%, at least 97%, at least 98%, at least 99% sequence identity to any of SEQ ID NO: 10, 12, 14, 16, 18, 20, 22, 24, 25, 26, 27, 28, 29, 30, 31, 32, 33, 34, 35, or 36.

13) The method of statement 1-10 or 11, wherein the metastatic chemotherapeutic agent is a composition comprising at least one antibody that binds with affinity to a STING, cGAS, NF-κB transcription factor p52, NF-κB transcription factor ReIB, ENPP1, LTβR, BAFFR, CD40, RANK, FN14, NIK (MAP3K14), or MST1 protein.

14) The method of statement 1-11 or 12, wherein the metastatic chemotherapeutic agent is a composition comprising an expression vector having a promoter operably linked to a nucleic acid segment encoding a kinesin-13 or MCAK protein with at least 90%, at least 91%, at least 92%, at least 93%, at least 94%, at least 95%, at least 96%, at least 97%, at least 98%, at least 99% sequence identity to any of SEQ ID NO:1, 3, 5, or 7.

15) The method of statement 1-12 or 13, wherein the metastatic chemotherapeutic agent is a composition comprising an agonist of kinesin-13 with the following structure, wherein X is a methyl group:

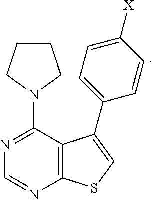

16) The method of statement 1-13 or 14, wherein the concentration or amount of cGAMP in the bodily fluid sample or the cell sample is quantified in a method comprising liquid chromatography (LC) with mass spectrometry (MS).

17) The method of statement 1-14 or 15, wherein the cGAMP in the bodily fluid sample or the cell sample is extracted and/or dissolved in an alcohol to produce an alcohol extract, the alcohol extract can be subjected to chromatography, and the effluent from the chromatography can be suspended in acetonitrile, water or a combination thereof before measuring the concentration or amount of the cGAMP.

18) A method comprising administering to a subject at least one kinesin-13 protein, at least one MACK protein, at least one agonist of kinesin-13, at least one agonist of MACK, or a combination thereof.

19) The method of statement 17, wherein the at least one kinesin-13 protein or MCAK has at least 90%, at least 91%, at least 92%, at least 93%, at least 94%, at least 95%, at least 96%, at least 97%, at least 98%, at least 99% sequence identity to any of SEQ ID NO:1, 3, 5, or 7.

20) The method of statement 17 or 18, wherein at least one agonist of kinesin-13 is the following, wherein X is a methyl group:

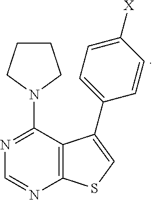

21) The method of statement 17, 18, or 19, further comprising administering an inhibitor of STING, cGAS, NF-κB transcription factor p52, NF-κB transcription factor ReIB, ENPP1, LTβR, BAFFR, CD40, RANK, FN14, NIK (MAP3K14), MST1, or any combination thereof to the subject.

22) A method comprising inhibiting STING, cGAS, NF-κB transcription factor p52, NF-κB transcription factor ReIB, ENPP1, LTβR, BAFFR, CD40, RANK, FN14, NIK (MAP3K14), MST1, or any combination thereof in a mammalian cell.

23) A method comprising administering to a subject an expression vector comprising a promoter operably linked to a nucleic acid segment encoding a kinesin-13 or MACK protein.

24) The method of statement 22, wherein the at least one kinesin-13 protein or MACK protein has at least 90%, at least 91%, at least 92%, at least 93%, at least 94%, at least 95%, at least 96%, at least 97%, at least 98%, at least 99% sequence identity to any of SEQ ID NO:1, 3, 5, or 7.

25) The method of statement 17-23 or 23, comprising administering an expression vector comprising a promoter operably linked to an inhibitory nucleic acid segment with at least 90%, at least 91%, at least 92%, at least 93%, at least 94%, at least 95%, at least 96%, at least 97%, at least 98%, at least 99% sequence identity or complementarity to any of SEQ ID NO: 9, 11, 13, or 15.

26) The method of statement 1-23, or 24, further comprising administering an inhibitor of STING, cGAS, NF-κB transcription factor p52, NF-κB transcription factor ReIB, ENPP1, LTβR, BAFFR, CD40, RANK, FN14, NIK (MAP3K14), MST1, or any combination thereof to the subject.

27) The method of statement 1-24 or 25, further comprising administering an agonist of ABCC4, ABCG2, or a combination thereof;
administering an expression cassette or vector comprising a promoter operably linked to a nucleic acid segment that encodes ABCC4 or ABCG2; or a combination thereof.

28) The method of statement 1-25, or 26, wherein cells in the patient exhibits chromosomal instability prior to administration.

29) The method of statement 1-26 or 27, wherein the patient is suspected of having cancer.

30) The method of statement 1-27 or 28, wherein the patient is suspected of developing cancer.

31) The method of statement 1-28 or 29, wherein the patient has cancer.

32) The method of statement 1-29 or 30, wherein the patient has metastatic cancer.

33) The method of statement 1-30 or 31, wherein the method inhibits metastasis of cancer in the subject.

34) The method of statement 1-31 or 32, wherein the method inhibits metastasis of cancer in the subject compared to a control subject that did not receive the protein or the expression vector.

35) The method of statement 1-32 or 33, wherein the method inhibits chromosomal instability.

36) A method comprising quantifying expression levels of at least one of the following genes in a test sample from a patient: PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, or FGF5, to generate at least one quantified expression level of at least one following genes in the test sample: PELI2, BMP2, SHH, TNS4, RAB3B, ROBO1, ARHGAP28, CHN2, CST1, F13A1, CPVL, SEMA6D, C9orf152, NHSL2, GTF2IP7, DPYSL3, PCDH7, KHDRBS3, TRAC, TMEM156, CST4, CD24, or FGF5.

37) The method of statement 35, further comprising determining at least one difference in at least one quantified expression level of at least one following genes in the test sample: PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, or FGF5 compared to a control expression level of at least one corresponding gene in a healthy or non-cancerous sample.

38) The method of statement 35 or 36, wherein the healthy or non-cancerous sample does not exhibit chromosomal instability.

39) The method of statement 35, 36, or 37, further comprising determining at least one difference in at least one quantified expression level of at least one following genes in the test sample: PELI2, BMP2, SHH, TNS4, RAB3B, ROBO1, ARHGAP28, CHN2, CST1, F13A1, CPVL, SEMA6D, C9orf152, NHSL2, GTF2IP7, DPYSL3, PCDH7, KHDRBS3, TRAC, TMEM156, CST4, CD24, or FGF5 compared to a control expression level of at least one corresponding gene in a sample (or set of samples) from a patient with metastatic cancer.

40) The method of statement 35-37, or 38, comprising quantifying expression levels of two or more, or three or more, or four or more, or five or more, or six or more, or seven or more, or eight or more, or nine or more, ten or more, or eleven or more, or twelve or more, or thirteen or more, or fourteen or more, or fifteen or more, or sixteen or more, or seventeen or more, or eighteen or more, or nineteen or more, or twenty or more, or twenty-one or more, or twenty-two or more of the following genes in the test sample: PELI2, BMP2, SHH, TNS4, RAB3B, ROBO1, ARHGAP28, CHN2, CST1, F13A1, CPVL, SEMA6D, C9orf152, NHSL2, GTF2IP7, DPYSL3, PCDH7, KHDRBS3, TRAC, TMEM156, CST4, CD24, or FGF5.

41) The method of statement 35-38, or 39, wherein the difference in at least one quantified expression level of at least one following genes in the test sample: PELI2, BMP2, SHH, TNS4, RAB3B, ROBO1, ARHGAP28, CHN2, CST1, F13A1, CPVL, SEMA6D, C9orf152, NHSL2, GTF2IP7, DPYSL3, PCDH7, KHDRBS3, TRAC, TMEM156, CST4, CD24, or FGF5 compared to a control expression level of at least one corresponding gene in a healthy or non-cancerous sample is at least a 10%, or 20% or 30%, or 40%, or 50%, or 60%, or 75%, or 100% increase in expression.

42) The method of statement 35-39, or 40, wherein the difference in at least one quantified expression level of at least one following genes in the test sample: PELI2, BMP2, SHH, TNS4, RAB3B, ROBO1, ARHGAP28, CHN2, CST1, F13A1, CPVL, SEMA6D, C9orf152, NHSL2, GTF2IP7, DPYSL3, PCDH7, KHDRBS3, TRAC, TMEM156, CST4, CD24, or FGF5 compared to a mean control expression level of at least one corresponding gene in a sample (or set of samples from one or more patients with metastatic cancer) is at least a 10%, or 20% or 30%, or 40%, or 50%, or 60%, or 75%, or 100% increase in expression.

43) The method of statement 35-40, or 41, wherein the difference in at least one quantified expression level of at least one following genes in the test sample: PELI2, BMP2, SHH, TNS4, RAB3B, ROBO1, ARHGAP28, CHN2, CST1, F13A1, CPVL, SEMA6D, C9orf152, NHSL2, GTF21P7, DPYSL3, PCDH7, KHDRBS3, TRAC, TMEM156, CST4, CD24, or FGF5 compared to a control expression level is at least an increase of expression of these corresponding genes of at least a 1.2-fold, or 1.5-fold, or 2-fold, or 3-fold, or 5-fold, or 7-fold, or 10-fold increase in expression.

44) A method comprising administering STING proteins to a subject or expressing STING proteins from an expression cassette or expression vector in a subject to restore and/or activate canonical pathways downstream of cytosolic DNA sensing as a therapeutic tool against chromosomally unstable tumor cells and induce cell-intrinsic cytotoxic pathways.

45) A method comprising administering on or more STING agonists to a subject to restore and/or activate canonical pathways downstream of cytosolic DNA sensing as a therapeutic tool against chromosomally unstable tumor cells and induce cell-intrinsic cytotoxic pathways.

46) The method of statement 43 or 44, which sensitizes tumor cells to immune therapies.

47) A composition comprising a carrier and a kinesin-13 protein with at least 90%, at least 91%, at least 92%, at least 93%, at least 94%, at least 95%, at least 96%, at least 97%, at least 98%, at least 99% sequence identity to any of SEQ ID NO:1, 3, or 5.

48) A composition comprising a carrier and a kinesin-13 nucleic acid comprising a sequence with at least 90%, at least 91%, at least 92%, at least 93%, at least 94%, at least 95%, at least 96%, at least 97%, at least 98%, at least 99% sequence identity to any of SEQ ID NO:2, 4, or 6.

49) The composition of statement 46 or 47, further comprising a MCAK protein with at least 90%, at least 91%, at least 92%, at least 93%, at least 94%, at least 95%, at least 96%, at least 97%, at least 98%, at least 99% sequence identity to SEQ ID NO: 7, or a MCAK nucleic acid with at least 90%, at least 91%, at least 92%, at least 93%, at least 94%, at least 95%, at least 96%, at least 97%, at least 98%, at least 99% sequence identity to SEQ ID NO:8.

50) The composition of statement 46, 47, or 48, comprising at least one STING, cGAS, NF-κB transcription factor p52, NF-κB transcription factor ReIB, ENPP1, LTPR, BAFFR, CD40, RANK, FN14, NIK (MAP3K14), or MST1 inhibitory nucleic acid.

51) The composition of statement 46-48 or 49, comprising at least one inhibitory nucleic acid having at least 90%, at least 91%, at least 92%, at least 93%, at least 94%, at least 95%, at least 96%, at least 97%, at least 98%, at least 99% sequence identity to any of SEQ ID NO: 10, 12, 14, 16, 18, 20, 22, 24, 25, 26, 27, 28, 29, 30, 31, 32, 33, 34, 35, or 36.

52) The composition of statement 46-49, or 50, comprising at least one antibody that binds with affinity to a STING, cGAS, NF-κB transcription factor p52, NF-κB transcription factor ReIB, ENPP1, LTβR, BAFFR, CD40, RANK, FN14, NIK (MAP3K14), or MST1 protein.

53) The composition of statement 46-50, or 51, comprising at least one antibody that binds with affinity to a protein with at least 90%, at least 91%, at least 92%, at least 93%, at least 94%, at least 95%, at least 96%, at least 97%, at least 98%, at least 99% sequence identity to any of SEQ ID NO: 9, 11, 13, 15, 17, 19, 21, or 23.

54) An expression vector comprising a promoter operably linked to a nucleic acid segment encoding a kinesin-13 or MCAK protein with at least 90%, at least 91%, at least 92%, at least 93%, at least 94%, at least 95%, at least 96%, at least 97%, at least 98%, at least 99% sequence identity to any of SEQ ID NO:1, 3, 5, or 7.

55) An expression vector comprising a promoter operably linked to an inhibitory nucleic acid segment with at least 90%, at least 91%, at least 92%, at least 93%, at least 94%, at least 95%, at least 96%, at least 97%, at least 98%, at least 99% sequence identity or complementarity to any of SEQ ID NO:10, 12, 14, 16, 18, 20, 22, 24, 25, 26, 27, 28, 29, 30, 31, 32, 33, 34, 35, or 36.

56) A method comprising: (a) mixing a test compound with cancer (or metastatic cancer) cells in a culture medium to produce a test assay; (b) incubating the test assay for a time and under conditions sufficient for the test compound to associate with or penetrate the cells; (c) measuring cGAMP amounts or concentrations in the culture medium, in the cells, or in a combination thereof to produce a test assay cGAMP value; and (d) selecting a test compound with a lower test assay cGAMP value than a reference cGAMP value to thereby produce an effective test compound.

57) The method of statement 55, wherein the reference cGAMP value is the amount or concentration of cGAMP in the culture medium, in the cells, or in a combination thereof of an assay mixture that does not contain a test compound.

58) A method comprising. (a) obtaining a cell or tissue sample from a patient: (b) measuring the amount or concentration of cGAMP produced from a known number or weight of cells or tissues from the sample to generate a reference cGAMP value: (c) mixing the same known number or weight of cells or tissues from the sample with a test compound to generate a test assay: (d) measuring the cGAMP amount or concentration in the test assay (either in the cell medium or in the cells or tissues) to generate a test assay cGAMP value; (e) optionally repeating steps (c) and (d); and selecting a test compound with a lower test assay cGAMP value than the reference cGAMP value to thereby identify an effective test compound.

59) The method of statement 55, 56 or 57, wherein the metastatic cancer cells or metastatic tissues are mixed in the culture medium to produce the test assay.

60) The method of statement 55-57 or 58, further comprising extracting the cell or tissue sample with an alcohol (e.g.: methanol, ethanol, or isopropanol) to produce an alcohol extract before measuring the cGAMP.

61) The method of statement 59, further comprising purifying the alcohol extract by Solid Phase Extraction (SPE) using one or more HyperSep aminopropyl solid phase columns to produce a semi-pure test sample before measuring the cGAMP of the semi-pure test sample

62) The method of statement 59 or 60, further comprising suspending the cGAMp in acetonitrile, water or a combination thereof before measuring the cGAMP.

63) The method of statement 55-60 or 61, wherein measuring cGAMP amounts or concentrations comprises liquid chromatography and/or mass spectroscopy to measure the level of cGAMP.

64) The method of statement 55-61 or 62, further comprising administering the effective test compound to an animal model, for example, to further evaluate the toxicity and/or efficacy of the effective test compound.

65) The method of statement 55-62 or 63, further comprising administering the effective test compound to a patient or to the patent from whom the cell or tissue sample as obtained.

66) An effective test compound produced by a method comprising: (a) mixing a test compound with cancer (or metastatic cancer) cells in a culture medium to produce a test assay; (b) incubating the test assay for a time and under conditions sufficient for the test compound to affect cGAMP production in the cells; (c) measuring cGAMP amounts or concentrations in the culture medium, in the cells, or in a combination thereof to produce a test assay cGAMP value; and (d) selecting a test compound with a lower test assay cGAMP value than a reference cGAMP value to thereby produce an effective test compound.

67) The effective test compound produced of statement 65, wherein the metastatic cancer cells or metastatic tissues are mixed in the culture medium to produce the test assay.

68) The effective test compound produced of statement 65 or 66, wherein the method further comprises extracting the cells with an alcohol (e.g. methanol, ethanol: or isopropanol) to produce an alcohol extract before measuring the cGAMP.

69) The effective test compound produced of statement 65, 66 or 67, wherein the method further comprises extracting the cell or tissue sample with methanol to produce a methanol extract and measuring the cGAMP in the methanol extract.

70) The effective test compound produced of statement 67 or 68, wherein the method further comprises purifying the alcohol extract or the methanol extract by Solid Phase Extraction (SPE) using one or more HyperSep aminopropyl solid phase columns to produce a semi-pure test sample before measuring the cGAMP of the semi-pure test sample.

71) The effective test compound produced of statement 65-68 or 69, wherein measuring cGAMP amounts or concentrations comprises liquid chromatography and/or mass spectroscopy to measure the level of cGAMP.

72) The effective test compound produced of statement 65-69 or 70, wherein the method further comprises administering the effective test compound to an animal model, for example, to further evaluate the toxicity and/or efficacy of the effective test compound.

73) The effective test compound produced of statement 65-70 or 71, wherein the method further comprises administering the effective test compound to a patient or to the patent from whom the cell or tissue sample as obtained.

74) A method comprising: (a) mixing a test compound with KIF2B or MCAK in a test assay mixture that contains 2-amino-6-mercapto-7-methylpurine ribonucleoside (MESG); (b) incubating the test assay mixture to produce an incubated test assay; (c) measuring an amount of inorganic phosphate to provide an inorganic phosphate test result; and (d) comparing the inorganic phosphate test result to a control or reference.

75) The method of statement 74, wherein the control is the amount of inorganic phosphate (Pi) present in a control assay that contains the KIF2B or MCAK and the 2-amino-6-mercapto-7-methylpurne ribonucleoside (MESG), but that does not contain the test compound.

76) The method of statement 74, wherein the reference is a mean amount of inorganic phosphate (Pi) present in two or more control assays that contain the KIF2B or MCAK and the 2-amino-6-mercapto-7-methylpurine ribonucleoside (MESG), but that does not contain the test compound.

77) The method of statement 74, 75 or 76, further comprising selecting a test compound that has an inorganic phosphate test result higher than the control or reference.

78) The method of statement 74-76 or 77, further comprising selecting a test compound that has an inorganic phosphate test result higher than the control or reference, and evaluating the test compound in a second assay to assess test compound as an activator of KIF2B or MCAK.

79) A method comprising: (a) mixing a test compound with cancer cells having γ-tubulin-labeled centrosomes to produce a test assay; (b) incubating the test assay for a time and under conditions sufficient for the test compound to penetrate the cancer cells to produce incubated test cancer cells; (c) measuring the distance between γ-tubulin-labeled centrosomes within a series of incubated test cancer cells to produce a mean distance result; and (d) comparing the mean distance result to a control or reference.

80) The method of statement 79, wherein the distance is measured by fluorescent in situ hybridization (FISH).

81) The method of statement 79 or 80, wherein the control is the distance between γ-tubulin-labeled centrosomes in cancer cells of a control assay that does not contain the test compound.

82) The method of statement 79 or 80, wherein the reference is a mean distance between γ-tubulin-labeled centrosomes within a series of γ-tubulin-labeled cancer cells in a control assay that does not contain the test compound.

83) The method of statement 79, 80 or 81, further comprising selecting a test compound that has a lower mean distance result than the control or reference.

84) The method of statement 74-76 or 77, further comprising selecting a test compound that has a lower mean distance result than the control or reference, and evaluating the test compound in a second assay to assess test compound as an activator of MCAK.

85) A method comprising (a) mixing NF-kB Inducing Kinase with a test compound, ATP, and an antibody with a fluorescent tracer (633 nm) bound to the antibody, where the antibody specifically recognizes ADP; (b) incubating the test assay mixture to produce an incubated test assay; (c) measuring an amount of fluorescence in the incubated test assay; and (d) comparing the amount of fluorescence in the incubated test assay to a control or reference.

86) The method of statement 85, wherein the control is the amount of fluorescence in a control assay that does not contain the test compound.

87) The method of statement 85, wherein the reference is a mean amount of fluorescence in a series of control assays that do not contain the test compound.

88) The method of statement 85, 86 or 87, further comprising selecting a test compound that has a higher amount of fluorescence in one or more incubated test assays than the control or reference.

89) The method of statement 85-87 or 88, further comprising selecting a test compound that has a higher amount of fluorescence in one or more incubated test assays than the control or reference, and evaluating the test compound in a second assay to assess the test compound as an inhibitor of NF-kB Inducing Kinase.

90) A method comprising: (a) mixing cancer cells with a test compound and an anti-lymphotoxin beta receptor (LT-βR) antibody; (b) incubating the test assay for a time and under conditions sufficient for the test compound to penetrate the cancer cells to produce incubated test cancer cells; (c) measuring the quantity of RELB translocation into nuclei of the incubated test cancer cells; and (d) comparing the amount quantity of RELB translocation into nuclei of the incubated test cancer cells to a control or reference.

91) The method of statement 90, wherein measuring the quantity of RELB translocation into nuclei of the incubated test cancer cells further comprises obtaining a ratio of the nuclear over cytoplasmic signal intensity.

92) The method of statement 90 or 91, wherein the control is the amount of RELB translocation into nuclei in a control assay that does not contain the test compound.

93) The method of statement 90 or 91, wherein the reference is a mean amount of RELB translocation into nuclei in a series of control assays that do not contain the test compound.

94) The method of statement 90-92 or 93, further comprising selecting a test compound that has a lower quantity of RELB translocation into nuclei of the incubated test cancer cells than the control or reference.

95) The method of statement 85-87 or 88, further comprising selecting a test compound that has a lower quantity of RELB translocation into nuclei of the incubated test cancer cells than the control or reference, and evaluating the test compound in a second assay to assess the test compound as an inhibitor of NF-kB Inducing Kinase.

96) An effective test compound produced by the method of statement 74-94 or 95.

97) The effective test compound of statement 96 wherein the method further comprises administering the effective test compound to an animal model, for example, to further evaluate the toxicity and/or efficacy of the effective test compound.

98) The effective test compound of statement 96 or 97, wherein the method further comprises administering the effective test compound to a patient or to the patent from whom the cancer cells were obtained.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “an expression cassette” or “a cell” includes a plurality of such nucleic acids, expression vectors or cells (for example, a solution or dried preparation of nucleic acids or expression cassettes, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1-7. (canceled)

8. A method comprising (a) obtaining a cell or tissue sample from a patient; (b) measuring: the amount or concentration of cGAMP produced from a known number or weight of cells or tissues from the sample to generate a reference cGAMP value; (c) mixing the same known number or weight of cells or tissues from the sample with a test compound to generate a test assay, (d) measuring the cGAMP amount or concentration in the test assay to generate a test assay cGAMP value, (e) optionally repeating steps (c) and (d); and selecting any test compound with a lower test assay cGAMP value than the reference cGAMP value to thereby identify at least one effective test compound.

9. The method of claim 8, wherein the sample comprises metastatic cancer cells or metastatic tissues.

10. The method of claim 8, further comprising extracting the cell or tissue sample with an alcohol to produce an alcohol extract before measuring the cGAMP.

11. The method of claim 8, further comprising extracting the cell or tissue sample with methanol to produce a methanol extract and measuring the cGAMP in the methanol extract.

12. The method of claim 10, further comprising purifying the alcohol extract or the methanol extract by Solid Phase Extraction (SPE) using one or more HyperSep aminopropyl solid phase columns to produce a semi-pure test sample before measuring the cGAMP of the semi-pure test sample.

13. The method of claim 8, wherein measuring cGAMP amounts or concentrations comprises liquid chromatography and/or mass spectroscopy.

14. The method of claim 8, further comprising administering the effective test compound to an animal cancer model.

15. The method of claim 8, further comprising administering the effective test compound to a patient or to the patent from whom the cell or tissue sample as obtained.

16. (canceled)

17. A method comprising administering a metastatic chemotherapeutic agent to a patient with a cell sample or bodily fluid sample:
a. having at least 10% detectable chromosomal missegregations within one or cells of the cell sample;
b. having at least 3% detectable micronuclei within one or cells of the cell sample;
c. having detectable cytosolic double-stranded DNA within one or cells of the cell sample; or
d. having at least 10% greater concentration or amount of cGAMP in the cell sample or bodily fluid sample.

18. The method of claim 17, comprising administering a metastatic chemotherapeutic agent to a patient
a. with 15-20% of chromosomes exhibiting missegregations in anaphase cells of the cell sample;
b. with 5-8% of cells in the cell sample exhibiting micronuclei;
c. with 1-fold to 2-fold increase in staining intensity within the cytosol compared to a normal non-cancer tissue; or
d. with 1-fold to 2-fold greater concentration or amount of cGAMP in the bodily fluid sample than a non-cancerous bodily fluid sample.

19. The method of 17, further comprising monitoring samples from the patient over time to quantify chromosomal missegregations, micronuclei, cytosolic double-stranded DNA, or cGAMP within cells or bodily fluids of the patient.

20. The method of claim 17, wherein the metastatic chemotherapeutic agent is a composition comprising one or more kinesin-13 protein(s), one or more MCAK protein(s), or a combination thereof.

21. The method of claim 17, wherein the metastatic chemotherapeutic agent is a composition comprising a kinesin-13 nucleic acid or an expression cassette having a promoter operably linked to a nucleic acid segment encoding a kinesin-13 protein.

22. A method comprising (a) quantifying expression levels of the following genes in a test sample from a patient: PELI2, BMP2, SHH, TNS4, RAB3B, ROBO1, ARHGAP28, CHN2, CST1, F13A1, CPVL, SEMA6D, C9orfl52, NHSL2, GTF2IP7, DPYSL3, PCDH7, KHDRBS3, TRAC, TMEM156, CST4, CD24, or FGF5, to generate quantified expression levels each of following genes in the test sample: PELI2, BMP2, SHH, TNS4, RAB3B, ROBO1, ARHGAP28, CHN2, CST1, F13A1, CPVL, SEMA6D, C9orfl52, NHSL2, GTF2IP7, DPYSL3, PCDH7, KHDRBS3, TRAC, TMEM156, CST4, CD24, or FGF5; and (b) informing the patient of longer metastasis-free survival when each quantified expression level is greater than a median reference expression level for each of these genes.

23. The method of claim 22, wherein the median reference expression level for each of these genes is the median expression of each of these genes in samples from a series of patients with metastatic cancer.

24. The method of claim 22, wherein the patient has breast cancer.

25. (canceled)


SEQUENCE LISTING (61 sequences)

SEQ ID NO: 1 - protein, 673 aa, Homo sapiens
MASQFCLPESPCLSPLKPLKPHFGDIQEGIYVAIQRSDKRIHLAVVTEINRENYWVTVEWVEKAVKKGKKIDLETILLLNPALDSAEHPMPPPPLSPLALAPSSAIRDQRTATKWVAMIPQKNQTASGDSLDVRVPSKPCLMKQKKSPCLWEIQKLQEQREKRRRLQQEIRARRALDVNTRNPNYEIMHMIEEYRRHLDSSKISVLEPPQEHRICVCVRKRPLNQRETTLKDLDIITVPSDNVVMVHESKQKVDLTRYLQNQTFCFDHAFDDKASNELVYQFTAQPLVESIFRKGMATCFAYGQTGSGKTYTMGGDFSGTAQDCSKGIYALVAQDVFLLLRNSTYEKLDLKVYGTFFEIYGGKVYDLLNWKKKLQVLEDGNQQIQVVGLQEKEVCCVEEVLNLVEIGNSCRTSRQTPVNAHSSRSHAVFQIILKSGRIMHGKFSLVDLAGNERGADTTKASRKRQLEGAEINKSLLALKECILALGQNKPHTPFRASKLTLVLRDSFIGQNSSTCMIATISPGMTSCENTLNTLRYANRVKKLNVDVRPYHRGHYPIGHEAPRMLKSHIGNSEMSLQRDEFIKIPYVQSEEQKEIEEVETLPTLLGKDTTISGKGSSQWLENIQERAGGVHHDIDFCIARSLSILEQKIDALTEIQKKLKLLLADLHVKSKVE

SEQ ID NO: 2 - protein, 2335 aa, Homo sapiens
GTAGTGGCCCCAGTCCGGGCCCCGGCGCGCTAGGCTCACAAAGGCAGGCACAGACTGCAACCCTGCTCAGTGCTCCGGGCGCTTCAGGCTGGCTTGGGTCCTGCTGCTCCAACCCCAAGGGCCCTGGAGCGCTCCCTGATACCTCCATCACTCACCATGGCCAGCCAGTTCTGCCTCCCTGAATCCCCATGTCTCTCGCCCCTGAAACCCTTGAAGCCACATTTCGGAGACATCCAAGAGGGCATCTACGTGGCGATCCAGCGCAGTGACAAGCGGATCCACCTCGCTGTGGTCACGGAGATCAACAGAGAAAACTATTGGGTCACGGTAGAGTGGGTGGAGAAAGCAGTCAAAAAAGGCAAGAAGATTGACCTGGAGACCATACTCCTGCTGAATCCAGCTCTGGACTCTGCTGAACACCCCATGCCGCCCCCGCCCTTATCCCCCTTGGCTCTGGCGCCCTCTTCGGCCATCAGGGACCAGCGTACCGCCACGAAATGGGTTGCGATGATCCCCCAGAAAAACCAAACAGCCTCAGGGGACAGCCTGGATGTGAGGGTCCCCAGCAAACCTTGTCTGATGAAGCAGAAAAAGTCTCCCTGCCTCTGGGAAATCCAGAAACTGCAGGAGCAGCGGGAAAAGCGCAGGCGGCTGCAGCAGGAGATCCGAGCTAGACGCGCCCTCGATGTCAATACCAGAAACCCCAACTACGAAATCATGCACATGATCGAAGAGTATCGCAGGCACCTGGACAGCAGCAAGATCTCAGTCCTGGAGCCCCCGCAAGAACATCGCATCTGCGTCTGCGTGAGGAAGCGGCCTCTCAACCAGCGAGAGACAACCTTAAAGGACCTGGATATCATCACCGTCCCCTCGGACAATGTGGTTATGGTGCATGAGTCCAAGCAAAAGGTGGACCTCACTCGCTACCTGCAGAACCAGACCTTCTGCTTCGACCATGCCTTCGATGACAAAGCCTCCAACGAGTTGGTGTACCAGTTCACCGCCCAGCCACTGGTGGAGTCCATCTTCCGCAAGGGCATGGCCACCTGCTTTGCCTATGGGCAGACGGGAAGTGGGAAGACGTACACCATGGGTGGAGACTTTTCAGGAACGGCCCAAGATTGTTCTAAGGGCATTTATGCTCTGGTGGCACAGGATGTCTTTCTCCTGCTCAGAAACTCCACATATGAGAAGCTGGACCTCAAAGTCTATGGGACATTTTTTGAGATTTATGGGGGCAAGGTGTATGATTTGTTGAACTGGAAGAAGAAGCTGCAAGTCCTTGAGGATGGCAATCAGCAAATCCAAGTGGTCGGGCTGCAGGAGAAAGAGGTGTGTTGTGTGGAGGAAGTGCTGAACCTGGTGGAAATAGGGAATAGCTGTCGGACTTCCAGGCAAACACCTGTCAACGCTCACTCATCCAGGAGCCATGCAGTGTTCCAGATCATCCTGAAGTCAGGACGGATAATGCATGGCAAGTTTTCCCTCGTTGATTTAGCTGGGAATGAAAGAGGAGCAGATACAACCAAGGCCAGCCGGAAAAGGCAGCTGGAAGGGGCAGAGATTAACAAGAGTCTTCTAGCCCTCAAAGAATGTATTCTGGCTTTGGGTCAGAACAAGCCTCACACCCCATTCAGAGCCAGCAAACTCACACTGGTGCTCCGGGACTCCTTTATAGGCCAGAACTCCTCCACTTGCATGATTGCTACCATCTCTCCGGGGATGACCTCTTGTGAAAACACTCTCAACACTTTAAGATATGCAAACAGAGTAAAAAAATTAAATGTAGATGTAAGGCCCTACCATCGTGGCCACTATCCGATTGGACATGAGGCACCAAGGATGTTAAAAAGTCACATCGGAAATTCAGAAATGTCCCTTCAGAGGGATGAATTTATTAAAATACCTTATGTACAGAGTGAGGAGCAGAAAGAGATTGAAGAGGTTGAAACATTACCCACTCTGTTAGGGAAGGATACCACAATTTCAGGGAAGGGATCTAGCCAATGGCTGGAAAACATCCAGGAGAGAGCTGGTGGAGTACACCATGATATTGATTTTTGCATTGCCCGGTCTTTGTCCATTTTGGAGCAGAAAATTGATGCTCTGACCGAGATCCAAAAGAAACTGAAATTATTACTAGCTGACCTCCACGTGAAGAGCAAGGTAGAGTGAAGCCAATGGCGAGAGATCAGGTCCGAAATGCTGCATTGCTGCAGTTTCCACCACTCTTATACAGGAAAACTGTCCAAATTATCTAAAGATCCTCCTGAGAAGCTTAAAACATCTTAAAATACACTGATGGGAAACATGCTCTTTCTTCTGCCTCTGT

SEQ ID NO: 3 - protein, 725 aa, Homo sapiens
MAMDSSLQARLFPGLAIKIQRSNGLIHSANVRTVNLEKSCVSVEWAEGGATKGKEIDFDDVAAINPELLQLLPLHPKDNLPLQENVTIQKQKRRSVNSKIPAPKESLRSRSTRMSTVSELRITAQENDMEVELPAAANSRKQFSVPPAPTRPSCPAVAEIPLRMVSEEMEEQVHSIRGSSSANPVNSVRRKSCLVKEVEKMKNKREEKKAQNSEMRMKRAQEYDSSFPNWEFARMIKEFRATLECHPLTMTDPIEEHRICVCVRKRPLNKQELAKKEIDVISIPSKCLLLVHEPKLKVDLTKYLENQAFCFDFAFDETASNEVVYRFTARPLVQTIFEGGKATCFAYGQTGSGKTHTMGGDLSGKAQNASKGIYAMASRDVFLLKNQPCYRKLGLEVYVTFFEIYNGKLFDLLNKKAKLRVLEDGKQQVQVVGLQEHLVNSADDVIKMLDMGSACRTSGQTFANSNSSRSHACFQIILRAKGRMHGKFSLVDLAGNERGADTSSADRQTRMEGAEINKSLLALKECIRALGQNKAHTPFRESKLTQVLRDSFIGENSRTCMIATISPGISSCEYTLNTLRYADRVKELSPHSGPSGEQLIQMETEEMEACSNGALIPGNLSKEEEELSSQMSSFNEAMTQIRELEEKAMEELKEIIQQGPDWLELSEMTEQPDYDLETFVNKAESALAQQAKHFSALRDVIKALRLAMQLEEQASRQISSKKRPQ

SEQ ID NO: 4 - DNA, 2886 bp, Homo sapiens
  source 1..2886: mol_type = other DNA
acgcttgcgcgcgggatttaaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgtggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgctcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcagacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttacgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagtcctagaggatccctactgttttctgttttatgtgtttatacattgtatgtaacaataaagagaaaaaataaaaaaaaaaaaaaaaaaaaaaaaaaaaaaaa

SEQ ID NO: 5 - protein, 1805 aa, Homo sapiens
MSDTKVKVAVRVRPMNRRELELNTKCVVEMEGNQTVLHPPPSNTKQGERKPPKVFAFDYCFWSMDESNTTKYAGQEVVFKCLGEGILEKAFQGYNACIFAYGQTGSGKSFSMMGHAEQLGLIPRLCCALFKRISLEQNESQTFKVEVSYMEIYNEKVRDLLDPKGSRQSLKVREHKVLGPYVDGLSQLAVTSFEDIESLMSEGNKSRTVAATNMNEESSRSHAVFNIIITQTLYDLQSGNSGEKVSKVSLVDLAGSERVSKTGAAGERLKEGSNINKSLTTLGLVISSLADQAAGKGKSKFVPYRDSVLTWLLKDNLGGNSQTSMIATISPAADNYEETLSTLRYADRAKRIVNHAVVNEDPNAKVIRELREEVEKLREQLSQAEAMKAPELKEKLEESEKLIKELTVTWEEKLRKTEEIAQERQRQLESMGISLEMSGIKVGDDKCYLVNLNADPALNELLVYYLKDHTRVGADTSQDIQLFGIGIQPQHCEIDIASDGDVTLTPKENARSCVNGTLVCSTTQLWHGDRILWGNNHFFRINLPKRKRRDWLKDFEKETGPPEHDLDAASEASSEPDYNYEFAQMEVIMKTLNSNDPVQNVVQVLEKQYLEEKRSALEEQRLMYERELEQLRQQLSPDRQPQSSGPDRLAYSSQTAQQKVTQWAEERDELFRQSLAKLREQLVKANTLVREANFLAEEMSKLTDYQVTLQIPAANLSANRKRGAIVSEPAIQVRRKGKSTQVWTIEKLENKLIDMRDLYQEWKEKVPEAKRLYGKRGDPFYEAQENHNLIGVANVFLECLFCDVKLQYAVPIISQQGEVAGRLHVEVMRVTGAVPERVVEDDSSENSSESGSLEVVDSSGEIIHRVKKLTCRVKIKEATGLPLNLSNFVFCQYTFWDQCESTVAAPVVDPEVPSPQSKDAQYTVTFSHCKDYVVNVTEEFLEFISDGALAIEVWGHRCAGNGSSIWEVDSLHAKTRTLHDRWNEVTRRIEMWISILELNELGEYAAVELHQAKDVNTGGIFQLRQGHSRRVQVTVKPVQHSGTLPLMVEAILSVSIGCVTARSTKLQRGLDSYQRDDEDGDDMDSYQEEDLNCVRERWSDALIKRREYLDEQIKKVSNKTEKTEDDVEREAQLVEQWVGLTEERNAVLVPAPGSGIPGAPADWIPPPGMETHIPVLFLDLNADDLSANEQLVGPHASGVNSILPKEHGSQFFYLPIIKHSDDEVSATASWDSSVHDSVHLNRVTPQNERIYLIVKTTVQLSHPAAMELVLRKRIAANIYNKQSFTQSLKRRISLKNIFYSCGVTYEIVSNIPKATEEIEDRETLALLAARSENEGTSDGETYIEKYTRGVLQVENILSLERLRQAVTVKEALSTKARHIRRSLSTPNVHNVSSSRPDLSGFDEDDKGWPENQLDMSDYSSSYQDVACYGTLPRDSPRRNKEGCTSETPHALTVSPFKAFSPQPPKFFKPLMPVKEEHKKRIALEARPLLSQESMPPPQAHNPGCIVPSGSNGSSMPVEHNSKREKKIDSEEEENELEAINRKLISSQPYVPVEFADFSVYNASLENREWFSSKVDLSNSRVLEKEVSRSPTTSSITSGYFSHSASNATLSDMVVPSSDSSDQLAIQTKDADSTEHSTPSLVHDFRPSSNKELTEVEKGLVKDKIIVVPLKENSALAKGSPSSQSIPEKNSKSLCRTGSCSELDACPSKISQPARGFCPREVTVEHTTNILEDHSFTEFMGVSEGKDFDGLTDSSAGELSSRRSLPNKTGGKTVSDGLHHPSQLHSKLENDQVIIPEAAFWVLCCQ

SEQ ID NO: 6 - DNA, 6005 bp, Homo sapiens
  source 1..6005: mol_type = other DNA
cgggatggcccgcgcgcctcggcgctgcctctcggagctcacggcggagcggcggcggccgcgctcgaggggcgcgcggctgcagcggcggcggcgccgcgcgtgaggggccgcctaaggccgagcgggcgcggcgagcggccgggcgagcgcagccaacatgtcggataccaaggtaaaagttgccgtccgggtccggcccatgaaccgacgagaactggaactgaacaccaagtgcgtggtggagatggaagggaatcaaacggtcctgcaccctcctccttctaacaccaaacagggagaaaggaaacctcccaaggtatttgcctttgattattgcttttggtccatggatgaatctaacactacaaaatacgctggtcaagaagtggttttcaagtgccttggggaaggaattcttgaaaaagcctttcaggggtataatgcgtgtatttttgcatatggacagacaggttcgggaaaatccttttccatgatgggccatgctgagcagctgggccttattccaaggctctgctgtgctttatttaaaaggatctctttggagcaaaatgagtcacagacctttaaagttgaagtgtcctatatggaaatttataatgagaaagttcgggatcttttagaccccaaagggagtagacagtctcttaaagttcgagaacataaagttttgggaccatatgtagatggtttatctcaactagctgtcactagttttgaggatattgagtcattgatgtctgagggaaataagtctcgaacggtagctgctaccaacatgaacgaagaaagcagccgctcccatgctgtgttcaacatcataatcacacagacactttatgacctgcagtctgggaattccggggagaaagtcagtaaggtcagcttggtagacctggcgggtagcgaaagagtatctaaaacaggagctgcaggagagcgactgaaagaaggcagcaacattaacaaatcgcttacaaccttggggttggttatatcatcactggctgaccaggcagctggcaagggtaaaagcaaatttgtgccttatcgagattcagtcctcacttggctgcttaaggacaacttggggggcaacagccaaacctctatgatagccacaatcagcccagccgcagacaactatgaagagaccctctccacattaagatatgcagaccgagccaaaaggattgtgaaccatgctgttgtgaatgaggaccccaacgcaaaagtgatccgagaactgcgggaggaagtcgagaaactgagagagcagctctctcaggcagaggccatgaaggcccctgaactgaaggagaagctcgaagagtctgaaaagctgataaaagaactaacagtgacttgggaagagaagctgagaaaaacagaagagatagcacaggaaagacaacgacaacttgaaagcatggggatttccctggagatgtccggtatcaaggtgggggatgacaaatgctacttagtcaatctgaatgcagaccctgctcttaacgaacttctggtttattatttaaaggatcacaccagggtgggtgcagatacctctcaagatatccagctttttggcataggaattcagcctcagcactgtgagattgacattgcatctgatggagacgtcactctcactccaaaagaaaatgcaaggtcctgtgtgaacggcacccttgtgtgcagtaccacccagctgtggcatggtgaccgaatcctatggggaaataatcacttttttagaataaacttacctaagaggaaacgtcgagattggttgaaagactttgaaaaagaaacgggcccgccagagcatgacctggatgcagccagtgaggcttcctctgaaccagactataactatgaatttgcacagatggaagttatcatgaaaaccctgaatagtaatgacccagttcaaaatgtggttcaggtcctggagaaacaatacctagaagaaaagagaagtgccctagaggagcagcggctcatgtatgagcgggaactggagcaactccgccagcagctctcccccgacaggcagccacagagtagcggccctgaccgcctggcctacagcagccagacagcgcagcagaaggtgacccagtgggcagaagagagggatgaactcttccgacaaagcctggcaaaactgcgagagcagctggttaaagctaataccttggtgagggaagcaaacttcctggctgaggaaatgagcaaactcaccgattaccaagtgactcttcagatccctgctgcaaacctcagtgccaataggaagagaggtgcaatagtgagtgaaccagctatccaagtgaggaggaaaggaaagagcacccaagtgtggaccattgagaagctggagaataaattaattgacatgagagacctttaccaagaatggaaggaaaaagttcctgaggcaaagagactctacggaaaacgaggtgaccctttctatgaagcccaagaaaatcacaacctcatcggggtggcgaatgtattcttggaatgcctcttctgtgatgtgaaacttcagtatgcagtccctatcatcagccagcagggggaggttgcagggcgtctccacgtggaagtgatgcgtgttacaggagctgttccagagcgtgtggtggaggatgactcttcggagaattccagtgaaagtgggagccttgaagtcgtagacagcagcggggaaatcattcaccgagtcaaaaagctgacatgtcgggtaaaaattaaagaagcaacggggctgcccttaaacctctcaaattttgtcttctgtcaatacacattctgggaccagtgtgagtctacggtggctgccccggtggtggaccccgaggtgccttcaccacagtccaaggatgcccagtacacagtgaccttctcccactgtaaggactatgtggtgaatgtaacagaagaatttctggagttcatttcagatggagcactggccattgaagtatggggccaccggtgtgctggaaatggcagctccatctgggaggtcgattctcttcatgctaagacaagaacactgcatgacaggtggaatgaagtaacgcgaagaatagaaatgtggatctccatattagaattgaatgagttaggagagtatgctgcagtggaacttcatcaggcaaaagatgtcaacacaggaggcatctttcaacttagacagggtcattcccgtagagtacaagtcacggtgaaacctgtgcagcattcagggacactgccacttatggttgaagccatcctgtcagtatccatcggctgtgtaactgccaggtccaccaaactccaaagagggctggacagttaccagagagatgatgaggatggtgatgatatggatagttatcaggaagaagacttaaactgcgtaagggagaggtggtcagatgcactcattaaacgacgagaatacctggatgaacagataaaaaaagtcagcaataaaacagagaaaacagaggacgatgtggagcgggaagcccagcttgtggagcagtgggtagggctgactgaggaaaggaatgctgtgctggtgccagccccaggcagtgggattcctggggcacctgccgactggatcccacctcctggaatggaaacccacataccagttctcttcctcgatttgaatgcggatgacctcagtgccaatgagcagcttgttggcccccatgcatccggcgtgaactccatcctgcccaaggagcatggcagccagtttttctacctgcccatcataaagcacagtgatgatgaggtttcagccacagcctcttgggattcctcggtgcatgattctgttcacttgaatagggtcacaccacagaatgaaaggatttacctaattgtgaaaaccacagttcaactcagccaccctgctgctatggagttagtattacgaaaacgaattgcagccaatatttacaacaaacagagtttcacgcagagtttgaagaggagaatatccctgaaaaatatattttattcctgtggtgtaacctatgaaatagtatccaatataccaaaggcaactgaggagatagaggaccgggaaacgctggctctcctggcagcaaggagtgaaaacgaaggcacatcagatggggagacgtacattgagaagtacactcgaggcgtgctgcaggtggaaaacattctgagtcttgaacggctccggcaggccgtcacagtcaaagaagcactttccaccaaagcccggcacattcggaggagcctcagtacaccaaatgttcataatgtctcttccagccgaccggacctttctggctttgatgaagatgacaagggttggccagagaaccagttggacatgtctgactatagctccagttaccaagatgtagcatgttatggaactttacccagggattctcctcgaaggaataaagaaggttgtacatcagagactcctcatgccttaaccgtcagcccttttaaagcattctctcctcagccaccaaagtttttcaagcccctaatgcctgtaaaagaggagcataagaaaaggatagccctggaagcaaggcctcttctaagccaggagagcatgcctccacctcaggcacataaccctggctgcattgtaccctcaggaagcaatggcagcagcatgccagtagaacacaatagcaaacgtgagaagaagattgactctgaggaggaagaaaatgagctggaagctattaacaggaagctaataagttcacagccttatgtacctgtggagtttgctgacttcagtgtttacaatgccagcttggagaacagggaatggttttcctctaaagtagatctgtcaaactcacgggtcttggagaaagaagtgtcccgtagccctaccaccagcagtattaccagtggctacttttcccacagtgcctccaatgccaccctgtctgacatggtggtcccttctagtgacagctcagaccagctggccattcagacgaaggatgcagactccaccgagcactccacaccatcgcttgtgcatgatttcaggccgtcctcaaacaaagagttgacagaagtcgaaaaaggcttggtaaaggacaagataattgtggtgccactcaaggaaaacagtgccttagccaaagggagcccatcatcccagagcatccctgagaaaaactccaaatcactgtgcaggactggctcatgttcagaactagatgcctgccccagcaaaattagccagccagccaggggattctgccccagggaggtgacggtagaacacaccaccaacatccttgaagaccattctttcacagaatttatgggagtgtcagagggaaaagattttgatggtttgacagattcttctgctggagagctttccagtaggaggagtctaccaaataaaacaggcggcaagactgtctccgatgggctccaccaccccagccagctgcattccaagttagagaatgaccaggtaataattccagaggcagccttttgggttctgtgctgtcaatgagtatgtctaactgtatgtcaaccccagaggcccttcaccgcaacaacttggtaggaaagattcatccagttgtttgtgacagcaaagatgagcccacagagaagcaggctcacttcctgcacagctgtctctgtcggagagcaagtctgttttgggaactagaacgcaattgtgaaattataagaccagtggatttttttacctggcacatgggttggtgttgaatgaagtgttcagatggataaggatcaatctcatattcattccctgggatgtttagttaccagttttcccaaagtgttctggtagcatctaccatatttcatcaaatctgtgattcctttgattattatatgaaccattattttatgtatcattaagaaaaaatactgccaattaaactctgtcatatcaacaaaaaaaaaaa

SEQ ID NO: 7 - protein, 725 aa, Homo sapiens
MAMDSSLQARLFPGLAIKIQRSNGLIHSANVRTVNLEKSCVSVEWAEGGATKGKEIDFDDVAAINPELLQLLPLHPKDNLPLQENVTIQKQKRRSVNSKIPAPKESLRSRSTRMSTVSELRITAQENDMEVELPAAANSRKQFSVPPAPTRPSCPAVAEIPLRMVSEEMEEQVHSIRGSSSANPVNSVRRKSCLVKEVEKMKNKREEKKAQNSEMRMKRAQEYDSSFPNWEFARMIKEFRATLECHPLTMTDPIEEHRICVCVRKRPLNKQELAKKEIDVISIPSKCLLLVHEPKLKVDLTKYLENQAFCFDFAFDETASNEVVYRFTARPLVQTIFEGGKATCFAYGQTGSGKTHTMGGDLSGKAQNASKGIYAMASRDVFLLKNQPCYRKLGLEVYVTFFEIYNGKLFDLLNKKAKLRVLEDGKQQVQVVGLQEHLVNSADDVIKMIDMGSACRTSGQTFANSNSSRSHACFQIILRAKGRMHGKFSLVDLAGNERGADTSSADRQTRMEGAEINKSLLALKECIRALGQNKAHTPFRESKLTQVLRDSFIGENSRTCMIATISPGISSCEYTLNTLRYADRVKELSPHSGPSGEQLIQMETEEMEACSNGALIPGNLSKEEEELSSQMSSFNEAMTQIRELEEKAMEELKEIIQQGPDWLELSEMTEQPDYDLETFVNKAESALAQQAKHFSALRDVIKALRLAMQLEEQASRQISSKKRPQ

SEQ ID NO: 8 - DNA, 2896 bp, Homo sapiens
  source 1..2896: mol_type = other DNA
acgcttgcgcgcgggatttaaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaaaaaaaaaaaaaaaaa

SEQ ID NO: 9 - protein, 859 aa, Homo sapiens
MLPVYQEVKPNPLQDANLCSRVFFWWLNPLFKIGHKRRLEEDDMYSVLPEDRSQHLGEELQGFWDKEVLRAENDAQKPSLTRAIIKCYWKSYLVLGIFTLIEESAKVIQPIFLGKIINYFENYDPMDSVALNTAYAYATVLTFCTLILAILHHLYFYHVQCAGMRLRVAMCHMIYRKALRLSNMAMGKTTTGQIVNLLSNDVNKFDQVTVFLHFLWAGPLQAIAVTALLWMEIGISCLAGMAVLIILLPLQSCFGKLFSSLRSKTATFTDARIRTMNEVITGIRIIKMYAWEKSFSNLITNLRKKEISKILRSSCLRGMNLASFFSASKIIVFVTFTTYVLLGSVITASRVFVAVTLYGAVRLTVTLFFPSAIERVSEAIVSIRRIQTFLLLDEISQRNRQLPSDGKKMVHVQDFTAFWDKASETPTLQGLSFTVRPGELLAVVGPVGAGKSSLLSAVLGELAPSHGLVSVHGRIAYVSQQPWVFSGTLRSNILFGKKYEKERYEKVIKACALKKDLQLLEDGDLTVIGDRGTTLSGGQKARVNLARAVYQDADIYLLDDPLSAVDAEVSRHLFELCICQILHEKITILVTHQLQYLKAASQILILKDGKMVQKGTYTEFLKSGIDFGSLLKKDNEESEQPPVPGTPTLRNRTFSESSVWSQQSSRPSLKDGALESQDTENVPVTLSEENRSEGKVGFQAYKNYFRAGAHWIVFIFLILLNTAAQVAYVLQDWWLSYWANKQSMLNVTVNGGGNVTEKLDLNWYLGIYSGLTVATVLFGIARSLLVFYVLVNSSQTLHNKMFESILKAPVLFFDRNPIGRILNRFSKDIGHLDDLLPLTFLDFIQRWDLAVLSWLVSNS

SEQ ID NO: 10 - DNA, 2890 bp, Homo sapiens
  source 1..2890: mol_type = other DNA
ggccggagccccagcatccctgcttgaggtccaggagcggagcccgcggccaccgccgcctgatcagcgcgaccccggcccgcgcccgccccgcccggcaagatgctgcccgtgtaccaggaggtgaagcccaacccgctgcaggacgcgaacctctgctcacgcgtgttcttctggtggctcaatcccttgtttaaaattggccataaacggagattagaggaagatgatatgtattcagtgctgccagaagaccgctcacagcaccttggagaggagttgcaagggttctgggataaagaagttttaagagctgagaatgacgcacagaagccttctttaacaagagcaatcataaagtgttactggaaatcttatttagttttgggaatttttacgttaattgaggaaagtgccaaagtaatccagcccatatttttgggaaaaattattaattattttgaaaattatgatcccatggattctgtggctttgaacacagcgtacgcctatgccacggtgctgactttttgcacgctcattttggctatactgcatcacttatatttttatcacgttcagtgtgctgggatgaggttacgagtagccatgtgccatatgatttatcggaaggcacttcgtcttagtaacatggccatggggaagacaaccacaggccagatagtcaatctgctgtccaatgatgtgaacaagtttgatcaggtgacagtgttcttacacttcctgtgggcaggaccactgcaggcgatcgcagtgactgccctactctggatggagataggaatatcgtgccttgctgggatggcagttctaatcattctcctgcccttgcaaagctgttttgggaagttgttctcatcactgaggagtaaaactgcaactttcacggatgccaggatcaggaccatgaatgaagttataactggtataaggataataaaaatgtacgcctgggaaaagtcattttcaaatcttattaccaatttgagaaagaaggagatttccaagattctgagaagttcctgcctcagggggatgaatttggcttcgtttttcagtgcaagcaaaatcatcgtgtttgtgaccttcaccacctacgtgctcctcggcagtgtgatcacagccagccgcgtgttcgtggcagtgacgctgtatggggctgtgcggctgacggttaccctcttcttcccctcagccattgagagggtgtcagaggcaatcgtcagcatccgaagaatccagacctttttgctacttgatgagatatcacagcgcaaccgtcagctgccgtcagatggtaaaaagatggtgcatgtgcaggattttactgctttttgggataaggcatcagagaccccaactctacaaggcctttcctttactgtcagacctggcgaattgttagctgtggtcggccccgtgggagcagggaagtcatcactgttaagtgccgtgctcggggaattggccccaagtcacgggctggtcagcgtgcatggaagaattgcctatgtgtctcagcagccctgggtgttctcgggaactctgaggagtaatattttatttgggaagaaatacgaaaaggaacgatatgaaaaagtcataaaggcttgtgctctgaaaaaggatttacagctgttggaggatggtgatctgactgtgataggagatcggggaaccacgctgagtggagggcagaaagcacgggtaaaccttgcaagagcagtgtatcaagatgctgacatctatctcctggacgatcctctcagtgcagtagatgcggaagttagcagacacttgttcgaactgtgtatttgtcaaattttgcatgagaagatcacaattttagtgactcatcagttgcagtacctcaaagctgcaagtcagattctgatattgaaagatggtaaaatggtgcagaaggggacttacactgagttcctaaaatctggtatagattttggctcccttttaaagaaggataatgaggaaagtgaacaacctccagttccaggaactcccacactaaggaatcgtaccttctcagagtcttcggtttggtctcaacaatcttctagaccctccttgaaagatggtgctctggagagccaagatacagagaatgtcccagttacactatcagaggagaaccgttctgaaggaaaagttggttttcaggcctataagaattacttcagagctggtgctcactggattgtcttcattttccttattctcctaaacactgcagctcaggttgcctatgtgcttcaagattggtggctttcatactgggcaaacaaacaaagtatgctaaatgtcactgtaaatggaggaggaaatgtaaccgagaagctagatcttaactggtacttaggaatttattcaggtttaactgtagctaccgttctttttggcatagcaagatctctattggtattctacgtccttgttaactcttcacaaactttgcacaacaaaatgtttgagtcaattctgaaagctccggtattattctttgatagaaatccaataggaagaattttaaatcgtttctccaaagacattggacacttggatgatttgctgccgctgacgtttttagatttcatccagagatgggatctcgctgtgttgtcctggctggtctcaaactcctaggctcaagcaatcctcctccctcctcaagcaaacctcagtgctgggattataggcatgagccactgtacctggctaaatgttgtttttttgatattcaatttttgtttatagaattttcatttgttttgctcttatacttttcatctttttatgtttattgaccaattaaatatcatttgggtaagcacctaaaaaaaaaaaaaaaaaa

SEQ ID NO: 11 - protein, 655 aa, Homo sapiens [sequence omitted]

SEQ ID NO: 12 - DNA, 2247 bp, Homo sapiens
  source 1..2247: mol_type = other DNA
accgtgcacatgcttggtggtcttgttaagtggaaactgctgctttagagtttgtttggaaggtccgggtgactcatcccaacatttacatccttaattgttaaagcgctgcctccgagcgcacgcatcctgagatcctgagcctttggttaagaccgagctctattaagctgaaaagataaaaactctccagatgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgaggatgttaccaagtattatatttacctgtatagtgtacttcatgttaggattgaaggcaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattcagtatgatttatcctcacataaaaaagaagcactttgattgaagtattcaaaaaaaaaaaaaaaaaaaaaa

SEQ ID NO: 13 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 14 - DNA, 2223 bp, Homo sapiens
  source 1..2223: mol_type = other DNA
tataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacccaaaaaaaaaaaaaaaaaa

SEQ ID NO: 15 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 16 - DNA, 1802 bp, Homo sapiens
  source 1..1802: mol_type = other DNA
agcctggggttccccttcgggtcgcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcac

SEQ ID NO: 17 - protein, 900 aa, Homo sapiens
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVISQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGGSLGFFPSSLAYSPYQSGAGPMGCYPGGGGGAQMAATVPSRDSGEEAAEPSAPSRTPQCEPQAPEMLQRAREYNARLFGLAQRSARALLDYGVTADARALLAGQRHLLTAQDENGDTPLHLAIIHGQTSVIEQIVYVIHHAQDLGVVNLTNHLHQTPLHLAVITGQTSVVSFLLRVGADPALLDRHGDSAMHLALRAGAGAPELLRALLQSGAPAVPQLLHMPDFEGLYPVHLAVRARSPECLDLLVDSGAEVEATERQGGRTALHLATEMEELGLVTHLVTKLRANVNARTFAGNTPLHLAAGLGYPTLTRLLLKAGADIHAENEEPLCPLPSPPTSDSDSDSEGPEKDTRSSFRGHTPLDLTCSTKVKTLLLNAAQNTMEPPLTPPSPAGPGLSLGDTALQNLEQLLDGPEAQGSWAELAERLGLRSLVDTYRQTTSPSGSLLRSYELAGGDLAGLLEALSDMGLEEGVRLLRGPETRDKLPSTAEVKEDSAYGSQSVEQEAEKLGPPPEPPGGLCHGHPQPQVH

SEQ ID NO: 18 - DNA, 3129 bp, Homo sapiens
  source 1..3129: mol_type = other DNA
gcctcccgcccctcccgtcgcgagggcggggccagtggcgtcatttccaggcccgccccctccggccccgcctccccttggtattttcgggactttcctaagctgctctaactttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 19 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 20 - DNA, 2297 bp, Homo sapiens
  source 1..2297: mol_type = other DNA
ggccccgcgccccgcgcagccccgggcgccgcgcgtcctgcccggcctgcggccccagcccttgcgccgctcgtccgacccgcgatcgtccaccagaccgtgcctcccggccgcccggccggcccgcgtgcatgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtagccccgcgatgccagaggaggggcactgggtggggagggaggtggaggagccgtgcaatcccaaccaggatgtctagcacccccatccccttggcccttcctcatgcttctgaagtggacatattcagccttggcgagaagctccgttgcacgggtttccccttgagcccattttacagatgaggaaactgagtccggagaggaaaagggacatggctcccgtgcactagcttgttacagctgcctctgtccccacatgtgggggcaccttctccagtaggattcggaaaagattgtacatatgggaggagggggcagattcctggccctccctccccagacttgaaggtggggggtaggttggttgttcagagtcttcccaataaagatgagtttttgagcctccgggaaaaaaaaaaaaaaaaaa

SEQ ID NO: 21 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 22 - DNA, 7442 bp, Homo sapiens
  source 1..7442: mol_type = other DNA
ccggagcggccggggccacgatggagcgcgacggctgcgcggggggcgggagccgcggcggcgagggcgggcgcgctccccgggagggcccggcggggaacggccgcgatcggggccgcagccacgctgccgaggcgcccggggacccgcaggcggccgcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagactgatatgttttttatccccaaacaccatgaatctttttgagagaaccttatattttatatagtcctctagctacactattgcattgttcagaaactgtcgaccagagttagaacggagccctcggtgatgcggacatctcagggaaacttgcgtactcagcacagcagtggagagtgttcctgttgaatcttgcacatatttgaatgtgtaagcattgtatacattgatcaagttcgggggaataaagacagaccacacctaaaactgcctttctgcttctcttaaaggagaagtagctgtgaacattgtctggataccagatatttgaatctttcttactattggtaataaaccttgatggcattgggcaaacagtagacttatagtagggttggggtagcccatgttatgtgactatctttatgagaattttaaagtggttctggatatcttttaacttggagtttcatttcttttcattgtaatcaaaaaaaaaattaacagaagccaaaatacttctgagaccttgtttcaatctttgctgtatatcccctcaaaatccaagttattaatcttatgtgttttctttttaattttttgattggatttctttagatttaatggttcaaatgagttcaactttgagggacgatctttgaatatacttacctattataaaatcttactttgtatttgtatttaaaaaagaaaaatattcctatcctgctcactggtaattaacataggtttaaaatggcttcaaatgtggccctatagacggttaaaattgtaccttatcttggcaaaacttcagagcaccagtcagtgcatgcaaggtgccattttttattgagatgcttagaatgtttctttctgtgcacaagacttaccctaccagcagcagagccattctctgttgagtggttcattttgaagttccacagattgaagagaacatgccaccaatcacctcacatcttcttggtggacatgataaatgacacaatgaacttgatttctttactaccttgactgtagctttttatccctacctgtgaaccttcaaagactgcattaacttttaggctacataggtccaattgaggtataatatcagtacaccaaagatttttatatgtccttcgtgtgaccattcttcaacggcctaagggccagctgcaaagacttttggaaaatacaatttacaactcaaaattatttaataatttaggaagttgctttttttttttttttttttcagtcctgcagtttcctgaagctctgtatatgatatttttttcagcctgcttctctctgttgttcagattaggtaattttattcttctgtctcgaagctcactgattctttattctgtctaatctgttctgctgttgagcccatttattcctgatttttatattttagttattgtatgttttatttctaaaatttccattcagtttttctttatatcttctatttgctgagaatttctgtctctttgctgagactttctacgttttcatttgtttcaagtgcatttatacttgcttgttgaagaatttttatgatggctgctgtaaaatccttatcagataattccaacatctgtcacctcattgtttgcatctactgatggtcttttttccattcggaaacattttcctgtttcttggtgtgtggaatgattttttattgaaacctggatatttttaggtattatgttatgagactatgggtcttatttaaaccttctgctttagccaactttctcagataccaccacagcaggggaattgggagcactgcttcattattaccaggtgtggctaggagtccaggttccccagtcagcctccctttatactgagtaacagggtcccctcattactactgggcaaggtgagaattcagtttcccattaggtctttattgattcttccctggctggaatgtgcagcggcaccttttggtgcaccctgggaatctccactaatgctatgggacagagtgaccaggaagagcttcattacaccaggtgggaatgaaattcccagtagcctacacagccttctccgacaccactctggagttgtattcttccagcacacaaacatacacaatttaactcaaagcatcttagcagagcttaattaaatggatagatgcctgttccctttgctggataccaagaatacaaaagtcagggagttggggcacctctttacagcttggtgagagtgtaagtctggactccccactcagcatttgctggtatgggtggggccatggtgtttttccatggtgtttggttggagtacagccttttttacccttgcttggctacccttttctggtcctttggcaggagagagcaggactctcttagggcttttttttcccctgcatttattgacatttccaggttgctgactttttcagctccaagttggaaatatatgagctgaaaagaaaatgtagggaactcatcacagtgttgttacttgggccccaatgttcctagcctattttctgtctactattcagagtcttgctgtgttttaatataatatccaggatttttatatgcatttagcagaaggatgtctactctgcctttgtagaagtgtctcactgatttttacatatttttccagcacacaaacatacacaatttaactcaaagcatcttagcagagcttaattaaatggatagatgtctgttccctttgctggacgccaagaatacaaaaaagaacaagtgacaattttctctgtcttagggagaagagacagcagaagtgtaaatgatccctaaagagtgatagatgttatcatgaagccacaggaggggtgccaggctgcacaaaagagacactggatgcttcttggtagtagaggcagtggcttcccagccttggggctaaggcttgtagggtgaattggaacttttcagatgagcaaggcaaagaagggaccttctaacattccttggatggaacatttttgacattttcccatttacagctacttatattttctacaagtgtcactgtgaccaacttatgtacacatactttttcttgcttagttataataatctgttcttaaagaaaatgtcagtctctacattctatgctgactgttaaggaaagagcacccacatctgctcctacttagctttttttctgtggttcttacacagtattcctttttttcttttcttgaaagagactcctcctttcttttcttttcttgaaagagttttaaacagataagatggcaaaagtgactgatctctactcccccagtttgaatggtaaatttgaatggtaaattcccatgaacatatatggaaatgtctttatcctactttctccaataaaggctgttcttagcttttcaaatgcaaagtgaaacctttatttatcttgatttcttttttttttttttttttttttttttttttgagatgctctgtcacccaggctggagtgcagtggcaagatcttggctcactgcaagctccgcctcccaggttcacgccattctcctggctcagcctcccgagtaactgggactacaggcacctgccgtcacgcctggctaattttttgtatttttagtagagaatggagtttcaccgtgttagccaggatggtctcgatctcctgaccttgtgatctgcccgcctcggcctcccaaagtgctgggattacaggctcgagccactgcctccagcctatcctgatttctactgtcatgcctcacatcagtccttttttttttttttgagacagagtctcgctctgtggcccaggctagactgcagtggcatgatctcggctcactgcaacctccacctccggggttctagcaattctcctgcctcagcctcctgagtagctgggattataggcgcatgccacacctggctttttgtattttagtggagatggggtttcactgtgttgctcaggctggtcttgatctcctgagctcagacaatccccccgccttggcctcccaaagtgctaggattataggcgagagctgctgtgtgcttcttaagtgaggtaagtaacttccatagaaaatttccatcagttcattcatgaaagaacaaagaacctggcaaaacttaaaaaaacgtttccaagaatcagataaaagaggacaaaccttagggagaagaaggcagctgctcatttccagcaggggaagtagctgcatagagtacaaggactggtaggcctgttggctgttcctgtttaaggagacaagatgggcatggaacagggaccaccccctcctctgggagaagctgttacccccttcacttttcctcctctgtcattacccacaatcactctccttctttgcgctatggtaggtgtttacccatcataggaatgggcatttgaactttgaaactgaatgtggtgattacacttcatgctgaagcttttcacatgagtgctttcataagcattaagtaaaattttataatgactgcagtccaaggacattttccctggtttttggccagtctaaatattgtaagagagagagaagaaaagtgtacggaatataattgtctctaagctaagaaatgtggatgttcaaataaaacatacgtacagaa

SEQ ID NO: 23 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 24 - DNA, 2283 bp, Homo sapiens
  source 1..2283: mol_type = other DNA
gctttcccggccgcccctcccgccccgcatcgaggcagacaagcctgttcctcttccctgggctgcgattgcgacaggccggcctggctcccagcgctccctgtccccgccccgcggccagctcgctccactcccacttcctgagctccgccatgggagccctggaggcccggcctggccgctcccggccctggggtgcacatcggccctgagtcccgtcccaggctctgggctcgggcagccgccgccaccgctgcccaggacgtcgggcctcctgccttcctcccaggcccccacgttgctggccgcctggccgagtggccgccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgactgagtctgagaaaaggcagaagaaggggggcacaagggcaccttctcccttgaggctgccctgcccacgtgggattcacaggggcctgagtagggcccggggaagcagagccctaagggattaaggctcagacacctctgagagcaggtgggcactggctgggtacggtgccctccacaggactctccctactgcctgagcaaacctgaggcctcccggcagacccacccaccccctggggctgctcagcctcaggcacggacagggcacatgataccaactgctgcccactacggcacgccgcaccggagcacggcaccgagggagccgccacacggtcacctgcaaggacgtcacgggcccctctaaaggattcgtggtgctcatccccaagcttcagagaccctttggggttccacacttcacgtggactgaggtagaccctgcatgaagatgaaattatagggaggacgctccttccctcccctcctagaggagaggaaagggagtcattaacaactagggggttgggtaggattcctaggtatggggaagagttttggaaggggaggaaaatggcaagtgtatttatattgtaaccacatgcaaataaaaagaatgggacctagataaaaaaaaaaaaaaaaa

SEQ ID NO: 25 - DNA, 22 bp, synthetic construct
  misc_feature 1..22: note = A synthetic oligonucleotide sequence
  source 1..22: mol_type = other DNA
ttcatattcaatttgctttgtc

SEQ ID NO: 26 - DNA, 22 bp, synthetic construct
  misc_feature 1..22: note = A synthetic oligonucleotide sequence
  source 1..22: mol_type = other DNA
ttagttttaaacaatctttcct

SEQ ID NO: 27 - DNA, 22 bp, synthetic construct
  misc_feature 1..22: note = A synthetic oligonucleotide sequence
  source 1..22: mol_type = other DNA
ttctaaaaactgactcagagga

SEQ ID NO: 28 - DNA, 22 bp, synthetic construct
  misc_feature 1..22: note = A synthetic oligonucleotide sequence
  source 1..22: mol_type = other DNA
ttcagttgcagaaacactgtta

SEQ ID NO: 29 - DNA, 22 bp, synthetic construct
  misc_feature 1..22: note = A synthetic oligonucleotide sequence
  source 1..22: mol_type = other DNA
tcatcatattcaataataccat

SEQ ID NO: 30 - DNA, 22 bp, synthetic construct
  misc_feature 1..22: note = A synthetic oligonucleotide sequence
  source 1..22: mol_type = other DNA
tgaagtttttgtatcatagtcc

SEQ ID NO: 31 - DNA, 22 bp, synthetic construct
  misc_feature 1..22: note = A synthetic oligonucleotide sequence
  source 1..22: mol_type = other DNA
ttcctcatctgtaaaatgggct

SEQ ID NO: 32 - DNA, 22 bp, synthetic construct
  misc_feature 1..22: note = A synthetic oligonucleotide sequence
  source 1..22: mol_type = other DNA
taatgattggggaacatgttgc

SEQ ID NO: 33 - DNA, 22 bp, synthetic construct
  misc_feature 1..22: note = A synthetic oligonucleotide sequence
  source 1..22: mol_type = other DNA
tttcttgtcatagacgggctcg

SEQ ID NO: 34 - DNA, 22 bp, synthetic construct
  misc_feature 1..22: note = A synthetic oligonucleotide sequence
  source 1..22: mol_type = other DNA
tcaaaaactcatctttattggg

SEQ ID NO: 35 - DNA, 22 bp, synthetic construct
  misc_feature 1..22: note = A synthetic oligonucleotide sequence
  source 1..22: mol_type = other DNA
ttatgatcccatttcacaggtt

SEQ ID NO: 36 - DNA, 22 bp, synthetic construct
  misc_feature 1..22: note = A synthetic oligonucleotide sequence
  source 1..22: mol_type = other DNA
tctcaagagaaatccgtgcgga

SEQ ID NO: 37 - protein, 420 aa, Homo sapiens
MFSPGQEEHCAPNKEPVKYGELVVLGYNGALPNGDRGRRKSRFALYKRPKANGVKPSTVHVISTPQASKAISCKGQHSISYTLSRNQTVVVEYTHDKDTDMFQVGRSTESPIDFVVTDTISGSQNTDEAQITQSTISRFACRIVCDRNEPYTARIFAAGFDSSKNIFLGEKAAKWKNPDGHMDGLTTNGVLVMHPRGGFTEESQPGVWREISVCGDVYTLRETRSAQQRGKLVESETNVLQDGSLIDLCGATLLWRTADGLFHTPTQKHIEALRQEINAARPQCPVGLNTLAFPSINRKEVVEEKQPWAYLSCGHVHGYHNWGHRSDTEANERECPMCRTVGPYVPLWLGCEAGFYVDAGPPTHAFTPCGHVCSEKSAKYWSQIPLPHGTHAFHAACPFCATQLVGEQNCIKLIFQGPID

SEQ ID NO: 38 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 39 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 40 - protein, 480 aa, Homo sapiens
MGSKASSPHGLGSPLVASPRLEKRLGGLAPQRGSRISVLSASPVSDVSYMFGSSQSLLHSSNSSHQSSSRSLESPANSSSSLHSLGSVSLCTRPSDFQAPRNPTLTMGQPRTPHSPPLAKEHASSCPPSITNSMVDIPIVLINGCPEPGSSPPQRTPGHQNSVQPGAASPSNPCPATRSNSQTLSDAPFTTCPEGPARDMQPTMKFVMDTSKYWFKPNITREQAIELLRKEEPGAFVIRDSSSYRGSFGLALKVQEVPASAQSRPGEDSNDLIRHFLIESSAKGVHLKGADEEPYFGSLSAFVCQHSIMALALPCKLTIPQRELGGADGASDSTDSPASCQKKSAGCHTLYLSSVSVETLTGALAVQKAISTTFERDILPTPTVVHFKVTEQGITLTDVQRKVFFRRHYPLTTLRFCGMDPEQRKWQKYCKPSWIFGFVAKSQTEPQENVCHLFAEYDMVQPASQVIGLVTALLQDAERM

SEQ ID NO: 41 - protein, 219 aa, Homo sapiens
MASVTDGKTGVKDASDQNFDYMFKLLIIGNSSVGKTSFLFRYADDTFTPAFVSTVGIDFKVKTVYRHEKRVKLQIWDTAGQERYRTITTAYYRGAMGFILMYDITNEESFNAVQDWATQIKTYSWDNAQVILVGNKCDMEEERVVPTEKGQLLAEQLGFDFFEASAKENISVRQAFERLVDAICDKMSDSLDTDPSMLGSSKNTRLSDTPPLLQQNCSC

SEQ ID NO: 42 - protein, 1607 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQVGSEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPAFIAGIGAACWIILMVFSIWLYRHRKKRNGLTSTYAGIRKVTYQRGGEAVSSGGRPGLLNISEPAAQPWLADTWPNTGNNHNDCSISCCTAGNGNSDSNLTTYSRPADCIANYNNQLDNKQTNLMLPESTVYGDVDLSNKINEMKTFNSPNLKDGRFVNPSGQPTPYATTQLIQSNLSNNMNNGSGDSGEKHWKPLGQQKQEVAPVQYNIVEQNKLNKDYRANDTVPPTIPYNQSYDQNTGGSYNSSDRGSSTSGSQGHKKGGARTPKVPKQGGMNWADLLPPPPAHPPPHSNSEEYNISVDESYDQEMPCPVPPARMYLQQDELEEEEDERGPTPPVRGAASSPAAVSYSHQSTATLTPSPQEELQPMLQDCPEETGHMQHQPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAAVMQKTRPAKKLKHQPGHLRRETYTDDLPPPPVPPPAIKSPTAQSKTQLEVRPVVVPKLPSMDARTDRSSDRKGSSYKGREVLDGRQVVDMRTNPGDPREAQEQQNDGKGRGNKAAKRDLPPAKTHLIQEDILPYCRPTFPTSNNPRDPSSSSSMSSRGSGSRQREQANVGRRNIAEMQVLGGYERGEDNNEELEETES

SEQ ID NO: 43 - protein, 545 aa, Homo sapiens
MNELPRDTCGNHTNQLDGTKEERELPRVIKTSGSMPDDASLNSTTLSDASQDKEGSFAVPRSDSVAILETIPVLPVHSNGSPEPGQPVQNAISDDDFLEKNIPPEAEELSFEVSYSEMVTEALKRNKLKKSEIKKEDYVLTKFNVQKTRFGLTEAGDLSAEDMKKIRHLSLIELTAFFDAFGIQLKRNKTEKVKGRDNGIFGVPLTVLLDGDRKKDPGVKVPLVLQKFFEKVEESGLESEGIFRLSGCTAKVKQYREELDAKFNADKFKWDKMCHREAAVMLKAFFRELPTSLFPVEYIPAFISLMERGPHVKVQFQALHLMVMALPDANRDAAQALMTFFNKVIANESKNRMSLWNISTVMAPNLFFSRSKHSDYEELLLANTAAHIIRLMLKYQKILWKVPSFLITQVRRMNEATMLLKKQLPSVRKLLRRKTLERETASPKTSKVLQKSPSARRMSDVPEGVIRVHAPLLSKVSMAIQLNNQTKAKDILAKFQYENRILHWQRAALSFLNGKWVKKEREESTETNRSPKHVFLFTIGLDIST

SEQ ID NO: 44 - protein, 468 aa, Homo sapiens
MAASSNSSLSGSSVSSDAEEYQPPIWKSYLYQLQQEAPRPKRIICPREVENRPKYYGREFHGIISREQADELLGGVEGAYILRESQRQPGCYTLALRFGNQTLNYRLFHDGKHFVGEKRFESIHDLVTDGLITLYIETKAAEYISKMTTNPIYEHIGYATLLREKVSRRLSRSKNEPRKTNVTHEEHTAVEKISSLVRRAALTHNDNHFNYEKTHNFKVHTFRGPHWCEYCANFMWGLIAQGVRCSDCGLNVHKQCSKHVPNDCQPDLKRIKKVYCCDLTTLVKAHNTQRPMVVDICIREIEARGLKSEGLYRVSGFTEHIEDVKMAFDRDGEKADISANVYPDINIITGALKLYFRDLPIPVITYDTYSKFIDAAKISNADERLEAVHEVLMLLPPAHYETLRYLMIHLKKVTMNEKDNFMNAENLGIVFGPTLMRPPEDSTLTTLHDMRYQKLIVQILIENEDVLF

SEQ ID NO: 45 - protein, 141 aa, Homo sapiens
MAQYLSTLLLLLATLAVALAWSPKEEDRIIPGGIYNADLNDEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQES

SEQ ID NO: 46 - protein, 732 aa, Homo sapiens
MSETSRTAFGGRRAVPPNNSNAAEDDLPTVELQGVVPRGVNLQEFLNVTSVHLFKERWDTNKVDHHTDKYENNKLIVRRGQSFYVQIDFSRPYDPRRDLFRVEYVIGRYPQENKGTYIPVPIVSELQSGKWGAKIVMREDRSVRLSIQSSPKCIVGKFRMYVAVWTPYGVLRTSRNPETDTYILFNPWCEDDAVYLDNEKEREEYVLNDIGVIFYGEVNDIKTRSWSYGQFEDGILDTCLYVMDRAQMDLSGRGNPIKVSRVGSAMVNAKDDEGVLVGSWDNIYAYGVPPSAWTGSVDILLEYRSSENPVRYGQCWVFAGVFNTFLRCLGIPARIVTNYFSAHDNDANLQMDIFLEEDGNVNSKLTKDSVWNYHCWNEAWMTRPDLPVGFGGWQAVDSTPQENSDGMYRCGPASVQAIKHGHVCFQFDAPFVFAEVNSDLIYITAKKDGTHVVENVDATHIGKLIVTKQIGGDGMMDITDTYKFQEGQEEERLALETALMYGAKKPLNTEGVMKSRSNVDMDFEVENAVLGKDFKLSITFRNNSHNRYTITAYLSANITFYTGVPKAEFKKETFDVTLEPLSFKKEAVLIQAGEYMGQLLEQASLHFFVTARINETRDVLAKQKSTVLTIPEIIIKVRGTQVVGSDMTVTVEFTNPLKETLRNVWVHLDGPGVTRPMKKMFREIRPNSTVQWEEVCRPWVSGHRKLIASMSSDSLRHVYGELDVQIQRRPSM

SEQ ID NO: 47 - protein, 476 aa, Homo sapiens
MVGAMWKVIVSLVLLMPGPCDGLFRSLYRSVSMPPKGDSGQPLFLTPYIEAGKIQKGRELSLVGPFPGLNMKSYAGFLTVNKTYNSNLFFWFFPAQIQPEDAPVVLWLQGGPGGSSMFGLFVEHGPYVVTSNMTLRDRDFPWTTTLSMLYIDNPVGTGFSFTDDTHGYAVNEDDVARDLYSALIQFFQIFPEYKNNDFYVTGESYAGKYVPAIAHLIHSLNPVREVKINLNGIAIGDGYSDPESIIGGYAEFLYQIGLLDEKQKKYFQKQCHECIEHIRKQNWFEAFEILDKLLDGDLTSDPSYFQNVTGCSNYYNFLRCTEPEDQLYYVKFLSLPEVRQAIHVGNQTFNDGTIVEKYLREDTVQSVKPWLTEIMNNYKVLIYNGQLDIIVAAALTERSLMGMDWKGSQEYKKAEKKVWKIFKSDSEVAGYIRQAGDFHQVIIRGGGHILPYDQPLRAFDMINRFIYGKGWDPYVG

SEQ ID NO: 48 - protein, 1011 aa, Homo sapiens
MRVFLLCAYILLLMVSQLRAVSFPEDDEPLNTVDYHYSRQYPVFRGRPSGNESQHRLDFQLMLKIRDTLYIAGRDQVYTVNLNEMPKTEVIPNKKLTWRSRQQDRENCAMKGKHKDECHNFIKVFVPRNDEMVFVCGTNAFNPMCRYYRLSTLEYDGEEISGLARCPFDARQTNVALFADGKLYSATVADFLASDAVIYRSMGDGSALRTIKYDSKWIKEPHFLHAIEYGNYVYFFFREIAVEHNNLGKAVYSRVARICKNDMGGSQRVLEKHWTSFLKARLNCSVPGDSFFYFDVLQSITDIIQINGIPTVVGVFTTQLNSIPGSAVCAFSMDDIEKVFKGRFKEQKTPDSVWTAVPEDKVPKPRPGCCAKHGLAEAYKTSIDFPDETLSFIKSHPLMDSAVPPIADEPWFTKTRVRYRLTAISVDHSAGPYQNYTVIFVGSEAGMVLKVLAKTSPFSLNDSVLLEEIEAYNHAKCSAENEEDKKVISLQLDKDHHALYVAFSSCIIRIPLSRCERYGSCKKSCIASRDPYCGWLSQGSCGRVTPGMLLLTEDFFAFHNHSAEGYEQDTEFGNTAHLGDCHGVRWEVQSGESNQMVHMNVLITCVFAAFVLGAFIAGVAVYCYRDMFVRKNRKIHKDAESAQSCTDSSGSFAKLNGLFDSPVKEYQQNIDSPKLYSNLLTSRKELPPNGDTKSMVMDHRGQPPELAALPTPESTPVLHQKTLQAMKSHSEKAHGHGASRKETPQFFPSSPPPHSPLSHGHIPSAIVLPNATHDYNTSFSNSNAHKAEKKLQNIDHPLTKSSSKRDHRRSVDSRNTLNDLLKHLNDPNSNPKAIMGDIQMAHQNLMLDPMGSMSEVPPKVPNREASLYSPPSTLPRNSPTKRVDVPTTPGVPMTSLERQRGYHKNSSQRHSISAMPKNLNSPNGVLLSRQPSMNRGGYMPTPTGAKVDYIQGTPVSVHLQPSLSRQSSYTSNGTLPRTGLKRTPSLKPDVPPKPSFVPQTPSVRPLNKYTY

SEQ ID NO: 49 - protein, 218 aa, Homo sapiens
MAEGSRTQAPGKGPPLSIQFLRAQYEGLKRQQRTQAHLLVLPKGGNTPAPAESMVNAVWINKERRSSLSLEEADSEVEGRLEEAAQGCLQAPKSPWHTHLEMHCLVQTSPQDTSHQVHHRGKLVGSDQRLPPEGDTHLFETNQMTQQGTGIPEAAQLPCQVGNTQTKAVESGLKFSTQCPLSIKNPHRSGKPAYYPFPQRKTPRISQAARNLGLYGSA

SEQ ID NO: 50 - protein, 709 aa, Homo sapiens
MESMGMVYSVPSSCNGPTESTFSTSWKGDAFTYMTPSATSQSNQVNENGKNPSCGNSWVSLNKVPPLVPKEAATLLVARDNPAGCSGSAGYPERLIQQRHMPERPSKIGLLTSGTSRLETGPGGASRFRERSLSVPTDSGTTDVDYDEEQKANEACALPFASTSSEGSNSADNIASLSAQQEAQHRRQRSKSISLRKAKKKPSPPTRSVSLVKDEPGLLPEGGSALPKDQRPKSLCLSLEHQGHHSSHPDAQGHPAIPNHKDPESTQFSHHWYLTDWKSGDTYQSLSSSSTATGTTVIECTQVQGSSESLASPSTSRATTPSQLSIEVEAREISSPGRPPGLMSPSSGYSSQSETPTPTVSMSLTLGHLPPPSSSVRVRPVVPERKSSLPPTSPMEKFPKSRLSFDLPLTSSPNLDLSGMSISIRSKTKVSRHHSETNFGVKLAQKTNPNQPIMPMVTQSDLRSVRLRSVSKSEPEDDIESPEYAEEPRAEEVFTLPERKTKPPVAEKPPVARRPPSLVHKPPSVPEEYALTSPTLAMPPRSSIQHARPLPQDSYTVVRKPKPSSFPDGRSPGESTAPSSLVFTPFASSSDAFFSGTQQPPQGSVEDEGPKVRVLPERISLQSQEEAEKKKGKIPPPVPKKPSVLYLPLTSPTAQMEAYVAEPRLPLSPIITLEEDTKCPATGDDLQSLGQRVTSTPQADSEREASPLG

SEQ ID NO: 51 - protein, 684 aa, Homo sapiens [sequence omitted]

SEQ ID NO: 52 - protein, 1069 aa, Homo sapiens
MLRMRTAGWARGWCLGCCLLLPLSLSLAAAKQLLRYRLAEEGPADVRIGNVASDLGIVTGSGEVTFSLESGSEYLKIDNLTGELSTSERRIDREKLPQCQMIFDENECFLDFEVSVIGPSQSWVDLFEGQVIVLDINDNTPTFPSPVLTLTVEENRPVGTLYLLPTATDRDFGRNGIERYELLQEPGGGGSGGESRRAGAADSAPYPGGGGNGASGGGSGGSKRRLDASEGGGGTNPGGRSSVFELQVADTPDGEKQPQLIVKGALDREQRDSYELTLRVRDGGDPPRSSQAILRVLITDVNDNSPRFEKSVYEADLAENSAPGTPILQLRAADLDVGVNGQIEYVFGAATESVRRLLRLDETSGWLSVLHRIDREEVNQLRFTVMARDRGQPPKTDKATVVLNIKDENDNVPSIEIRKIGRIPLKDGVANVAEDVLVDTPIALVQVSDRDQGENGVVTCTVVGDVPFQLKPASDTEGDQNKKKYFLHTSTPLDYEATREFNVVIVAVDSGSPSLSSNNSLIVKVGDTNDNPPMFGQSVVEVYFPENNIPGERVATVLATDADSGKNAEIAYSLDSSVMGIFAIDPDSGDILVNTVLDREQTDRYEFKVNAKDKGIPVLQGSTTVIVQVADKNDNDPKFMQDVFTFYVKENLQPNSPVGMVTVMDADKGRNAEMSLYIEENNNIFSIENDTGTIYSTMSFDREHQTTYTFRVKAVDGGDPPRSATATVSLFVMDENDNAPTVTLPKNISYTLLPPSSNVRTVVATVLATDSDDGINADLNYSIVGGNPFKLFEIDPTSGVVSLVGKLTQKHYGLHRLVVQVNDSGQPSQSTTTLVHVFVNESVSNATAIDSQIARSLHIPLTQDIAGDPSYEISKQRLSIVIGVVAGIMTVILIILIVVMARYCRSKNKNGYEAGKKDHEDFFTPQQHDKSKKPKKDKKNKKSKQPLYSSIVTVEASKPNGQRYDSVNEKLSDSPSMGRYRSVNGGPGSPDLARHYKSSSPLPTVQLHPQSPTAGKKHQAVQDLPPANTFVGAGDNISIGSDHCSEYSCQTNNKYSKQMRLHPYITVFG

SEQ ID NO: 53 - protein, 345 aa, Homo sapiens
MEEKYLPELMAEKDSLDPSFTHALRLVNQEIEKFQKGEGKEEKYIDVVINKNMKLGQKVLIPVKQFPKFNFVGKLLGPRGNSLKRLQEETLTKMSILGKGSMRDKAKEEELRKSGEAKYFHLNDDLHVLIEVFAPPAEAYARMGHALEEIKKFLIPDYNDEIRQAQLQELTYLNGGSENADVPVVRGKPTLRTRGVPAPAITRGRGGVTARPVGVVVPRGTPTPRGVLSTRGPVSRGRGLLTPRARGVPPTGYRPPPPPPTQETYGEYDYDDGYGTAYDEQSYDSYDNSYSTPAQSGADYYDYGHGLSEETYDSYGQEEWTNSRHKAPSARTAKGVYRDQPYGRY

SEQ ID NO: 54 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 296 aa, Homo sapiens [sequence omitted]

SEQ ID NO: 56 - protein, 141 aa, Homo sapiens
MARPLCTLLLLMATLAGALASSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

SEQ ID NO: 57 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 58 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 59 - protein, 579 aa, Homo sapiens; identical to SEQ ID NO: 19

SEQ ID NO: 60 - skipped

SEQ ID NO: 61 - DNA, 750 bp, Homo sapiens
  source 1..750: mol_type = other DNA
tgcctccagaaagggttgagaagataatggatcagattgaaaagtacatcatgactcatctctgtaaatatgcgttctgtccagaaccccagtgagcctggaagactgggtgctatgggaaatgtcatcaatccaatgctagtgaaagatgtgactggggaatgctgaaaaatgcgcacccctgggaggaatgaggaaagatgacatccactgacttgttatttttttgagaaggagtcttgctctgttgcccaggctggagtgtggtggcacgatctcggctcactgatgatgagaagaaagatcttgccattcaaaagaggatcagagtggaagcttctctgtggtgtcagaaagacgaggaatgtggtgacctcacctatccatctccctcaaggcagcttttgacctgaactggttatttcctacttgcctcctggagttgctaataaaataaacactaaagcttcgcagtttctaaaaagaccatgaagccctgagagtaatgaaaaggttcctgaaattgaggtcactgtggaaggagaatctaatgcctgatgatctgtcactatctcccatcacccccagatgggaccatctagttgcaggaaaagaaggtcaagactcccagtcattctacattatgcctcagccaagatgtctcaccccactctctctgatgcaacaagaagcccctggagaacgtttcagtcccattttgtacttctgtcatgtgctcatcacagtctg